(19) United States
(12) Patent Application Publication
(10) Pub. No.: US 2013/0316946 A1
(43) Pub. Date: Nov. 28, 2013

(54) EXTENDED RECOMBINANT POLYPEPTIDE-MODIFIED C-PEPTIDE

(71) Applicant: Cebix, Inc., La Jolla, CA (US)

(72) Inventor: Sheri BARRACK, La Jolla, CA (US)

(73) Assignee: Cebix, Inc., La Jolla, CA (US)

(21) Appl. No.: 13/902,618

(22) Filed: May 24, 2013

Related U.S. Application Data
(60) Provisional application No. 61/651,373, filed on May 24, 2012.

(51) Int. Cl. C07K 14/47; A61K 38/28; A61K 38/17

(52) U.S. Cl. CPC C07K 14/47; A61K 38/1709; A61K 38/28
USPC 514/6.5; 530/350; 514/6.9; 514/21.2; 536/23.4; 435/320.1; 435/252.33; 435/358

(57) ABSTRACT

The present invention relates to modified forms of C-peptide, and methods for their use. In one aspect, the modified forms of C-peptide comprise modified C-peptide derivatives which exhibit superior pharmacokinetic and biological activity in vivo.

### CROSS-REFERENCE TO RELATED APPLICATIONS

This application claims priority from U.S. provisional application 61/651,373 filed 24 May 2012. The contents of this document are incorporated herein by reference.

### FIELD OF THE INVENTION

The present invention relates to modified forms of C-peptide, and methods for their use.

### BACKGROUND OF THE INVENTION

C-peptide is the linking peptide between the A- and B-chains in the proinsulin molecule. After cleavage and processing in the endoplasmic reticulum of pancreatic islet β-cells, insulin and C-peptide are generated. C-peptide is co-secreted with insulin in equimolar amounts from the pancreatic islet β-cells into the portal circulation. Besides its contribution to the folding of the two-chain insulin structure, further biologic activity of C-peptide was questioned for many years after its discovery.

Type 1 diabetes, or insulin-dependent diabetes mellitus, is generally characterized by insulin and C-peptide deficiency, due to an autoimmune destruction of the pancreatic islet β-cells. The patients are therefore dependent on exogenous insulin to sustain life. Several factors may be of importance for the pathogenesis of the disease, e.g., genetic background, environmental factors, and an aggressive autoimmune reaction following a temporary infection (Akerblom H K et al.: Annual Medicine 29(5): 383-385, (1997)). Currently insulin-dependent diabetics are provided with exogenous insulin which has been separated from the C-peptide, and thus do not receive exogenous C-peptide therapy. By contrast most type 2 diabetics initially still produce both insulin and C-peptide endogenously, but are generally characterized by insulin resistance in skeletal muscle and adipose tissue.

Type 1 diabetics suffer from a constellation of long-term complications of diabetes that are in many cases more severe and widespread than in type 2 diabetes. Specifically, for example microvascular complications involving the retina, kidneys, and nerves are a major cause of morbidity and mortality in patients with type 1 diabetes.

There is increasing support for the concept that C-peptide deficiency may play a role in the development of the long-term complications of insulin-dependent diabetics. Additionally, in vivo as well as in vitro studies, in diabetic animal models and in patients with type 1 diabetes, demonstrate that C-peptide possesses hormonal activity (Wahren J et al.: American Journal of Physiology 278: E759-E768, (2000); Wahren J et al.: In International Textbook of Diabetes Mellitus Ferranninni E, Zimmet P, De Fronzo R A, Keen H, Eds. Chichester, John Wiley & Sons, (2004), p. 165-182). Thus, C-peptide used as a complement to regular insulin therapy may provide an effective approach to the management of type 1 diabetes long-term complications.

Studies to date suggest that C-peptide's therapeutic activity involves the binding of C-peptide to a G-protein-coupled membrane receptor, activation of Ca²⁺-dependent intracellular signalling pathways, and phosphorylation of the MAP-kinase system, eliciting increased activities of sodium/potassium ATPase and endothelial nitric oxide synthase (eNOS). Despite the promise of using C-peptide to treat and prevent the long-term complications of insulin-dependent diabetes, the short biological half-life and requirement to dose C-peptide multiple times per day via subcutaneous injection, or intravenous (I.V.) administration, has hindered commercial development.

The present invention is focused on the development of modified versions of C-peptide that retain the biological activity of the native C-peptide and exhibit superior pharmacokinetic properties. These improved therapeutic forms of C-peptide enable the development of more effective therapeutic regimens for the treatment of the long-term complications of diabetes, and require significantly less frequent administration.

In one aspect, these therapies are targeted to diabetic patients, and in a further aspect to insulin-dependent patients. In one aspect, the insulin-dependent patients are suffering from one or more long-term complications of diabetes.

These improved methods are based on modifications of C-peptide that result in versions of C-peptide that retain the biological activity of the native molecule, while exhibiting superior pharmacokinetic characteristics.

### SUMMARY OF THE INVENTION

In one embodiment, the modified C-peptide disclosed is fusion protein comprising C-peptide linked to an extended recombinant polypeptide (XTEN).

In further embodiments, the C-peptide exhibits at least 90% sequence identity with a protein sequence selected from the group consisting of SEQ ID NOS:1-33.

In further embodiments, the C-peptide protein sequence comprises SEQ ID NO:1.

In further embodiments, the C-peptide protein sequence comprises the pentapeptide sequence (EGSLQ) (SEQ ID NO:31).

In further embodiments, the XTEN comprises greater than about 400 to about 3000 amino acid residues, and the XTEN is characterized in that:

a. the sum of glycine (G), alanine (A), serine (S), threonine (T), glutamate (E) and proline (P) residues constitutes more than about 80% of the total amino acid sequence of the XTEN;

b. the XTEN sequence is substantially non-repetitive;

c. the XTEN sequence lacks a predicted T-cell epitope when analyzed by TEPITOPE algorithm, wherein the TEPITOPE algorithm prediction for epitopes within the XTEN sequence is based on a score of −9 or greater;

d. the XTEN sequence has greater than 90% random coil formation as determined by GOR algorithm; and

e. the XTEN sequence has less than 2% alpha helices and 2% beta-sheets as determined by Chou-Fasman algorithm.

In further embodiments, the XTEN is further characterized in that:

a. the sum of asparagine and glutamine residues is less than 10% of the total amino acid sequence of the XTEN; and/or

b. the sum of methionine and tryptophan residues is less than 2% of the total amino acid sequence of the XTEN.

In further embodiments, the XTEN is further characterized in that:

a. no one type of amino acid constitutes more than 30% of the XTEN sequence;

b. the XTEN comprises a sequence in which no three contiguous amino acids are identical unless the amino acid is serine, in which case no more than three contiguous amino acids are serine residues; and/or

c. the XTEN sequence has a subsequence score of less than 10.

In further embodiments, the XTEN is further characterized in that:

a. at least about 80% of the XTEN sequence consists of non-overlapping sequence motifs wherein each of the sequence motifs has about 9 to about 14 amino acid residues and wherein the sequence of any two contiguous amino acid residues does not occur more than twice in each of the sequence motifs;

b. the sequence motifs consist of four to six types of amino acids selected from glycine (G), alanine (A), serine (S), threonine (T), glutamate (E) and proline (P); and

c. the XTEN enhances the pharmacokinetic properties the C-peptide wherein the pharmacokinetic properties are ascertained by measuring the blood concentration of the fusion protein after administration of a therapeutically effective dose to a subject in comparison to the corresponding C-peptide not linked to XTEN and administered to a subject at a comparable dose.

In further embodiments, the enhanced pharmacokinetic property is selected from an increase in terminal half-life of at least three-fold and blood concentrations that remain within the therapeutic window for the fusion protein for a period at least about three-fold longer compared to the corresponding C-peptide not linked to XTEN.

In further embodiments, the sequence motifs are selected from one or more sequences of Table 2.

In further embodiments, the XTEN polypeptide is selected from one or more sequences of Table 3.

In further embodiments, the fusion protein further comprises a spacer sequence between the C-peptide and XTEN, wherein the spacer sequence comprises between 1 to about 50 amino acid residues that optionally comprises a cleavage sequence.

In further embodiments, the cleavage sequence is susceptible to cleavage by a protease selected from FXIa, FXIIa, kallikrein, FVIIa, FIXa, FXa, thrombin, elastase-2, granzyme B, MMP-12, MMP-13, MMP-17 or MMP-20, TEY, enterokinase, rhinovirus 3C protease, and sortase A.

In further embodiments, the fusion protein has substantially the same secondary structure as unmodified C-peptide, as determined via UV circular dichroism analysis.

In further embodiments, the fusion protein has a plasma or sera pharmacokinetic area under curve (AUC) profile at least 10-fold greater than unmodified C-peptide when subcutaneously administered to dogs.

In further embodiments, the fusion protein retains at least about 50% of the biological activity of the unmodified C-peptide.

In further embodiments, the fusion protein retains at least about 75% of the biological activity of the unmodified C-peptide.

In certain embodiments, disclosed herein is a method for maintaining C-peptide levels above the minimum effective therapeutic level in a patient in need thereof, comprising administering to the patient a therapeutic dose of the fusion protein disclosed herein.

In certain embodiments, disclosed herein is a method for treating one or more long-term complications of diabetes in a patient in need thereof, comprising administering to the patient a therapeutic dose of the fusion protein disclosed herein.

In further embodiments, the long-term complications of diabetes are selected from the group consisting of retinopathy, peripheral neuropathy, autonomic neuropathy, and nephropathy.

In further embodiments, the long-term complication of diabetes is peripheral neuropathy.

In further embodiments, the peripheral neuropathy is established peripheral neuropathy.

In further embodiments, treatment results in an improvement of at least 10% in nerve conduction velocity compared to nerve conduction velocity prior to starting fusion protein therapy.

In certain embodiments, disclosed herein is a method for treating a patient with diabetes comprising administering to the patient a therapeutic dose of the fusion protein of any of claims 1 to 2 in combination with insulin.

In certain embodiments, disclosed herein is a method for treating an insulin-dependent human patient, comprising the steps of:

a. administering insulin to the patient, wherein the patient has neuropathy;

b. administering subcutaneously to the patient a therapeutic dose of the fusion protein of any of claims 1 to 12 in a different site as that used for the patient's insulin administration;

c. adjusting the dosage amount, type, or frequency of insulin administered based on monitoring the patient's altered insulin requirements resulting from the therapeutic dose of the fusion protein, wherein the adjusted dose of insulin reduces the risk, incidence, or severity of hypoglycemia, wherein the adjusted dose of insulin is at least 10% less than the patient's insulin dose prior to starting the fusion protein treatment.

In further embodiments, the insulin is administered subcutaneously at a different depot site compared to that most recently used for the fusion protein.

In further embodiments, the modified C-peptide is administered with a dosing interval of about 3 days or longer.

In further embodiments, the modified C-peptide is administered with a dosing interval of about 5 days or longer.

In further embodiments, the modified C-peptide is administered with a dosing interval of about 7 days or longer.

In further embodiments, the therapeutic dose of modified C-peptide is administered subcutaneously.

In certain embodiments, disclosed herein is a method of reducing insulin usage in an insulin-dependent human patient, comprising the steps of:

a. administering insulin to the patient;

b. administering subcutaneously to the patient a therapeutic dose of the fusion protein of any of claims 1 to 12 in a different site as that used for the patient's insulin administration;

c. adjusting the dosage amount, type, or frequency of insulin administered based on monitoring the patient's altered insulin requirements resulting from the therapeutic dose of modified C-peptide, wherein the adjusted dose of insulin does not induce hypoglycemia, wherein the adjusted dose of insulin is at least 10% less than the patient's insulin dose prior to starting the fusion protein treatment.

In certain embodiments, disclosed herein is a pharmaceutical composition comprising a fusion protein disclosed herein and a pharmaceutically acceptable carrier or excipient.

In certain embodiments, disclosed herein is a pharmaceutical composition comprising a fusion protein disclosed herein and insulin.

In certain embodiments, disclosed herein is an isolated nucleic acid comprising a polynucleotide sequence selected from:

a. a polynucleotide encoding the fusion protein of claim 1, or

b. the complement of the polynucleotide of (a).

In certain embodiments, disclosed herein is an expression vector comprising a polynucleotide sequence disclosed herein.

In further embodiments, the expression vector further comprises a recombinant regulatory sequence operably linked to the polynucleotide sequence.

In further embodiments, the polynucleotide sequence is fused in frame to a polynucleotide encoding a secretion signal sequence.

In further embodiments, the secretion signal sequence is a prokaryotic signal sequence.

In certain embodiments, disclosed herein is a host cell, comprising an expression vector disclosed herein, wherein the host cell is selected from a prokaryotic cell or a eukaryotic cell.

In further embodiments, the prokaryotic host cell is E. coli.

In further embodiments, the eukaryotic host cell is CHO.

In certain aspects of any of the claimed modified C-peptides, the modified C-peptide has a plasma or sera pharmacokinetic AUC profile at least about 5-fold greater than unmodified C-peptide when subcutaneously administered to dogs.

In certain aspects of any of the claimed modified C-peptides, the modified C-peptide has a plasma or sera pharmacokinetic AUC profile at least about 6-fold greater than unmodified C-peptide when subcutaneously administered to dogs.

In certain aspects of any of the claimed modified C-peptides, the modified C-peptide has a plasma or sera pharmacokinetic AUC profile at least about 7-fold greater than unmodified C-peptide when subcutaneously administered to dogs.

In certain aspects of any of the claimed modified C-peptides, the modified C-peptide has a plasma or sera pharmacokinetic AUC profile at least about 8-fold greater than unmodified C-peptide when subcutaneously administered to dogs.

In certain aspects of any of the claimed modified C-peptides, the modified C-peptide has a plasma or sera pharmacokinetic AUC profile at least about 10-fold greater than unmodified C-peptide when subcutaneously administered to dogs.

In certain aspects of any of the claimed modified C-peptides, the modified C-peptide has a plasma or sera pharmacokinetic AUC profile at least about 15-fold greater than unmodified C-peptide when subcutaneously administered to dogs.

In certain aspects of any of the claimed modified C-peptides, the modified C-peptide has a plasma or sera pharmacokinetic AUC profile at least about 20-fold greater than unmodified C-peptide when subcutaneously administered to dogs.

In certain aspects of any of the claimed modified C-peptides, the modified C-peptide has a plasma or sera pharmacokinetic AUC profile at least about 25-fold greater than unmodified C-peptide when subcutaneously administered to dogs.

In certain aspects of any of the claimed modified C-peptides, the modified C-peptide has a plasma or sera pharmacokinetic AUC profile at least about 50-fold greater than unmodified C-peptide when subcutaneously administered to dogs.

In certain aspects of any of the claimed modified C-peptides, the modified C-peptide has a plasma or sera pharmacokinetic AUC profile at least about 75-fold greater than unmodified C-peptide when subcutaneously administered to dogs.

In certain aspects of any of the claimed modified C-peptides, the modified C-peptide has a plasma or sera pharmacokinetic AUC profile at least about 100-fold greater than unmodified C-peptide when subcutaneously administered to dogs.

In certain aspects of any of the claimed modified C-peptides, the modified C-peptide has an equipotent biological activity with the unmodified C-peptide. In certain aspects of any of the claimed modified C-peptides, the modified C-peptide retains at least about 95% of the biological activity of the unmodified C-peptide. In certain aspects of any of the claimed modified C-peptides, the modified C-peptide retains at least about 90% of the biological activity of the unmodified C-peptide. In certain aspects of any of the claimed modified C-peptides, the modified C-peptide retains at least about 80% of the biological activity of the unmodified C-peptide. In another aspect of any of the claimed modified C-peptides, the modified C-peptide retains at least about 70% of the biological activity of the unmodified C-peptide. In another aspect of any of the claimed modified C-peptides, the modified C-peptide retains at least about 60% of the biological activity of the unmodified C-peptide. In another aspect of any of the claimed modified C-peptides, the modified C-peptide retains at least about 50% of the biological activity of the unmodified C-peptide. In another aspect of any of the claimed modified C-peptides, the modified C-peptide retains at least about 40% of the biological activity of the unmodified C-peptide. In another aspect of any of the claimed modified C-peptides, the modified C-peptide retains at least about 30% of the biological activity of the unmodified C-peptide. In another aspect of any of the claimed modified C-peptides, the modified C-peptide retains at least about 20% of the biological activity of the unmodified C-peptide. In another aspect of any of the claimed modified C-peptides, the modified C-peptide retains at least about 10% of the biological activity of the unmodified C-peptide. In another aspect of any of the claimed modified C-peptides, the modified C-peptide retains at least about 5% of the biological activity of the unmodified C-peptide.

In another embodiment, the present invention includes a dosing regimen which maintains an average steady-state concentration of modified C-peptide in the patient's plasma of between about 0.2 nM and about 6 nM when using a dosing interval of 3 days or longer, comprising administering to the patient a therapeutic dose of modified C-peptide of any of the claimed modified C-peptides.

In another embodiment, the present invention includes a dosing regimen which maintains an average steady-state concentration of modified C-peptide in the patient's plasma of between about 0.4 nM and about 6 nM when using a dosing interval of 3 days or longer, comprising administering to the patient a therapeutic dose of modified C-peptide of any of the claimed modified C-peptides.

In another embodiment, the present invention includes a dosing regimen which maintains an average steady-state concentration of modified C-peptide in the patient's plasma of between about 0.6 nM and about 8 nM when using a dosing interval of 3 days or longer, comprising administering to the patient a therapeutic dose of modified C-peptide of any of the claimed modified C-peptides.

In another embodiment, the present invention includes a dosing regimen which maintains an average steady-state concentration of modified C-peptide in the patient's plasma of between about 0.8 nM and about 10 nM when using a dosing interval of 3 days or longer, comprising administering to the patient a therapeutic dose of modified C-peptide of any of the claimed modified C-peptides.

In another embodiment, the present invention includes a method for maintaining C-peptide levels above the minimum effective therapeutic level in a patient in need thereof, comprising administering to the patient a therapeutic dose of any of the claimed modified C-peptides.

In another aspect of any of the claimed modified C-peptides, the modified C-peptide is substantially free of adverse side effects when subcutaneously administered to a mammal at an effective therapeutic dose.

In another embodiment, the present invention includes a method for treating one or more long-term complications of diabetes in a patient in need thereof, comprising administering to the patient a therapeutic dose of any of the claimed modified C-peptides.

In another embodiment, the present invention includes a method for treating a patient with diabetes comprising administering to the patient a therapeutic dose of modified C-peptide of any of the claimed modified C-peptides in combination with insulin.

In one aspect of any of these methods, the modified C-peptide is administered with a dosing interval of about 3 days or longer. In one aspect of any of these methods, the modified C-peptide is administered with a dosing interval of about 4 days or longer. In one aspect of any of these methods, the modified C-peptide is administered with a dosing interval of about 5 days or longer. In one aspect of any of these methods, the modified C-peptide is administered with a dosing interval of about 6 days or longer. In one aspect of any of these methods, the modified C-peptide is administered with a dosing interval of about 7 days or longer.

In certain embodiments, treatment results in an improvement of at least 10% in nerve conduction velocity compared to nerve conduction velocity prior to starting modified C-peptide therapy.

In another aspect of any of these methods, the plasma concentration of modified C-peptide is maintained above about 0.1 nM. In another aspect of any of these methods, the plasma concentration of modified C-peptide is maintained above about 0.2 nM. In another aspect of any of these methods, the plasma concentration of modified C-peptide is maintained above about 0.3 nM. In another aspect of any of these methods, the plasma concentration of modified C-peptide is maintained above about 0.4 nM.

In another aspect of any of these methods, the therapeutic dose of modified C-peptide is administered subcutaneously. In another aspect of any of these methods, the therapeutic dose of modified C-peptide is administered orally.

In another embodiment, the present invention includes the use of any of the claimed modified C-peptides as a C-peptide replacement therapy in a patient in need thereof.

In another embodiment, the present invention includes the use of any of the claimed modified C-peptides for treating one or more long-term complications of diabetes in a patient in need thereof. In certain embodiments, the long-term complications of diabetes are selected from the group consisting of retinopathy, peripheral neuropathy, autonomic neuropathy, nephropathy and erectile dysfunction. In certain embodiments, the long-term complication of diabetes is peripheral neuropathy. In certain embodiments, the peripheral neuropathy is established peripheral neuropathy. In certain embodiments, treatment results in an improvement of at least 10% in nerve conduction velocity compared to nerve conduction velocity prior to starting modified C-peptide therapy.

In another embodiment, the present invention includes a pharmaceutical composition comprising any of the claimed modified C-peptides and a pharmaceutically acceptable carrier or excipient. In certain embodiments, the pharmaceutically acceptable carrier or excipient is sorbitol. In certain embodiments, the sorbitol is present at a concentration of about 2% to about 8% wt/wt. In certain embodiments, the sorbitol is present at a concentration of about 4.7%. In certain embodiments, the pharmaceutical composition is buffered to a pH within the range of about pH 5.5 to about pH 6.5. In certain embodiments, the pharmaceutical composition is buffered to a pH of about 6.0. In certain embodiments, the pharmaceutical composition is buffered with a phosphate buffer at a concentration of about 5 mM to about 25 mM. In certain embodiments, the pharmaceutical composition is buffered with a phosphate buffer at a concentration of about 10 mM. In one aspect of any of these embodiments, the pharmaceutical composition is characterized by improved stability of any of the claimed modified C-peptides compared to a pharmaceutical composition comprising the same modified C-peptide and 0.9% saline at pH 7.0, wherein the stability is determined after incubation for a predetermined time at 40° C. In different embodiments, the pre-determined time is about one week, about 2 weeks, about three weeks, about four weeks, or about five weeks, or about six weeks.

In another embodiment, the present invention includes a pharmaceutical composition comprising any of the claimed modified C-peptides and insulin.

Certain embodiments include the use of any of the disclosed modified C-peptides to reduce the risk of hypoglycemia in a human patient with insulin dependent diabetes, in a regimen which additionally comprises the administration of insulin, comprising; a) administering insulin to the patient; b) administering a therapeutic dose of the modified C-peptide in a different site as that used for the patient's insulin administration; c) adjusting the dosage amount, type, or frequency of insulin administered based on the patient's altered insulin requirements resulting from the therapeutic dose of the modified C-peptide.

In some embodiments, the patient has at least one long term complications of diabetes.

Certain embodiments include a method for treating an insulin-dependent human patient, comprising the steps of; a) administering insulin to the patient, wherein the patient has neuropathy; b) administering subcutaneously to the patient a therapeutic dose of any of the disclosed modified C-peptides in a different site as that used for the patient's insulin administration; c) adjusting the dosage amount, type, or frequency of insulin administered based on monitoring the patient's altered insulin requirements resulting from the therapeutic dose of modified C-peptide, wherein the adjusted dose of insulin reduces the risk, incidence, or severity of hypoglycemia, wherein the adjusted dose of insulin is at least 10% less than the patient's insulin dose prior to starting modified C-peptide treatment.

Certain embodiments include a method of reducing insulin usage in an insulin-dependent human patient, comprising the steps of; a) administering insulin to the patient; b) administering subcutaneously to the patient a therapeutic dose any of the disclosed modified C-peptides in a different site as that used for the patient's insulin administration; c) adjusting the dosage amount, type, or frequency of insulin administered based on monitoring the patient's altered insulin requirements resulting from the therapeutic dose of modified C-peptide, wherein the adjusted dose of insulin does not induce hypoglycemia, wherein the adjusted dose of insulin is at least 10% less than the patient's insulin dose prior to starting the modified C-peptide treatment.

### DETAILED DESCRIPTION OF THE INVENTION

### I. Definitions

The term “Cₘₐₓ” as used herein is the maximum serum or plasma concentration of drug which occurs during the period of release which is monitored.

The term “Cₘᵢₙ” as used herein is the minimum serum or plasma concentration of drug which occurs during the period of release during the treatment period.

The term “Cₐᵥₑ” as used herein is the average serum or plasma concentration of drug derived by dividing the area under the curve (AUC) of the release profile by the duration of the release.

The term “Cₛₛ₋ₐᵥₑ” as used herein is the average steady-state concentration of drug obtained during a multiple dosing regimen after dosing for at least five elimination half-lives. It will be appreciated that drug concentrations are fluctuating within dosing intervals even once an average steady-state concentration of drug has been obtained.

The term “tₘₐₓ” as used herein is the time post-dose at which Cₘₐₓ is observed.

The term “AUC” as used herein means “area under curve” for the serum or plasma concentration-time curve, as calculated by the trapezoidal rule over the complete sample collection interval.

The term “bioavailability” refers to the amount of drug that reaches the circulation system expressed in percent of that administered. The amount of bioavailable material can be defined as the calculated AUC for the release profile of the drug during the time period starting at post-administration and ending at a predetermined time point. As is understood in the art, a release profile is generated by graphing the serum levels of a biologically active agent in a subject (Y-axis) at predetermined time points (X-axis). Bioavailability is often referred to in terms of % bioavailability, which is the bioavailability achieved for a drug (such as C-peptide) following administration of a sustained release composition of that drug divided by the bioavailability achieved for the drug following intravenous administration of the same equivalent dose of the drug, multiplied by 100.

The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz G E and R H Schirmer, Principles of Protein Structure, Springer-Verlag (1979)). According to such analyses, groups of amino acids can be defined where amino acids within a group exchange preferentially with each other, and therefore resemble each other most in their impact on the overall protein structure (Schulz G E and R H Schirmer, Principles of Protein Structure, Springer-Verlag (1979)).

Examples of amino acid groups defined in this manner include: a “charged/polar group,” consisting of Glu, Asp, Asn, Gln, Lys, Arg, and His; an “aromatic or cyclic group,” consisting of Pro, Phe, Tyr, and Trp; and an “aliphatic group,” consisting of Gly, Ala, Val, Leu, Ile, Met, Ser, Thr, and Cys.

Within each group, subgroups can also be identified, e.g., the group of charged/polar amino acids can be sub-divided into the subgroups consisting of the “positively-charged subgroup,” consisting of Lys, Arg, and His; the “negatively-charged subgroup,” consisting of Glu and Asp, and the “polar subgroup” consisting of Asn and Gln. The aromatic or cyclic group can be sub-divided into the subgroups consisting of the “nitrogen ring subgroup,” consisting of Pro, His, and Trp; and the “phenyl subgroup” consisting of Phe and Tyr. The aliphatic group can be sub-divided into the subgroups consisting of the “large aliphatic non-polar subgroup,” consisting of Val, Leu, and Ile; the “aliphatic slightly-polar subgroup,” consisting of Met, Ser, Thr, and Cys; and the “small-residue sub-group,” consisting of Gly and Ala.

Examples of conservative mutations include amino acid substitutions of amino acids within the subgroups above, e.g., Lys for Arg and vice versa such that a positive charge can be maintained; Glu for Asp and vice versa such that a negative charge can be maintained; Ser for Thr such that a free —OH can be maintained; and Gln for Asn such that a free —NH₂ can be maintained. “Semi-conservative mutations” include amino acid substitutions of amino acids with the same groups listed above, which do not share the same subgroup. For example, the mutation of Asp for Asn, or Asn for Lys, all involve amino acids within the same group, but different subgroups.

“Non-conservative mutations” involve amino acid substitutions between different groups, e.g., Lys for Leu, Phe for Ser.

The terms “Dalton”, “Da”, or “D” refers to an arbitrary unit of mass, being 1/12 the mass of the nuclide of carbon-12, equivalent to 1.657×10⁻²⁴ g. The term “kDa” is for kilo Dalton (i.e., 1000 Daltons).

The terms “diabetes”, “diabetes mellitus”, or “diabetic condition”, unless specifically designated otherwise, encompass all forms of diabetes. The term “type 1 diabetic” or “type 1 diabetes” refers to a patient with a fasting plasma glucose concentration of greater than about 7.0 mmoL/L and a fasting C-peptide level of about, or less than about 0.2 nmoL/L. The term “type 1.5 diabetic” or “type 1.5 diabetes” refers to a patient with a fasting plasma glucose concentration of greater than about 7.0 mmoL/L and a fasting C-peptide level of about, or less than about 0.4 nmoL/L. The term “type 2 diabetic” or “type 2 diabetes” generally refers to a patient with a fasting plasma glucose concentration of greater than about 7.0 mmoL/L and fasting C-peptide level that is within or higher than the normal physiological range of C-peptide levels (about 0.47 to 2.5 nmoL/L). It will be appreciated that a patient initially diagnosed as a type 2 diabetic may subsequently develop insulin-dependent diabetes, and may remain diagnosed as a type 2 patient, even though their C-peptide levels drop to those of a type 1.5 or type 1 diabetic patient (<0.2 nmol/L).

The terms “insulin-dependent patient” or “insulin-dependent diabetes” encompass all forms of diabetics/diabetes who/that require insulin administration to adequately maintain normal glucose levels unless specified otherwise.

Diabetes is frequently diagnosed by measuring fasting blood glucose, insulin, or glycated hemoglobin levels (which are typically referred to as hemoglobin A1c, Hb_{1c}, Hb_{A1c}, or A1C). Normal adult glucose levels are 60-126 mg/dL. Normal insulin levels are 30-60 pmoL/L. Normal HbA1c levels are generally less than 6%. The World Health Organization defines the diagnostic value of fasting plasma glucose concentration to 7.0 mmoL/L (126 mg/dL) and above for diabetes mellitus (whole blood 6.1 mmoL/L or 110 mg/dL), or 2-hour glucose level greater than or equal to 11.1 mmoL/L (greater than or equal to 200 mg/dL). Other values suggestive of or indicating high risk for diabetes mellitus include elevated arterial pressure greater than or equal to 140/90 mm Hg; elevated plasma triglycerides (greater than or equal to 1.7 mmoL/L [150 mg/dL]) and/or low HDL-cholesterol (less than 0.9 mmoL/L [35 mg/dL] for men; and less than 1.0 mmoL/L [39 mg/dL] for women); central obesity (BMI exceeding 30 kg/m²); microalbuminuria, where the urinary albumin excretion rate is greater than or equal to 20 μg/min or the albumin creatinine ratio is greater than or equal to 30 mg/g.

The term “delivery agent” refers to carrier compounds or carrier molecules that are effective in the oral delivery of therapeutic agents, and may be used interchangeably with “carrier”.

The term “homology” describes a mathematically-based comparison of sequence similarities which is used to identify genes or proteins with similar functions or motifs. The nucleic acid and protein sequences of the present invention can be used as a “query sequence” to perform a search against public databases to, e.g., identify other family members, related sequences, or homologs. Such searches can be performed using the NBLAST and XBLAST programs (version 2.0) of Altschul et al.: J. Mol. Biol. 215: 403-410, (1990). BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al.: Nucleic Acids Res. 25(17): 3389-3402, (1997). When utilizing BLAST and Gapped BLAST programs, the default parameters of the respective programs (e.g., XBLAST and BLAST) can be used (see the World Wide Web at ncbi.nlm.nih.gov).

The term “homologous” refers to the relationship between two proteins that possess a “common evolutionary origin”, including proteins from superfamilies (e.g., the immunoglobulin superfamily) in the same species of animal, as well as homologous proteins from different species of animal (e.g., myosin light chain polypeptide; see Reeck et al.: Cell 50: 667, (1987)). Such proteins (and their encoding nucleic acids) have sequence homology, as reflected by their sequence similarity, whether in terms of percent identity or by the presence of specific residues or motifs and conserved positions. In specific embodiments, two nucleic acid sequences are “substantially homologous” or “substantially similar” when at least about 85%, and more preferably at least about 90% or at least about 95% of the nucleotides match over a defined length of the nucleic acid sequences, as determined by a sequence comparison algorithm known such as BLAST, FASTA, DNA Strider, CLUSTAL, etc. An example of such a sequence is an allelic or species variant of the specific genes of the present invention. Sequences that are substantially homologous may also be identified by hybridization, e.g., in a Southern hybridization experiment under, e.g., stringent conditions as defined for that particular system.

Similarly, in particular embodiments of the invention, two amino acid sequences are “substantially homologous” or “substantially similar” when greater than 80% of the amino acid residues are identical, or when greater than about 90% of the amino acid residues are similar (i.e., are functionally identical). Preferably the similar or homologous polypeptide sequences are identified by alignment using, e.g., the GCG (Genetics Computer Group, version 7, Madison, Wis.) pileup program, or using any of the programs and algorithms described above. The program may use the local homology algorithm of Smith and Waterman with the default values: gap creation penalty=−(1+⅓k), k being the gap extension number, average match=1, average mismatch=−0.333.

As used herein, “identity” means the percentage of identical nucleotide or amino acid residues at corresponding positions in two or more sequences when the sequences are aligned to maximize sequence matching, i.e., taking into account gaps and insertions. Identity can be readily calculated by known methods, including but not limited to those described in Computational Molecular Biology, Lesk A M, Ed., Oxford University Press, New York, (1988); Biocomputing: Informatics and Genome Projects, Smith D W, Ed., Academic Press, New York, (1993); Computer Analysis of Sequence Data, Part I, Griffin A M and Griffin H G, Eds., Humana Press, New Jersey, (1994); Sequence Analysis in Molecular Biology, von Heinje G, Academic Press, (1987); and Sequence Analysis Primer, Gribskov M and Devereux J, Eds., M Stockton Press, New York, (1991); and Carillo H and Lipman D, SIAM J. Applied Math., 48: 1073 (1988). Methods to determine identity are designed to give the largest match between the sequences tested. Moreover, methods to determine identity are codified in publicly available computer programs. Computer program methods to determine identity between two sequences include, but are not limited to, the GCG program package (Devereux J et al.: Nucleic Acids Res. 12(1): 387, (1984)), BLASTP, BLASTN, and FASTA (Altschul S F et al.: J. Molec. Biol. 215: 403-410, (1990) and Altschul S F et al.: Nucleic Acids Res. 25: 3389-3402, (1997)). The BLAST X program is publicly available from NCBI and other sources (BLAST Manual, Altschul S F et al., NCBI NLM NIH Bethesda, Md. 20894; Altschul S F et al., J. Mol. Biol. 215: 403-410, (1990)). The well-known Smith Waterman algorithm (Smith T F, Waterman M S: J. Mol. Biol. 147(1): 195-197, (1981)) can also be used to determine similarity between sequences.

The term “insulin” includes all forms of insulin including, without limitation, rapid-acting forms, such as Insulin Lispro rDNA origin: HUMALOG (1.5 mL, 10 mL, Eli Lilly and Company, Indianapolis, Ind.), Insulin Injection (Regular Insulin) from beef and pork (regular ILETIN I, Eli Lilly), human: rDNA: HUMULIN R (Eli Lilly), NOVOLIN R (Novo Nordisk, New York, N.Y.), Semi synthetic: VELOSULIN Human (Novo Nordisk), rDNA Human, Buffered: VELOSULIN BR, pork: regular Insulin (Novo Nordisk), purified pork: Pork Regular ILETIN II (Eli Lilly), Regular Purified Pork Insulin (Novo Nordisk), and Regular (Concentrated) ILETIN II U-500 (500 units/mL, Eli Lilly); intermediate-acting forms such as Insulin Zinc Suspension, beef and pork: LENTE ILETIN G I (Eli Lilly), Human, rDNA: HUMULIN L (Eli Lilly), NOVOLIN L (Novo Nordisk), purified pork: LENTE ILETIN II (Eli Lilly), Isophane Insulin Suspension (NPH): beef and pork: NPH ILETIN I (Eli Lilly), Human, rDNA: HUMULIN N (Eli Lilly), Novolin N (Novo Nordisk), purified pork: Pork NPH Eetin II (Eli Lilly), NPH-N (Novo Nordisk); and long-acting forms such as Insulin zinc suspension, extended (ULTRALENTE, Eli Lilly), human, rDNA: HUMULIN U (Eli Lilly).

The terms “measuring” or “measurement” mean assessing the presence, absence, quantity, or amount (which can be an effective amount) of either a given substance within a clinical- or patient-derived sample, including the derivation of qualitative or quantitative concentration levels of such substances, or otherwise evaluating the values or categorization of a patient's clinical parameters.

The term “meal” as used herein means a standard and/or a mixed meal.

The term “mean”, when preceding a pharmacokinetic value (e.g., mean tₘₐₓ), represents the arithmetic mean value of the pharmacokinetic value unless otherwise specified.

The term “mean baseline level” as used herein means the measurement, calculation, or level of a certain value that is used as a basis for comparison, which is the mean value over a statistically significant number of subjects, e.g., across a single clinical study or a combination of more than one clinical study.

The term “multiple dose” means that the patient has received at least two doses of the drug composition in accordance with the dosing interval for that composition.

The term “neuropathy” in the context of a “patient with neuropathy” or a patient that “has neuropathy”, means that the patient meets at least one of the four criteria outlined in the San Antonio Conference on diabetic neuropathy (report and recommendations of the San Antonio Conference on diabetic neuropathy. Ann. Neurol. 24 99-104 (1988)), which in brief include 1) clinical signs of polyneuropathy, 2) symptoms of nerve dysfunction, 3) nerve conduction deficits in at least two nerves, or 4) quantitative sensory deficits. The term “established neuropathy” means that the patient meets at least two of the four criteria outlined in the San Antonio Conference on diabetic neuropathy. The term “incipient neuropathy” refers to a patient that exhibits only nerve conduction deficits, and no other symptoms of neuropathy.

The term “normal glucose levels” is used interchangeably with the term “normoglycemic” and “normal” and refers to a fasting venous plasma glucose concentration of less than about 6.1 mmoL/L (110 mg/dL). Sustained glucose levels above normoglycemic are considered a pre-diabetic condition.

As used herein, the term “patient” in the context of the present invention is preferably a mammal. The mammal can be a human, non-human primate, mouse, rat, dog, cat, horse, or cow, but are not limited to these examples. Mammals other than humans can be advantageously used as patients that represent animal models of insulin-dependent diabetes mellitus, or diabetic conditions. A patient can be male or female. A patient can be one who has been previously diagnosed or identified as having insulin-dependent diabetes, or a diabetic condition, and optionally has already undergone, or is undergoing, a therapeutic intervention for the diabetes. A patient can also be one who is suffering from a long-term complication of diabetes. Preferably the patient is human.

The term “replacement dose” in the context of a replacement therapy for C-peptide refers to a dose of C-peptide or modified C-peptide that maintains C-peptide or modified C-peptide levels in the blood within a desirable range, particularly at a level which is at or above the minimum effective therapeutic level. In certain aspects, the replacement dose maintains the average steady-state concentration C-peptide or modified C-peptide levels above a minimum level of about 0.1 nM between dosing intervals. In certain aspects, the replacement dose maintains the average steady-state concentration C-peptide or modified C-peptide levels above a minimum level of about 0.2 nM between dosing intervals. In certain aspects, the replacement dose maintains the average steady-state concentration C-peptide or modified C-peptide levels above a minimum level of about 0.4 nM between dosing intervals.

The terms “subcutaneous” or “subcutaneously” or “S.C.” in reference to a mode of administration of insulin or modified C-peptide, refers to a drug that is administered as a bolus injection, or via an implantable device into the area in, or below the subcutis, the layer of skin directly below the dermis and epidermis, collectively referred to as the cutis. Preferred sites for subcutaneous administration and/or implantation include the outer area of the upper arm, just above and below the waist, except the area right around the navel (a 2-inch circle). The upper area of the buttock, just behind the hipbone. The front of the thigh, midway to the outer side, 4 inches below the top of the thigh to 4 inches above the knee.

The term “single dose” means that the patient has received a single dose of the drug composition or that the repeated single doses have been administered with washout periods in between. Unless specifically designated as “single dose” or at “steady-state” the pharmacokinetic parameters disclosed and claimed herein encompass both single-dose and multiple-dose conditions.

The term “sequence similarity” refers to the degree of identity or correspondence between nucleic acid or amino acid sequences that may or may not share a common evolutionary origin (see Reeck et al., supra). However, in common usage and in the present application, the term “homologous”, when modified with an adverb such as “highly”, may refer to sequence similarity and may or may not relate to a common evolutionary origin.

By “statistically significant”, it is meant that the result was unlikely to have occurred by chance. Statistical significance can be determined by any method known in the art. Commonly used measures of significance include the p-value, which is the frequency or probability with which the observed event would occur, if the null hypothesis were true. If the obtained p-value is smaller than the significance level, then the null hypothesis is rejected. In simple cases, the significance level is defined at a p-value of 0.05 or less.

As defined herein, the terms “sustained release”, “extended release”, or “depot formulation” refers to the release of a drug such as modified C-peptide from the sustained release composition or sustained release device which occurs over a period which is longer than that period during which the drug would be available following direct I.V. or S.C. administration of a single dose of drug. In one aspect, sustained release will be a release that occurs over a period of at least about one to two weeks, about two to four weeks, about one to two months, about two to three months, or about three to six months. In certain aspects, sustained release will be a release that occurs over a period of about six months to about one year. The continuity of release and level of release can be affected by the type of sustained release device (e.g., programmable pump or osmotically-driven pump) or sustained release composition, and type of modified C-peptides used (e.g., monomer ratios, molecular weight, block composition, and varying combinations of polymers), polypeptide loading, and/or selection of excipients to produce the desired effect, as more fully described herein.

Various sustained release profiles can be provided in accordance with any of the methods of the present invention. “Sustained release profile” means a release profile in which less than 50% of the total release of drug that occurs over the course of implantation/insertion or other method of administering the drug in the body occurs within the first 24 hours of administration. In a preferred embodiment of the present invention, the extended release profile is selected from the group consisting of; a) the 50% release point occurring at a time that is between 48 and 72 hours after implantation/insertion or other method of administration; b) the 50% release point occurring at a time that is between 72 and 96 hours after implantation/insertion or other method of administration; c) the 50% release point occurring at a time that is between 96 and 110 hours after implantation/insertion or other method of administration; d) the 50% release point occurring at a time that is between 1 and 2 weeks after implantation/insertion or other method of administration; e) the 50% release point occurring at a time that is between 2 and 4 weeks after implantation/insertion or other method of administration; f) the 50% release point occurring at a time that is between 4 and 8 weeks after implantation/insertion or other method of administration; g) the 50% release point occurring at a time that is between 8 and 16 weeks after implantation/insertion or other method of administration; h) the 50% release point occurring at a time that is between 16 and 52 weeks (1 year) after implantation/insertion or other method of administration; and i) the 50% release point occurring at a time that is between 52 and 104 weeks after implantation/insertion or other method of administration.

Additionally, use of a sustained release composition can reduce the “degree of fluctuation” (“DFL”) of the drugs plasma concentration. DFL is a measurement of how much the plasma levels of a drug vary over the course of a dosing interval (Cₘₐₓ−Cₘᵢₙ/Cₘᵢₙ). For simple cases, such as I.V. administration, fluctuation is determined by the relationship between the elimination half-life (T_{1/2}) and dosing interval. If the dosing interval is equal to the half-life then the trough concentration is exactly half of the peak concentration, and the degree of fluctuation is 100%. Thus a sustained release composition with a reduced DFL (for the same dosing interval) signifies that the difference in peak and trough plasma levels has been reduced. Preferably, the patients receiving a sustained release composition of modified C-peptide have a DFL approximately 50%, 40%, or 30% of the DFL in patients receiving a non-extended release composition with the same dosing interval.

The terms “treating” or “treatment” means to relieve, alleviate, delay, reduce, reverse, improve, manage, or prevent at least one symptom of a condition in a patient. The term “treating” may also mean to arrest, delay the onset (i.e., the period prior to clinical manifestation of a disease), and/or reduce the risk of developing or worsening a condition.

As used herein, the terms “therapeutically effective amount”, “prophylactically effective amount”, or “diagnostically effective amount” is the amount of the drug, e.g., insulin or modified C-peptide, needed to elicit the desired biological response following administration.

The term “unit-dose forms” refers to physically discrete units suitable for human and animal patients and packaged individually as is known in the art. It is contemplated for purposes of the present invention that dosage forms of the present invention comprising therapeutically effective amounts of drug may include one or more unit doses (e.g., tablets, capsules, powders, semisolids [e.g., gelcaps or films], liquids for oral administration, ampoules or vials for injection, loaded syringes) to achieve the therapeutic effect. It is further contemplated for the purposes of the present invention that a preferred embodiment of the dosage form is a subcutaneously injectable dosage form.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviations, per practice in the art. Alternatively, “about” with respect to the compositions can mean plus or minus a range of up to 20%, preferably up to 10%, more preferably up to 5%.

As used herein and in the appended claims, the singular forms “a”, “an”, and “the” include plural referents unless the context clearly indicates otherwise. Thus, for example, reference to “a molecule” includes one or more of such molecules, “a reagent” includes one or more of such different reagents, reference to “an antibody” includes one or more of such different antibodies, and reference to “the method” includes reference to equivalent steps and methods known to those of ordinary skill in the art that could be modified or substituted for the methods and pharmaceutical compositions described herein.

Unless defined otherwise, all technical and scientific terms used herein have the same meaning as commonly understood by one of ordinary skill in the art to which the invention belongs. The following abbreviations are used in certain sections of the disclosure:
ADA Anti-drug antibody
AUC Area under the curve
AUC₍₀₋₇₎ Area under the plasma concentration-time curve from time zero to Day 7
AUC₍₀₋₁₄₎ Area under the plasma concentration-time curve from time zero to Day 14
AUC₍₀₋ₜ₎/ACUₜₐᵤ Area under the plasma concentration-time curve from time zero to the time of the last quantifiable concentration
AUC_{(0-inf)}/A_{inf} Area under the plasma concentration-time curve from time zero to infinity
Conc. Concentration
Cₛₛ Concentration at steady state
CL/F Apparent clearance uncorrected for bioavailability (F)
CLₛₛ/F Apparent clearance uncorrected for bioavailability (F) at steady state
Cₘₐₓ Maximum observed concentration
ELISA Enzyme-linked immunosorbent assay
F Bioavailability or female
Fᵣₑₗ Relative bioavailability
GLP Good Laboratory Practice
h Hours
i.v. Intravenous
kg Kilogram
L Liter
M Male
mg Milligram
mL Milliliter
min Minutes
MTD maximum tolerated dose
ND Not determined
ng Nanogram
NOEL no observed effect level.
nM/nmol/L Nanomolar
nnol Nanomole
QC Quality control
RIA Radioimmunoassay
s.c./S.C. Subcutaneous
SD Standard deviation
T_{1/2} Terminal elimination half-life
Tₘₐₓ Time to reach Cₘₐₓ
Vd/F Apparent volume of distribution following subcutaneous administration, uncorrected for bioavailability (F)
Vdₛₛ/F Apparent volume of distribution following subcutaneous administration, uncorrected for bioavailability (F) at steady state
wk Week

Although any methods, compositions, reagents, cells, similar or equivalent to those described herein can be used in the practice or testing of the invention, the preferred methods and materials are described herein.

All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; Handbook of Drug Screening, edited by Ramakrishna Seethala, Prabhavathi B. Fernandes (2001, New York, N.Y., Marcel Dekker, ISBN 0-8247-0562-9); and Lab Ref: A Handbook of Recipes, Reagents, and Other Reference Tools for Use at the Bench, edited by Jane Roskams and Linda Rodgers, 2002, Cold Spring Harbor Laboratory, ISBN 0-87969-630-3. Each of these general texts is herein incorporated by reference.

The publications discussed above are provided solely for their disclosure before the filing date of the present application. Nothing herein is to be construed as an admission that the invention is not entitled to antedate such disclosure by virtue of prior invention.

### II. Therapeutic Forms of C-Peptide

The terms “C-peptide” or “proinsulin C-peptide” as used herein includes all naturally-occurring and synthetic forms of C-peptide that retain C-peptide activity. Such C-peptides include the human peptide, as well as peptides derived from other animal species and genera, preferably mammals. Preferably, “C-peptide” refers to human C-peptide having the amino acid sequence EAEDLQVGQVELGGGPGAGSLQPLALEGSLQ (SEQ ID NO:1 in Table 1).

In certain embodiments, “C-peptide” refers to the C-terminal pentapeptide sequence (EGSLQ) (SEQ ID NO:31), or other variants which retain the C-terminal pentapeptide sequence (EGSLQ) (SEQ ID NO:31) and retain substantially the same biological activity of naturally occurring human C-peptide.

C-peptides from a number of different species have been sequenced, and are known in the art to be at least partially functionally interchangeable. It would thus be a routine matter to select a variant being a C-peptide from a species or genus other than human. Several such variants of C-peptide (i.e., representative C-peptides from other species) are shown in Table 1 (see SEQ ID NOS:1-29).

TABLE 1
C-peptide Variants

| | | |
|---|---|---|
| human M- | Human | gb\|AAA72531.1\| |
| proinsulin | EAEDLQVGQVELGGGPGAGSLQPLALEGSLQ | dbj\|BAH59081.1\| |
| | (SEQ ID NO: 1) | |
| Pan | (SEQ ID NO: 1) EAEDLQVGQVELGGGPGAGSLQPLALEGSLQ | NP_001008996.1\| |
| troglodytes | Alignment EAEDLQVGQVELGGGPGAGSLQPLALEGSLQ | emb\|CAA43403.1\| |
| | (SEQ ID NO: 2) EAEDLQVGQVELGGGPGAGSLQPLALEGSLQ | GENE ID: 449570 |
| | Identities = 31/31 (100%), Positives = 31/31 (100%), Gaps = 0/31 (0%) | INS |
| Gorilla | (SEQ ID NO: 1) EAEDLQVGQVELGGGPGAGSLQPLALEGSLQ | gb\|AAN06935.1\| |
| gorilla | Alignment EAEDLQVGQVELGGGPGAGSLQPLALEGSLQ | |
| | (SEQ ID NO: 3) EAEDLQVGQVELGGGPGAGSLQPLALEGSLQ | |
| | Identities = 31/31 (100%), Positives = 31/31 (100%), Gaps = 0/31 (0%) | |
| Pongo | (SEQ ID NO: 1) EAEDLQVGQVELGGGPGAGSLQPLALEGSLQ | gb\|AAN06937.1\| |
| pygmaeus | EAEDLQVGQVELGGGPGAGSLQPLALEGSLQ | |
| (Bornean | (SEQ ID NO: 4) EAEDLQVGQVELGGGPGAGSLQPLALEGSLQ | |
| orangutan) | Identities = 31/31 (100%), Positives = 31/31 (100%), Gaps = 0/31 (0%) | |
| Chloro- | (SEQ ID NO: 1) EAEDLQVGQVELGGGPGAGSLQPLALEGSLQ | emb\|CAA43405.1\| |
| cebus | EAED QVGQVELGGGPGAGSLQPLALEGSLQ | |
| aethiops | (SEQ ID NO: 5) EAEDPQVGQVELGGGPGAGSLQPLALEGSLQ | |
| (Monkey) | Identities = 30/31 (96%), Positives = 30/31 (96%), Gaps = 0/31 (0%) | |
| Canis | (SEQ ID NO: 1) EAEDLQVGQVELGGGPGAGSLQPLALEGSLQ | ref\|NP_001123565.1\| |
| lupus | EEDLQVVELGPGGLQPLALEG + LQ | sp\|P01321.1\|INS_ |
| familiaris | (SEQ ID NO: 6) EVEDLQVRDVELAGAPGEGGLQPLALEGALQ | CANFAemb\| |
| (Dog) | Identities = 23/31 (74%), Positives = 24/31 (77%), Gaps = 0/31 (0%) | CAA23475.1\| GENE |
| | | ID: 483665 INS |
| Oryctola- | (SEQ ID NO: 1) EAEDLQVGQVELGGGPGAGSLQPLALEGSLQ | gb\|ACK44319.1\| |
| gus | EE + LQVGQELGGGPAGLQPALE + LQ | |
| cuniculus | (SEQ ID NO: 7) EVEELQVGQAELGGGPDAGGLQPSALELALQ | |
| (Rabbit) | Identities = 23/31 (74%), Positives = 25/31 (80%), Gaps = 0/31 (0%) | |
| Rattus | (SEQ ID NO: 1) EAEDLQVGQVELGGGPGAGSLQPLALEGSLQ | ref\|NP_062003.1\| |
| norvegicus | EEDQVQ + ELGGGPGAGLQLALE + Q | sp\|P01323.1\|INS2_ |
| | (SEQ ID NO: 8) EVEDPQVAQLELGGGPGAGDLQTLALEVARQ | RAT |
| | Identities = 22/31 (70%), Positives = 24/31 (77%), Gaps = 0/31 (0%) | emb\|CAA24560.1\| |
| | | GENE ID: 24506 |
| | | Ins2 |
| Apodemus | (SEQ ID NO: 1) EAEDLQVGQVELGGGPGAGSLQPLALEGSLQ | gb\|ABB89748.1\| |
| semotus | EEDQVQ + ELGGGPGAGLQLALE + Q | |
| (Taiwan | (SEQ ID NO: 9) EVEDPQVAQLELGGGPGAGDLQTLALEVARQ | |
| field | Identities = 22/31 (70%), Positives = 24/31 (77%), Gaps = 0/31 (0%) | |
| mouse) | | |
| Geodia | (SEQ ID NO: 1) EAEDLQVGQVELGGGPGAGSLQPLALEGSLQ | pirl\|IS09278 |
| cydonium | EEDQVGQVELGGPGAGSQLALE + Q | |
| sponge | (SEQ ID NO: 10) EVEDPQVGQVELGAGPGAGSEQTLALEVARQ | |
| | Identities = 23/31 (74%), Positives = 24/31 (77%), Gaps = 0/31 (0%) | |
| Mus | (SEQ ID NO: 1) EAEDLQVGQVELGGGPGAGSLQPLALE | ref\|NP_032413.1\| |
| musculus | EEDQVQ + ELGGGPGAGLQLALE | sp\|P01326.1\|INS2_ |
| | (SEQ ID NO: 11) EVEDPQVAQLELGGGPGAGDLQTLALE | MOUSEemb\| |
| | Identities = 21/27 (77%), Positives = 22/27 (81%), Gaps = 0/27 (0%) | CAA28433.11 |
| | | GENE ID: 16334 |
| | | Ins2 |
| Mus caroli | (SEQ ID NO: 1) EAEDLQVGQVELGGGPGAGSLQPLALE | gb\|ABB89749.1\| |
| (Ryukyu | EEDQVQ + ELGGGPGAGLQLALE | |
| mouse) | (SEQ ID NO: 12) EVEDPQVAQLELGGGPGAGDLQTLALE | |
| | Identities = 21/27 (77%), Positives = 22/27 (81%), Gaps = 0/27 (0%) | |
| Rattus | (SEQ ID NO: 1) EAEDLQVGQVELGGGPGAGSLQPLALEGSLQ | prfl\|720460B |
| norvegicus | EEDQVQ + ELGGGPGAGLQLALE + Q | |
| | (SEQ ID NO: 13) EVEDPQVPQLELGGGPGAGDLQTLALEVARQ | |
| | Identities = 22/31 (70%), Positives = 24/31 (77%), Gaps = 0/31 (0%) | |
| Rattus | (SEQ ID NO: 1) EAEDLQVGQVELGGGPGAGSLQPLALEGSLQ | gb\|ABB89747.1\| |
| losea | EEDQVQELGGGPGAGLQLALE + Q | |
| | (SEQ ID NO: 14) EVEDPQVAQQELGGGPGAGDLQTLALEVARQ | |
| | Identities = 22/31 (70%), Positives = 23/31 (74%), Gaps = 0/31 (0%) | |
| Niviventer | (SEQ ID NO: 1) EAEDLQVGQVELGGGPGAGSLQPLALEGSLQ | gb\|ABB89750.1\| |
| coxingi | EEDQVQ + ELGGGPGGLQLALE + Q | |
| (Coxing's | (SEQ ID NO: 15) EVEDPQVPQLELGGGPGTGDLQTLALEVARQ | |
| white | Identities = 21/31 (67%), Positives = 23/31 (74%), Gaps = 0/31 (0%) | |
| bellied rat) | | |
| Microtus | (SEQ ID NO: 1) AEDLQVGQVELGGGPGAGSLQPLALE | gb\|ABB89752.1\| |
| kikuchii | EDQVQ + ELGGGPGAGLQLALE | |
| (Taiwan | (SEQ ID NO: 16) VEDPQVAQLELGGGPGAGDLQTLALE | |
| vole) | Identities = 20/26 (76%), Positives = 21/26 (80%), Gaps = 0/26 (0%) | |
| Rattus | (SEQ ID NO: 1) EAEDLQVGQVELGGGPGAGSLQPLALEGSLQ | ref\|NP_062002.1\| |
| norvegicus | EEDQVQ + ELGGGPAGLQLALE + Q | gb\|AAA41439.1\| |
| insulin 1 | (SEQ ID NO: 17) EVEDPQVPQLELGGGPEAGDLQTLALEVARQ | gb\|AAA41442.1\| |
| precursor | Identities = 21/31 (67%), Positives = 23/31 (74%), Gaps = 0/31 (0%) | emb\|CAA24559.1\| |
| | | gb\|EDL94407.1\| |
| | | GENE ID: 24505 |
| | | Ins1 |
| Felis catus | (SEQ ID NO: 1) EAEDLQVGQVELGGGPGAGSLQPLALEGSLQ | ref\|NP_001009272.1\| |
| (Domestic | EAEDLQELGPGAGLQPALELQ | sp\|P06306.2\|INS_ |
| cat) | (SEQ ID NO: 18) EAEDLQGKDAELGEAPGAGGLQPSALEAPLQ | FELCA |
| | Identities = 21/31 (67%), Positives = 21/31 (67%), Gaps = 0/31 (0%) | dbj\|BAB84110.1\| |
| | | GENE ID: 493804 |
| | | INS |
| Golden | (SEQ ID NO: 1) AEDLQVGQVELGGGPGAGSLQPLALE | sp\|P01313.2\|INS_ |
| hamste | EDQVQ + ELGGGPGALQLALE | CRILO |
| | (SEQ ID NO: 19) VEDPQVAQLELGGGPGADDLQTLALE | pirl\|I48166 |
| | Identities = 19/26 (73%), Positives = 20/26 (76%), Gaps = 0/26 (0%) | gb\|AAA37089.1\| |
| Niviventer | (SEQ ID NO: 1) EAEDLQVGQVELGGGPGAGSLQPLALEGSLQ | gb\|ABB89746.1\| |
| coxingi | EEDQVQ + ELGGPAGLQLALE + Q | |
| (Coxing's | (SEQ ID NO: 20) EVEDPQVAQLELGEGPEAGDLQTLALEVARQ | |
| white | Identities = 20/31 (64%), Positives = 22/31 (70%), Gaps = 0/31 (0%) | |
| bellied rat) | | |
| Apodemus | (SEQ ID NO: 1) EAEDLQVGQVELGGGPGAGSLQPLALEGSLQ | gb\|ABB89744.1\| |
| semotus | EEDQVQ + ELGGPGGL + LALE + Q | |
| (Taiwan | (SEQ ID NO: 21) EVEDPQVEQLELGGAPGTGDLETLALEVARQ | |
| field | Identities = 19/31 (61%), Positives = 22/31 (70%), Gaps = 0/31 (0%) | |
| mouse) | | |
| Rattus | (SEQ ID NO: 1) EAEDLQVGQVELGGGPGAGSLQPLALEGSLQ | gb\|ABB89743.1\| |
| losea | EEDQVQ + ELGGPAGLQLALE + Q | |
| | (SEQ ID NO: 22) EVEDPQVPQLELGGSPEAGDLQTLALEVARQ | |
| | Identities = 20/31 (64%), Positives = 22/31 (70%), Gaps = 0/31 (0%) | |
| Meriones | (SEQ ID NO: 1) AEDLQVGQVELGGGPGAGSLQPLALEGSLQ | gb\|ABB89751.1\| |
| unguicu- | EDQ + Q + ELGGPGAGLQLALE + Q | |
| latus | (SEQ ID NO: 23) VEDPQMPQLELGGSPGAGDLQALALEVARQ | |
| (Mongolian | Identities = 19/30 (63%), Positives = 22/30 (73%), Gaps = 0/30 (0%) | |
| gerbil) | | |
| Psammo- | (SEQ ID NO: 1) AEDLQVGQVELGGGPGAGSLQPLALEGSLQ + | sp\|Q62587.1\|INS_ |
| mys obesus | DQ + Q + ELGGPGAGL + LALE + Q | PSAOB |
| (Fat sand | (SEQ ID NO: 24) VDDPQMPQLELGGSPGAGDLRALALEVARQ | emb\|CAA66897.1\| |
| rat) | Identities = 17/30 (56%), Positives = 22/30 (73%), Gaps = 0/30 (0%) | |
| Sus scrofa | (SEQ ID NO: 1) EAEDLQVGQVELGGGPGAGSLQPLALEG | ref\|NP_001103242.1\| |
| (Pig) | EAE + QGVELGGGGLQLALEG | |
| | (SEQ ID NO: 25) EAENPQAGAVELGG--GLGGLQALALEG | |
| | Identities = 19/28 (67%), Positives = 20/28 (71 %), Gaps = 2/28 (7%) | |
| Rhinolo- | (SEQ ID NO: 26) EVEDPQAGQVELGGGPGTGGLQSLALEGPPQ | gb\|ACC68945.1\| |
| phus | | |
| ferrum- | | |
| equinum | | |
| Equus | (SEQ ID NO: 27) EAEDPQVGEVELGGGPGLGGLQPLALAGPQQ | GENE ID: |
| przewalskii | | 100060077 |
| (Horse) | | LOC100060077 |
| | | gb\|AAB25818.1\| |
| Bos Taurus | (SEQ ID NO: 28) EVEGPQVGALELAGGPGAGGLEGPPQ | gb\|AAI42035.1\| |
| (Bovine) | | |
| Otolemur | (SEQ ID NO: 29) DTEDPQVGQVGLGGSPITGDLQSLALDVPPQ | gb\|ACH53103.1\| |
| garnettii | | |
| (Small- | | |
| eared | | |
| galago) | | |

Thus all such homologues, orthologs, and naturally-occurring isoforms of C-peptide from human as well as other species (SEQ. ID NOS:1-29) are included in any of the methods and pharmaceutical compositions of the invention, as long as they retain detectable C-peptide activity.

The C-peptides may be in their native form, i.e., as different variants as they appear in nature in different species which may be viewed as functionally equivalent variants of human C-peptide, or they may be functionally equivalent natural derivatives thereof, which may differ in their amino acid sequence, e.g., by truncation (e.g., from the N- or C-terminus or both) or other amino acid deletions, additions, insertions, substitutions, or post-translational modifications. Naturally-occurring chemical derivatives, including post-translational modifications and degradation products of C-peptide, are also specifically included in any of the methods and pharmaceutical compositions of the invention including, e.g., pyroglutamyl, iso-aspartyl, proteolytic, phosphorylated, glycosylated, oxidatized, isomerized, and deaminated variants of C-peptide.

It is known in the art to synthetically modify the sequences of proteins or peptides, while retaining their useful activity, and this may be achieved using techniques which are standard in the art and widely described in the literature, e.g., random or site-directed mutagenesis, cleavage, and ligation of nucleic acids, or via the chemical synthesis or modification of amino acids or polypeptide chains. Similarly it is within the skill in the art to address and/or mitigate immunogenicity concerns if they arise using C-peptide variants, e.g., by the use of automated computer recognition programs to identify potential T cell epitopes, and directed evolution approaches to identify less immunogenic forms.

Any such modifications, or combinations thereof, may be made and used in any of the methods and pharmaceutical compositions of the invention, as long as activity is retained. The C-terminal end of the molecule is known to be important for activity. Preferably, therefore, the C-terminal end of the C-peptide should be preserved in any such C-peptide variants or derivatives, more preferably the C-terminal pentapeptide of C-peptide (EGSLQ) (SEQ ID NO:31) should be preserved or sufficient (see Henriksson M et al.: Cell Mol. Life Sci. 62: 1772-1778, (2005)). As mentioned above, modification of an amino acid sequence may be by amino acid substitution, e.g., an amino acid may be replaced by another that preserves the physicochemical character of the peptide (e.g., A may be replaced by G or vice versa, V by A or L; E by D or vice versa; and Q by N). Generally, the substituting amino acid has similar properties, e.g., hydrophobicity, hydrophilicity, electronegativity, bulky side chains, etc., to the amino acid being replaced.

Modifications to the mid-part of the C-peptide sequence (e.g., to residues 13 to 25 of human C-peptide) allow the production of functional derivatives or variants of C-peptide. Thus, C-peptides which may be used in any of the methods or pharmaceutical compositions of the invention may have amino acid sequences which are substantially homologous, or substantially similar to the native C-peptide amino acid sequences, e.g., to the human C-peptide sequence of SEQ ID NO:1 or any of the other native C-peptide sequences shown in Table 1. Alternatively, the C-peptide may have an amino acid sequence having at least 30% preferably at least 40, 50, 60, 70, 75, 80, 85, 90, 95, 98, or 99% identity with the amino acid sequence of any one of SEQ ID NOS:1-29 as shown in Table 1, preferably with the native human sequence of SEQ ID NO:1. In a preferred embodiment, the C-peptide for use in any of the methods or pharmaceutical compositions of the present invention is at least 80% identical to a sequence selected from Table 1. In another aspect, the C-peptide for use in any of the methods or pharmaceutical compositions of the invention is at least 80% identical to human C-peptide (SEQ ID NO:1). Although any amino acid of C-peptide may be altered as described above, it is preferred that one or more of the glutamic acid residues at positions 3, 11, and 27 of human C-peptide (SEQ ID NO:1) or corresponding or equivalent positions in C-peptide of other species, are conserved. Preferably, all of the glutamic acid residues at positions 3, 11, and 27 (or corresponding Glu residues) of SEQ ID NO:1 are conserved. Alternatively, it is preferred that Glu27 of human C-peptide (or a corresponding Glu residue of a non-human C-peptide) is conserved. An exemplary functional equivalent form of C-peptide which may be used in any of the methods or pharmaceutical compositions of the invention includes the amino acid sequences:

(SEQ ID NO: 30)
EXEXXQXXXXELXXXXXXXXXXXXALBXXXQ.
(SEQ ID NO: 31)
GXEXXQXXXXELXXXXXXXXXXXXALBXXXQ.

As used herein, X is any amino acid. The N-terminal residue may be either Glu or Gly (SEQ ID NO:30 or SEQ ID NO:33, respectively). Functionally equivalent derivatives or variants of native C-peptide sequences may readily be prepared according to techniques well-known in the art, and include peptide sequences having a functional, e.g., a biological activity of a native C-peptide.

Fragments of native or synthetic C-peptide sequences may also have the desirable functional properties of the peptide from which they were derived and may be used in any of the methods or pharmaceutical compositions of the invention. The term “fragment” as used herein thus includes fragments of a C-peptide provided that the fragment retains the biological or therapeutically beneficial activity of the whole molecule. The fragment may also include a C-terminal fragment of C-peptide. Preferred fragments comprise residues 15-31 of native C-peptide, more especially residues 20-31. Peptides comprising the pentapeptide EGSLQ (SEQ ID NO:31) (residues 27-31 of native human C-peptide) are also preferred. The fragment may thus vary in size from, e.g., 4 to 30 amino acids or 5 to 20 residues. Suitable fragments are disclosed in WO98/13384 the contents of which are incorporated herein by reference.

The fragment may also include an N-terminal fragment of C-peptide, typically having the sequence EAEDLQVGQVEL (SEQ ID NO:32), or a fragment thereof which comprises 2 acidic amino acid residues, capable of adopting a conformation where said two acidic amino acid residues are spatially separated by a distance of 9-14 A between the alpha-carbons thereof. Also included are fragments having N- and/or C-terminal extensions or flanking sequences. The length of such extended peptides may vary, but typically are not more than 50, 30, 25, or 20 amino acids in length. Representative suitable fragments are described in U.S. Pat. No. 6,610,649, which is hereby incorporated by reference in its entirety.

In such a case it will be appreciated that the extension or flanking sequence will be a sequence of amino acids which is not native to a naturally-occurring or native C-peptide, and in particular a C-peptide from which the fragment is derived. Such a N- and/or C-terminal extension or flanking sequence may comprise, e.g., from 1 to 10, 1 to 6, 1 to 5, 1 to 4, or 1 to 3 amino acids.

The term “derivative” as used herein thus refers to C-peptide sequences or fragments thereof, which have modifications as compared to the native sequence. Such modifications may be one or more amino acid deletions, additions, insertions, and/or substitutions. These may be contiguous or non-contiguous. Representative variants may include those having 1 to 6, or more preferably 1 to 4, 1 to 3, or 1 or 2 amino acid substitutions, insertions, and/or deletions as compared to any of SEQ ID NOS:1-33. The substituted amino acid may be any amino acid, particularly one of the well-known 20 conventional amino acids (Ala (A); Cys (C); Asp (D); Glu (E); Phe (F); Gly (G); His (H); Ile (I); Lys (K); Leu (L); Met (M); Asn (N); Pro (P); Gin (Q); Arg (R); Ser (S); Thr (T); Val (V); Trp (W); and Tyr (Y)). Any such variant or derivative of C-peptide may be used in any of the methods or pharmaceutical compositions of the invention.

Isomers of the native L-amino acids, e.g., D-amino acids may be incorporated in any of the above forms of C-peptide, and used in any of the methods or pharmaceutical compositions of the invention. Additional variants may include amino and/or carboxyl terminal fusions as well as intrasequence insertions of single or multiple amino acids. Longer peptides may comprise multiple copies of one or more of the C-peptide sequences, such as any of SEQ ID NOS:1-33. Insertional amino acid sequence variants are those in which one or more amino acid residues are introduced at a site in the protein. Deletional variants are characterized by the removal of one or more amino acids from the sequence. Variants may include, e.g., different allelic variants as they appear in nature, e.g., in other species or due to geographical variation. All such variants, derivatives, fusion proteins, or fragments of C-peptide are included, may be used in any of the methods claims or pharmaceutical compositions disclosed herein, and are subsumed under the term “C-peptide”.

The modified forms of C-peptide, C-peptide variants, derivatives, and fragments thereof are functionally equivalent in that they have detectable C-peptide activity. More particularly, they exhibit at least about 1%, at least about 5%, at least about 10%, at least about 15%, at least about 20%, at least about 30%, at least about 40%, at least about 50%, at least about 60%, at least about 70%, at least about 80%, at least about 90%, at least about 100%, or higher than 100% of the activity of native proinsulin C-peptide, particularly human C-peptide. Thus, they are capable of functioning as proinsulin C-peptide, i.e., can substitute for C-peptide itself. Such activity means any activity exhibited by a native C-peptide, whether a physiological response exhibited in an in vivo or in vitro test system, or any biological activity or reaction mediated by a native C-peptide, e.g., in an enzyme assay or in binding to test tissues, membranes, or metal ions. Thus, it is known that C-peptide causes an influx of calcium and initiates a range of intracellular signalling cascades such as phosphorylation of the MAP-kinase pathway including phosphorylation of ERK 1 and 2, CREB, PKC, GSK3, PI3K, NF-kappaB, and PPARgamma, resulting in an increased expression of eNOS, Na+K+ATPase and a wide range of transcription factors. An assay for C-peptide activity can thus be made by assaying for the activation or up-regulation of any of these pathways upon addition or administration of the peptide (e.g., fragment or derivative) in question to cells from relevant target tissues including endothelial, kidney, fibroblast and immune cells. Such assays are described in, e.g., Ohtomo Y et al. (Diabetologia 39: 199-205, (1996)), Kunt T et al. (Diabetologia 42(4): 465-471, (1999)), Shafqat J et al. (Cell Mol. Life Sci. 59: 1185-1189, (2002)). Kitamura T et al. (Biochem. J. 355: 123-129, (2001)), Hills and Brunskill (Exp Diab Res 2008), as described in WO98/13384 or in Ohtomo Y et al. (supra) or Ohtomo Y et al. (Diabetologia 41: 287-291, (1998)). An assay for C-peptide activity based on endothelial nitric oxide synthase (eNOS) activity is also described in Kunt T et al. (supra) using bovine aortic cells and a reporter cell assay. Binding to particular cells may also be used to assess or assay for C-peptide activity, e.g., to cell membranes from human renal tubular cells, skin fibroblasts, and saphenous vein endothelial cells using fluorescence correlation spectroscopy, as described, e.g., in Rigler R et al. (PNAS USA 96: 13318-13323, (1999)), Henriksson M et al. (Cell Mol. Life Sci. 57: 337-342, (2000)) and Pramanik A et al. (Biochem Biophys. Res. Commun. 284: 94-98, (2001)).

In another aspect of any of the claimed modified C-peptides, the modified C-peptide has a plasma or sera pharmacokinetic AUC profile at least about 5-fold greater than unmodified C-peptide when subcutaneously administered to a mammal.

In another aspect of any of the claimed modified C-peptides, the modified C-peptide has a plasma or sera pharmacokinetic AUC profile at least about 6-fold greater than unmodified C-peptide when subcutaneously administered to a mammal.

In another aspect of any of the claimed modified C-peptides, the modified C-peptide has a plasma or sera pharmacokinetic AUC profile at least about 7-fold greater than unmodified C-peptide when subcutaneously administered to a mammal.

In another aspect of any of the claimed modified C-peptides, the modified C-peptide has a plasma or sera pharmacokinetic AUC profile at least about 8-fold greater than unmodified C-peptide when subcutaneously administered to a mammal.

In another aspect of any of the claimed modified C-peptides, the modified C-peptide has a plasma or sera pharmacokinetic AUC profile at least about 10-fold greater than unmodified C-peptide when subcutaneously administered to a mammal.

In another aspect of any of the claimed modified C-peptides, the modified C-peptide has a plasma or sera pharmacokinetic AUC profile at least about 15-fold greater than unmodified C-peptide when subcutaneously administered to a mammal.

In another aspect of any of the claimed modified C-peptides, the modified C-peptide has a plasma or sera pharmacokinetic AUC profile at least about 20-fold greater than unmodified C-peptide when subcutaneously administered to a mammal.

In another aspect of any of the claimed modified C-peptides, the modified C-peptide has a plasma or sera pharmacokinetic AUC profile at least about 25-fold greater than unmodified C-peptide when subcutaneously administered to a mammal.

In another aspect of any of the claimed modified C-peptides, the modified C-peptide has a plasma or sera pharmacokinetic AUC profile at least about 50-fold greater than unmodified C-peptide when subcutaneously administered to a mammal.

In another aspect of any of the claimed modified C-peptides, the modified C-peptide has a plasma or sera pharmacokinetic AUC profile at least about 75-fold greater than unmodified C-peptide when subcutaneously administered to a mammal.

In another aspect of any of the claimed modified C-peptides, the modified C-peptide has a plasma or sera pharmacokinetic AUC profile at least about 100-fold greater than unmodified C-peptide when subcutaneously administered to a mammal.

In one aspect the mammal is a dog. In one aspect the mammal is a rat. In one aspect the mammal is a human.

### II. Extended Recombinant Polypeptides (XTENs)

The present invention provides compositions comprising extended recombinant polypeptides (“XTEN” or “XTENs”). In some embodiments, XTEN are generally extended length polypeptides with non-naturally occurring, substantially non-repetitive sequences that are composed mainly of small hydrophilic amino acids, with the sequence having a low degree or no secondary or tertiary structure under physiologic conditions.

In one aspect of the invention, XTEN polypeptide compositions are disclosed that are useful as fusion partners that can be linked to C-peptide, resulting in a C-peptide-XTEN fusion protein (e.g., monomeric fusion). XTENs can have utility as fusion protein partners in that they can confer certain chemical and pharmaceutical properties when linked to a biologically active protein to a create a fusion protein. Such desirable properties include but are not limited to enhanced pharmacokinetic parameters and solubility characteristics, amongst other properties described below. Such fusion protein compositions may have utility to treat certain diseases, disorders or conditions, as described herein. As used herein, “XTEN” specifically excludes antibodies or antibody fragments such as single-chain antibodies, Fe fragments of a light chain or a heavy chain.

In some embodiments, XTEN are long polypeptides having greater than about 100 to about 3000 amino acid residues, preferably greater than 400 to about 3000 residues when used as a single sequence, and cumulatively have greater than about 400 to about 3000 amino acid residues when more than one XTEN unit is used in a single fusion protein or conjugate. In other cases, where an increase in half-life of the fusion protein is not needed but where an increase in solubility or other physico/chemical property for the C-peptide fusion partner is desired, an XTEN sequence shorter than 100 amino acid residues, such as about 96, or about 84, or about 72, or about 60, or about 48, or about 36 amino acid residues may be incorporated into a fusion protein composition with the C-peptide to effect the property.

The selection criteria for the XTEN to be linked to the C-peptide to create the inventive fusion proteins generally relate to attributes of physical/chemical properties and conformational structure of the XTEN that can be, in turn, used to confer enhanced pharmaceutical and pharmacokinetic properties to the fusion proteins. The XTEN of the present invention may exhibit one or more of the following advantageous properties: conformational flexibility, enhanced aqueous solubility, high degree of protease resistance, low immunogenicity, low binding to mammalian receptors, and increased hydrodynamic (or Stokes) radii; properties that can make them particularly useful as fusion protein partners. Non-limiting examples of the properties of the fusion proteins comprising C-peptide that may be enhanced by XTEN include increases in the overall solubility and/or metabolic stability, reduced susceptibility to proteolysis, reduced immunogenicity, reduced rate of absorption when administered subcutaneously or intramuscularly, and enhanced pharmacokinetic properties such as terminal half-life and area under the curve (AUC), slower absorption after subcutaneous or intramuscular injection (compared to C-peptide not linked to XTEN) such that the Cₘₐₓ is lower, which may, in turn, result in reductions in adverse effects of the C-peptide that, collectively, can result in an increased period of time that a fusion protein of a C-peptide-XTEN composition administered to a subject remains within a therapeutic window, compared to the corresponding C-peptide component not linked to XTEN.

A variety of methods and assays are known in the art for determining the physical/chemical properties of proteins such as the fusion protein compositions comprising the inventive XTEN; properties such as secondary or tertiary structure, solubility, protein aggregation, melting properties, contamination and water content. Such methods include analytical centrifugation, EPR, HPLC-ion exchange, HPLC-size exclusion, HPLC-reverse phase, light scattering, capillary electrophoresis, circular dichroism, differential scanning calorimetry, fluorescence, HPLC-ion exchange, HPLC-size exclusion, IR, NMR, Raman spectroscopy, refractometry, and UVNisible spectroscopy. Additional methods are disclosed in Arnau et al., Prot. Expr. and Purif., (2006) 48, 1-13. Application of these methods to the invention would be within the grasp of a person skilled in the art.

Typically, the XTEN component of the fusion proteins are designed to behave like denatured peptide sequences under physiological conditions, despite the extended length of the polymer. Denatured describes the state of a peptide in solution that is characterized by a large conformational freedom of the peptide backbone. Most peptides and proteins adopt a denatured conformation in the presence of high concentrations of denaturants or at elevated temperature. Peptides in denatured conformation have, for example, characteristic circular dichroism (CD) spectra and are characterized by a lack of long-range interactions as determined by NMR. “Denatured conformation” and “unstructured conformation” are used synonymously herein. In some cases, the invention provides XTEN sequences that, under physiologic conditions, can resemble denatured sequences largely devoid in secondary structure. In other cases, the XTEN sequences can be substantially devoid of secondary structure under physiologic conditions. “Largely devoid,” as used in this context, means that less than 50% of the XTEN amino acid residues of the XTEN sequence contribute to secondary structure as measured or determined by the means described herein. “Substantially devoid,” as used in this context, means that at least about 60%, or about 70%, or about 80%, or about 90%, or about 95%, or at least about 99% of the XTEN amino acid residues of the XTEN sequence do not contribute to secondary structure, as measured or determined by the means described herein.

A variety of methods have been established in the art to discern the presence or absence of secondary and tertiary structures in a given polypeptide. In particular, secondary structure can be measured spectrophotometrically, e.g., by circular dichroism spectroscopy in the “far-UV” spectral region (190-250 nm). Secondary structure elements, such as alpha-helix and beta-sheet, each give rise to a characteristic shape and magnitude of CD spectra. Secondary structure can also be predicted for a polypeptide sequence via certain computer programs or algorithms, such as the well-known ChouFasman algorithm (Chou, P. Y., et al., (1974) Biochemistry, 13: 222-45) and the Garnier-Osguthorpe-Robson (“GOR”) algorithm (Gamier J., Gibrat J. F., Robson B. (1996), GOR method for predicting protein secondary structure from amino acid sequence. Methods Enzymol 266:540-553), as described in US Patent Application Publication No. 20030228309A1. For a given sequence, the algorithms can predict whether there exists some or no secondary structure at all, expressed as the total and/or percentage of residues of the sequence that form, for example, alpha-helices or beta-sheets or the percentage of residues of the sequence predicted to result in random coil formation (which lacks secondary structure).

In some cases, the XTEN sequences used in the inventive fusion protein compositions can have an alpha-helix percentage ranging from 0% to less than about 5% as determined by a Chou-Fasman algorithm. In other cases, the XTEN sequences of the fusion protein compositions can have a beta-sheet percentage ranging from 0% to less than about 5% as determined by a Chou-Fasman algorithm. In some cases, the XTEN sequences of the fusion protein compositions can have an alpha-helix percentage ranging from 0% to less than about 5% and a beta-sheet percentage ranging from 0% to less than about 5% as determined by a Chou-Fasman algorithm. In preferred embodiments, the XTEN sequences of the fusion protein compositions will have an alpha-helix percentage less than about 2% and a beta-sheet percentage less than about 2%. In other cases, the XTEN sequences of the fusion protein compositions can have a high degree of random coil percentage, as determined by a GOR algorithm. In some embodiments, an XTEN sequence can have at least about 80%, more preferably at least about 90%, more preferably at least about 91%, more preferably at least about 92%, more preferably at least about 93%, more preferably at least about 94%, more preferably at least about 95%, more preferably at least about 96%, more preferably at least about 97%, more preferably at least about 98%, and most preferably at least about 99% random coil, as determined by a GOR algorithm.

XTEN sequences of the subject compositions can be substantially non-repetitive. In general, repetitive amino acid sequences have a tendency to aggregate or form higher order structures, as exemplified by natural repetitive sequences such as collagens and leucine zippers, or form contacts resulting in crystalline or pseudo-crystalline structures. In contrast, the low tendency of non-repetitive sequences to aggregate enables the design of long-sequence XTENs with a relatively low frequency of charged amino acids that would be likely to aggregate if the sequences were otherwise repetitive. Typically, the C-peptide-XTEN fusion proteins comprise XTEN sequences of greater than about 100 to about 3000 amino acid residues, preferably greater than 400 to about 3000 residues, wherein the sequences are substantially non-repetitive. In one embodiment, the XTEN sequences can have greater than about 100 to about 3000 amino acid residues, preferably greater than 400 to about 3000 amino acid residues, in which no three contiguous amino acids in the sequence are identical amino acid types unless the amino acid is serine, in which case no more than three contiguous amino acids are serine residues. In the foregoing embodiment, the XTEN sequence would be substantially non-repetitive.

The degree of repetitiveness of a polypeptide or a gene can be measured by computer programs or algorithms or by other means known in the art. Repetitiveness in a polypeptide sequence can, for example, be assessed by determining the number of times shorter sequences of a given length occur within the polypeptide. For example, a polypeptide of 200 amino acid residues has 192 overlapping 9-amino acid sequences (or 9-mer “frames”) and 198 3-mer frames, but the number of unique 9-mer or 3-mer sequences will depend on the amount of repetitiveness within the sequence. A score can be generated (hereinafter “subsequence score”) that is reflective of the degree of repetitiveness of the subsequences in the overall polypeptide sequence. In the context of the present invention, “subsequence score” means the sum of occurrences of each unique 3-mer frame across a 200 consecutive amino acid sequence of the polypeptide divided by the absolute number of unique 3-mer subsequences within the 200 amino acid sequence. Examples of such subsequence scores derived from the first 200 amino acids of repetitive and non-repetitive polypeptides are presented in Example 41. In some embodiments, the present invention provides C-peptide-XTEN each comprising XTEN in which the XTEN can have a subsequence score less than 12, more preferably less than 10, more preferably less than 9, more preferably less than 8, more preferably less than 7, more preferably less than 6, and most preferably less than 5. In the embodiments hereinabove described in this paragraph, an XTEN with a subsequence score less than about 10 (i.e., 9, 8, 7, etc.) would be “substantially non-repetitive.”

The non-repetitive characteristic of XTEN can impart to fusion proteins with C-peptide a greater degree of solubility and less tendency to aggregate compared to polypeptides having repetitive sequences. These properties can facilitate the formulation of XTEN-comprising pharmaceutical preparations containing extremely high drug concentrations, in some cases exceeding 100 mg/ml.

Furthermore, the XTEN polypeptide sequences of the embodiments are designed to have a low degree of internal repetitiveness in order to reduce or substantially eliminate immunogenicity when administered to a mammal. Polypeptide sequences composed of short, repeated motifs largely limited to three amino acids, such as glycine, serine and glutamate, may result in relatively high antibody titers when administered to a mammal despite the absence of predicted T-cell epitopes in these sequences. This may be caused by the repetitive nature of polypeptides, as it has been shown that immunogens with repeated epitopes, including protein aggregates, cross-linked immunogens, and repetitive carbohydrates are highly immunogenic and can, for example, result in the cross-linking of B-cell receptors causing B-cell activation. (Johansson, J., et al. (2007) Vaccine, 25 :1676-82; Yankai, Z., et al. (2006) Biochem. Biophys. Res. Commun., 345:1365-71; Hsu, C. T., et al. (2000) Cancer Res, 60:370151; Bachmann M. F., et al., Eur. J. Immunol. (1995) 25(12):34453451).

The present invention encompasses XTEN that can comprise multiple units of shorter sequences, or motifs, in which the amino acid sequences of the motifs are non-repetitive. In designing XTEN sequences, it was discovered that the non-repetitive criterion may be met despite the use of a “building block” approach using a library of sequence motifs that are multimerized to create the XTEN sequences. Thus, while an XTEN sequence may consist of multiple units of as few as four different types of sequence motifs, because the motifs themselves generally consist of non-repetitive amino acid sequences, the overall XTEN sequence is rendered substantially non-repetitive.

In one embodiment, XTEN can have a non-repetitive sequence of greater than about 100 to about 3000 amino acid residues, preferably greater than 400 to about 3000 residues, wherein at least about 80%, or at least about 85%, or at least about 90%, or at least about 95%, or at least about 97%, or about 100% of the XTEN sequence consists of non-overlapping sequence motifs, wherein each of the motifs has about 9 to 36 amino acid residues. In other embodiments, at least about 80%, or at least about 85%, or at least about 90%, or at least about 95%, or at least about 97%, or about 100% of the XTEN sequence consists of non-overlapping sequence motifs wherein each of the motifs has 9 to 14 amino acid residues. In still other embodiments, at least about 80%, or at least about 85%, or at least about 90%, or at least about 95%, or at least about 97%, or about 100% of the XTEN sequence component consists of non-overlapping sequence motifs wherein each of the motifs has 12 amino acid residues. In these embodiments, it is preferred that the sequence motifs be composed mainly of small hydrophilic amino acids, such that the overall sequence has an unstructured, flexible characteristic. Examples of amino acids that can be included in XTEN, are, e.g., arginine, lysine, threonine, alanine, asparagine, glutamine, aspartate, glutamate, serine, and glycine. As a result of testing variables such as codon optimization, assembly polynucleotides encoding sequence motifs, expression of protein, charge distribution and solubility of expressed protein, and secondary and tertiary structure, it was discovered that XTEN compositions with enhanced characteristics mainly include glycine (G), alanine (A), serine (S), threonine (T), glutamate (E) and proline (P) residues wherein the sequences are designed to be substantially non-repetitive. In a preferred embodiment, XTEN sequences have predominately four to six types of amino acids selected from glycine (G), alanine (A), serine (S), threonine (T), glutamate (E) or proline (P) that are arranged in a substantially non-repetitive sequence that is greater than about 100 to about 3000 amino acid residues, preferably greater than 400 to about 3000 residues in length. In some embodiments, XTEN can have sequences of greater than about 100 to about 3000 amino acid residues, preferably greater than 400 to about 3000 residues, wherein at least about 80% of the sequence consists of non-overlapping sequence motifs wherein each of the motifs has 9 to 36 amino acid residues wherein each of the motifs consists of 4 to 6 types of amino acids selected from glycine (G), alanine (A), serine (S), threonine (T), glutamate (E) and proline (P), and wherein the content of anyone amino acid type in the full-length XTEN does not exceed 30%. In other embodiments, at least about 90% of the XTEN sequence consists of non-overlapping sequence motifs wherein each of the motifs has 9 to 36 amino acid residues wherein the motifs consist of 4 to 6 types of amino acids selected from glycine (G), alanine (A), serine (S), threonine (T), glutamate (E) and proline (P), and wherein the content of anyone amino acid type in the full-length XTEN does not exceed 30%. In other embodiments, at least about 90% of the XTEN sequence consists of non-overlapping sequence motifs wherein each of the motifs has 12 amino acid residues consisting of 4 to 6 types of amino acids selected from glycine (G), alanine (A), serine (S), threonine (T), glutamate (E) and proline (P), and wherein the content of anyone amino acid type in the full length XTEN does not exceed 30%. In yet other embodiments, at least about 90%, or about 91%, or about 92%, or about 93%, or about 94%, or about 95%, or about 96%, or about 97%, or about 98%, or about 99%, to about 100% of the XTEN sequence consists of non-overlapping sequence motifs wherein each of the motifs has 12 amino acid residues consisting of glycine (G), alanine (A), serine (S), threonine (T), glutamate (E) and proline (P), and wherein in the content of any one amino acid type in the full-length XTEN does not exceed 30%.

In still other embodiments, XTENs comprise non-repetitive sequences of greater than about 100 to about 3000 amino acid residues, preferably greater than 400 to about 3000 amino acid residues wherein at least about 80%, or at least about 90%, or about 91%, or about 92%, or about 93%, or about 94%, or about 95%, or about 96%, or about 97%, or about 98%, or about 99% of the sequence consists of non-overlapping sequence motifs of 9 to 14 amino acid residues wherein the motifs consist of 4 to 6 types of amino acids selected from glycine (G), alanine (A), serine (S), threonine (T), glutamate (E) and proline (P), and wherein the sequence of any two contiguous amino acid residues in anyone motif is not repeated more than twice in the sequence motif. In other embodiments, at least about 90%, or about 91%, or about 92%, or about 93%, or about 94%, or about 95%, or about 96%, or about 97%, or about 98%, or about 99% of an XTEN sequence consists of non-overlapping sequence motifs of 12 amino acid residues wherein the motifs consist of 4 to 6 types of amino acids selected from glycine (G), alanine (A), serine (S), threonine (T), glutamate (E) and proline (P), and wherein the sequence of any two contiguous amino acid residues in anyone sequence motif is not repeated more than twice in the sequence motif. In other embodiments, at least about 90%, or about 91%, or about 92%, or about 93%, or about 94%, or about 95%, or about 96%, or about 97%, or about 98%, or about 99% of an XTEN sequence consists of non-overlapping sequence motifs of 12 amino acid residues wherein the motifs consist of glycine (G), alanine (A), serine (S), threonine (T), glutamate (E) and proline (P), and wherein the sequence of any two contiguous amino acid residues in anyone sequence motif is not repeated more than twice in the sequence motif. In yet other embodiments, XTENs consist of 12 amino acid sequence motifs wherein the amino acids are selected from glycine (G), alanine (A), serine (S), threonine (T), glutamate (E) and proline (P), and wherein the sequence of any two contiguous amino acid residues in anyone sequence motif is not repeated more than twice in the sequence motif, and wherein the content of anyone amino acid type in the full length XTEN does not exceed 30%.

In the foregoing embodiments hereinabove described in this paragraph, the XTEN sequences would be substantially non-repetitive.

In some cases, the invention provides compositions comprising a non-repetitive XTEN sequence of greater than about 100 to about 3000 amino acid residues, preferably greater than 400 to about 3000 residues, wherein at least about 80%, or at least about 90%, or about 91%, or about 92%, or about 93%, or about 94%, or about 95%, or about 96%, or about 97%, or about 98%, or about 99% to about 100% of the sequence consists of multiple units of two or more non-overlapping sequence motifs selected from the amino acid sequences of Table 2. In some cases, the XTEN comprises non-overlapping sequence motifs in which about 80%, or at least about 90%, or about 91%, or about 92%, or about 93%, or about 94%, or about 95%, or about 96%, or about 97%, or about 98%, or about 99% to about 100% of the sequence consists of two or more non-overlapping sequences selected from a single motif family of Table 2, resulting in a “family” sequence in which the overall sequence remains substantially non-repetitive. Accordingly, in these embodiments, an XTEN sequence can comprise multiple units of non-overlapping sequence motifs of the AD motif family, or the AE motif family, or the AF motif family, or the AG motif family, or the AM motif family, or the AQ motif family, or the BC family, or the BD family of sequences of Table 2. In other cases, the XTEN comprises motif sequences from two or more of the motif families of Table 2.

TABLE 2
XTEN Sequence Motifs of 12 Amino Acids and Motif
Families

| | SEQ | |
|---|---|---|
| Motif Family* | ID NO: | Motif Sequence |
| AD | 34 | GESPGGSSGSES |
| AD | 35 | GSEGSSGPGESS |
| AD | 36 | GSSESGSSEGGP |
| AD | 37 | GSGGEPSESGSS |
| AE, AM | 38 | GSPAGSPTSTEE |
| AE, AM, AQ | 39 | GSEPATSGSETP |
| AE, AM, AQ | 40 | GTSESATPESGP |
| AE, AM, AQ | 41 | GTSTEPSEGSAP |
| AF, AM | 42 | GSTSESPSGTAP |
| AF, AM | 43 | GTSTPESGSASP |
| AF, AM | 44 | GTSPSGESSTAP |
| AF, AM | 45 | GSTSSTAESPGP |
| AG, AM | 46 | GTPGSGTASSSP |
| AG, AM | 47 | GSSTPSGATGSP |
| AG, AM | 48 | GSSPSASTGTGP |
| AG, AM | 49 | GASPGTSSTGSP |
| AQ | 50 | GEPAGSPTSTSE |
| AQ | 51 | GTGEPSSTPASE |
| AQ | 52 | GSGPSTESAPTE |
| AQ | 53 | GSETPSGPSETA |
| AQ | 54 | GPSETSTSEPGA |
| AQ | 55 | GSPSEPTEGTSA |
| BC | 56 | GSGASEPTSTEP |
| BC | 57 | GSEPATSGTEPS |
| BC | 58 | GTSEPSTSEPGA |
| BC | 59 | GTSTEPSEPGSA |
| BD | 60 | GSTAGSETSTEA |
| BD | 61 | GSETATSGSETA |
| BD | 62 | GTSESATSESGA |
| BD | 63 | GTSTEASEGSAS |

*Denotes individual motif sequences that, when used together in various permutations, result in a “family sequence”

In other cases, C-peptide-XTEN composition can comprise a non-repetitive XTEN sequence of greater than about 100 to about 3000 amino acid residues, preferably greater than 400 to about 3000 residues, wherein at least about 80%, or at least about 90%, or about 91%, or about 92%, or about 93%, or about 94%, or about 95%, or about 96%, or about 97%, or about 98%, or about 99% to about 100% of the sequence consists of non-overlapping 36 amino acid sequence motifs selected from one or more of the polypeptide sequences of Tables 12-15 of US2010/0239554, which is hereby incorporated by reference. See Examples 1-4 for the construction of the 36 amino acid sequence motifs.

In those embodiments wherein the XTEN component of the C-peptide-XTEN fusion protein has less than 100% of its amino acids consisting of four to six amino acid selected from glycine (G), alanine (A), serine (S), threonine (T), glutamate (E) and proline (P), or less than 100% of the sequence consisting of the sequence motifs of Table 2 or the polypeptide sequences to Tables 12-15 of US 2010/0239554, which is hereby incorporated by reference, or less than 100% sequence identity with an XTEN from Table 2, the other amino acid residues can be selected from any other of the 14 natural L-amino acids. The other amino acids may be interspersed throughout the XTEN sequence, may be located within or between the sequence motifs, or may be concentrated in one or more short stretches of the XTEN sequence. In such cases where the XTEN component of the C-peptide-XTEN comprises amino acids other than glycine (G), alanine (A), serine (S), threonine (T), glutamate (E) and proline (P), it is preferred that the amino acids not be hydrophobic residues and should not substantially confer secondary structure of the XTEN component. Thus, in a preferred embodiment of the foregoing, the XTEN component of the C-peptide-XTEN fusion protein comprising other amino adds in addition to glycine (G), alanine (A), serine (S), threonine (T), glutamate (E) and proline (P) would have a sequence with less than 5% of the residues contributing to alpha-helices and beta-sheets as measured by Chou-Fasman algorithm and would have at least 90% random coil formation as measured by GOR algorithm (see, e.g., Example 40).

In a particular feature, the invention encompasses C-peptide-XTEN compositions comprising XTEN polypeptides with extended length sequences. Increasing the length of non-repetitive, unstructured polypeptides enhances the unstructured nature of the XTENs and the biological and pharmacokinetic properties of fusion proteins comprising the XTEN. Proportional increases in the length of the XTEN, even if created by a fixed repeat order of single family sequence motifs (e.g., the four AE motifs of Table 2), can result in a sequence with a higher percentage of random coil formation, as determined by GOR algorithm, compared to shorter XTEN lengths. In addition, increasing the length of the unstructured polypeptide fusion partner can result in a fusion protein with a disproportional increase in terminal half-life compared to fusion proteins with unstructured polypeptide partners with shorter sequence lengths. See Examples 5-13 for construction of XTEN polypeptides with extended length sequences.

Non-limiting examples of XTEN contemplated for inclusion in the C-peptide-XTEN of the invention are presented in Table 3. Accordingly, the invention provides C-peptide-XTEN compositions wherein the XTEN sequence length of the fusion protein(s) is greater than about 100 to about 3000 amino acid residues, and in some cases is greater than 400 to about 3000 amino acid residues, wherein the XTEN confers enhanced pharmacokinetic properties on the C-peptide-XTEN in comparison to payloads not linked to XTEN. In some cases, the XTEN sequences of the C-peptide-XTEN compositions of the present invention can be about 100, or about 144, or about 288, or about 401, or about 500, or about 600, or about 700, or about 800, or about 900, or about 1000, or about 1500, or about 2000, or about 2500 or up to about 3000 amino acid residues in length. In other cases, the XTEN sequences can be about 100 to 150, about 150 to 250, about 250 to 400, 401 to about 500, about 500 to 900, about 900 to 1500, about 1500 to 2000, or about 2000 to about 3000 amino acid residues in length. In one embodiment, the C-peptide-XTEN can comprise an XTEN sequence wherein the sequence exhibits at least about 80% sequence identity, or alternatively 81%, 82%, 83%, 84%, 85%, 86%, 87%, 88%, 89%, 90%, 91%, 92%, 93%, 94%, 95%, 96%, 97%, 98%, 99%, or 100% sequence identity to a XTEN selected from Table 3. In some cases, the XTEN sequence is designed for optimized expression as the N-terminal component of the C-peptide-XTEN. In one embodiment of the foregoing, the XTEN sequence has at least 90% sequence identity to the sequence of AE912 or AM923. In another embodiment of the foregoing, the XTEN has the N-terminal residues described in Examples 14-17.

In other cases, the C-peptide-XTEN fusion protein can comprise a first and a second XTEN sequence, wherein the cumulative total of the residues in the XTEN sequences is greater than about 400 to about 3000 amino acid residues. In embodiments of the foregoing, the C-peptide-XTEN fusion protein can comprise a first and a second XTEN sequence wherein the sequences each exhibit at least about 80% sequence identity, or alternatively 81%, 82%, 83%, 84%, 85%, 86%, 87%, 88%, 89%, 90%, 91%, 92%, 93%, 94%, 95%, 96%, 97%, 98%, 99%, or 100% sequence identity to at least a first or additionally a second XTEN selected from Table 3. Examples where more than one XTEN is used in a C-peptide-XTEN composition include, but are not limited to constructs with an XTEN linked to both the N- and C-termini of at least one C-peptide.

As described more fully below, the invention provides methods in which the C-peptide-XTEN is designed by selecting the length of the XTEN to confer a target half-life on a fusion protein administered to a subject. In general, longer XTEN lengths incorporated into the C-peptide-XTEN compositions result in longer half-life compared to shorter XTEN. However, in another embodiment, C-peptide-XTEN fusion proteins can be designed to comprise XTEN with a longer sequence length that is selected to confer slower rates of systemic absorption after subcutaneous or intramuscular administration to a subject. In such cases, the Cₘₐₓ is reduced in comparison to a comparable dose of a C-peptide not linked to XTEN, thereby contributing to the ability to keep the C-peptide-XTEN within the therapeutic window for the composition. Thus, the XTEN confers the property of a depot to the administered C-peptide-XTEN, in addition to the other physical/chemical properties described herein.

TABLE 3
XTEN Polypeptides

| XTEN | SEQ | |
|---|---|---|
| Name | ID NO | Amino Acid Sequence |
| AF504 | 64 | GASPGTSSTGSPGSSPSASTGTGPGSSPSASTGTGPGTPGSGTASSSPGSSTPSGATG |
| | | SPGSXPSASTGTGPGASPGTSSTGSPGTPGSGTASSSPGSSTPSGATGSPGTPGSGTA |
| | | SSSPGASPGTSSTGSPGASPGTSSTGSPGTPGSGTASSSPGSSTPSGATGSPGASPGT |
| | | SSTGSPGTPGSGTASSSPGSSTPSGATGSPGSXPSASTGTGPGSSPSASTGTGPGSST |
| | | PSGATGSPGSSTPSGATGSPGASPGTSSTGSPGASPGTSSTGSPGASPGTSSTGSPGT |
| | | PGSGTASSSPGASPGTSSTGSPGASPGTSSTGSPGASPGTSSTGSPGSSPSASTGTGP |
| | | GTPGSGTASSSPGASPGTSSTGSPGASPGTSSTGSPGASPGTSSTGSPGSSTPSGATG |
| | | SPGSSTPSGATGSPGASPGTSSTGSPGTPGSGTASSSPGSSTPSGATGSPGSSTPSGA |
| | | TGSPGSSTPSGATGSPGSSPSASTGTGPGASPGTSSTGSP |
| AF540 | 65 | GSTSSTAESPGPGSTSSTAESPGPGSTSESPSGTAPGSTSSTAESPGPGSTSSTAESP |
| | | GPGTSTPESGSASPGSTSESPSGTAPGTSPSGESSTAPGSTSESPSGTAPGSTSESPS |
| | | GTAPGTSPSGESSTAPGSTSESPSGTAPGSTSESPSGTAPGTSPSGESSTAPGSTSES |
| | | PSGTAPGSTSESPSGTAPGSTSESPSGTAPGTSTPESGSASPGSTSESPSGTAPGTST |
| | | PESGSASPGSTSSTAESPGPGSTSSTAESPGPGTSTPESGSASPGTSTPESGSASPGS |
| | | TSESPSGTAPGTSTPESGSASPGTSTPESGSASPGSTSESPSGTAPGSTSESPSGTAP |
| | | GSTSESPSGTAPGSTSSTAESPGPGTSTPESGSASPGTSTPESGSASPGSTSESPSGT |
| | | APGSTSESPSGTAPGTSTPESGSASPGSTSESPSGTAPGSTSESPSGTAPGTSTPESG |
| | | SASPGTSPSGESSTAPGSTSSTAESPGPGTSPSGESSTAPGSTSSTAESPGPGTSTPE |
| | | SGSASPGSTSESPSGTAP |
| AD576 | 66 | GSSESGSSEGGPGSGGEPSESGSSGSSESGSSEGGPGSSESGSSEGGPGSSESGSSEG |
| | | GPGSSESGSSEGGPGSSESGSSEGGPGESPGGSSGSESGSEGSSGPGESSGSSESGSS |
| | | EGGPGSSESGSSEGGPGSSESGSSEGGPGSGGEPSESGSSGESPGGSSGSESGESPGG |
| | | SSGSESGSGGEPSESGSSGSSESGSSEGGPGSGGEPSESGSSGSGGEPSESGSSGSEG |
| | | SSGPGESSGESPGGSSGSESGSGGEPSESGSSGSGGEPSESGSSGSGGEPSESGSSGS |
| | | SESGSSEGGPGESPGGSSGSESGESPGGSSGSESGESPGGSSGSESGESPGGSSGSES |
| | | GESPGGSSGSESGSSESGSSEGGPGSGGEPSESGSSGSEGSSGPGESSGSSESGSSEG |
| | | GPGSGGEPSESGSSGSSESGSSEGGPGSGGEPSESGSSGESPGGSSGSESGESPGGSS |
| | | GSESGSSESGSSEGGPGSGGEPSESGSSGSSESGSSEGGPGSGGEPSESGSSGSGGEP |
| | | SESGSSGESPGGSSGSESGSEGSSGPGESSGSSESGSSEGGPGSEGSSGPGESS |
| AE576 | 67 | GSPAGSPTSTEEGTSESATPESGPGTSTEPSEGSAPGSPAGSPTSTEEGTSTEPSEGS |
| | | APGTSTEPSEGSAPGTSESATPESGPGSEPATSGSETPGSEPATSGSETPGSPAGSPT |
| | | STEEGTSESATPESGPGTSTEPSEGSAPGTSTEPSEGSAPGSPAGSPTSTEEGTSTEP |
| | | SEGSAPGTSTEPSEGSAPGTSESATPESGPGTSTEPSEGSAPGTSESATPESGPGSEP |
| | | ATSGSETPGTSTEPSEGSAPGTSTEPSEGSAPGTSESATPESGPGTSESATPESGPGS |
| | | PAGSPTSTEEGTSESATPESGPGSEPATSGSETPGTSESATPESGPGTSTEPSEGSAP |
| | | GTSTEPSEGSAPGTSTEPSEGSAPGTSTEPSEGSAPGTSTEPSEGSAPGTSTEPSEGS |
| | | APGSPAGSPTSTEEGTSTEPSEGSAPGTSESATPESGPGSEPATSGSETPGTSESATP |
| | | ESGPGSEPATSGSETPGTSESATPESGPGTSTEPSEGSAPGTSESATPESGPGSPAGS |
| | | PTSTEEGSPAGSPTSTEEGSPAGSPTSTEEGTSESATPESGPGTSTEPSEGSAP |
| AF576 | 68 | GSTSSTAESPGPGSTSSTAESPGPGSTSESPSGTAPGSTSSTAESPGPGSTSSTAESP |
| | | GPGTSTPESGSASPGSTSESPSGTAPGTSPSGESSTAPGSTSESPSGTAPGSTSESPS |
| | | GTAPGTSPSGESSTAPGSTSESPSGTAPGSTSESPSGTAPGTSPSGESSTAPGSTSES |
| | | PSGTAPGSTSESPSGTAPGSTSESPSGTAPGTSTPESGSASPGSTSESPSGTAPGTST |
| | | PESGSASPGSTSSTAESPGPGSTSSTAESPGPGTSTPESGSASPGTSTPESGSASPGS |
| | | TSESPSGTAPGTSTPESGSASPGTSTPESGSASPGSTSESPSGTAPGSTSESPSGTAP |
| | | GSTSESPSGTAPGSTSSTAESPGPGTSTPESGSASPGTSTPESGSASPGSTSESPSGT |
| | | APGSTSESPSGTAPGTSTPESGSASPGSTSESPSGTAPGSTSESPSGTAPGTSTPESG |
| | | SASPGTSPSGESSTAPGSTSSTAESPGPGTSPSGESSTAPGSTSSTAESPGPGTSTPE |
| | | SGSASPGSTSESPSGTAPGSTSSTAESPGPGTSTPESGSASPGTSTPESGSASP |
| AD836 | 69 | GSSESGSSEGGPGSSESGSSEGGPGESPGGSSGSESGSGGEPSESGSSGESPGGSSGS |
| | | ESGESPGGSSGSESGSSESGSSEGGPGSSESGSSEGGPGSSESGSSEGGPGESPGGSS |
| | | GSESGESPGGSSGSESGESPGGSSGSESGSSESGSSEGGPGSSESGSSEGGPGSSESG |
| | | SSEGGPGSSESGSSEGGPGSSESGSSEGGPGSSESGSSEGGPGSGGEPSESGSSGESP |
| | | GGSSGSESGESPGGSSGSESGSGGEPSESGSSGSEGSSGPGESSGSSESGSSEGGPGS |
| | | GGEPSESGSSGSEGSSGPGESSGSSESGSSEGGPGSGGEPSESGSSGESPGGSSGSES |
| | | GSGGEPSESGSSGSGGEPSESGSSGSSESGSSEGGPGSGGEPSESGSSGSGGEPSESG |
| | | SSGSEGSSGPGESSGESPGGSSGSESGSEGSSGPGESSGSEGSSGPGESSGSGGEPSE |
| | | SGSSGSSESGSSEGGPGSSESGSSEGGPGESPGGSSGSESGSGGEPSESGSSGSEGSS |
| | | GPGESSGESPGGSSGSESGSEGSSGPGSSESGSSEGGPGSGGEPSESGSSGSEGSSGP |
| | | GESSGSEGSSGPGESSGSEGSSGPGESSGSGGEPSESGSSGSGGEPSESGSSGESPGG |
| | | SSGSESGESPGGSSGSESGSGGEPSESGSSGSEGSSGPGESSGESPGGSSGSESGSSE |
| | | SGSSEGGPGSSESGSSEGGPGSSESGSSEGGPGSGGEPSESGSSGSSESGSSEGGPGE |
| | | SPGGSSGSESGSGGEPSESGSSGSSESGSSEGGPGESPGGSSGSESGSGGEPSESGSS |
| | | GESPGGSSGSESGSGGEPSESGSS |
| AE864 | 70 | GSPAGSPTSTEEGTSESATPESGPGTSTEPSEGSAPGSPAGSPTSTEEGTSTEPSEGS |
| | | APGTSTEPSEGSAPGTSESATPESGPGSEPATSGSETPGSEPATSGSETPGSPAGSPT |
| | | STEEGTSESATPESGPGTSTEPSEGSAPGTSTEPSEGSAPGSPAGSPTSTEEGTSTEP |
| | | SEGSAPGTSTEPSEGSAPGTSESATPESGPGTSTEPSEGSAPGTSESATPESGPGSEP |
| | | ATSGSETPGTSTEPSEGSAPGTSTEPSEGSAPGTSESATPESGPGTSESATPESGPGS |
| | | PAGSPTSTEEGTSESATPESGPGSEPATSGSETPGTSESATPESGPGTSTEPSEGSAP |
| | | GTSTEPSEGSAPGTSTEPSEGSAPGTSTEPSEGSAPGTSTEPSEGSAPGTSTEPSEGS |
| | | APGSPAGSPTSTEEGTSTEPSEGSAPGTSESATPESGPGSEPATSGSETPGTSESATP |
| | | ESGPGSEPATSGSETPGTSESATPESGPGTSTEPSEGSAPGTSESATPESGPGSPAGS |
| | | PTSTEEGSPAGSPTSTEEGSPAGSPTSTEEGTSESATPESGPGTSTEPSEGSAPGTSE |
| | | SATPESGPGSEPATSGSETPGTSESATPESGPGSEPATSGSETPGTSESATPESGPGT |
| | | STEPSEGSAPGSPAGSPTSTEEGTSESATPESGPGSEPATSGSETPGTSESATPESGP |
| | | GSPAGSPTSTEEGSPAGSPTSTEEGTSTEPSEGSAPGTSESATPESGPGTSESATPES |
| | | GPGTSESATPESGPGSEPATSGSETPGSEPATSGSETPGSPAGSPTSTEEGTSTEPSE |
| | | GSAPGTSTEPSEGSAPGSEPATSGSETPGTSESATPESGPGTSTEPSEGSAP |
| AF864 | 71 | GSTSESPSGTAPGTSPSGESSTAPGSTSESPSGTAPGSTSESPSGTAPGTSTPESGSA |
| | | SPGTSTPESGSASPGSTSESPSGTAPGSTSESPSGTAPGTSPSGESSTAPGSTSESPS |
| | | GTAPGTSPSGESSTAPGTSPSGESSTAPGSTSSTAESPGPGTSPSGESSTAPGTSPSG |
| | | ESSTAPGSTSSTAESPGPGTSTPESGSASPGTSTPESGSASPGSTSESPSGTAPGSTS |
| | | ESPSGTAPGTSTPESGSASPGSTSSTAESPGPGTSTPESGSASPGSTSESPSGTAPGT |
| | | SPSGESSTAPGSTSSTAESPGPGTSPSGESSTAPGTSTPESGSASPGSTSSTAESPGP |
| | | GSTSSTAESPGPGSTSSTAESPGPGSTSSTAESPGPGTSPSGESSTAPGSTSESPSGT |
| | | APGSTSESPSGTAPGTSTPESGPXXXGASASGAPSTXXXXSESPSGTAPGSTSESPSG |
| | | TAPGSTSESPSGTAPGSTSESPSGTAPGSTSESPSGTAPGSTSESPSGTAPGTSTPES |
| | | GSASPGTSPSGESSTAPGTSPSGESSTAPGSTSSTAESPGPGTSPSGESSTAPGTSTP |
| | | ESGSASPGSTSESPSGTAPGSTSESPSGTAPGTSPSGESSTAPGSTSESPSGTAPGTS |
| | | TPESGSASPGTSTPESGSASPGSTSESPSGTAPGTSTPESGSASPGSTSSTAESPGPG |
| | | STSESPSGTAPGSTSESPSGTAPGTSPSGESSTAPGSTSSTAESPGPGTSPSGESSTA |
| | | PGTSTPESGSASPGTSPSGESSTAPGTSPSGESSTAPGTSPSGESSTAPGSTSSTAES |
| | | PGPGSTSSTAESPGPGTSPSGESSTAPGSSPSASTGTGPGSSTPSGATGSPGSSTPSG |
| | | ATGSP |
| AG864 | 72 | GASPGTSSTGSPGSSPSASTGTGPGSSPSASTGTGPGTPGSGTASSSPGSSTPSGATG |
| | | SPGSSPSASTGTGPGASPGTSSTGSPGTPGSGTASSSPGSSTPSGATGSPGTPGSGTA |
| | | SSSPGASPGTSSTGSPGASPGTSSTGSPGTPGSGTASSSPGSSTPSGATGSPGASPGT |
| | | SSTGSPGTPGSGTASSSPGSSTPSGATGSPGSSPSASTGTGPGSSPSASTGTGPGSST |
| | | PSGATGSPGSSTPSGATGSPGASPGTSSTGSPGASPGTSSTGSPGASPGTSSTGSPGT |
| | | PGSGTASSSPGASPGTSSTGSPGASPGTSSTGSPGASPGTSSTGSPGSSPSASTGTGP |
| | | GTPGSGTASSSPGASPGTSSTGSPGASPGTSSTGSPGASPGTSSTGSPGSSTPSGATG |
| | | SPGSSTPSGATGSPGASPGTSSTGSPGTPGSGTASSSPGSSTPSGATGSPGSSTPSGA |
| | | TGSPGSSTPSGATGSPGSSPSASTGTGPGASPGTSSTGSPGASPGTSSTGSPGTPGSG |
| | | TASSSPGASPGTSSTGSPGASPGTSSTGSPGASPGTSSTGSPGASPGTSSTGSPGTPG |
| | | SGTASSSPGSSTPSGATGSPGTPGSGTASSSPGSSTPSGATGSPGTPGSGTASSSPGS |
| | | STPSGATGSPGSSTPSGATGSPGSSPSASTGTGPGSSPSASTGTGPGASPGTSSTGSP |
| | | GTPGSGTASSSPGSSTPSGATGSPGSSPSASTGTGPGSSPSASTGTGPGASPGTSSTG |
| | | SPGASPGTSSTGSPGSSTPSGATGSPGSSPSASTGTGPGASPGTSSTGSPGSSPSAST |
| | | GTGPGTPGSGTASSSPGSSTPSGATGSPGSSTPSGATGSPGASPGTSSTGSP |
| AM875 | 73 | GTSTEPSEGSAPGSEPATSGSETPGSPAGSPTSTEEGSTSSTAESPGPGTSTPESGSA |
| | | SPGSTSESPSGTAPGSTSESPSGTAPGTSTPESGSASPGTSTPESGSASPGSEPATSG |
| | | SETPGTSESATPESGPGSPAGSPTSTEEGTSTEPSEGSAPGTSESATPESGPGTSTEP |
| | | SEGSAPGTSTEPSEGSAPGSPAGSPTSTEEGTSTEPSEGSAPGTSTEPSEGSAPGTSE |
| | | SATPESGPGTSESATPESGPGTSTEPSEGSAPGTSTEPSEGSAPGTSESATPESGPGT |
| | | STEPSEGSAPGSEPATSGSETPGSPAGSPTSTEEGSSTPSGATGSPGTPGSGTASSSP |
| | | GSSTPSGATGSPGTSTEPSEGSAPGTSTEPSEGSAPGSEPATSGSETPGSPAGSPTST |
| | | EEGSPAGSPTSTEEGTSTEPSEGSAPGASASGAPSTGGTSESATPESGPGSPAGSPTS |
| | | TEEGSPAGSPTSTEEGSTSSTAESPGPGSTSESPSGTAPGTSPSGESSTAPGTPGSGT |
| | | ASSSPGSSTPSGATGSPGSSPSASTGTGPGSEPATSGSETPGTSESATPESGPGSEPA |
| | | TSGSETPGSTSSTAESPGPGSTSSTAESPGPGTSPSGESSTAPGSEPATSGSETPGSE |
| | | PATSGSETPGTSTEPSEGSAPGSTSSTAESPGPGTSTPESGSASPGSTSESPSGTAPG |
| | | TSTEPSEGSAPGTSTEPSEGSAPGTSTEPSEGSAPGSSTPSGATGSPGSSPSASTGTG |
| | | PGASPGTSSTGSPGSEPATSGSETPGTSESATPESGPGSPAGSPTSTEEGSSTPSGAT |
| | | GSPGSSPSASTGTGPGASPGTSSTGSPGTSESATPESGPGTSTEPSEGSAPGTSTEPS |
| | | EGSAP |
| AE912 | 74 | MAEPAGSPTSTEEGTPGSGTASSSPGSSTPSGATGSPGASPGTSSTGSPGSPAGSPTS |
| | | TEEGTSESATPESGPGTSTEPSEGSAPGSPAGSPTSTEEGTSTEPSEGSAPGTSTEPS |
| | | EGSAPGTSESATPESGPGSEPATSGSETPGSEPATSGSETPGSPAGSPTSTEEGTSES |
| | | ATPESGPGTSTEPSEGSAPGTSTEPSEGSAPGSPAGSPTSTEEGTSTEPSEGSAPGTS |
| | | TEPSEGSAPGTSESATPESGPGTSTEPSEGSAPGTSESATPESGPGSEPATSGSETPG |
| | | TSTEPSEGSAPGTSTEPSEGSAPGTSESATPESGPGTSESATPESGPGSPAGSPTSTE |
| | | EGTSESATPESGPGSEPATSGSETPGTSESATPESGPGTSTEPSEGSAPGTSTEPSEG |
| | | SAPGTSTEPSEGSAPGTSTEPSEGSAPGTSTEPSEGSAPGTSTEPSEGSAPGSPAGSP |
| | | TSTEEGTSTEPSEGSAPGTSESATPESGPGSEPATSGSETPGTSESATPESGPGSEPA |
| | | TSGSETPGTSESATPESGPGTSTEPSEGSAPGTSESATPESGPGSPAGSPTSTEEGSP |
| | | AGSPTSTEEGSPAGSPTSTEEGTSESATPESGPGTSTEPSEGSAPGTSESATPESGPG |
| | | SEPATSGSETPGTSESATPESGPGSEPATSGSETPGTSESATPESGPGTSTEPSEGSA |
| | | PGSPAGSPTSTEEGTSESATPESGPGSEPATSGSETPGTSESATPESGPGSPAGSPTS |
| | | TEEGSPAGSPTSTEEGTSTEPSEGSAPGTSESATPESGPGTSESATPESGPGTSESAT |
| | | PESGPGSEPATSGSETPGSEPATSGSETPGSPAGSPTSTEEGTSTEPSEGSAPGTSTE |
| | | PSEGSAPGSEPATSGSETPGTSESATPESGPGTSTEPSEGSAP |
| AM923 | 75 | MAEPAGSPTSTEEGASPGTSSTGSPGSSTPSGATGSPGSSTPSGATGSPGTSTEPSEG |
| | | SAPGSEPATSGSETPGSPAGSPTSTEEGSTSSTAESPGPGTSTPESGSASPGSTSESP |
| | | SGTAPGSTSESPSGTAPGTSTPESGSASPGTSTPESGSASPGSEPATSGSETPGTSES |
| | | ATPESGPGSPAGSPTSTEEGTSTEPSEGSAPGTSESATPESGPGTSTEPSEGSAPGTS |
| | | TEPSEGSAPGSPAGSPTSTEEGTSTEPSEGSAPGTSTEPSEGSAPGTSESATPESGPG |
| | | TSESATPESGPGTSTEPSEGSAPGTSTEPSEGSAPGTSESATPESGPGTSTEPSEGSA |
| | | PGSEPATSGSETPGSPAGSPTSTEEGSSTPSGATGSPGTPGSGTASSSPGSSTPSGAT |
| | | GSPGTSTEPSEGSAPGTSTEPSEGSAPGSEPATSGSETPGSPAGSPTSTEEGSPAGSP |
| | | TSTEEGTSTEPSEGSAPGASASGAPSTGGTSESATPESGPGSPAGSPTSTEEGSPAGS |
| | | PTSTEEGSTSSTAESPGPGSTSESPSGTAPGTSPSGESSTAPGTPGSGTASSSPGSST |
| | | PSGATGSPGSSPSASTGTGPGSEPATSGSETPGTSESATPESGPGSEPATSGSETPGS |
| | | TSSTAESPGPGSTSSTAESPGPGTSPSGESSTAPGSEPATSGSETPGSEPATSGSETP |
| | | GTSTEPSEGSAPGSTSSTAESPGPGTSTPESGSASPGSTSESPSGTAPGTSTEPSEGS |
| | | APGTSTEPSEGSAPGTSTEPSEGSAPGSSTPSGATGSPGSSPSASTGTGPGASPGTSS |
| | | TGSPGSEPATSGSETPGTSESATPESGPGSPAGSPTSTEEGSSTPSGATGSPGSSPSA |
| | | STGTGPGASPGTSSTGSPGTSESATPESGPGTSTEPSEGSAPGTSTEPSEGSAP |
| AM1296 | 76 | GTSTEPSEGSAPGSEPATSGSETPGSPAGSPTSTEEGSTSSTAESPGPGTSTPESGSA |
| | | SPGSTSESPSGTAPGSTSESPSGTAPGTSTPESGSASPGTSTPESGSASPGSEPATSG |
| | | SETPGTSESATPESGPGSPAGSPTSTEEGTSTEPSEGSAPGTSESATPESGPGTSTEP |
| | | SEGSAPGTSTEPSEGSAPGSPAGSPTSTEEGTSTEPSEGSAPGTSTEPSEGSAPGTSE |
| | | SATPESGPGTSESATPESGPGTSTEPSEGSAPGTSTEPSEGSAPGTSESATPESGPGT |
| | | STEPSEGSAPGSEPATSGSETPGSPAGSPTSTEEGSSTPSGATGSPGTPGSGTASSSP |
| | | GSSTPSGATGSPGTSTEPSEGSAPGTSTEPSEGSAPGSEPATSGSETPGSPAGSPTST |
| | | EEGSPAGSPTSTEEGTSTEPSEGSAPGPEPTGPAPSGGSEPATSGSETPGTSESATPE |
| | | SGPGSPAGSPTSTEEGTSESATPESGPGSPAGSPTSTEEGSPAGSPTSTEEGTSESAT |
| | | PESGPGSPAGSPTSTEEGSPAGSPTSTEEGSTSSTAESPGPGSTSESPSGTAPGTSPS |
| | | GESSTAPGSTSESPSGTAPGSTSESPSGTAPGTSPSGESSTAPGTSTEPSEGSAPGTS |
| | | ESATPESGPGTSESATPESGPGSEPATSGSETPGTSESATPESGPGTSESATPESGPG |
| | | TSTEPSEGSAPGTSESATPESGPGTSTEPSEGSAPGTSPSGESSTAPGTSPSGESSTA |
| | | PGTSPSGESSTAPGTSTEPSEGSAPGSPAGSPTSTEEGTSTEPSEGSAPGSSPSASTG |
| | | TGPGSSTPSGATGSPGSSTPSGATGSPGSSTPSGATGSPGSSTPSGATGSPGASPGTS |
| | | STGSPGASASGAPSTGGTSPSGESSTAPGSTSSTAESPGPGTSPSGESSTAPGTSESA |
| | | TPESGPGTSTEPSEGSAPGTSTEPSEGSAPGSSPSASTGTGPGSSTPSGATGSPGASP |
| | | GTSSTGSPGTSTPESGSASPGTSPSGESSTAPGTSPSGESSTAPGTSESATPESGPGS |
| | | EPATSGSETPGTSTEPSEGSAPGSTSESPSGTAPGSTSESPSGTAPGTSTPESGSASP |
| | | GSPAGSPTSTEEGTSESATPESGPGTSTEPSEGSAPGSPAGSPTSTEEGTSESATPES |
| | | GPGSEPATSGSETPGSSTPSGATGSPGASPGTSSTGSPGSSTPSGATGSPGSTSESPS |
| | | GTAPGTSPSGESSTAPGSTSSTAESPGPGSSTPSGATGSPGASPGTSSTGSPGTPGSG |
| | | TASSSPGSPAGSPTSTEEGSPGSPTSTEEGTSTEPSEGSAP |
| BC864 | 77 | GTSTEPSEPGSAGTSTEPSEPGSAGSEPATSGTEPSGSGASEPTSTEPGSEPATSGTE |
| | | PSGSEPATSGTEPSGSEPATSGTEPSGSGASEPTSTEPGTSTEPSEPGSAGSEPATSG |
| | | TEPSGTSTEPSEPGSAGSEPATSGTEPSGSEPATSGTEPSGTSTEPSEPGSAGTSTEP |
| | | SEPGSAGSEPATSGTEPSGSEPATSGTEPSGTSEPSTSEPGAGSGASEPTSTEPGTSE |
| | | PSTSEPGAGSEPATSGTEPSGSEPATSGTEPSGTSTEPSEPGSAGTSTEPSEPGSAGS |
| | | GASEPTSTEPGSEPATSGTEPSGSEPATSGTEPSGSEPATSGTEPSGSEPATSGTEPS |
| | | GTSTEPSEPGSAGSEPATSGTEPSGSGASEPTSTEPGTSTEPSEPGSAGSEPATSGTE |
| | | PSGSGASEPTSTEPGTSTEPSEPGSAGSGASEPTSTEPGSEPATSGTEPSGSGASEPT |
| | | STEPGSEPATSGTEPSGSGASEPTSTEPGTSTEPSEPGSAGSEPATSGTEPSGSGASE |
| | | PTSTEPGTSTEPSEPGSAGSEPATSGTEPSGTSTEPSEPGSAGSEPATSGTEPSGTST |
| | | EPSEPGSAGTSTEPSEPGSAGTSTEPSEPGSAGTSTEPSEPGSAGTSTEPSEPGSAGT |
| | | STEPSEPGSAGTSEPSTSEPGAGSGASEPTSTEPGTSTEPSEPGSAGTSTEPSEPGSA |
| | | GTSTEPSEPGSAGSEPATSGTEPSGSGASEPTSTEPGSEPATSGTEPSGSEPATSGTE |
| | | PSGSEPATSGTEPSGSEPATSGTEPSGTSEPSTSEPGAGSEPATSGTEPSGSGASEPT |
| | | STEPGTSTEPSEPGSAGSEPATSGTEPSGSGASEPTSTEPGTSTEPSEPGSA |
| BD864 | 78 | GSETATSGSETAGTSESATSESGAGSTAGSETSTEAGTSESATSESGAGSETATSGSE |
| | | TAGSETATSGSETAGTSTEASEGSASGTSTEASEGSASGTSESATSESGAGSETATSG |
| | | SETAGTSTEASEGSASGSTAGSETSTEAGTSESATSESGAGTSESATSESGAGSETAT |
| | | SGSETAGTSESATSESGAGTSTEASEGSASGSETATSGSETAGSETATSGSETAGTST |
| | | EASEGSASGSTAGSETSTEAGTSESATSESGAGTSTEASEGSASGSETATSGSETAGS |
| | | TAGSETSTEAGSTAGSETSTEAGSETATSGSETAGTSESATSESGAGTSESATSESGA |
| | | GSETATSGSETAGTSESATSESGAGTSESATSESGAGSETATSGSETAGSETATSGSE |
| | | TAGTSTEASEGSASGSTAGSETSTEAGSETATSGSETAGTSESATSESGAGSTAGSET |
| | | STEAGSTAGSETSTEAGSTAGSETSTEAGTSTEASEGSASGSTAGSETSTEAGSTAGS |
| | | ETSTEAGTSTEASEGSASGSTAGSETSTEAGSETATSGSETAGTSTEASEGSASGTSE |
| | | SATSESGAGSETATSGSETAGTSESATSESGAGTSESATSESGAGSETATSGSETAGT |
| | | SESATSESGAGSETATSGSETAGTSTEASEGSASGTSTEASEGSASGSTAGSETSTEA |
| | | GSTAGSETSTEAGSETATSGSETAGTSESATSESGAGTSESATSESGAGSETATSGSE |
| | | TAGSETATSGSETAGSETATSGSETAGTSTEASEGSASGTSESATSESGAGSETATSG |
| | | SETAGSETATSGSETAGTSESATSESGAGTSESATSESGAGSETATSGSETA |

In other cases, the XTEN polypeptides can have an unstructured characteristic imparted by incorporation of amino acid residues with a net charge and/or reducing the proportion of hydrophobic amino acids in the XTEN sequence. The overall net charge and net charge density may be controlled by modifying the content of charged amino acids in the XTEN sequences. In some cases, the net charge density of the XTEN of the compositions may be above +0.1 or below −0.1 charges/residue. In other cases, the net charge of a XTEN can be about 0%, about 1%, about 2%, about 3%, about 4%, about 5%, about 6%, about 7%, about 8%, about 9%, about 10% about 11%, about 12%, about 13%, about 14%, about 15%, about 16%, about 17%, about 18%, about 19%, or about 20% or more.

Since most tissues and surfaces in a human or animal have a net negative charge, the XTEN sequences can be designed to have a net negative charge to minimize nonspecific interactions between the XTEN containing compositions and various surfaces such as blood vessels, healthy tissues, or various receptors. Not to be bound by a particular theory, the XTEN can adopt open conformations due to electrostatic repulsion between individual amino acids of the XTEN polypeptide that individually carry a high net negative charge and that are distributed across the sequence of the XTEN polypeptide. Such a distribution of net negative charge in the extended sequence lengths of XTEN can lead to an unstructured conformation that, in turn, can result in an effective increase in hydrodynamic radius. Accordingly, in one embodiment the invention provides XTEN in which the XTEN sequences contain about 8, 10, 15, 20, 25, or even about 30% glutamic acid. The XTEN of the compositions of the present invention generally have no or a low content of positively charged amino acids. In some cases the XTEN may have less than about 10% amino acid residues with a positive charge, or less than about 7%, or less than about 5%, or less than about 2% amino acid residues with a positive charge. However, the invention contemplates constructs where a limited number of amino acids with a positive charge, such as lysine, may be incorporated into XTEN to permit conjugation between the epsilon amine of the lysine and a reactive group on a peptide, a linker bridge, or a reactive group on a drug or small molecule to be conjugated to the XTEN backbone. In the foregoing, fusion proteins can be constructed that comprises XTEN, a C-peptide, plus a chemotherapeutic agent useful in the treatment of metabolic diseases or disorders, wherein the maximum number of molecules of the agent incorporated into the XTEN component is determined by the numbers of lysines or other amino acids with reactive side chains (e.g., cysteine) incorporated into the XTEN.

In some cases, an XTEN sequence may comprise charged residues separated by other residues such as serine or glycine, which may lead to better expression or purification behavior. Based on the net charge, XTENs of the subject compositions may have an isoelectric point (PI) of 1.0, 1.5, 2.0, 2.5, 3.0, 3.5, 4.0, 4.5, 5.0, 5.5, 6.0, or even 6.5. In preferred embodiments, the XTEN will have an isoelectric point between 1.5 and 4.5. In these embodiments, the XTEN incorporated into the C-peptide-XTEN fusion protein compositions of the present invention would carry a net negative charge under physiologic conditions that may contribute to the unstructured conformation and reduced binding of the XTEN component to mammalian proteins and tissues.

As hydrophobic amino acids can impart structure to a polypeptide, the invention provides that the content of hydrophobic amino acids in the XTEN will typically be less than 5%, or less than 2%, or less than 1% hydrophobic amino acid content. In one embodiment, the amino acid content of methionine and tryptophan in the XTEN component of a C-peptide-XTEN fusion protein is typically less than 5%, or less than 2%, and most preferably less than 1%. In another embodiment, the XTEN will have a sequence that has less than 10% amino acid residues with a positive charge, or less than about 7%, or less that about 5%, or less than about 2% amino acid residues with a positive charge, the sum of methionine and tryptophan residues will be less than 2%, and the sum of asparagine and glutamine residues will be less than 10% of the total XTEN sequence.

In another aspect, the invention provides compositions in which the XTEN sequences have a low degree of immunogenicity or are substantially non-immunogenic. Several factors can contribute to the low immunogenicity of XTEN, e.g., the non-repetitive sequence, the unstructured conformation, the high degree of solubility, the low degree or lack of self-aggregation, the low degree or lack of proteolytic sites within the sequence, and the low degree or lack of epitopes in the XTEN sequence.

Conformational epitopes are formed by regions of the protein surface that are composed of multiple discontinuous amino acid sequences of the protein antigen. The precise folding of the protein brings these sequences into a well-defined, stable spatial configurations, or epitopes, that can be recognized as “foreign” by the host humoral immune system, resulting in the production of antibodies to the protein or triggering a cell-mediated immune response. In the latter case, the immune response to a protein in an individual is heavily influenced by T-cell epitope recognition that is a function of the peptide binding specificity of that individual's HLA-DR allotype. Engagement of a MHC Class II peptide complex by a cognate T-cell receptor on the surface of the T-cell, together with the cross-binding of certain other co-receptors such as the CD4 molecule, can induce an activated state within the T-cell. Activation leads to the release of cytokines further activating other lymphocytes such as B cells to produce antibodies or activating T killer cells as a full cellular immune response.

The ability of a peptide to bind a given MHC Class II molecule for presentation on the surface of an APC (antigen presenting cell) is dependent on a number of factors; most notably its primary sequence. In one embodiment, a lower degree of immunogenicity may be achieved by designing XTEN sequences that resist antigen processing in antigen presenting cells, and/or choosing sequences that do not bind MHC receptors well. The invention provides C-peptide-XTEN fusion proteins with substantially non-repetitive XTEN polypeptides designed to reduce binding with MHC II receptors, as well as avoiding formation of epitopes for T-cell receptor or antibody binding, resulting in a low degree of immunogenicity. Avoidance of immunogenicity is, in part, a direct result of the conformational flexibility of XTEN sequences; i.e., the lack of secondary structure due to the selection and order of amino acid residues. For example, of particular interest are sequences having a low tendency to adapt compactly folded conformations in aqueous solution or under physiologic conditions that could result in conformational epitopes. The administration of fusion proteins comprising XTEN, using conventional therapeutic practices and dosing, would generally not result in the formation of neutralizing antibodies to the XTEN sequence, and may also reduce the immunogenicity of the C-peptide fusion partner in the C-peptide-XTEN compositions.

In one embodiment, the XTEN sequences utilized in the subject fusion proteins can be substantially free of epitopes recognized by human T cells. The elimination of such epitopes for the purpose of generating less immunogenic proteins has been disclosed previously; see for example WO 98/52976, WO 02/079232, and WO 00/3317 which are incorporated by reference herein. Assays for human T cell epitopes have been described (Stickler, M., et al., (2003) J. Immunol. Methods, 281: 95-108). Of particular interest are peptide sequences that can be oligomerized without generating T cell epitopes or non-human sequences. This can be achieved by testing direct repeats of these sequences for the presence of T-cell epitopes and for the occurrence of 6 to 15-mer and, in particular, 9-mer sequences that are not human, and then altering the design of the XTEN sequence to eliminate or disrupt the epitope sequence. In some cases, the XTEN sequences are substantially non-immunogenic by the restriction of the numbers of epitopes of the XTEN predicted to bind MHC receptors. With a reduction in the numbers of epitopes capable of binding to MHC receptors, there is a concomitant reduction in the potential for T cell activation as well as T cell helper function, reduced B cell activation or up-regulation and reduced antibody production. The low degree of predicted T-cell epitopes can be determined by epitope prediction algorithms such as, e.g., TEPITOPE (Sturniolo, T., et al., (1999) Nat. Biotechnol., 17: 555-61), as shown in Example 42. The TEPITOPE score of a given peptide frame within a protein is the log of the K_{d} (dissociation constant, affinity, off-rate) of the binding of that peptide frame to multiple of the most common human MHC alleles, as disclosed in Sturniolo, T. et al., (1999) Nature Biotechnology 17:555). The score ranges over at least 20 logs, from about 10 to about −10 (corresponding to binding constraints of 10e¹⁰ K_{d} to 1 10e⁻¹⁰ K_{d}, and can be reduced by avoiding hydrophobic amino acids that can serve as anchor residues during peptide display on MHC, such as M, I, L, V, F. In some embodiments, an XTEN component incorporated into a C-peptide-XTEN does not have a predicted or greater, T-cell epitope at a TEPITOPE score of about −5 or greater, or −6 or greater, or −7 or greater, or −8 or greater, or at a TEPITOPE score of −9 or greater. As used herein, a score of “−9 or greater” would encompass TEPITOPE scores of 10 to −9, inclusive, but would not encompass a score of −10, as −10 is less than −9.

In another embodiment, the inventive XTEN sequences, including those incorporated into the subject C-peptide-XTEN fusion proteins, can be rendered substantially non-immunogenic by the restriction of known proteolytic sites from the sequence of the XTEN, reducing the processing of XTEN into small peptides that can bind to MHC II receptors. In another embodiment, the XTEN sequence can be rendered substantially non-immunogenic by the use a sequence that is substantially devoid of secondary structure, conferring resistance to many proteases due to the high entropy of the structure. Accordingly, the reduced TEPITOPE score and elimination of known proteolytic sites from the XTEN may render the XTEN compositions, including the XTEN of the C-peptide-XTEN fusion protein compositions, substantially unable to be bound by mammalian receptors, including those of the immune system. In one embodiment, an XTEN of a C-peptide-XTEN fusion protein can have >100 nM K_{d} binding to a mammalian receptor, or greater than 500 nM K_{d}, or greater than 1 μM K_{d} towards a mammalian cell surface or circulating polypeptide receptor.

Additionally, the non-repetitive sequence and corresponding lack of epitopes of XTEN can limit the ability of B cells to bind to or be activated by XTEN. A repetitive sequence is recognized and can form multivalent contacts with even a few B cells and, as a consequence of the cross-linking of multiple T-cell independent receptors, can stimulate B cell proliferation and antibody production. In contrast, while a XTEN can make contacts with many different B cells over its extended sequence, each individual B cell may only make one or a small number of contacts with an individual XTEN due to the lack of repetitiveness of the sequence. As a result, XTENs typically may have a much lower tendency to stimulate proliferation of B cells and thus an immune response. In one embodiment, the C-peptide-XTEN may have reduced immunogenicity as compared to the corresponding C-peptide that is not fused. In one embodiment, the administration of up to three parenteral doses of a C-peptide-XTEN to a mammal may result in detectable anti-C-peptide-XTEN IgG at a serum dilution of 1:100 but not at a dilution of 1:1000. In another embodiment, the administration of up to three parenteral doses of an C-peptide-XTEN to a mammal may result in detectable anti-C-peptide IgG at a serum dilution of 1:100 but not at a dilution of 1:1000. In another embodiment, the administration of up to three parenteral doses of an C-peptide-XTEN to a mammal may result in detectable anti-XTEN IgG at a serum dilution of 1:100 but not at a dilution of 1:1000. In the foregoing embodiments, the mammal can be a mouse, a rat, a rabbit, or a cynomolgus monkey. An additional feature of XTENs with non-repetitive sequences relative to sequences with a high degree of repetitiveness can be that non-repetitive XTENs form weaker contacts with antibodies. Antibodies are multivalent molecules. For instance, IgGs have two identical binding sites and IgMs contain 10 identical binding sites. Thus antibodies against repetitive sequences can form multivalent contacts with such repetitive sequences with high avidity, which can affect the potency and/or elimination of such repetitive sequences. In contrast, antibodies against non-repetitive XTENs may yield monovalent interactions, resulting in less likelihood of immune clearance such that the C-peptide-XTEN compositions can remain in circulation for an increased period of time.

In another aspect, the present invention provides XTEN in which the XTEN polypeptides can have a high hydrodynamic radius that confers a corresponding increased Apparent Molecular Weight to the C-peptide-XTEN fusion protein incorporating the XTEN. As detailed in Example 19, the linking of XTEN to C-peptide sequences can result in C-peptide-XTEN compositions that can have increased hydrodynamic radii, increased Apparent Molecular Weight, and increased Apparent Molecular Weight Factor compared to a C-peptide not linked to an XTEN. For example, in therapeutic applications in which prolonged half-life is desired, compositions in which a XTEN with a high hydrodynamic radius is incorporated into a fusion protein comprising one or more C-peptide can effectively enlarge the hydrodynamic radius of the composition beyond the glomerular pore size of approximately 3-5 nm (corresponding to an apparent molecular weight of about 70 kDA) (Caliceti, 2003, “Pharmacokinetic and biodistribution properties of poly(ethylene glycol)-protein conjugates,” Adv. Drug Deliv. Rev., 55:1261-1277), resulting in reduced renal clearance of circulating proteins. The hydrodynamic radius of a protein is determined by its molecular weight as well as by its structure, including shape and compactness. Not to be bound by a particular theory, the XTEN can adopt open conformations due to electrostatic repulsion between individual charges of the peptide or the inherent flexibility imparted by the particular amino acids in the sequence that lack potential to confer secondary structure. The open, extended and unstructured conformation of the XTEN polypeptide can have a greater proportional hydrodynamic radius compared to polypeptides of a comparable sequence length and/or molecular weight that have secondary and/or tertiary structure, such as typical globular proteins. Methods for determining the hydrodynamic radius are well known in the art, such as by the use of size exclusion chromatography (SEC), as described in U.S. Pat. Nos. 6,406,632 and 7,294,513. As the results of Example 19 demonstrate, the addition of increasing lengths of XTEN results in proportional increases in the parameters of hydrodynamic radius, Apparent Molecular Weight, and Apparent Molecular Weight Factor, permitting the tailoring of C-peptide-XTEN to desired characteristic cut-off Apparent Molecular Weights or hydrodynamic radii. Accordingly, in certain embodiments, the C-peptide-XTEN fusion protein can be configured with an XTEN such that the fusion protein can have a hydrodynamic radius of at least about 5 nm, or at least about 8 nm, or at least about 10 nm, or 12 nm, or at least about 15 nm. In the foregoing embodiments, the large hydrodynamic radius conferred by the XTEN in a C-peptide-XTEN fusion protein can lead to reduced renal clearance of the resulting fusion protein, leading to a corresponding increase in terminal half-life, an increase in mean residence time, and/or a decrease in renal clearance rate.

In another embodiment, an XTEN of a chosen length and sequence can be selectively incorporated into a C-peptide-XTEN to create a fusion protein that will have, under physiologic conditions, an Apparent Molecular Weight of at least about 150 kDa, or at least about 300 kDa, or at least about 400 kDa, or at least about 500 kDA, or at least about 600 kDa, or at least about 700 kDA, or at least about 800 kDa, or at least about 900 kDa, or at least about 1000 kDa, or at least about 1200 kDa, or at least about 1500 kDa, or at least about 1800 kDa, or at least about 2000 kDa, or at least about 2300 kDa or more. In another embodiment, an XTEN of a chosen length and sequence can be selectively linked to a C-peptide to result in a C-peptide-XTEN fusion protein that has, under physiologic conditions, an Apparent Molecular Weight Factor of at least three, alternatively of at least four, alternatively of at least five, alternatively of at least six, alternatively of at least eight, alternatively of at least 10, alternatively of at least 15, or an Apparent Molecular Weight Factor of at least 20 or greater. In another embodiment, the C-peptide-XTEN fusion protein has, under physiologic conditions, an Apparent Molecular Weight Factor that is about 4 to about 20, or is about 6 to about 15, or is about 8 to about 12, or is about 9 to about 10 relative to the actual molecular weight of the fusion protein.

The invention provides C-peptide-XTEN fusion protein compositions comprising C-peptide linked to one or more XTEN polypeptides useful for preventing, treating, mediating, or ameliorating a disease, disorder or condition. In some cases, the C-peptide-XTEN is a monomeric fusion protein with a C-peptide linked to one or more XTEN polypeptides. In other cases, the C-peptide-XTEN composition can include two C-peptide molecules linked to one or more XTEN polypeptides. The invention contemplates C-peptide-XTEN comprising, but not limited to C-peptide selected from Table 1 (or fragments or sequence variants thereof), and XTEN selected from Table 3 or sequence variants thereof. In some cases, at least a portion of the biological activity of the respective C-peptide is retained by the intact C-peptide-XTEN. In other cases, the C-peptide component either becomes biologically active or has an increase in activity upon its release from the XTEN by cleavage of an optional cleavage sequence incorporated within spacer sequences into the C-peptide-XTEN, described more fully below.

In one embodiment of the C-peptide-XTEN composition, the invention provides a fusion protein of formula I:

(C-peptide)-(S)ₓ-(XTEN) I

wherein independently for each occurrence, S is a spacer sequence having between 1 to about 50 amino acid residues that can optionally include a cleavage sequence (as described more fully below): x is either 0 or 1; and XTEN is an extended recombinant polypeptide as described hereinabove.

In another embodiment of the C-peptide-XTEN composition, the invention provides a fusion protein of formula II (components as described above):

(XTEN)-(S)ₓ-(C-peptide) II

wherein independently for each occurrence, S is a spacer sequence having between 1 to about 50 amino acid residues that can optionally include a cleavage sequence (as described more fully below); x is either 0 or 1; and XTEN is an extended recombinant polypeptide as described hereinabove.

Thus, the C-peptide-XTEN having a single C-peptide and a single XTEN can have at least the following permutations of configurations, each listed in an N- to C-terminus orientation: C-peptide-XTEN; XTEN-C-peptide; C-peptide-S-XTEN; or XTEN-S-C-peptide.

In another embodiment, the invention provides an isolated fusion protein, wherein the fusion protein is of formula III:

(C-peptide)-(S)ₓ-(XTEN)-(S)_{y}-(C-peptide)-(S)_{z}-(XTEN)_{z} III

wherein independently for each occurrence, S is a spacer sequence having between 1 to about 50 amino acid residues that can optionally include a cleavage sequence (as described more fully below); x is either 0 or I; y is either 0 or 1; z is either 0 or 1; and XTEN is an extended recombinant polypeptide as described hereinabove.

In another embodiment, the invention provides an isolated fusion protein, wherein the fusion protein is of formula IV (components as described above):

(XTEN)ₓ-(S)_{y}-(C-peptide)-(S)_{z}-(XTEN)-(C-peptide) IV

In another embodiment, the invention provides an isolated fusion protein, wherein the fusion protein is of formula V (components as described above):

(C-peptide)ₓ-(S)ₓ-(C-peptide)-(S)_{y}-(XTEN) V

In another embodiment, the invention provides an isolated fusion protein, wherein the fusion protein is of formula VI (components as described above):

(XTEN)-(S)ₓ-(C-peptide)-(S)_{y}-(C-peptide) VI

In another embodiment, the invention provides an isolated fusion protein, wherein the fusion protein is of formula VII (components as described above):

(XTEN)-(S)ₓ-(C-peptide)-(S)_{y}-(C-peptide)-(XTEN) VII

In the foregoing embodiments of fusion proteins of formulas I-VII, administration of a therapeutically effective dose of a fusion protein of an embodiment to a subject in need thereof can result in a gain in time of at least two-fold, or at least three-fold, or at least four-fold, or at least five-fold or more spent within a therapeutic window for the fusion protein compared to the corresponding C-peptide not linked to the XTEN of and administered at a comparable dose to a subject.

Any spacer sequence group is optional in the fusion proteins encompassed by the invention. The spacer may be provided to enhance expression of the fusion protein from a host cell or to decrease steric hindrance such that the C-peptide component may assume its desired tertiary structure and/or interact appropriately with its target molecule. For spacers and methods of identifying desirable spacers, see, for example, George, et al., (2003) Protein Engineering 15:871-879, specifically incorporated by reference herein. In one embodiment, the spacer comprises one or more peptide sequences that are between 1-50 amino acid residues in length, or about 1-25 residues, or about 1-10 residues in length. Spacer sequences, exclusive of cleavage sites, can comprise any of the 20 natural L amino acids, and will preferably comprise hydrophilic amino acids that are sterically unhindered that can include, but not be limited to, glycine (G), alanine (A), serine (S), threonine (T), glutamate (E) and proline (P). In some cases, the spacer can be polyglycines or polyalanines, or is predominately a mixture of combinations of glycine and alanine residues. The spacer polypeptide exclusive of a cleavage sequence is largely to substantially devoid of secondary structure. In one embodiment, one or both spacer sequences in a C-peptide-XTEN fusion protein composition may each further contain a cleavage sequence, which may be identical or may be different, wherein the cleavage sequence may be acted on by a protease to release the C-peptide from the fusion protein.

In some cases, the incorporation of the cleavage sequence into the C-peptide-XTEN is designed to permit release of C-peptide that becomes active or more active upon its release from the XTEN. The cleavage sequences are located sufficiently close to the C-peptide sequences, generally within 18, or within 12, or within 6, or within 2 amino acids of the C-peptide sequence terminus, such that any remaining residues attached to the C-peptide after cleavage do not appreciably interfere with the activity (e.g., such as binding to a receptor) of the C-peptide, yet provide sufficient access to the protease to be able to effect cleavage of the cleavage sequence. In some embodiments, the cleavage site is a sequence that can be cleaved by a protease endogenous to the mammalian subject such that the C-peptide-XTEN can be cleaved after administration to a subject. In such cases, the C-peptide-XTEN can serve as a prodrug or a circulating depot for the C-peptide. Examples of cleavage sites contemplated by the invention include, but are not limited to, a polypeptide sequence cleavable by a mammalian endogenous protease selected from FXIa, FXIIa, kallikrein, FVIIa, FIXa, FXa, FIIa (thrombin), Elastase-2, granzyme B, MMP-12, MMP-13, MMP-17 or MMP-20, or by non-mammalian proteases such as TEY, enterokinase, PreScission™ protease (rhinovirus 3C protease), and sortase A. Sequences known to be cleaved by the foregoing proteases are known in the art. Exemplary cleavage sequences and cut sites within the sequences are presented in Table 4, as well as sequence variants. For example, thrombin (activated clotting factor II) acts on the sequence LTPRSLLV (SEQ ID NO:79) (Rawlings N. D., et al., (2008) Nucleic Acids Res., 36: D320), which would be cut after the arginine at position 4 in the sequence. Active FIIa is produced by cleavage of FII by FXa in the presence of phospholipids and calcium and is downstream from factor IX in the coagulation pathway. Once activated its natural role in coagulation is to cleave fibrinogen, which then in turn, begins clot formation. FIIa activity is tightly controlled and only occurs when coagulation is necessary for proper hemostasis. However, as coagulation is an on-going process in mammals, by incorporation of the LTPRSLLV (SEQ ID NO:80) sequence into the C-peptide-XTEN between the C-peptide and the XTEN, the XTEN domain would be removed from the adjoining C-peptide concurrent with activation of either the extrinsic or intrinsic coagulation pathways when coagulation is required physiologically, thereby releasing C-peptide over time. Similarly, incorporation of other sequences into C-peptide-XTEN that are acted upon by endogenous proteases would provide for sustained release of C-peptide that may, in certain cases, provide a higher degree of activity for the C-peptide from the “prodrug” form of the C-peptide-XTEN.

In some cases, only the two or three amino acids flanking both sides of the cut site (four to six amino acids total) would be incorporated into the cleavage sequence. In other cases, the known cleavage sequence can have one or more deletions or insertions or one or two or three amino acid substitutions for anyone or two or three amino acids in the known sequence, wherein the deletions, insertions or substitutions result in reduced or enhanced susceptibility but not an absence of susceptibility to the protease, resulting in an ability to tailor the rate of release of the C-peptide from the XTEN.

Exemplary substitutions are shown in Table 4.

TABLE 4
Protease Cleavage Sequences

| | SEQ | Exemplary | |
|---|---|---|---|
| Protease Acting | ID | Cleavage | |
| Upon Sequence | NO | Sequence | Minimal Cut Site* |
| FXIa | 81 | KLTR↓VVGG | KD/FL/T/R↓VA/VE/GT/GV |
| FXIIa | 82 | TMTR↓IVGG | NA |
| Kallikrein | 83 | SPFR↓STGG | —/—/FL/RY↓SR/RT/—/— |
| FVIIa | 84 | LQVR↓IVGG | NA |
| FIXa | 85 | PLGR↓IVGG | —/—/G/R↓—/—/—/— |
| FXa | 86 | IEGR↓TVGG | IA/E/GFP/R↓STI/VFS/—/G |
| FIIa (thrombin) | 87 | LTPR↓SLLV | —/—/PLA/R↓SAG/—/—/— |
| Elastase-2 | 88 | LGPV↓SGVP | —/—/—VIAT↓—/—/—/— |
| Granzyme-B | 89 | VAGD↓SLEE | V/—/—/D↓—/—/—/— |
| MMP-12 | 90 | GPAG↓LGGA | G/PA/—/G↓L/—/G/— |
| MMP-13 | 91 | GPAG↓LRGA | G/P/—/G↓L/—/GA/— |
| MMP-17 | 92 | APLG↓LRLR | —/PS/—/—↓LQ/—/LT/— |
| MMP-20 | 93 | PALP↓LVAQ | NA |
| TEV | 94 | ENLYFQ↓G | ENLYFQ↓G/S |
| Enterokinase | 95 | DDDK↓IVGG | DDDK↓IVGG |
| Protease 3C | 96 | LEVLFQ↓GP | LEVLFQ↓GP |
| (PreScission ™) | | | |
| Sortase A | 97 | LPKT↓GSES | L/P/KEAD/T↓G/—/EKS/S |

↓indicates cleavage site
NA: not applicable
*the listing of multiple amino acids before, between, or after a slash indicate alternative amino acids that can be substituted at the position; “—” indicates that any amino acid may be substituted for the corresponding amino acid indicated in the middle column

In one embodiment, a C-peptide incorporated into a C-peptide-XTEN fusion protein can have a sequence that exhibits at least about 80% sequence identity to a sequence from Table 1, alternatively at least about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99%, or about 100% sequence identity as compared with a sequence from Table 1. The C-peptide of the foregoing embodiment can be evaluated for activity using assays or measured or determined parameters as described herein, and those sequences that retain at least about 40%, or about 50%, or about 55%, or about 60%, or about 70%, or about 80%, or about 90%, or about 95%, or about or more activity compared to the corresponding native C-peptide sequence would be considered suitable for inclusion in the subject C-peptide-XTEN. The C-peptide found to retain a suitable level of activity can be linked to one or more XTEN polypeptides described hereinabove. In one embodiment, a C-peptide found to retain a suitable level of activity can be linked to one or more XTEN polypeptides having at least about 80% sequence identity to a sequence from Table 3, alternatively at least about 81%, 82%, 83%, 84%, 85%, 86%, 87%, 88%, 89%, 90%, 91%, 92%, 93%, 94%, 95%, 96%, 97%, 98%, 99%, or about 100% sequence identity as compared with a sequence of Table 1, resulting in a chimeric fusion protein.

The invention provides C-peptide-XTEN fusion proteins with enhanced pharmacokinetics compared to the C-peptide not linked to XTEN that, when used at the dose determined for the composition by the methods described herein, can achieve a circulating concentration resulting in a pharmacologic effect, yet stay within the safety range for the C-peptide component of the composition for an extended period of time compared to a comparable dose of the C-peptide not linked to XTEN. In such cases, the C-peptide-XTEN remains within the therapeutic window for the fusion protein composition for the extended period of time. As used herein, a “comparable dose” means a dose with an equivalent moles/kg for the active C-peptide pharmacophore that is administered to a subject in a comparable fashion. It will be understood in the art that a “comparable dosage” of C-peptide-XTEN fusion protein would represent a greater weight of agent but would have essentially the same mole-equivalents of C-peptide in the dose of the fusion protein and/or would have the same approximate molar concentration relative to the C-peptide.

The pharmacokinetic properties of a C-peptide that can be enhanced by linking a given XTEN to the C-peptide include terminal half-life, area under the curve (AUC), Cₘₐₓ, volume of distribution, and bioavailability.

As described more fully in the Examples pertaining to pharmacokinetic characteristics of fusion proteins comprising XTEN, it was surprisingly discovered that increasing the length of the XTEN sequence could confer a disproportionate increase in the terminal half-life of a fusion protein comprising the XTEN. Accordingly, the invention provides C-peptide-XTEN fusion proteins comprising XTEN wherein the XTEN can be selected to provide a targeted half-life for the C-peptide-XTEN composition administered to a subject. In some embodiments, the invention provides monomeric fusion proteins comprising XTEN wherein the XTEN is selected to confer an increase in the terminal half-life for the administered C-peptide-XTEN, compared to the corresponding C-peptide not linked to the fusion protein, of at least about two-fold longer, or at least about three-fold, or at least about four-fold, or at least about five-fold, or at least about six-fold, or at least about seven-fold, or at least about eight-fold, or at least about nine-fold, or at least about ten-fold, or at least about 15-fold, or at least a 20-fold or greater an increase in terminal half-life compared to the C-peptide not linked to the fusion protein. Similarly, the C-peptide-XTEN fusion proteins can have an increase in AUC of at least about 50%, or at least about 60%, or at least about 70%, or at least about 80%, or at least about 90%, or at least about 100%, or at least about 150%, or at least about 200%, or at least about 300% increase in AUC compared to the corresponding C-peptide not linked to the fusion protein. The pharmacokinetic parameters of a C-peptide-XTEN can be determined by standard methods involving dosing, the taking of blood samples at time intervals, and the assaying of the protein using ELISA, HPLC, radioassay, or other methods known in the art or as described herein, followed by standard calculations of the data to derive the half-life and other PK parameters.

The invention further provides C-peptide-XTEN comprising a first and optionally a second C-peptide molecule, optionally separated by a spacer sequence that may further comprise a cleavage sequence, or separated by a second XTEN sequence. In one embodiment, the C-peptide has less activity when linked to the fusion protein compared to a corresponding C-peptide not linked to the fusion protein. In such case, the C-peptide-XTEN can be designed such that upon administration to a subject, the C-peptide component is gradually released by cleavage of the cleavage sequences), whereupon it regains activity or the ability to bind to its target receptor or ligand. Accordingly, the C-peptide-XTEN of the foregoing serves as a prodrug or a circulating depot, resulting in a longer terminal half-life compared to C-peptide not linked to the fusion protein.

The present invention provides C-peptide-XTEN compositions comprising C-peptide covalently linked to XTEN that can have enhanced properties compared to C-peptide not linked to XTEN, as well as methods to enhance the therapeutic and/or biologic activity or effect of the respective two C-peptide components of the compositions. In addition, the invention provides C-peptide-XTEN compositions with enhanced properties compared to those art-known fusion proteins containing immunoglobulin polypeptide partners, polypeptides of shorter length and/or polypeptide partners with repetitive sequences. In addition, C-peptide-XTEN fusion proteins provide significant advantages over chemical conjugates, such as PEGylated constructs, notably the fact that recombinant C-peptide-XTEN fusion proteins can be made in bacterial cell expression systems, which can reduce time and cost at both the research and development and manufacturing stages of a product, as well as result in a more homogeneous, defined product with less toxicity for both the product and metabolites of the C-peptide-XTEN compared to PEGylated conjugates.

As therapeutic agents, the C-peptide-XTEN may possess a number of advantages over therapeutics not comprising XTEN including, for example, increased solubility, increased thermal stability, reduced immunogenicity, increased apparent molecular weight, reduced renal clearance, reduced proteolysis, reduced metabolism, enhanced therapeutic efficiency, a lower effective therapeutic dose, increased bioavailability, increased time between dosages to maintain blood levels within the therapeutic window for the C-peptide, a “tailored” rate of absorption, enhanced lyophilization stability, enhanced serum/plasma stability, increased terminal half-life, increased solubility in blood stream, decreased binding by neutralizing antibodies, decreased receptor-mediated clearance, reduced side effects, retention of receptor/ligand binding affinity or receptor/ligand activation, stability to degradation, stability to freeze-thaw, stability to proteases, stability to ubiquitination, ease of administration, compatibility with other pharmaceutical excipients or carriers, persistence in the subject, increased stability in storage (e.g., increased shelf-life), reduced toxicity in an organism or environment and the like. The net effect of the enhanced properties is that the C-peptide-XTEN may result in enhanced therapeutic and/or biologic effect when administered to a subject with a metabolic disease or disorder.

In other cases where, where enhancement of the pharmaceutical or physicochemical properties of the C-peptide is desirable, (such as the degree of aqueous solubility or stability), the length and/or the motif family composition of the first and the second XTEN sequences of the first and the second fusion protein may each be selected to confer a different degree of solubility and/or stability on the respective fusion proteins such that the overall pharmaceutical properties of the C-peptide-XTEN composition are enhanced. The C-peptide-XTEN fusion proteins can be constructed and assayed, using methods described herein, to confirm the physicochemical properties and the XTEN adjusted, as needed, to result in the desired properties. In one embodiment, the XTEN sequence of the C-peptide-XTEN is selected such that the fusion protein has an aqueous solubility that is within at least about 25% greater compared to a C-peptide not linked to the fusion protein, or at least about 30%, or at least about 40%, or at least about 50%, or at least about 75%, or at least about 100%, or at least about 200%, or at least about 300%, or at least about 400%, or at least about 500%, or at least about 1000% greater than the corresponding C-peptide not linked to the fusion protein. In the embodiments hereinabove described in this paragraph, the XTEN of the fusion proteins can have at least about 80% sequence identity, or about 90%, or about 91%, or about 92%, or about 93%, or about 94%, or about 95%, or about 96%, or about 97%, or about 98%, or about 99%, to about 100% sequence identity to an XTEN selected from Table 3.

In one embodiment, the invention provides C-peptide-XTEN compositions that can maintain the C-peptide component within a therapeutic window for a greater period of time compared to comparable dosages of the corresponding C-peptide not linked to XTEN. It will be understood in the art that a “comparable dosage” of C-peptide-XTEN fusion protein would represent a greater weight of agent but would have the same approximate mole-equivalents of C-peptide in the dose of the fusion protein and/or would have the same approximate molar concentration relative to the C-peptide.

The invention also provides methods to select the XTEN appropriate for conjugation to provide the desired pharmacokinetic properties that, when matched with the selection of dose, enable increased efficacy of the administered composition by maintaining the circulating concentrations of the C-peptide within the therapeutic window for an enhanced period of time. As used herein, “therapeutic window” means that amount of drug or biologic as a blood or plasma concentration range, that provides efficacy or a desired pharmacologic effect over time for the disease or condition without unacceptable toxicity; the range of the circulating blood concentrations between the minimal amount to achieve any positive therapeutic effect and the maximum amount which results in a response that is the response immediately before toxicity to the subject (at a higher dose or concentration). Additionally, therapeutic window generally encompasses an aspect of time; the maximum and minimum concentration that results in a desired pharmacologic effect over time that does not result in unacceptable toxicity or adverse events. A dosed composition that stays within the therapeutic window for the subject could also be said to be within the “safety range.”

Dose optimization is important for all drugs, especially for those with a narrow therapeutic window. For example, many peptides have a narrow therapeutic window. For a C-peptide with a narrow therapeutic window, such as insulin or C-peptide, a standardized single dose for all patients presenting with a variety of symptoms may not always be effective. Since different peptides are often used together in the treatment of diabetic subjects, the potency of each and the interactive effects achieved by combining and dosing them together must also be taken into account. A consideration of these factors is well within the purview of the ordinarily skilled clinician for the purpose of determining the therapeutically or pharmacologically effective amount of the C-peptide-XTEN, versus that amount that would result in unacceptable toxicity and place it outside of the safety range.

In many cases, the therapeutic window for the C-peptide components of the subject compositions have been established and are available in published literature or are stated on the drug label for approved products containing the C-peptide. In other cases, the therapeutic window can be established. The methods for establishing the therapeutic window for a given composition are known to those of skill in the art (see, e.g., Goodman & Gilman's The Pharmacological Basis of Therapeutics, 11th Edition, McGraw-Hill (2005)). For example, by using dose-escalation studies in subjects with the target disease or disorder to determine efficacy or a desirable pharmacologic effect, appearance of adverse events, and determination of circulating blood levels, the therapeutic window for a given subject or population of subjects can be determined for a given drug or biologic, or combinations of biologics or drugs. The dose escalation studies can evaluate the activity of a C-peptide-XTEN through metabolic studies in a subject or group of subjects that monitor physiological or biochemical parameters, as known in the art or as described herein for one or more parameters associated with the metabolic disease or disorder, or clinical parameters associated with a beneficial outcome for the particular indication, together with observations and/or measured parameters to determine the no effect dose, adverse events, maximum tolerated dose and the like, together with measurement of pharmacokinetic parameters that establish the determined or derived circulating blood levels. The results can then be correlated with the dose administered and the blood concentrations of the therapeutic that are coincident with the foregoing determined parameters or effect levels. By these methods, a range of doses and blood concentrations can be correlated to the minimum effective dose as well as the maximum dose and blood concentration at which a desired effect occurs and above which toxicity occurs, thereby establishing the therapeutic window for the dosed therapeutic. Blood concentrations of the fusion protein (or as measured by the C-peptide component) above the maximum would be considered outside the therapeutic window or safety range. Thus, by the foregoing methods, a Cₘᵢₙ blood level would be established, below which the C-peptide-XTEN fusion protein would not have the desired pharmacologic effect, and a Cₘₐₓ blood level would be established that would represent the highest circulating concentration before reaching a concentration that would elicit unacceptable side effects, toxicity or adverse events, placing it outside the safety range for the C-peptide-XTEN. With such concentrations established, the frequency of dosing and the dosage can be further refined by measurement of the Cₘₐₓ and Cₘᵢₙ to provide the appropriate dose and dose frequency to keep the fusion protein(s) within the therapeutic window. One of skill in the art can, by the means disclosed herein or by other methods known in the art, confirm that the administered C-peptide-XTEN remains in the therapeutic window for the desired interval or requires adjustment in dose or length or sequence of XTEN. Further, the determination of the appropriate dose and dose frequency to keep the C-peptide-XTEN within the therapeutic window establishes the therapeutically effective dose regimen; the schedule for administration of multiple consecutive doses using a therapeutically effective dose of the fusion protein to a subject in need thereof, resulting in consecutive Cₘₐₓ peaks and/or Cₘᵢₙ troughs that remain within the therapeutic window and results in an improvement in at least one measured parameter relevant for the target disease, disorder or condition. In some cases, the C-peptide-XTEN administered at an appropriate dose to a subject may result in blood concentrations of the C-peptide-XTEN fusion protein that remains within the therapeutic window for a period at least about two-fold longer compared to the corresponding C-peptide not linked to XTEN and administered at a comparable dose; alternatively at least about three-fold longer; alternatively at least about four-fold longer; alternatively at least about five-fold longer; alternatively at least about six-fold longer; alternatively at least about seven-fold longer; alternatively at least about eight-fold longer; alternatively at least about nine-fold longer or at least about ten-fold longer or greater compared to the corresponding C-peptide not linked to XTEN and administered at a comparable dose. As used herein, an “appropriate dose” means a dose of a drug or biologic that, when administered to a subject, would result in a desirable therapeutic or pharmacologic effect and a blood concentration within the therapeutic window. In one embodiment, the C-peptide-XTEN administered at a therapeutically effective dose regimen results in a gain in time of at least about three-fold longer; alternatively at least about four-fold longer; alternatively at least about five-fold longer; alternatively at least about six-fold longer; alternatively at least about seven-fold longer; alternatively at least about eight-fold longer; alternatively at least about nine-fold longer or at least about ten-fold longer between at least two consecutive Cₘₐₓ peaks and/or Cₘᵢₙ troughs for blood levels of the fusion protein compared to the corresponding C-peptide portion of the fusion protein not linked to the fusion protein and administered at a comparable dose regimen to a subject. In another embodiment, the C-peptide-XTEN administered at a therapeutically effective dose regimen results in a comparable improvement in one, or two, or three or more measured parameter using less frequent dosing or a lower total dosage in moles of the fusion protein of the pharmaceutical composition compared to the corresponding C-peptide component not linked to the fusion protein and administered to a subject using a therapeutically effective dose regimen for the C-peptide. The measured parameters may include any of the clinical, biochemical, or physiological parameters disclosed herein, or others known in the art for assessing subjects with glucose- or insulin-related disorders window.

The activity of the C-peptide-XTEN compositions of the invention, including functional characteristics or biologic and pharmacologic activity and parameters that result, may be determined by any suitable screening assay known in the art for measuring the desired characteristic. The activity and structure of the C-peptide-XTEN polypeptides comprising C-peptide components may be measured by assays described herein; e.g., one or more assays disclosed herein, or by methods known in the art to ascertain the degree of solubility, structure and retention of biologic activity. Assays can be conducted that allow determination of binding characteristics of the C-peptide-XTEN for C-peptide receptors or a ligand, including binding constant (K_{d}), EC₅₀ values, as well as their half-life of dissociation of the ligand-receptor complex (T_{1/2}). Binding affinity can be measured, for example, by a competition-type binding assay that detects changes in the ability to specifically bind to a receptor or ligand. Additionally, techniques such as flow cytometry or surface plasmon resonance can be used to detect binding events. The assays may comprise soluble receptor molecules, or may determine the binding to cell-expressed receptors. Such assays may include cell-based assays, including assays for proliferation, cell death, apoptosis and cell migration. Other possible assays may determine receptor binding of expressed polypeptides, wherein the assay may comprise soluble receptor molecules, or may determine the binding to cell-expressed receptors. The binding affinity of a C-peptide-XTEN for the target receptors or ligands of the corresponding C-peptide can be assayed using binding or competitive binding assays, such as Biacore assays with chip-bound receptors or binding proteins or ELISA assays, as described in U.S. Pat. No. 5,534,617, assays described in the Examples herein, radio-receptor assays, or other assays known in the art. In addition, C-peptide sequence variants (assayed as single components or as C-peptide-XTEN fusion proteins) can be compared to the native C-peptide using a competitive ELISA binding assay to determine whether they have the same binding specificity and affinity as the native C-peptide, or some fraction thereof such that they are suitable for inclusion in C-peptide-XTEN.

The invention provides isolated C-peptide-XTEN in which the binding affinity for C-peptide target receptors or ligands by the C-peptide-XTEN can be at least about 10%, or at least about 20%, or at least about 30%, or at least about 40%, or at least about 50%, or at least about 60%, or at least about 70%, or at least about 80%, or at least about 90%, or at least about 95%, or at least about 99%, or at least about 100% or more of the affinity of a native C-peptide not bound to XTEN for the target receptor or ligand. In some cases, the binding affinity K, between the subject C-peptide-XTEN and a native receptor or ligand of the C-peptide-XTEN is at least about 10⁻⁴ M, alternatively at least about 10⁻⁵ M, alternatively at least about 10⁻⁶ M, or at least about 10⁻⁷ M of the affinity between the C-peptide-XTEN and a native receptor or ligand.

In some cases, the C-peptide-XTEN fusion proteins of the invention retain at least about 10%, or about 20%, 30%, 40%, 50%, 60%, 70%, 80%, 90%, 95%, 98%, or 99% percent of the biological activity of the corresponding C-peptide not linked to the fusion protein with regard to an in vitro biologic activity or pharmacologic effect known or associated with the use of the native C-peptide in the treatment and prevention of disorders. In some cases of the foregoing embodiment, the activity of the C-peptide component may be manifest by the intact C-peptide-XTEN fusion protein, while in other cases the activity of the C-peptide component would be primarily manifested upon cleavage and release of the C-peptide from the fusion protein by action of a protease that acts on a cleavage sequence incorporated into the C-peptide-XTEN fusion protein. In the foregoing, the C-peptide-XTEN can be designed to reduce the binding affinity of the C-peptide-component for the receptor or ligand when linked to the XTEN but have increased affinity when released from XTEN through the cleavage of cleavage sequence(s) incorporated into the C-peptide-XTEN sequence, as described more fully above.

In other cases, the C-peptide-XTEN are designed to reduce the binding affinity of the C-peptide component when linked to the XTEN to, for example, increase the terminal half-life of C-peptide-XTEN administered to a subject by reducing receptor-mediated clearance or to reduce toxicity or side effects due to the administered composition. Where the toxicological no-effect dose or blood concentration of a C-peptide not linked to an XTEN is low (meaning that the native peptide has a high potential to result in side effects), the invention provides C-peptide-XTEN fusion proteins in which the fusion protein is configured to reduce the biologic potency or activity of the C-peptide component.

In some cases, it has been found that a C-peptide-XTEN can be configured to have a substantially reduced binding affinity (expressed as K_{d}) and a corresponding reduced bioactivity, compared to the activity of a C-peptide-XTEN wherein the configuration does not result in reduced binding affinity of the corresponding C-peptide component, and that such configuration is advantageous in terms of having a composition that displays both a long terminal half-life and retains a sufficient degree of bioactivity.

Specific in vivo and ex vivo biological assays may also be used to assess the biological activity of each configured C-peptide-XTEN and/or C-peptide component to be incorporated into C-peptide-XTEN.

Specific assays and methods for measuring the physical and structural properties of expressed proteins are known in the art, including methods for determining properties such as protein aggregation, solubility, secondary and tertiary structure, melting properties, contamination and water content, etc. Such methods include analytical centrifugation, EPR, HPLC-ion exchange, HPLC-size exclusion, HPLC-reverse phase, light scattering, capillary electrophoresis, circular dichroism, differential scanning calorimetry, fluorescence, HPLC-ion exchange, HPLC-size exclusion, IR, NMR, Raman spectroscopy, refractometry, and UV-visible spectroscopy (see, e.g., Examples 19, 21, 26 and 27). Additional methods are disclosed in Arnau et al., Prot. Expr. and Purif. (2006) 48:1-13. Application of these methods to the invention would be within the grasp of a person skilled in the art.

In another aspect, the invention provides a method of designing the C-peptide-XTEN compositions with desired pharmacologic or pharmaceutical properties. The C-peptide-XTEN fusion proteins are designed and prepared with various objectives in mind (compared to the C-peptide components not linked to the fusion protein), including improving the therapeutic efficacy for the treatment of metabolic diseases or disorders, enhancing the pharmacokinetic characteristics of the fusion proteins compared to the C-peptide, lowering the dose or frequency of dosing required to achieve a pharmacologic effect, enhancing the pharmaceutical properties, and to enhance the ability of the C-peptide components to remain within the therapeutic window for an extended period of time.

In general, the steps in the design and production of the fusion proteins and the inventive compositions may include: (1) the selection of C-peptides (e.g., native proteins, analogs, or derivatives with activity, peptide fragments, etc.) to treat the particular disease, disorder or condition; (2) selecting the XTEN that will confer the desired PK and physicochemical characteristics on the resulting C-peptide-XTEN (e.g., the administration of the composition to a subject results in the fusion protein being maintained within the therapeutic window for a greater period compared to C-peptide not linked to XTEN); (3) establishing a desired N- to C-terminus configuration of the C-peptide-XTEN to achieve the desired efficacy or PK parameters; (4) establishing the design of the expression vector encoding the configured C-peptide-XTEN; (5) transforming a suitable host with the expression vector; and (6) expression and recovery of the resultant fusion protein. For those C-peptide-XTEN for which an increase in half-life (greater than 16 h) or an increased period of time spent within a therapeutic window is desired, the XTEN chosen for incorporation will generally have at least about 500, or about 576, or about 864, or about 875, or about 913, or about 924 amino acid residues where a single XTEN is to be incorporated into the C-peptide-XTEN. In another embodiment, the C-peptide-XTEN can comprise a first XTEN of the foregoing lengths, and a second XTEN of about 144, or about 288, or about 576, or about 864, or about 875, or about 913, or about 924 amino acid residues.

In other cases, where in increase in half-life is not required, but an increase in a pharmaceutical property (e.g., solubility) is desired, a C-peptide-XTEN can be designed to include XTEN of shorter lengths. In some embodiments of the foregoing, the C-peptide-XTEN can comprise a C-peptide linked to an XTEN having at least about 24, or about 36, or about 48, or about 60, or about 72, or about 84, or about 96 amino acid residues, in which the solubility of the fusion protein under physiologic conditions is at least three-fold greater than the corresponding C-peptide not linked to XTEN, or alternatively, at least four-fold, or five-fold, or six-fold, or seven-fold, or eight-fold, or nine-fold, or at least 10-fold, or at least 20-fold, or at least 30-fold, or at least 50-fold, or at least 60-fold or greater than glucagon not linked to XTEN.

In another aspect, the invention provides methods of making C-peptide-XTEN compositions to improve ease of manufacture, result in increased stability, increased water solubility, and/or ease of formulation, as compared to the native C-peptide. In one embodiment, the invention includes a method of increasing the water solubility of a C-peptide comprising the step of linking the C-peptide to one or more XTEN such that a higher concentration in soluble form of the resulting C-peptide-XTEN can be achieved, under physiologic conditions, compared to the C-peptide in an un-fused state. Factors that contribute to the property of XTEN to confer increased water solubility of C-peptide when incorporated into a fusion protein include the high solubility of the XTEN fusion partner and the low degree of self-aggregation between molecules of XTEN in solution. In some embodiments, the method results in a C-peptide-XTEN fusion protein wherein the water solubility is at least about 50%, or at least about 60% greater, or at least about 70% greater, or at least about 80% greater, or at least about 90% greater, or at least about 100% greater, or at least about 150% greater, or at least about 200% greater, or at least about 400% greater, or at least about 600% greater, or at least about 800% greater, or at least about 1000% greater, or at least about 2000% greater, or at least about 4000% greater, or at least about 6000% greater under physiologic conditions, compared to the un-fused C-peptide.

In another embodiment, the invention includes a method of enhancing the shelf-life of a C-peptide comprising the step of linking the C-peptide with one or more XTEN selected such that the shelf-life of the resulting C-peptide-XTEN is extended compared to the C-peptide in an un-fused state. As used herein, shelf-life refers to the period of time over which the functional activity of a C-peptide or C-peptide-XTEN that is in solution or in some other storage formulation remains stable without undue loss of activity. As used herein, “functional activity” refers to a pharmacologic effect or biological activity, such as the ability to bind a receptor or ligand, or an enzymatic activity, or to display one or more known functional activities associated with C-peptide, as known in the art. C-peptide that degrades or aggregates generally has reduced functional activity or reduced bioavailability compared to one that remains in solution. Factors that contribute to the ability of the method to extend the shelf life of C-peptide when incorporated into a fusion protein include the increased water solubility, reduced self-aggregation in solution, and increased heat stability of the XTEN fusion partner. In particular, the low tendency of XTEN to aggregate facilitates methods of formulating pharmaceutical preparations containing higher drug concentrations of C-peptide, and the heat stability of XTEN contributes to the property of C-peptide-XTEN fusion proteins to remain soluble and functionally active for extended periods. In one embodiment, the method results in C-peptide-XTEN fusion proteins with “prolonged” or “extended” shelf-life that exhibit greater activity relative to a standard that has been subjected to the same storage and handling conditions. The standard may be the un-fused full-length C-peptide. In one embodiment, the method includes the step of formulating the isolated C-peptide-XTEN with one or more pharmaceutically acceptable excipients that enhance the ability of the XTEN to retain its unstructured conformation and for the C-peptide-XTEN to remain soluble in the formulation for a time that is greater than that of the corresponding un-fused C-peptide. In one embodiment, the method encompasses linking a C-peptide to an XTEN to create a C-peptide-XTEN fusion protein results in a solution that retains greater than about 100% of the functional activity, or greater than about 105%, 110%, 120%, 130%, 150% or 200% of the functional activity of a standard when compared at a given time point and when subjected to the same storage and handling conditions as the standard, thereby enhancing its shelf-life.

Shelf-life may also be assessed in terms of functional activity remaining after storage, normalized to functional activity when storage began. C-peptide-XTEN fusion proteins of the invention with prolonged or extended shelf-life as exhibited by prolonged or extended functional activity may retain about 50% more functional activity, or about 60%, 70%, 80%, or 90% more of the functional activity of the equivalent C-peptide not linked to XTEN when subjected to the same conditions for the same period of time. In some embodiments, the C-peptide-XTEN retains at least about 50%, preferably at least about 60%, or at least about 70%, or at least about 80%, or alternatively at least about 90% or more of its original activity in solution when heated or maintained at 37° C. for about 7 days. In another embodiment, C-peptide-XTEN fusion protein retains at least about 80% or more of its functional activity after exposure to a temperature of about 30° C. to about 70° C. over a period of time of about one hour to about 18 hours. In the foregoing embodiments hereinabove described in this paragraph, the retained activity of the C-peptide-XTEN would be at least about two-fold, or at least about three-fold, or at least about four-fold, or at least about five-fold, or at least about six-fold greater at a given time point than that of the corresponding C-peptide not linked to the fusion protein.

The present invention provides isolated polynucleic acids encoding C-peptide-XTEN chimeric polypeptides and sequences complementary to polynucleic acid molecules encoding C-peptide-XTEN chimeric polypeptides, including homologous variants. In another aspect, the invention encompasses methods to produce polynucleic acids encoding C-peptide-XTEN chimeric polypeptides and sequences complementary to polynucleic acid molecules encoding C-peptide-XTEN chimeric polypeptides, including homologous variants. In general, the methods of producing a polynucleotide sequence coding for a C-peptide-XTEN fusion protein and expressing the resulting gene product include assembling nucleotides encoding C-peptide and XTEN, linking the components in frame, incorporating the encoding gene into an appropriate expression vector, transforming an appropriate host cell with the expression vector, and causing the fusion protein to be expressed in the transformed host cell, thereby producing the biologically-active C-peptide-XTEN polypeptide. Standard recombinant techniques in molecular biology can be used to make the polynucleotides and expression vectors of the present invention.

In accordance with the invention, nucleic acid sequences that encode C-peptide-XTEN may be used to generate recombinant DNA molecules that direct the expression of C-peptide-XTEN fusion proteins in appropriate host cells (see, e.g., Examples 18 and 22). Several cloning strategies are envisioned to be suitable for performing the present invention, many of which can be used to generate a construct that comprises a gene coding for a fusion protein of the C-peptide-XTEN composition of the present invention, or its complement. In one embodiment, the cloning strategy would be used to create a gene that encodes a monomeric C-peptide-XTEN that comprises at least a first C-peptide and at least a first XTEN polypeptide, or its complement. In another embodiment, the cloning strategy would be used to create a gene that encodes a monomeric C-peptide-XTEN that comprises a first and a second molecule of the one C-peptide and at least a first XTEN (or its complement) that would be used to transform a host cell for expression of the fusion protein used to formulate a C-peptide-XTEN composition. In the foregoing embodiments hereinabove described in this paragraph, the gene can further comprise nucleotides encoding spacer sequences that may also encode cleavage sequence(s).

In designing a desired XTEN sequences, it was discovered that the non-repetitive nature of the XTEN of the inventive compositions can be achieved despite use of a “building block” molecular approach in the creation of the XTEN-encoding sequences. This was achieved by the use of a library of polynucleotides encoding sequence motifs that are then multimerized to create the genes encoding the XTEN sequences. Thus, while the expressed XTEN may consist of multiple units of as few as four different sequence motifs, because the motifs themselves consist of non-repetitive amino acid sequences, the overall XTEN sequence is rendered non-repetitive. Accordingly, in one embodiment, the XTEN-encoding polynucleotides comprise multiple polynucleotides that encode non-repetitive sequences, or motifs, operably linked in frame and in which the resulting expressed XTEN amino acid sequences are non-repetitive.

In one approach, a construct is first prepared containing the DNA sequence corresponding to C-peptide-XTEN fusion protein. DNA encoding the C-peptide of the compositions may be obtained from a cDNA library prepared using standard methods from tissue or isolated cells believed to possess C-peptide mRNA and to express it at a detectable level. If necessary, the coding sequence can be obtained using conventional primer extension procedures as described in Sambrook, et al., supra, to detect precursors and processing intermediates of mRNA that may not have been reverse-transcribed into cDNA. Accordingly, DNA can be conveniently obtained from a cDNA library prepared from such sources. The C-peptide encoding gene(s) may also be obtained from a genomic library or created by standard synthetic procedures known in the art (e.g., automated nucleic acid synthesis).

A gene or polynucleotide encoding the C-peptide portion of the subject C-peptide-XTEN protein, in the case of an expressed fusion protein that will comprise a single C-peptide can be then be cloned into a construct, which can be a plasmid or other vector under control of appropriate transcription and translation sequences for high level protein expression in a biological system. In a later step, a second gene or polynucleotide coding for the XTEN is genetically fused to the nucleotides encoding the N- and/or C-terminus of the C-peptide gene by cloning it into the construct adjacent and in frame with the gene(s) coding for the C-peptide. This second step can occur through a ligation or multimerization step. In the foregoing embodiments hereinabove described in this paragraph, it is to be understood that the gene constructs that are created can alternatively be the complement of the respective genes that encode the respective fusion proteins.

The gene encoding for the XTEN can be made in one or more steps, either fully synthetically or by synthesis combined with enzymatic processes, such as restriction enzyme-mediated cloning, PCR and overlap extension. XTEN polypeptides can be constructed such that the XTEN encoding gene has low repetitiveness while the encoded amino acid sequence has a degree of repetitiveness. Genes encoding XTEN with non-repetitive sequences can be assembled from oligonucleotides using standard techniques of gene synthesis. The gene design can be performed using algorithms that optimize codon usage and amino acid composition. In one method of the invention, a library of relatively short XTEN-encoding polynucleotide constructs is created and then assembled. This can be a pure codon library such that each library member has the same amino acid sequence but many different coding sequences are possible. Such libraries can be assembled from partially randomized oligonucleotides and used to generate large libraries of XTEN segments comprising the sequence motifs. The randomization scheme can be optimized to control amino acid choices for each position as well as codon usage.

In another aspect, the invention provides libraries of polynucleotides that encode XTEN sequences that can be used to assemble genes that encode XTEN of a desired length and sequence.

In certain embodiments, the XTEN-encoding library constructs comprise polynucleotides that encode polypeptide segments of a fixed length. As an initial step, a library of oligonucleotides that encode motifs of 9-14 amino acid residues can be assembled. In a preferred embodiment, libraries of oligonucleotides that encode motifs of 12 amino acids are assembled.

The XTEN-encoding sequence segments can be dimerized or multimerized into longer encoding sequences. Dimerization or multimerization can be performed by ligation, overlap extension, PCR assembly or similar cloning techniques known in the art. This process of can be repeated multiple times until the resulting XTEN-encoding sequences have reached the organization of sequence and desired length, providing the XTEN-encoding genes. As will be appreciated, a library of polynucleotides that encodes 12 amino acids can be dimerized into a library of polynucleotides that encode 36 amino acids. In turn, the library of polynucleotides that encode 36 amino acids can be serially dimerized into a library containing successively longer lengths of polynucleotides that encode XTEN sequences. In some embodiments, libraries can be assembled of polynucleotides that encode amino acids that are limited to specific sequence XTEN families; e.g., AD, AE, AF, AG, AM, or AQ sequences of Table 2. In other embodiments, libraries can comprise sequences that encode two or more of the motif family sequences from Table 2. The names and sequences of representative, non-limiting polynucleotide sequences of libraries that encode 36 mers are presented in Tables 12-15 of US2010/0239554, which are hereby incorporated by reference, and the methods used to create them are described more fully in the Examples. The libraries can be used, in turn, for serial dimerization or ligation to achieve polynucleotide sequence libraries that encode XTEN sequences, for example, of 72, 144, 288, 576, 864, 912, 923, 1296 amino acids, or up to a total length of about 3000 amino acids, as well as intermediate lengths. In some cases, the polynucleotide library sequences may also include additional bases used as “sequencing islands,” described more fully below.

Representative, non-limiting steps in the assembly of a XTEN polynucleotide construct and a C-peptide-XTEN polynucleotide construct are as follows: Individual oligonucleotides can be annealed into sequence motifs such as a 12 amino acid motif (“12-mer”), which is subsequently ligated with an oligo containing BbsI, and KpnI restriction sites. Additional sequence motifs from a library are annealed to the 12-mer until the desired length of the XTEN gene is achieved. The XTEN gene is cloned into a stuffer vector. The vector can optionally encode a Flag sequence followed by a stuffer sequence that is flanked by BsaI, BbsI, and KpnI sites and, in this case, a single C-peptide gene, resulting in the gene encoding a C-peptide-XTEN comprising a single C-peptide. A non-exhaustive list of the XTEN names and SEQ ID NOS. for polynucleotides encoding XTEN and precursor sequences is provided in Table 5.

TABLE 5
DNA sequences of XTEN and precursor sequences

| XTEN | SEQ ID | |
|---|---|---|
| Name | NO | DNA Sequence |
| AE144 | 98 | GGTAGCGAACCGGCAACTTCCGGCTCTGAAACCCCAGGTACTTCTGAAAGCGCTAC |
| | | TCCTGAGTCTGGCCCAGGTAGCGAACCTGCTACCTCTGGCTCTGAAACCCCAGGTA |
| | | GCCCGGCAGGCTCTCCGACTTCCACCGAGGAAGGTACCTCTACTGAACCTTCTGAG |
| | | GGTAGCGCTCCAGGTAGCGAACCGGCAACCTCTGGCTCTGAAACCCCAGGTAGCGA |
| | | ACCTGCTACCTCCGGCTCTGAAACTCCAGGTAGCGAACCGGCTACTTCCGGTTCTG |
| | | AAACTCCAGGTACCTCTACCGAACCTTCCGAAGGCAGCGCACCAGGTACTTCTGAA |
| | | AGCGCAACCCCTGAATCCGGTCCAGGTAGCGAACCGGCTACTTCTGGCTCTGAGAC |
| | | TCCAGGTACTTCTACCGAACCGTCCGAAGGTAGCGCACCA |
| AF144 | 99 | GGTACTTCTACTCCGGAAAGCGGTTCCGCATCTCCAGGTACTTCTCCTAGCGGTGA |
| | | ATCTTCTACTGCTCCAGGTACCTCTCCTAGCGGCGAATCTTCTACTGCTCCAGGTT |
| | | CTACCAGCTCTACCGCTGAATCTCCTGGCCCAGGTTCTACCAGCGAATCCCCGTCT |
| | | GGCACCGCACCAGGTTCTACTAGCTCTACCGCAGAATCTCCGGGTCCAGGTACTTC |
| | | CCCTAGCGGTGAATCTTCTACTGCTCCAGGTACCTCTACTCCGGAAAGCGGCTCCG |
| | | CATCTCCAGGTTCTACTAGCTCTACTGCTGAATCTCCTGGTCCAGGTACCTCCCCT |
| | | AGCGGCGAATCTTCTACTGCTCCAGGTACCTCTCCTAGCGGCGAATCTTCTACCGC |
| | | TCCAGGTACCTCCCCTAGCGGTGAATCTTCTACCGCACCA |
| AE288 | 100 | GGTACCTCTGAAAGCGCAACTCCTGAGTCTGGCCCAGGTAGCGAACCTGCTACCTC |
| | | CGGCTCTGAGACTCCAGGTACCTCTGAAAGCGCAACCCCGGAATCTGGTCCAGGTA |
| | | GCGAACCTGCAACCTCTGGCTCTGAAACCCCAGGTACCTCTGAAAGCGCTACTCCT |
| | | GAATCTGGCCCAGGTACTTCTACTGAACCGTCCGAGGGCAGCGCACCAGGTAGCCC |
| | | TGCTGGCTCTCCAACCTCCACCGAAGAAGGTACCTCTGAAAGCGCAACCCCTGAAT |
| | | CCGGCCCAGGTAGCGAACCGGCAACCTCCGGTTCTGAAACCCCAGGTACTTCTGAA |
| | | AGCGCTACTCCTGAGTCCGGCCCAGGTAGCCCGGCTGGCTCTCCGACTTCCACCGA |
| | | GGAAGGTAGCCCGGCTGGCTCTCCAACTTCTACTGAAGAAGGTACTTCTACCGAAC |
| | | CTTCCGAGGGCAGCGCACCAGGTACTTCTGAAAGCGCTACCCCTGAGTCCGGCCCA |
| | | GGTACTTCTGAAAGCGCTACTCCTGAATCCGGTCCAGGTACTTCTGAAAGCGCTAC |
| | | CCCGGAATCTGGCCCAGGTAGCGAACCGGCTACTTCTGGTTCTGAAACCCCAGGTA |
| | | GCGAACCGGCTACCTCCGGTTCTGAAACTCCAGGTAGCCCAGCAGGCTCTCCGACT |
| | | TCCACTGAGGAAGGTACTTCTACTGAACCTTCCGAAGGCAGCGCACCAGGTACCTC |
| | | TACTGAACCTTCTGAGGGCAGCGCTCCAGGTAGCGAACCTGCAACCTCTGGCTCTG |
| | | AAACCCCAGGTACCTCTGAAAGCGCTACTCCTGAATCTGGCCCAGGTACTTCTACT |
| | | GAACCGTCCGAGGGCAGCGCACCA |
| AE576 | 101 | GGTAGCCCGGCTGGCTCTCCTACCTCTACTGAGGAAGGTACTTCTGAAAGCGCTAC |
| | | TCCTGAGTCTGGTCCAGGTACCTCTACTGAACCGTCCGAAGGTAGCGCTCCAGGTA |
| | | GCCCAGCAGGCTCTCCGACTTCCACTGAGGAAGGTACTTCTACTGAACCTTCCGAA |
| | | GGCAGCGCACCAGGTACCTCTACTGAACCTTCTGAGGGCAGCGCTCCAGGTACTTC |
| | | TGAAAGCGCTACCCCGGAATCTGGCCCAGGTAGCGAACCGGCTACTTCTGGTTCTG |
| | | AAACCCCAGGTAGCGAACCGGCTACCTCCGGTTCTGAAACTCCAGGTAGCCCGGCA |
| | | GGCTCTCCGACCTCTACTGAGGAAGGTACTTCTGAAAGCGCAACCCCGGAGTCCGG |
| | | CCCAGGTACCTCTACCGAACCGTCTGAGGGCAGCGCACCAGGTACTTCTACCGAAC |
| | | CGTCCGAGGGTAGCGCACCAGGTAGCCCAGCAGGTTCTCCTACCTCCACCGAGGAA |
| | | GGTACTTCTACCGAACCGTCCGAGGGTAGCGCACCAGGTACCTCTACTGAACCTTC |
| | | TGAGGGCAGCGCTCCAGGTACTTCTGAAAGCGCTACCCCGGAGTCCGGTCCAGGTA |
| | | CTTCTACTGAACCGTCCGAAGGTAGCGCACCAGGTACTTCTGAAAGCGCAACCCCT |
| | | GAATCCGGTCCAGGTAGCGAACCGGCTACTTCTGGCTCTGAGACTCCAGGTACTTC |
| | | TACCGAACCGTCCGAAGGTAGCGCACCAGGTACTTCTACTGAACCGTCTGAAGGTA |
| | | GCGCACCAGGTACTTCTGAAAGCGCAACCCCGGAATCCGGCCCAGGTACCTCTGAA |
| | | AGCGCAACCCCGGAGTCCGGCCCAGGTAGCCCTGCTGGCTCTCCAACCTCCACCGA |
| | | AGAAGGTACCTCTGAAAGCGCAACCCCTGAATCCGGCCCAGGTAGCGAACCGGCAA |
| | | CCTCCGGTTCTGAAACCCCAGGTACCTCTGAAAGCGCTACTCCGGAGTCTGGCCCA |
| | | GGTACCTCTACTGAACCGTCTGAGGGTAGCGCTCCAGGTACTTCTACTGAACCGTC |
| | | CGAAGGTAGCGCACCAGGTACTTCTACCGAACCGTCCGAAGGCAGCGCTCCAGGTA |
| | | CCTCTACTGAACCTTCCGAGGGCAGCGCTCCAGGTACCTCTACCGAACCTTCTGAA |
| | | GGTAGCGCACCAGGTACTTCTACCGAACCGTCCGAGGGTAGCGCACCAGGTAGCCC |
| | | AGCAGGTTCTCCTACCTCCACCGAGGAAGGTACTTCTACCGAACCGTCCGAGGGTA |
| | | GCGCACCAGGTACCTCTGAAAGCGCAACTCCTGAGTCTGGCCCAGGTAGCGAACCT |
| | | GCTACCTCCGGCTCTGAGACTCCAGGTACCTCTGAAAGCGCAACCCCGGAATCTGG |
| | | TCCAGGTAGCGAACCTGCAACCTCTGGCTCTGAAACCCCAGGTACCTCTGAAAGCG |
| | | CTACTCCTGAATCTGGCCCAGGTACTTCTACTGAACCGTCCGAGGGCAGCGCACCA |
| | | GGTACTTCTGAAAGCGCTACTCCTGAGTCCGGCCCAGGTAGCCCGGCTGGCTCTCC |
| | | GACTTCCACCGAGGAAGGTAGCCCGGCTGGCTCTCCAACTTCTACTGAAGAAGGTA |
| | | GCCCGGCAGGCTCTCCGACCTCTACTGAGGAAGGTACTTCTGAAAGCGCAACCCCG |
| | | GAGTCCGGCCCAGGTACCTCTACCGAACCGTCTGAGGGCAGCGCACCA |
| AF576 | 102 | GGTTCTACTAGCTCTACCGCTGAATCTCCTGGCCCAGGTTCCACTAGCTCTACCGC |
| | | AGAATCTCCGGGCCCAGGTTCTACTAGCGAATCCCCTTCTGGTACCGCTCCAGGTT |
| | | CTACTAGCTCTACCGCTGAATCTCCGGGTCCAGGTTCTACCAGCTCTACTGCAGAA |
| | | TCTCCTGGCCCAGGTACTTCTACTCCGGAAAGCGGTTCCGCTTCTCCAGGTTCTAC |
| | | CAGCGAATCTCCTTCTGGCACCGCTCCAGGTACCTCTCCTAGCGGCGAATCTTCTA |
| | | CCGCTCCAGGTTCTACTAGCGAATCTCCTTCTGGCACTGCACCAGGTTCTACCAGC |
| | | GAATCTCCTTCTGGCACCGCTCCAGGTACCTCTCCTAGCGGCGAATCTTCTACCGC |
| | | TCCAGGTTCTACTAGCGAATCTCCTTCTGGCACTGCACCAGGTTCTACCAGCGAAT |
| | | CTCCTTCTGGCACCGCTCCAGGTACCTCTCCTAGCGGCGAATCTTCTACCGCTCCA |
| | | GGTTCTACTAGCGAATCTCCTTCTGGCACTGCACCAGGTTCTACTAGCGAATCTCC |
| | | TTCTGGCACTGCACCAGGTTCTACCAGCGAATCTCCGTCTGGCACTGCACCAGGTA |
| | | CCTCTACCCCTGAAAGCGGTTCCGCTTCTCCAGGTTCTACTAGCGAATCTCCTTCT |
| | | GGTACCGCTCCAGGTACTTCTACCCCTGAAAGCGGCTCCGCTTCTCCAGGTTCCAC |
| | | TAGCTCTACCGCTGAATCTCCGGGTCCAGGTTCTACTAGCTCTACTGCAGAATCTC |
| | | CTGGCCCAGGTACCTCTACTCCGGAAAGCGGCTCTGCATCTCCAGGTACTTCTACC |
| | | CCTGAAAGCGGTTCTGCATCTCCAGGTTCTACTAGCGAATCCCCGTCTGGTACCGC |
| | | ACCAGGTACTTCTACCCCGGAAAGCGGCTCTGCTTCTCCAGGTACTTCTACCCCGG |
| | | AAAGCGGCTCCGCATCTCCAGGTTCTACTAGCGAATCTCCTTCTGGTACCGCTCCA |
| | | GGTTCTACCAGCGAATCCCCGTCTGGTACTGCTCCAGGTTCTACCAGCGAATCTCC |
| | | TTCTGGTACTGCACCAGGTTCTACTAGCTCTACTGCAGAATCTCCTGGCCCAGGTA |
| | | CCTCTACTCCGGAAAGCGGCTCTGCATCTCCAGGTACTTCTACCCCTGAAAGCGGT |
| | | TCTGCATCTCCAGGTTCTACTAGCGAATCTCCTTCTGGCACTGCACCAGGTTCTAC |
| | | CAGCGAATCTCCGTCTGGCACTGCACCAGGTACCTCTACCCCTGAAAGCGGTTCCG |
| | | CTTCTCCAGGTTCTACTAGCGAATCTCCTTCTGGCACTGCACCAGGTTCTACCAGC |
| | | GAATCTCCGTCTGGCACTGCACCAGGTACCTCTACCCCTGAAAGCGGTTCCGCTTC |
| | | TCCAGGTACTTCTCCGAGCGGTGAATCTTCTACCGCACCAGGTTCTACTAGCTCTA |
| | | CCGCTGAATCTCCGGGCCCAGGTACTTCTCCGAGCGGTGAATCTTCTACTGCTCCA |
| | | GGTTCCACTAGCTCTACTGCTGAATCTCCTGGCCCAGGTACTTCTACTCCGGAAAG |
| | | CGGTTCCGCTTCTCCAGGTTCTACTAGCGAATCTCCGTCTGGCACCGCACCAGGTT |
| | | CTACTAGCTCTACTGCAGAATCTCCTGGCCCAGGTACCTCTACTCCGGAAAGCGGC |
| | | TCTGCATCTCCAGGTACTTCTACCCCTGAAAGCGGTTCTGCATCTCCA |
| AM875 | 103 | GGTACTTCTACTGAACCGTCTGAAGGCAGCGCACCAGGTAGCGAACCGGCTACTTC |
| | | CGGTTCTGAAACCCCAGGTAGCCCAGCAGGTTCTCCAACTTCTACTGAAGAAGGTT |
| | | CTACCAGCTCTACCGCAGAATCTCCTGGTCCAGGTACCTCTACTCCGGAAAGCGGC |
| | | TCTGCATCTCCAGGTTCTACTAGCGAATCTCCTTCTGGCACTGCACCAGGTTCTAC |
| | | TAGCGAATCCCCGTCTGGTACTGCTCCAGGTACTTCTACTCCTGAAAGCGGTTCCG |
| | | CTTCTCCAGGTACCTCTACTCCGGAAAGCGGTTCTGCATCTCCAGGTAGCGAACCG |
| | | GCAACCTCCGGCTCTGAAACCCCAGGTACCTCTGAAAGCGCTACTCCTGAATCCGG |
| | | CCCAGGTAGCCCGGCAGGTTCTCCGACTTCCACTGAGGAAGGTACCTCTACTGAAC |
| | | CTTCTGAGGGCAGCGCTCCAGGTACTTCTGAAAGCGCTACCCCGGAGTCCGGTCCA |
| | | GGTACTTCTACTGAACCGTCCGAAGGTAGCGCACCAGGTACTTCTACCGAACCGTC |
| | | CGAGGGTAGCGCACCAGGTAGCCCAGCAGGTTCTCCTACCTCCACCGAGGAAGGTA |
| | | CTTCTACCGAACCGTCCGAGGGTAGCGCACCAGGTACTTCTACCGAACCTTCCGAG |
| | | GGCAGCGCACCAGGTACTTCTGAAAGCGCTACCCCTGAGTCCGGCCCAGGTACTTC |
| | | TGAAAGCGCTACTCCTGAATCCGGTCCAGGTACCTCTACTGAACCTTCCGAAGGCA |
| | | GCGCTCCAGGTACCTCTACCGAACCGTCCGAGGGCAGCGCACCAGGTACTTCTGAA |
| | | AGCGCAACCCCTGAATCCGGTCCAGGTACTTCTACTGAACCTTCCGAAGGTAGCGC |
| | | TCCAGGTAGCGAACCTGCTACTTCTGGTTCTGAAACCCCAGGTAGCCCGGCTGGCT |
| | | CTCCGACCTCCACCGAGGAAGGTAGCTCTACCCCGTCTGGTGCTACTGGTTCTCCA |
| | | GGTACTCCGGGCAGCGGTACTGCTTCTTCCTCTCCAGGTAGCTCTACCCCTTCTGG |
| | | TGCTACTGGCTCTCCAGGTACCTCTACCGAACCGTCCGAGGGTAGCGCACCAGGTA |
| | | CCTCTACTGAACCGTCTGAGGGTAGCGCTCCAGGTAGCGAACCGGCAACCTCCGGT |
| | | TCTGAAACTCCAGGTAGCCCTGCTGGCTCTCCGACTTCTACTGAGGAAGGTAGCCC |
| | | GGCTGGTTCTCCGACTTCTACTGAGGAAGGTACTTCTACCGAACCTTCCGAAGGTA |
| | | GCGCTCCAGGTGCAAGCGCAAGCGGCGCGCCAAGCACGGGAGGTACTTCTGAAAGC |
| | | GCTACTCCTGAGTCCGGCCCAGGTAGCCCGGCTGGCTCTCCGACTTCCACCGAGGA |
| | | AGGTAGCCCGGCTGGCTCTCCAACTTCTACTGAAGAAGGTTCTACCAGCTCTACCG |
| | | CTGAATCTCCTGGCCCAGGTTCTACTAGCGAATCTCCGTCTGGCACCGCACCAGGT |
| | | ACTTCCCCTAGCGGTGAATCTTCTACTGCACCAGGTACCCCTGGCAGCGGTACCGC |
| | | TTCTTCCTCTCCAGGTAGCTCTACCCCGTCTGGTGCTACTGGCTCTCCAGGTTCTA |
| | | GCCCGTCTGCATCTACCGGTACCGGCCCAGGTAGCGAACCGGCAACCTCCGGCTCT |
| | | GAAACTCCAGGTACTTCTGAAAGCGCTACTCCGGAATCCGGCCCAGGTAGCGAACC |
| | | GGCTACTTCCGGCTCTGAAACCCCAGGTTCCACCAGCTCTACTGCAGAATCTCCGG |
| | | GCCCAGGTTCTACTAGCTCTACTGCAGAATCTCCGGGTCCAGGTACTTCTCCTAGC |
| | | GGCGAATCTTCTACCGCTCCAGGTAGCGAACCGGCAACCTCTGGCTCTGAAACTCC |
| | | AGGTAGCGAACCTGCAACCTCCGGCTCTGAAACCCCAGGTACTTCTACTGAACCTT |
| | | CTGAGGGCAGCGCACCAGGTTCTACCAGCTCTACCGCAGAATCTCCTGGTCCAGGT |
| | | ACCTCTACTCCGGAAAGCGGCTCTGCATCTCCAGGTTCTACTAGCGAATCTCCTTC |
| | | TGGCACTGCACCAGGTACTTCTACCGAACCGTCCGAAGGCAGCGCTCCAGGTACCT |
| | | CTACTGAACCTTCCGAGGGCAGCGCTCCAGGTACCTCTACCGAACCTTCTGAAGGT |
| | | AGCGCACCAGGTAGCTCTACTCCGTCTGGTGCAACCGGCTCCCCAGGTTCTAGCCC |
| | | GTCTGCTTCCACTGGTACTGGCCCAGGTGCTTCCCCGGGCACCAGCTCTACTGGTT |
| | | CTCCAGGTAGCGAACCTGCTACCTCCGGTTCTGAAACCCCAGGTACCTCTGAAAGC |
| | | GCAACTCCGGAGTCTGGTCCAGGTAGCCCTGCAGGTTCTCCTACCTCCACTGAGGA |
| | | AGGTAGCTCTACTCCGTCTGGTGCAACCGGCTCCCCAGGTTCTAGCCCGTCTGCTT |
| | | CCACTGGTACTGGCCCAGGTGCTTCCCCGGGCACCAGCTCTACTGGTTCTCCAGGT |
| | | ACCTCTGAAAGCGCTACTCCGGAGTCTGGCCCAGGTACCTCTACTGAACCGTCTGA |
| | | GGGTAGCGCTCCAGGTACTTCTACTGAACCGTCCGAAGGTAGCGCACCA |
| AE864 | 104 | GGTAGCCCGGCTGGCTCTCCTACCTCTACTGAGGAAGGTACTTCTGAAAGCGCTAC |
| | | TCCTGAGTCTGGTCCAGGTACCTCTACTGAACCGTCCGAAGGTAGCGCTCCAGGTA |
| | | GCCCAGCAGGCTCTCCGACTTCCACTGAGGAAGGTACTTCTACTGAACCTTCCGAA |
| | | GGCAGCGCACCAGGTACCTCTACTGAACCTTCTGAGGGCAGCGCTCCAGGTACTTC |
| | | TGAAAGCGCTACCCCGGAATCTGGCCCAGGTAGCGAACCGGCTACTTCTGGTTCTG |
| | | AAACCCCAGGTAGCGAACCGGCTACCTCCGGTTCTGAAACTCCAGGTAGCCCGGCA |
| | | GGCTCTCCGACCTCTACTGAGGAAGGTACTTCTGAAAGCGCAACCCCGGAGTCCGG |
| | | CCCAGGTACCTCTACCGAACCGTCTGAGGGCAGCGCACCAGGTACTTCTACCGAAC |
| | | CGTCCGAGGGTAGCGCACCAGGTAGCCCAGCAGGTTCTCCTACCTCCACCGAGGAA |
| | | GGTACTTCTACCGAACCGTCCGAGGGTAGCGCACCAGGTACCTCTACTGAACCTTC |
| | | TGAGGGCAGCGCTCCAGGTACTTCTGAAAGCGCTACCCCGGAGTCCGGTCCAGGTA |
| | | CTTCTACTGAACCGTCCGAAGGTAGCGCACCAGGTACTTCTGAAAGCGCAACCCCT |
| | | GAATCCGGTCCAGGTAGCGAACCGGCTACTTCTGGCTCTGAGACTCCAGGTACTTC |
| | | TACCGAACCGTCCGAAGGTAGCGCACCAGGTACTTCTACTGAACCGTCTGAAGGTA |
| | | GCGCACCAGGTACTTCTGAAAGCGCAACCCCGGAATCCGGCCCAGGTACCTCTGAA |
| | | AGCGCAACCCCGGAGTCCGGCCCAGGTAGCCCTGCTGGCTCTCCAACCTCCACCGA |
| | | AGAAGGTACCTCTGAAAGCGCAACCCCTGAATCCGGCCCAGGTAGCGAACCGGCAA |
| | | CCTCCGGTTCTGAAACCCCAGGTACCTCTGAAAGCGCTACTCCGGAGTCTGGCCCA |
| | | GGTACCTCTACTGAACCGTCTGAGGGTAGCGCTCCAGGTACTTCTACTGAACCGTC |
| | | CGAAGGTAGCGCACCAGGTACTTCTACCGAACCGTCCGAAGGCAGCGCTCCAGGTA |
| | | CCTCTACTGAACCTTCCGAGGGCAGCGCTCCAGGTACCTCTACCGAACCTTCTGAA |
| | | GGTAGCGCACCAGGTACTTCTACCGAACCGTCCGAGGGTAGCGCACCAGGTAGCCC |
| | | AGCAGGTTCTCCTACCTCCACCGAGGAAGGTACTTCTACCGAACCGTCCGAGGGTA |
| | | GCGCACCAGGTACCTCTGAAAGCGCAACTCCTGAGTCTGGCCCAGGTAGCGAACCT |
| | | GCTACCTCCGGCTCTGAGACTCCAGGTACCTCTGAAAGCGCAACCCCGGAATCTGG |
| | | TCCAGGTAGCGAACCTGCAACCTCTGGCTCTGAAACCCCAGGTACCTCTGAAAGCG |
| | | CTACTCCTGAATCTGGCCCAGGTACTTCTACTGAACCGTCCGAGGGCAGCGCACCA |
| | | GGTACTTCTGAAAGCGCTACTCCTGAGTCCGGCCCAGGTAGCCCGGCTGGCTCTCC |
| | | GACTTCCACCGAGGAAGGTAGCCCGGCTGGCTCTCCAACTTCTACTGAAGAAGGTA |
| | | GCCCGGCAGGCTCTCCGACCTCTACTGAGGAAGGTACTTCTGAAAGCGCAACCCCG |
| | | GAGTCCGGCCCAGGTACCTCTACCGAACCGTCTGAGGGCAGCGCACCAGGTACCTC |
| | | TGAAAGCGCAACTCCTGAGTCTGGCCCAGGTAGCGAACCTGCTACCTCCGGCTCTG |
| | | AGACTCCAGGTACCTCTGAAAGCGCAACCCCGGAATCTGGTCCAGGTAGCGAACCT |
| | | GCAACCTCTGGCTCTGAAACCCCAGGTACCTCTGAAAGCGCTACTCCTGAATCTGG |
| | | CCCAGGTACTTCTACTGAACCGTCCGAGGGCAGCGCACCAGGTAGCCCTGCTGGCT |
| | | CTCCAACCTCCACCGAAGAAGGTACCTCTGAAAGCGCAACCCCTGAATCCGGCCCA |
| | | GGTAGCGAACCGGCAACCTCCGGTTCTGAAACCCCAGGTACTTCTGAAAGCGCTAC |
| | | TCCTGAGTCCGGCCCAGGTAGCCCGGCTGGCTCTCCGACTTCCACCGAGGAAGGTA |
| | | GCCCGGCTGGCTCTCCAACTTCTACTGAAGAAGGTACTTCTACCGAACCTTCCGAG |
| | | GGCAGCGCACCAGGTACTTCTGAAAGCGCTACCCCTGAGTCCGGCCCAGGTACTTC |
| | | TGAAAGCGCTACTCCTGAATCCGGTCCAGGTACTTCTGAAAGCGCTACCCCGGAAT |
| | | CTGGCCCAGGTAGCGAACCGGCTACTTCTGGTTCTGAAACCCCAGGTAGCGAACCG |
| | | GCTACCTCCGGTTCTGAAACTCCAGGTAGCCCAGCAGGCTCTCCGACTTCCACTGA |
| | | GGAAGGTACTTCTACTGAACCTTCCGAAGGCAGCGCACCAGGTACCTCTACTGAAC |
| | | CTTCTGAGGGCAGCGCTCCAGGTAGCGAACCTGCAACCTCTGGCTCTGAAACCCCA |
| | | GGTACCTCTGAAAGCGCTACTCCTGAATCTGGCCCAGGTACTTCTACTGAACCGTC |
| | | CGAGGGCAGCGCACCA |
| A1864 | 105 | GGTTCTACCAGCGAATCTCCTTCTGGCACCGCTCCAGGTACCTCTCCTAGCGGCGA |
| | | ATCTTCTACCGCTCCAGGTTCTACTAGCGAATCTCCTTCTGGCACTGCACCAGGTT |
| | | CTACTAGCGAATCCCCGTCTGGTACTGCTCCAGGTACTTCTACTCCTGAAAGCGGT |
| | | TCCGCTTCTCCAGGTACCTCTACTCCGGAAAGCGGTTCTGCATCTCCAGGTTCTAC |
| | | CAGCGAATCTCCTTCTGGCACCGCTCCAGGTTCTACTAGCGAATCCCCGTCTGGTA |
| | | CCGCACCAGGTACTTCTCCTAGCGGCGAATCTTCTACCGCACCAGGTTCTACTAGC |
| | | GAATCTCCGTCTGGCACTGCTCCAGGTACTTCTCCTAGCGGTGAATCTTCTACCGC |
| | | TCCAGGTACTTCCCCTAGCGGCGAATCTTCTACCGCTCCAGGTTCTACTAGCTCTA |
| | | CTGCAGAATCTCCGGGCCCAGGTACCTCTCCTAGCGGTGAATCTTCTACCGCTCCA |
| | | GGTACTTCTCCGAGCGGTGAATCTTCTACCGCTCCAGGTTCTACTAGCTCTACTGC |
| | | AGAATCTCCTGGCCCAGGTACCTCTACTCCGGAAAGCGGCTCTGCATCTCCAGGTA |
| | | CTTCTACCCCTGAAAGCGGTTCTGCATCTCCAGGTTCTACTAGCGAATCTCCTTCT |
| | | GGCACTGCACCAGGTTCTACCAGCGAATCTCCGTCTGGCACTGCACCAGGTACCTC |
| | | TACCCCTGAAAGCGGTTCCGCTTCTCCAGGTTCTACCAGCTCTACCGCAGAATCTC |
| | | CTGGTCCAGGTACCTCTACTCCGGAAAGCGGCTCTGCATCTCCAGGTTCTACTAGC |
| | | GAATCTCCTTCTGGCACTGCACCAGGTACTTCTCCGAGCGGTGAATCTTCTACCGC |
| | | ACCAGGTTCTACTAGCTCTACCGCTGAATCTCCGGGCCCAGGTACTTCTCCGAGCG |
| | | GTGAATCTTCTACTGCTCCAGGTACCTCTACTCCTGAAAGCGGTTCTGCATCTCCA |
| | | GGTTCCACTAGCTCTACCGCAGAATCTCCGGGCCCAGGTTCTACTAGCTCTACTGC |
| | | TGAATCTCCTGGCCCAGGTTCTACTAGCTCTACTGCTGAATCTCCGGGTCCAGGTT |
| | | CTACCAGCTCTACTGCTGAATCTCCTGGTCCAGGTACCTCCCCGAGCGGTGAATCT |
| | | TCTACTGCACCAGGTTCTACTAGCGAATCTCCTTCTGGCACTGCACCAGGTTCTAC |
| | | CAGCGAATCTCCGTCTGGCACTGCACCAGGTACCTCTACCCCTGAAAGCGGTCCXX |
| | | XXXXXXXXXXTGCAAGCGCAAGCGGCGCGCCAAGCACGGGAXXXXXXXXTAGCGAA |
| | | TCTCCTTCTGGTACCGCTCCAGGTTCTACCAGCGAATCCCCGTCTGGTACTGCTCC |
| | | AGGTTCTACCAGCGAATCTCCTTCTGGTACTGCACCAGGTTCTACTAGCGAATCTC |
| | | CTTCTGGTACCGCTCCAGGTTCTACCAGCGAATCCCCGTCTGGTACTGCTCCAGGT |
| | | TCTACCAGCGAATCTCCTTCTGGTACTGCACCAGGTACTTCTACTCCGGAAAGCGG |
| | | TTCCGCATCTCCAGGTACTTCTCCTAGCGGTGAATCTTCTACTGCTCCAGGTACCT |
| | | CTCCTAGCGGCGAATCTTCTACTGCTCCAGGTTCTACCAGCTCTACTGCTGAATCT |
| | | CCGGGTCCAGGTACTTCCCCGAGCGGTGAATCTTCTACTGCACCAGGTACTTCTAC |
| | | TCCGGAAAGCGGTTCCGCTTCTCCAGGTTCTACCAGCGAATCTCCTTCTGGCACCG |
| | | CTCCAGGTTCTACTAGCGAATCCCCGTCTGGTACCGCACCAGGTACTTCTCCTAGC |
| | | GGCGAATCTTCTACCGCACCAGGTTCTACTAGCGAATCCCCGTCTGGTACCGCACC |
| | | AGGTACTTCTACCCCGGAAAGCGGCTCTGCTTCTCCAGGTACTTCTACCCCGGAAA |
| | | GCGGCTCCGCATCTCCAGGTTCTACTAGCGAATCTCCTTCTGGTACCGCTCCAGGT |
| | | ACTTCTACCCCTGAAAGCGGCTCCGCTTCTCCAGGTTCCACTAGCTCTACCGCTGA |
| | | ATCTCCGGGTCCAGGTTCTACCAGCGAATCTCCTTCTGGCACCGCTCCAGGTTCTA |
| | | CTAGCGAATCCCCGTCTGGTACCGCACCAGGTACTTCTCCTAGCGGCGAATCTTCT |
| | | ACCGCACCAGGTTCTACCAGCTCTACTGCTGAATCTCCGGGTCCAGGTACTTCCCC |
| | | GAGCGGTGAATCTTCTACTGCACCAGGTACTTCTACTCCGGAAAGCGGTTCCGCTT |
| | | CTCCAGGTACCTCCCCTAGCGGCGAATCTTCTACTGCTCCAGGTACCTCTCCTAGC |
| | | GGCGAATCTTCTACCGCTCCAGGTACCTCCCCTAGCGGTGAATCTTCTACCGCACC |
| | | AGGTTCTACTAGCTCTACTGCTGAATCTCCGGGTCCAGGTTCTACCAGCTCTACTG |
| | | CTGAATCTCCTGGTCCAGGTACCTCCCCGAGCGGTGAATCTTCTACTGCACCAGGT |
| | | TCTAGCCCTTCTGCTTCCACCGGTACCGGCCCAGGTAGCTCTACTCCGTCTGGTGC |
| | | AACTGGCTCTCCAGGTAGCTCTACTCCGTCTGGTGCAACCGGCTCCCCA |
| | | XXXX was inserted in two areas where no sequence |
| | | information is available. |
| AG864 | 106 | GGTGCTTCCCCGGGCACCAGCTCTACTGGTTCTCCAGGTTCTAGCCCGTCTGCTTC |
| | | TACTGGTACTGGTCCAGGTTCTAGCCCTTCTGCTTCCACTGGTACTGGTCCAGGTA |
| | | CCCCGGGTAGCGGTACCGCTTCTTCTTCTCCAGGTAGCTCTACTCCGTCTGGTGCT |
| | | ACCGGCTCTCCAGGTTCTAACCCTTCTGCATCCACCGGTACCGGCCCAGGTGCTTC |
| | | TCCGGGCACCAGCTCTACTGGTTCTCCAGGTACCCCGGGCAGCGGTACCGCATCTT |
| | | CTTCTCCAGGTAGCTCTACTCCTTCTGGTGCAACTGGTTCTCCAGGTACTCCTGGC |
| | | AGCGGTACCGCTTCTTCTTCTCCAGGTGCTTCTCCTGGTACTAGCTCTACTGGTTC |
| | | TCCAGGTGCTTCTCCGGGCACTAGCTCTACTGGTTCTCCAGGTACCCCGGGTAGCG |
| | | GTACTGCTTCTTCCTCTCCAGGTAGCTCTACCCCTTCTGGTGCAACCGGCTCTCCA |
| | | GGTGCTTCTCCGGGCACCAGCTCTACCGGTTCTCCAGGTACCCCGGGTAGCGGTAC |
| | | CGCTTCTTCTTCTCCAGGTAGCTCTACTCCGTCTGGTGCTACCGGCTCTCCAGGTT |
| | | CTAACCCTTCTGCATCCACCGGTACCGGCCCAGGTTCTAGCCCTTCTGCTTCCACC |
| | | GGTACTGGCCCAGGTAGCTCTACCCCTTCTGGTGCTACCGGCTCCCCAGGTAGCTC |
| | | TACTCCTTCTGGTGCAACTGGCTCTCCAGGTGCATCTCCGGGCACTAGCTCTACTG |
| | | GTTCTCCAGGTGCATCCCCTGGCACTAGCTCTACTGGTTCTCCAGGTGCTTCTCCT |
| | | GGTACCAGCTCTACTGGTTCTCCAGGTACTCCTGGCAGCGGTACCGCTTCTTCTTC |
| | | TCCAGGTGCTTCTCCTGGTACTAGCTCTACTGGTTCTCCAGGTGCTTCTCCGGGCA |
| | | CTAGCTCTACTGGTTCTCCAGGTGCTTCCCCGGGCACTAGCTCTACCGGTTCTCCA |
| | | GGTTCTAGCCCTTCTGCATCTACTGGTACTGGCCCAGGTACTCCGGGCAGCGGTAC |
| | | TGCTTCTTCCTCTCCAGGTGCATCTCCGGGCACTAGCTCTACTGGTTCTCCAGGTG |
| | | CATCCCCTGGCACTAGCTCTACTGGTTCTCCAGGTGCTTCTCCTGGTACCAGCTCT |
| | | ACTGGTTCTCCAGGTAGCTCTACTCCGTCTGGTGCAACCGGTTCCCCAGGTAGCTC |
| | | TACTCCTTCTGGTGCTACTGGCTCCCCAGGTGCATCCCCTGGCACCAGCTCTACCG |
| | | GTTCTCCAGGTACCCCGGGCAGCGGTACCGCATCTTCCTCTCCAGGTAGCTCTACC |
| | | CCGTCTGGTGCTACCGGTTCCCCAGGTAGCTCTACCCCGTCTGGTGCAACCGGCTC |
| | | CCCAGGTAGCTCTACTCCGTCTGGTGCAACCGGCTCCCCAGGTTCTAGCCCGTCTG |
| | | CTTCCACTGGTACTGGCCCAGGTGCTTCCCCGGGCACCAGCTCTACTGGTTCTCCA |
| | | GGTGCATCCCCGGGTACCAGCTCTACCGGTTCTCCAGGTACTCCTGGCAGCGGTAC |
| | | TGCATCTTCCTCTCCAGGTGCTTCTCCGGGCACCAGCTCTACTGGTTCTCCAGGTG |
| | | CATCTCCGGGCACTAGCTCTACTGGTTCTCCAGGTGCATCCCCTGGCACTAGCTCT |
| | | ACTGGTTCTCCAGGTGCTTCTCCTGGTACCAGCTCTACTGGTTCTCCAGGTACCCC |
| | | TGGTAGCGGTACTGCTTCTTCCTCTCCAGGTAGCTCTACTCCGTCTGGTGCTACCG |
| | | GTTCTCCAGGTACCCCGGGTAGCGGTACCGCATCTTCTTCTCCAGGTAGCTCTACC |
| | | CCGTCTGGTGCTACTGGTTCTCCAGGTACTCCGGGCAGCGGTACTGCTTCTTCCTC |
| | | TCCAGGTAGCTCTACCCCTTCTGGTGCTACTGGCTCTCCAGGTAGCTCTACCCCGT |
| | | CTGGTGCTACTGGCTCCCCAGGTTCTAGCCCTTCTGCATCCACCGGTACCGGTCCA |
| | | GGTTCTAGCCCGTCTGCATCTACTGGTACTGGTCCAGGTGCATCCCCGGGCACTAG |
| | | CTCTACCGGTTCTCCAGGTACTCCTGGTAGCGGTACTGCTTCTTCTTCTCCAGGTA |
| | | GCTCTACTCCTTCTGGTGCTACTGGTTCTCCAGGTTCTAGCCCTTCTGCATCCACC |
| | | GGTACCGGCCCAGGTTCTAGCCCGTCTGCTTCTACCGGTACTGGTCCAGGTGCTTC |
| | | TCCGGGTACTAGCTCTACTGGTTCTCCAGGTGCATCTCCTGGTACTAGCTCTACTG |
| | | GTTCTCCAGGTAGCTCTACTCCGTCTGGTGCAACCGGCTCTCCAGGTTCTAGCCCT |
| | | TCTGCATCTACCGGTACTGGTCCAGGTGCATCCCCTGGTACCAGCTCTACCGGTTC |
| | | TCCAGGTTCTAGCCCTTCTGCTTCTACCGGTACCGGTCCAGGTACCCCTGGCAGCG |
| | | GTACCGCATCTTCCTCTCCAGGTAGCTCTACTCCGTCTGGTGCAACCGGTTCCCCA |
| | | GGTAGCTCTACTCCTTCTGGTGCTACTGGCTCCCCAGGTGCATCCCCTGGCACCAG |
| | | CTCTACCGGTTCTCCA |
| AM923 | 107 | ATGGCTGAACCTGCTGGCTCTCCAACCTCCACTGAGGAAGGTGCATCCCCGGGCAC |
| | | CAGCTCTACCGGTTCTCCAGGTAGCTCTACCCCGTCTGGTGCTACCGGCTCTCCAG |
| | | GTAGCTCTACCCCGTCTGGTGCTACTGGCTCTCCAGGTACTTCTACTGAACCGTCT |
| | | GAAGGCAGCGCACCAGGTAGCGAACCGGCTACTTCCGGTTCTGAAACCCCAGGTAG |
| | | CCCAGCAGGTTCTCCAACTTCTACTGAAGAAGGTTCTACCAGCTCTACCGCAGAAT |
| | | CTCCTGGTCCAGGTACCTCTACTCCGGAAAGCGGCTCTGCATCTCCAGGTTCTACT |
| | | AGCGAATCTCCTTCTGGCACTGCACCAGGTTCTACTAGCGAATCCCCGTCTGGTAC |
| | | TGCTCCAGGTACTTCTACTCCTGAAAGCGGTTCCGCTTCTCCAGGTACCTCTACTC |
| | | CGGAAAGCGGTTCTGCATCTCCAGGTAGCGAACCGGCAACCTCCGGCTCTGAAACC |
| | | CCAGGTACCTCTGAAAGCGCTACTCCTGAATCCGGCCCAGGTAGCCCGGCAGGTTC |
| | | TCCGACTTCCACTGAGGAAGGTACCTCTACTGAACCTTCTGAGGGCAGCGCTCCAG |
| | | GTACTTCTGAAAGCGCTACCCCGGAGTCCGGTCCAGGTACTTCTACTGAACCGTCC |
| | | GAAGGTAGCGCACCAGGTACTTCTACCGAACCGTCCGAGGGTAGCGCACCAGGTAG |
| | | CCCAGCAGGTTCTCCTACCTCCACCGAGGAAGGTACTTCTACCGAACCGTCCGAGG |
| | | GTAGCGCACCAGGTACTTCTACCGAACCTTCCGAGGGCAGCGCACCAGGTACTTCT |
| | | GAAAGCGCTACCCCTGAGTCCGGCCCAGGTACTTCTGAAAGCGCTACTCCTGAATC |
| | | CGGTCCAGGTACCTCTACTGAACCTTCCGAAGGCAGCGCTCCAGGTACCTCTACCG |
| | | AACCGTCCGAGGGCAGCGCACCAGGTACTTCTGAAAGCGCAACCCCTGAATCCGGT |
| | | CCAGGTACTTCTACTGAACCTTCCGAAGGTAGCGCTCCAGGTAGCGAACCTGCTAC |
| | | TTCTGGTTCTGAAACCCCAGGTAGCCCGGCTGGCTCTCCGACCTCCACCGAGGAAG |
| | | GTAGCTCTACCCCGTCTGGTGCTACTGGTTCTCCAGGTACTCCGGGCAGCGGTACT |
| | | GCTTCTTCCTCTCCAGGTAGCTCTACCCCTTCTGGTGCTACTGGCTCTCCAGGTAC |
| | | CTCTACCGAACCGTCCGAGGGTAGCGCACCAGGTACCTCTACTGAACCGTCTGAGG |
| | | GTAGCGCTCCAGGTAGCGAACCGGCAACCTCCGGTTCTGAAACTCCAGGTAGCCCT |
| | | GCTGGCTCTCCGACTTCTACTGAGGAAGGTAGCCCGGCTGGTTCTCCGACTTCTAC |
| | | TGAGGAAGGTACTTCTACCGAACCTTCCGAAGGTAGCGCTCCAGGTGCAAGCGCAA |
| | | GCGGCGCGCCAAGCACGGGAGGTACTTCTGAAAGCGCTACTCCTGAGTCCGGCCCA |
| | | GGTAGCCCGGCTGGCTCTCCGACTTCCACCGAGGAAGGTAGCCCGGCTGGCTCTCC |
| | | AACTTCTACTGAAGAAGGTTCTACCAGCTCTACCGCTGAATCTCCTGGCCCAGGTT |
| | | CTACTAGCGAATCTCCGTCTGGCACCGCACCAGGTACTTCCCCTAGCGGTGAATCT |
| | | TCTACTGCACCAGGTACCCCTGGCAGCGGTACCGCTTCTTCCTCTCCAGGTAGCTC |
| | | TACCCCGTCTGGTGCTACTGGCTCTCCAGGTTCTAGCCCGTCTGCATCTACCGGTA |
| | | CCGGCCCAGGTAGCGAACCGGCAACCTCCGGCTCTGAAACTCCAGGTACTTCTGAA |
| | | AGCGCTACTCCGGAATCCGGCCCAGGTAGCGAACCGGCTACTTCCGGCTCTGAAAC |
| | | CCCAGGTTCCACCAGCTCTACTGCAGAATCTCCGGGCCCAGGTTCTACTAGCTCTA |
| | | CTGCAGAATCTCCGGGTCCAGGTACTTCTCCTAGCGGCGAATCTTCTACCGCTCCA |
| | | GGTAGCGAACCGGCAACCTCTGGCTCTGAAACTCCAGGTAGCGAACCTGCAACCTC |
| | | CGGCTCTGAAACCCCAGGTACTTCTACTGAACCTTCTGAGGGCAGCGCACCAGGTT |
| | | CTACCAGCTCTACCGCAGAATCTCCTGGTCCAGGTACCTCTACTCCGGAAAGCGGC |
| | | TCTGCATCTCCAGGTTCTACTAGCGAATCTCCTTCTGGCACTGCACCAGGTACTTC |
| | | TACCGAACCGTCCGAAGGCAGCGCTCCAGGTACCTCTACTGAACCTTCCGAGGGCA |
| | | GCGCTCCAGGTACCTCTACCGAACCTTCTGAAGGTAGCGCACCAGGTAGCTCTACT |
| | | CCGTCTGGTGCAACCGGCTCCCCAGGTTCTAGCCCGTCTGCTTCCACTGGTACTGG |
| | | CCCAGGTGCTTCCCCGGGCACCAGCTCTACTGGTTCTCCAGGTAGCGAACCTGCTA |
| | | CCTCCGGTTCTGAAACCCCAGGTACCTCTGAAAGCGCAACTCCGGAGTCTGGTCCA |
| | | GGTAGCCCTGCAGGTTCTCCTACCTCCACTGAGGAAGGTAGCTCTACTCCGTCTGG |
| | | TGCAACCGGCTCCCCAGGTTCTAGCCCGTCTGCTTCCACTGGTACTGGCCCAGGTG |
| | | CTTCCCCGGGCACCAGCTCTACTGGTTCTCCAGGTACCTCTGAAAGCGCTACTCCG |
| | | GAGTCTGGCCCAGGTACCTCTACTGAACCGTCTGAGGGTAGCGCTCCAGGTACTTC |
| | | TACTGAACCGTCCGAAGGTAGCGCACCA |
| AE912 | 108 | ATGGCTGAACCTGCTGGCTCTCCAACCTCCACTGAGGAAGGTACCCCGGGTAGCGG |
| | | TACTGCTTCTTCCTCTCCAGGTAGCTCTACCCCTTCTGGTGCAACCGGCTCTCCAG |
| | | GTGCTTCTCCGGGCACCAGCTCTACCGGTTCTCCAGGTAGCCCGGCTGGCTCTCCT |
| | | ACCTCTACTGAGGAAGGTACTTCTGAAAGCGCTACTCCTGAGTCTGGTCCAGGTAC |
| | | CTCTACTGAACCGTCCGAAGGTAGCGCTCCAGGTAGCCCAGCAGGCTCTCCGACTT |
| | | CCACTGAGGAAGGTACTTCTACTGAACCTTCCGAAGGCAGCGCACCAGGTACCTCT |
| | | ACTGAACCTTCTGAGGGCAGCGCTCCAGGTACTTCTGAAAGCGCTACCCCGGAATC |
| | | TGGCCCAGGTAGCGAACCGGCTACTTCTGGTTCTGAAACCCCAGGTAGCGAACCGG |
| | | CTACCTCCGGTTCTGAAACTCCAGGTAGCCCGGCAGGCTCTCCGACCTCTACTGAG |
| | | GAAGGTACTTCTGAAAGCGCAACCCCGGAGTCCGGCCCAGGTACCTCTACCGAACC |
| | | GTCTGAGGGCAGCGCACCAGGTACTTCTACCGAACCGTCCGAGGGTAGCGCACCAG |
| | | GTAGCCCAGCAGGTTCTCCTACCTCCACCGAGGAAGGTACTTCTACCGAACCGTCC |
| | | GAGGGTAGCGCACCAGGTACCTCTACTGAACCTTCTGAGGGCAGCGCTCCAGGTAC |
| | | TTCTGAAAGCGCTACCCCGGAGTCCGGTCCAGGTACTTCTACTGAACCGTCCGAAG |
| | | GTAGCGCACCAGGTACTTCTGAAAGCGCAACCCCTGAATCCGGTCCAGGTAGCGAA |
| | | CCGGCTACTTCTGGCTCTGAGACTCCAGGTACTTCTACCGAACCGTCCGAAGGTAG |
| | | CGCACCAGGTACTTCTACTGAACCGTCTGAAGGTAGCGCACCAGGTACTTCTGAAA |
| | | GCGCAACCCCGGAATCCGGCCCAGGTACCTCTGAAAGCGCAACCCCGGAGTCCGGC |
| | | CCAGGTAGCCCTGCTGGCTCTCCAACCTCCACCGAAGAAGGTACCTCTGAAAGCGC |
| | | AACCCCTGAATCCGGCCCAGGTAGCGAACCGGCAACCTCCGGTTCTGAAACCCCAG |
| | | GTACCTCTGAAAGCGCTACTCCGGAGTCTGGCCCAGGTACCTCTACTGAACCGTCT |
| | | GAGGGTAGCGCTCCAGGTACTTCTACTGAACCGTCCGAAGGTAGCGCACCAGGTAC |
| | | TTCTACCGAACCGTCCGAAGGCAGCGCTCCAGGTACCTCTACTGAACCTTCCGAGG |
| | | GCAGCGCTCCAGGTACCTCTACCGAACCTTCTGAAGGTAGCGCACCAGGTACTTCT |
| | | ACCGAACCGTCCGAGGGTAGCGCACCAGGTAGCCCAGCAGGTTCTCCTACCTCCAC |
| | | CGAGGAAGGTACTTCTACCGAACCGTCCGAGGGTAGCGCACCAGGTACCTCTGAAA |
| | | GCGCAACTCCTGAGTCTGGCCCAGGTAGCGAACCTGCTACCTCCGGCTCTGAGACT |
| | | CCAGGTACCTCTGAAAGCGCAACCCCGGAATCTGGTCCAGGTAGCGAACCTGCAAC |
| | | CTCTGGCTCTGAAACCCCAGGTACCTCTGAAAGCGCTACTCCTGAATCTGGCCCAG |
| | | GTACTTCTACTGAACCGTCCGAGGGCAGCGCACCAGGTACTTCTGAAAGCGCTACT |
| | | CCTGAGTCCGGCCCAGGTAGCCCGGCTGGCTCTCCGACTTCCACCGAGGAAGGTAG |
| | | CCCGGCTGGCTCTCCAACTTCTACTGAAGAAGGTAGCCCGGCAGGCTCTCCGACCT |
| | | CTACTGAGGAAGGTACTTCTGAAAGCGCAACCCCGGAGTCCGGCCCAGGTACCTCT |
| | | ACCGAACCGTCTGAGGGCAGCGCACCAGGTACCTCTGAAAGCGCAACTCCTGAGTC |
| | | TGGCCCAGGTAGCGAACCTGCTACCTCCGGCTCTGAGACTCCAGGTACCTCTGAAA |
| | | GCGCAACCCCGGAATCTGGTCCAGGTAGCGAACCTGCAACCTCTGGCTCTGAAACC |
| | | CCAGGTACCTCTGAAAGCGCTACTCCTGAATCTGGCCCAGGTACTTCTACTGAACC |
| | | GTCCGAGGGCAGCGCACCAGGTAGCCCTGCTGGCTCTCCAACCTCCACCGAAGAAG |
| | | GTACCTCTGAAAGCGCAACCCCTGAATCCGGCCCAGGTAGCGAACCGGCAACCTCC |
| | | GGTTCTGAAACCCCAGGTACTTCTGAAAGCGCTACTCCTGAGTCCGGCCCAGGTAG |
| | | CCCGGCTGGCTCTCCGACTTCCACCGAGGAAGGTAGCCCGGCTGGCTCTCCAACTT |
| | | CTACTGAAGAAGGTACTTCTACCGAACCTTCCGAGGGCAGCGCACCAGGTACTTCT |
| | | GAAAGCGCTACCCCTGAGTCCGGCCCAGGTACTTCTGAAAGCGCTACTCCTGAATC |
| | | CGGTCCAGGTACTTCTGAAAGCGCTACCCCGGAATCTGGCCCAGGTAGCGAACCGG |
| | | CTACTTCTGGTTCTGAAACCCCAGGTAGCGAACCGGCTACCTCCGGTTCTGAAACT |
| | | CCAGGTAGCCCAGCAGGCTCTCCGACTTCCACTGAGGAAGGTACTTCTACTGAACC |
| | | TTCCGAAGGCAGCGCACCAGGTACCTCTACTGAACCTTCTGAGGGCAGCGCTCCAG |
| | | GTAGCGAACCTGCAACCTCTGGCTCTGAAACCCCAGGTACCTCTGAAAGCGCTACT |
| | | CCTGAATCTGGCCCAGGTACTTCTACTGAACCGTCCGAGGGCAGCGCACCA |
| AM1296 | 109 | GGTACTTCTACTGAACCGTCTGAAGGCAGCGCACCAGGTAGCGAACCGGCTACTTC |
| | | CGGTTCTGAAACCCCAGGTAGCCCAGCAGGTTCTCCAACTTCTACTGAAGAAGGTT |
| | | CTACCAGCTCTACCGCAGAATCTCCTGGTCCAGGTACCTCTACTCCGGAAAGCGGC |
| | | TCTGCATCTCCAGGTTCTACTAGCGAATCTCCTTCTGGCACTGCACCAGGTTCTAC |
| | | TAGCGAATCCCCGTCTGGTACTGCTCCAGGTACTTCTACTCCTGAAAGCGGTTCCG |
| | | CTTCTCCAGGTACCTCTACTCCGGAAAGCGGTTCTGCATCTCCAGGTAGCGAACCG |
| | | GCAACCTCCGGCTCTGAAACCCCAGGTACCTCTGAAAGCGCTACTCCTGAATCCGG |
| | | CCCAGGTAGCCCGGCAGGTTCTCCGACTTCCACTGAGGAAGGTACCTCTACTGAAC |
| | | CTTCTGAGGGCAGCGCTCCAGGTACTTCTGAAAGCGCTACCCCGGAGTCCGGTCCA |
| | | GGTACTTCTACTGAACCGTCCGAAGGTAGCGCACCAGGTACTTCTACCGAACCGTC |
| | | CGAGGGTAGCGCACCAGGTAGCCCAGCAGGTTCTCCTACCTCCACCGAGGAAGGTA |
| | | CTTCTACCGAACCGTCCGAGGGTAGCGCACCAGGTACTTCTACCGAACCTTCCGAG |
| | | GGCAGCGCACCAGGTACTTCTGAAAGCGCTACCCCTGAGTCCGGCCCAGGTACTTC |
| | | TGAAAGCGCTACTCCTGAATCCGGTCCAGGTACCTCTACTGAACCTTCCGAAGGCA |
| | | GCGCTCCAGGTACCTCTACCGAACCGTCCGAGGGCAGCGCACCAGGTACTTCTGAA |
| | | AGCGCAACCCCTGAATCCGGTCCAGGTACTTCTACTGAACCTTCCGAAGGTAGCGC |
| | | TCCAGGTAGCGAACCTGCTACTTCTGGTTCTGAAACCCCAGGTAGCCCGGCTGGCT |
| | | CTCCGACCTCCACCGAGGAAGGTAGCTCTACCCCGTCTGGTGCTACTGGTTCTCCA |
| | | GGTACTCCGGGCAGCGGTACTGCTTCTTCCTCTCCAGGTAGCTCTACCCCTTCTGG |
| | | TGCTACTGGCTCTCCAGGTACCTCTACCGAACCGTCCGAGGGTAGCGCACCAGGTA |
| | | CCTCTACTGAACCGTCTGAGGGTAGCGCTCCAGGTAGCGAACCGGCAACCTCCGGT |
| | | TCTGAAACTCCAGGTAGCCCTGCTGGCTCTCCGACTTCTACTGAGGAAGGTAGCCC |
| | | GGCTGGTTCTCCGACTTCTACTGAGGAAGGTACTTCTACCGAACCTTCCGAAGGTA |
| | | GCGCTCCAGGTCCAGAACCAACGGGGCCGGCCCCAAGCGGAGGTAGCGAACCGGCA |
| | | ACCTCCGGCTCTGAAACCCCAGGTACCTCTGAAAGCGCTACTCCTGAATCCGGCCC |
| | | AGGTAGCCCGGCAGGTTCTCCGACTTCCACTGAGGAAGGTACTTCTGAAAGCGCTA |
| | | CTCCTGAGTCCGGCCCAGGTAGCCCGGCTGGCTCTCCGACTTCCACCGAGGAAGGT |
| | | AGCCCGGCTGGCTCTCCAACTTCTACTGAAGAAGGTACTTCTGAAAGCGCTACTCC |
| | | TGAGTCCGGCCCAGGTAGCCCGGCTGGCTCTCCGACTTCCACCGAGGAAGGTAGCC |
| | | CGGCTGGCTCTCCAACTTCTACTGAAGAAGGTTCTACCAGCTCTACCGCTGAATCT |
| | | CCTGGCCCAGGTTCTACTAGCGAATCTCCGTCTGGCACCGCACCAGGTACTTCCCC |
| | | TAGCGGTGAATCTTCTACTGCACCAGGTTCTACCAGCGAATCTCCTTCTGGCACCG |
| | | CTCCAGGTTCTACTAGCGAATCCCCGTCTGGTACCGCACCAGGTACTTCTCCTAGC |
| | | GGCGAATCTTCTACCGCACCAGGTACTTCTACCGAACCTTCCGAGGGCAGCGCACC |
| | | AGGTACTTCTGAAAGCGCTACCCCTGAGTCCGGCCCAGGTACTTCTGAAAGCGCTA |
| | | CTCCTGAATCCGGTCCAGGTAGCGAACCGGCAACCTCTGGCTCTGAAACCCCAGGT |
| | | ACCTCTGAAAGCGCTACTCCGGAATCTGGTCCAGGTACTTCTGAAAGCGCTACTCC |
| | | GGAATCCGGTCCAGGTACCTCTACTGAACCTTCTGAGGGCAGCGCTCCAGGTACTT |
| | | CTGAAAGCGCTACCCCGGAGTCCGGTCCAGGTACTTCTACTGAACCGTCCGAAGGT |
| | | AGCGCACCAGGTACCTCCCCTAGCGGCGAATCTTCTACTGCTCCAGGTACCTCTCC |
| | | TAGCGGCGAATCTTCTACCGCTCCAGGTACCTCCCCTAGCGGTGAATCTTCTACCG |
| | | CACCAGGTACTTCTACCGAACCGTCCGAGGGTAGCGCACCAGGTAGCCCAGCAGGT |
| | | TCTCCTACCTCCACCGAGGAAGGTACTTCTACCGAACCGTCCGAGGGTAGCGCACC |
| | | AGGTTCTAGCCCTTCTGCTTCCACCGGTACCGGCCCAGGTAGCTCTACTCCGTCTG |
| | | GTGCAACTGGCTCTCCAGGTAGCTCTACTCCGTCTGGTGCAACCGGCTCCCCAGGT |
| | | AGCTCTACCCCGTCTGGTGCTACCGGCTCTCCAGGTAGCTCTACCCCGTCTGGTGC |
| | | AACCGGCTCCCCAGGTGCATCCCCGGGTACTAGCTCTACCGGTTCTCCAGGTGCAA |
| | | GCGCAAGCGGCGCGCCAAGCACGGGAGGTACTTCTCCGAGCGGTGAATCTTCTACC |
| | | GCACCAGGTTCTACTAGCTCTACCGCTGAATCTCCGGGCCCAGGTACTTCTCCGAG |
| | | CGGTGAATCTTCTACTGCTCCAGGTACCTCTGAAAGCGCTACTCCGGAGTCTGGCC |
| | | CAGGTACCTCTACTGAACCGTCTGAGGGTAGCGCTCCAGGTACTTCTACTGAACCG |
| | | TCCGAAGGTAGCGCACCAGGTTCTAGCCCTTCTGCATCTACTGGTACTGGCCCAGG |
| sd-617485 | | TAGCTCTACTCCTTCTGGTGCTACCGGCTCTCCAGGTGCTTCTCCGGGTACTAGCT |
| | | CTACCGGTTCTCCAGGTACTTCTACTCCGGAAAGCGGTTCCGCATCTCCAGGTACT |
| BC864 | 110 | GGTACTTCCACCGAACCATCCGAACCAGGTAGCGCAGGTACTTCCACCGAACCATC |
| | | CGAACCTGGCAGCGCAGGTAGCGAACCGGCAACCTCTGGTACTGAACCATCAGGTA |
| | | GCGGCGCATCCGAGCCTACCTCTACTGAACCAGGTAGCGAACCGGCTACCTCCGGT |
| | | ACTGAGCCATCAGGTAGCGAACCGGCAACTTCCGGTACTGAACCATCAGGTAGCGA |
| | | ACCGGCAACTTCCGGCACTGAACCATCAGGTAGCGGTGCATCTGAGCCGACCTCTA |
| | | CTGAACCAGGTACTTCTACTGAACCATCTGAGCCGGGCAGCGCAGGTAGCGAACCA |
| | | GCTACTTCTGGCACTGAACCATCAGGTACTTCTACTGAACCATCCGAACCAGGTAG |
| | | CGCAGGTAGCGAACCTGCTACCTCTGGTACTGAGCCATCAGGTAGCGAACCGGCTA |
| | | CCTCTGGTACTGAACCATCAGGTACTTCTACCGAACCATCCGAGCCTGGTAGCGCA |
| | | GGTACTTCTACCGAACCATCCGAGCCAGGCAGCGCAGGTAGCGAACCGGCAACCTC |
| | | TGGCACTGAGCCATCAGGTAGCGAACCAGCAACTTCTGGTACTGAACCATCAGGTA |
| | | CTAGCGAGCCATCTACTTCCGAACCAGGTGCAGGTAGCGGCGCATCCGAACCTACT |
| | | TCCACTGAACCAGGTACTAGCGAGCCATCCACCTCTGAACCAGGTGCAGGTAGCGA |
| | | ACCGGCAACTTCCGGCACTGAACCATCAGGTAGCGAACCGGCTACCTCTGGTACTG |
| | | AACCATCAGGTACTTCTACCGAACCATCCGAGCCTGGTAGCGCAGGTACTTCTACC |
| | | GAACCATCCGAGCCAGGCAGCGCAGGTAGCGGTGCATCCGAGCCGACCTCTACTGA |
| | | ACCAGGTAGCGAACCAGCAACTTCTGGCACTGAGCCATCAGGTAGCGAACCAGCTA |
| | | CCTCTGGTACTGAACCATCAGGTAGCGAACCGGCTACTTCCGGCACTGAACCATCA |
| | | GGTAGCGAACCAGCAACCTCCGGTACTGAACCATCAGGTACTTCCACTGAACCATC |
| | | CGAACCGGGTAGCGCAGGTAGCGAACCGGCAACTTCCGGCACTGAACCATCAGGTA |
| | | GCGGTGCATCTGAGCCGACCTCTACTGAACCAGGTACTTCTACTGAACCATCTGAG |
| | | CCGGGCAGCGCAGGTAGCGAACCTGCAACCTCCGGCACTGAGCCATCAGGTAGCGG |
| | | CGCATCTGAACCAACCTCTACTGAACCAGGTACTTCCACCGAACCATCTGAGCCAG |
| | | GCAGCGCAGGTAGCGGCGCATCTGAACCAACCTCTACTGAACCAGGTAGCGAACCA |
| | | GCAACTTCTGGTACTGAACCATCAGGTAGCGGCGCATCTGAGCCTACTTCCACTGA |
| | | ACCAGGTAGCGAACCGGCAACTTCCGGCACTGAACCATCAGGTAGCGGTGCATCTG |
| | | AGCCGACCTCTACTGAACCAGGTACTTCTACTGAACCATCTGAGCCGGGCAGCGCA |
| | | GGTAGCGAACCGGCAACTTCCGGCACTGAACCATCAGGTAGCGGTGCATCTGAGCC |
| | | GACCTCTACTGAACCAGGTACTTCTACTGAACCATCTGAGCCGGGCAGCGCAGGTA |
| | | GCGAACCAGCTACTTCTGGCACTGAACCATCAGGTACTTCTACTGAACCATCCGAA |
| | | CCAGGTAGCGCAGGTAGCGAACCTGCTACCTCTGGTACTGAGCCATCAGGTACTTC |
| | | TACTGAACCATCCGAGCCGGGTAGCGCAGGTACTTCCACTGAACCATCTGAACCTG |
| | | GTAGCGCAGGTACTTCCACTGAACCATCCGAACCAGGTAGCGCAGGTACTTCTACT |
| | | GAACCATCCGAGCCGGGTAGCGCAGGTACTTCCACTGAACCATCTGAACCTGGTAG |
| | | CGCAGGTACTTCCACTGAACCATCCGAACCAGGTAGCGCAGGTACTAGCGAACCAT |
| | | CCACCTCCGAACCAGGCGCAGGTAGCGGTGCATCTGAACCGACTTCTACTGAACCA |
| | | GGTACTTCCACTGAACCATCTGAGCCAGGTAGCGCAGGTACTTCCACCGAACCATC |
| | | CGAACCAGGTAGCGCAGGTACTTCCACCGAACCATCCGAACCTGGCAGCGCAGGTA |
| | | GCGAACCGGCAACCTCTGGTACTGAACCATCAGGTAGCGGTGCATCCGAGCCGACC |
| | | TCTACTGAACCAGGTAGCGAACCAGCAACTTCTGGCACTGAGCCATCAGGTAGCGA |
| | | ACCAGCTACCTCTGGTACTGAACCATCAGGTAGCGAACCGGCAACCTCTGGCACTG |
| | | AGCCATCAGGTAGCGAACCAGCAACTTCTGGTACTGAACCATCAGGTACTAGCGAG |
| | | CCATCTACTTCCGAACCAGGTGCAGGTAGCGAACCTGCAACCTCCGGCACTGAGCC |
| | | ATCAGGTAGCGGCGCATCTGAACCAACCTCTACTGAACCAGGTACTTCCACCGAAC |
| | | CATCTGAGCCAGGCAGCGCAGGTAGCGAACCTGCAACCTCCGGCACTGAGCCATCA |
| | | GGTAGCGGCGCATCTGAACCAACCTCTACTGAACCAGGTACTTCCACCGAACCATC |
| | | TGAGCCAGGCAGCGCA |
| BD864 | 111 | GGTAGCGAAACTGCTACTTCCGGCTCTGAGACTGCAGGTACTAGTGAATCCGCAAC |
| | | TAGCGAATCTGGCGCAGGTAGCACTGCAGGCTCTGAGACTTCCACTGAAGCAGGTA |
| | | CTAGCGAGTCCGCAACCAGCGAATCCGGCGCAGGTAGCGAAACTGCTACCTCTGGC |
| | | TCCGAGACTGCAGGTAGCGAAACTGCAACCTCTGGCTCTGAAACTGCAGGTACTTC |
| | | CACTGAAGCAAGTGAAGGCTCCGCATCAGGTACTTCCACCGAAGCAAGCGAAGGCT |
| | | CCGCATCAGGTACTAGTGAGTCCGCAACTAGCGAATCCGGTGCAGGTAGCGAAACC |
| | | GCTACCTCTGGTTCCGAAACTGCAGGTACTTCTACCGAGGCTAGCGAAGGTTCTGC |
| | | ATCAGGTAGCACTGCTGGTTCCGAGACTTCTACTGAAGCAGGTACTAGCGAATCTG |
| | | CTACTAGCGAATCCGGCGCAGGTACTAGCGAATCCGCTACCAGCGAATCCGGCGCA |
| | | GGTAGCGAAACTGCAACCTCTGGTTCCGAGACTGCAGGTACTAGCGAGTCCGCTAC |
| | | TAGCGAATCTGGCGCAGGTACTTCCACTGAAGCTAGTGAAGGTTCTGCATCAGGTA |
| | | GCGAAACTGCTACTTCTGGTTCCGAAACTGCAGGTAGCGAAACCGCTACCTCTGGT |
| | | TCCGAAACTGCAGGTACTTCTACCGAGGCTAGCGAAGGTTCTGCATCAGGTAGCAC |
| | | TGCTGGTTCCGAGACTTCTACTGAAGCAGGTACTAGCGAGTCCGCTACTAGCGAAT |
| | | CTGGCGCAGGTACTTCCACTGAAGCTAGTGAAGGTTCTGCATCAGGTAGCGAAACT |
| | | GCTACTTCTGGTTCCGAAACTGCAGGTAGCACTGCTGGCTCCGAGACTTCTACCGA |
| | | AGCAGGTAGCACTGCAGGTTCCGAAACTTCCACTGAAGCAGGTAGCGAAACTGCTA |
| | | CCTCTGGCTCTGAGACTGCAGGTACTAGCGAATCTGCTACTAGCGAATCCGGCGCA |
| | | GGTACTAGCGAATCCGCTACCAGCGAATCCGGCGCAGGTAGCGAAACTGCAACCTC |
| | | TGGTTCCGAGACTGCAGGTACTAGCGAATCTGCTACTAGCGAATCCGGCGCAGGTA |
| | | CTAGCGAATCCGCTACCAGCGAATCCGGCGCAGGTAGCGAAACTGCAACCTCTGGT |
| | | TCCGAGACTGCAGGTAGCGAAACCGCTACCTCTGGTTCCGAAACTGCAGGTACTTC |
| | | TACCGAGGCTAGCGAAGGTTCTGCATCAGGTAGCACTGCTGGTTCCGAGACTTCTA |
| | | CTGAAGCAGGTAGCGAAACTGCTACTTCCGGCTCTGAGACTGCAGGTACTAGTGAA |
| | | TCCGCAACTAGCGAATCTGGCGCAGGTAGCACTGCAGGCTCTGAGACTTCCACTGA |
| | | AGCAGGTAGCACTGCTGGTTCCGAAACCTCTACCGAAGCAGGTAGCACTGCAGGTT |
| | | CTGAAACCTCCACTGAAGCAGGTACTTCCACTGAGGCTAGTGAAGGCTCTGCATCA |
| | | GGTAGCACTGCTGGTTCCGAAACCTCTACCGAAGCAGGTAGCACTGCAGGTTCTGA |
| | | AACCTCCACTGAAGCAGGTACTTCCACTGAGGCTAGTGAAGGCTCTGCATCAGGTA |
| | | GCACTGCAGGTTCTGAGACTTCCACCGAAGCAGGTAGCGAAACTGCTACTTCTGGT |
| | | TCCGAAACTGCAGGTACTTCCACTGAAGCTAGTGAAGGTTCCGCATCAGGTACTAG |
| | | TGAGTCCGCAACCAGCGAATCCGGCGCAGGTAGCGAAACCGCAACCTCCGGTTCTG |
| | | AAACTGCAGGTACTAGCGAATCCGCAACCAGCGAATCTGGCGCAGGTACTAGTGAG |
| | | TCCGCAACCAGCGAATCCGGCGCAGGTAGCGAAACCGCAACCTCCGGTTCTGAAAC |
| | | TGCAGGTACTAGCGAATCCGCAACCAGCGAATCTGGCGCAGGTAGCGAAACTGCTA |
| | | CTTCCGGCTCTGAGACTGCAGGTACTTCCACCGAAGCAAGCGAAGGTTCCGCATCA |
| | | GGTACTTCCACCGAGGCTAGTGAAGGCTCTGCATCAGGTAGCACTGCTGGCTCCGA |
| | | GACTTCTACCGAAGCAGGTAGCACTGCAGGTTCCGAAACTTCCACTGAAGCAGGTA |
| | | GCGAAACTGCTACCTCTGGCTCTGAGACTGCAGGTACTAGCGAATCTGCTACTAGC |
| | | GAATCCGGCGCAGGTACTAGCGAATCCGCTACCAGCGAATCCGGCGCAGGTAGCGA |
| | | AACTGCAACCTCTGGTTCCGAGACTGCAGGTAGCGAAACTGCTACTTCCGGCTCCG |
| | | AGACTGCAGGTAGCGAAACTGCTACTTCTGGCTCCGAAACTGCAGGTACTTCTACT |
| | | GAGGCTAGTGAAGGTTCCGCATCAGGTACTAGCGAGTCCGCAACCAGCGAATCCGG |
| | | CGCAGGTAGCGAAACTGCTACCTCTGGCTCCGAGACTGCAGGTAGCGAAACTGCAA |
| | | CCTCTGGCTCTGAAACTGCAGGTACTAGCGAATCTGCTACTAGCGAATCCGGCGCA |
| | | GGTACTAGCGAATCCGCTACCAGCGAATCCGGCGCAGGTAGCGAAACTGCAACCTC |
| | | TGGTTCCGAGACTGCA |

One may clone the library of XTEN-encoding genes into one or more expression vectors known in the art. To facilitate the identification of well-expressing library members, one can construct the library as fusion to a reporter, protein. Non-limiting examples of suitable reporter genes are green fluorescent protein, luciferase, alkaline phosphatase, and beta-galactosidase. By screening, one can identify short XTEN sequences that can be expressed in high concentration in the host organism of choice. Subsequently, one can generate a library of random XTEN dimers and repeat the screen for high level of expression. Subsequently, one can screen the resulting constructs for a number of properties such as level of expression, protease stability, or binding to antiserum.

One aspect of the invention is to provide polynucleotide sequences encoding the components of the fusion protein wherein the creation of the sequence has undergone codon optimization. Of particular interest is codon optimization with the goal of improving expression of the polypeptide compositions and to improve the genetic stability of the encoding gene in the production hosts. For example, codon optimization is of particular importance for XTEN sequences that are rich in glycine or that have very repetitive amino acid sequences. Codon optimization can be performed using computer programs (Gustafsson, C., et al., (2004) Trends Biotechnol, 22: 346-353), some of which minimize ribosomal pausing (Coda Genomics Inc.). In one embodiment, one can perform codon optimization by constructing codon libraries where all members of the library encode the same amino acid sequence but where codon usage is varied. Such libraries can be screened for highly expressing and genetically stable members that are particularly suitable for the large-scale production of XTEN-containing products. When designing XTEN sequences one can consider a number of properties. One can minimize the repetitiveness in the encoding DNA sequences. In addition, one can avoid or minimize the use of codons that are rarely used by the production host (e.g. the AGG and AGA arginine codons and one leucine codon in E. coli). In the case of E. coli, two glycine codons, GGA and GGG, are rarely used in highly expressed proteins. Thus codon optimization of the gene encoding XTEN sequences can be very desirable. DNA sequences that have a high level of glycine tend to have a high GC content that can lead to instability or low expression levels. Thus, when possible, it is preferred to choose codons such that the GC-content of XTEN-encoding sequence is suitable for the production organism that will be used to manufacture the XTEN.

Optionally, the full-length XTEN-encoding gene may comprise one or more sequencing islands. In this context, sequencing islands are short-stretch sequences that are distinct from the XTEN library construct sequences and that include a restriction site not present or expected to be present in the full-length XTEN-encoding gene. In one embodiment, a sequencing island is the sequence

(SEQ ID NO: 112)
5′-AGGTGCAAGCGCAAGCGGCGCGCCAAGCACGGGAGGT-3′

In another embodiment, a sequencing island is the sequence

(SEQ ID NO: 113)
5′-AGGTCCAGAACCAACGGGGCCGGCCCCAAGCGGAGGT-3′

As an alternative, one can construct codon libraries where all members of the library encode the same amino acid sequence but where codon usage is varied. Such libraries can be screened for highly expressing and genetically stable members that are particularly suitable for the large-scale production of XTEN-containing products.

Optionally, one can sequence clones in the library to eliminate isolates that contain undesirable sequences. The initial library of short XTEN sequences can allow some variation in amino acid sequence. For instance one can randomize some codons such that a number of hydrophilic amino acids can occur in a particular position.

During the process of iterative multimerization one can screen the resulting library members for other characteristics like solubility or protease resistance in addition to a screen for high-level expression.

Once the gene that encodes the XTEN of desired length and properties is selected, it is genetically fused to the nucleotides encoding the N- and/or the C-terminus of the C-peptide gene(s) by cloning it into the construct adjacent and in frame with the gene coding for C-peptide or adjacent to a spacer sequence. The invention provides various permutations of the foregoing, depending on the C-peptide-XTEN to be encoded. For example, a gene encoding a C-peptide-XTEN fusion protein comprising two C-peptides such as embodied by formula III or IV, as depicted above, the gene would have polynucleotides encoding two C-peptides, at least a first XTEN, and optionally a second XTEN and/or spacer sequences. The step of cloning the C-peptide genes into the XTEN construct can occur through a ligation or multimerization step. Constructs encoding C-peptide-XTEN fusion proteins can be designed in different configurations of the components XTEN, C-peptide, and spacer sequences. In one embodiment, the construct comprises polynucleotide sequences complementary to, or those that encode a monomeric polypeptide of components in the following order (5′ to 3′) C-peptide and XTEN, or the reverse order. In another embodiment, the construct comprises polynucleotide sequences complementary to, or those that encode a monomeric polypeptide of components in the following order (5′ to 3′) C-peptide, spacer sequence, and XTEN, or the reverse order. In another embodiment, the construct encodes a monomeric C-peptide-XTEN comprising polynucleotide sequences complementary to, or those that encode components in the following order (5′ to 3′): two molecules of C-peptide and XTEN, or the reverse order. In another embodiment, the construct comprises polynucleotide sequences complementary to, or those that encode a monomeric polypeptide of components in the following order (5′ to 3′): two molecules of C-peptide, spacer sequence, and XTEN, or the reverse order. In another embodiment, the construct comprises polynucleotide sequences complementary to, or those that encode a monomeric polypeptide of components in the following order (5′ to 3′): C-peptide, spacer sequence, a second molecule of C-peptide, and XTEN 202, or the reverse order. In another embodiment, the construct comprises polynucleotide sequences complementary to, or those that encode a monomeric polypeptide of components in the following order (5′ to 3′): C-peptide, XTEN, C-peptide, and a second XTEN, or the reverse sequence. The spacer polynucleotides can optionally comprise sequences encoding cleavage sequences. As will be apparent to those of skill in the art, other permutations of the foregoing are possible.

The invention also encompasses polynucleotides comprising XTEN-encoding polynucleotide variants that have a high percentage of sequence identity to (a) a polynucleotide sequence from Table 5, or (b) sequences that are complementary to the polynucleotides of (a). A polynucleotide with a high percentage of sequence identity is one that has at least about an 80% nucleic acid sequence identity, alternatively at least about 81%, alternatively at least about 82%, alternatively at least about 83%, alternatively at least about 84%, alternatively at least about 85%, alternatively at least about 86%, alternatively at least about 87%, alternatively at least about 88%, alternatively at least about 89%, alternatively at least about 90%, alternatively at least about 91%, alternatively at least about 92%, alternatively at least about 93%, alternatively at least about 94%, alternatively at least about 95%, alternatively at least about 96%, alternatively at least about 97%, alternatively at least about 98%, and alternatively at least about 99% nucleic acid sequence identity to (a) or (b) of the foregoing, or that can hybridize with the target polynucleotide or its complement under stringent conditions. Homology, sequence similarity or sequence identity of nucleotide or amino acid sequences may also be determined conventionally by using known software or computer programs such as the BestFit or Gap pairwise comparison programs (GCG Wisconsin Package, Genetics Computer Group, 575 Science Drive, Madison, Wis. 53711). BestFit uses the local homology algorithm of Smith and Waterman (Advances in Applied Mathematics. 1981. 2: 482-489), to find the best segment of identity or similarity between two sequences. Gap performs global alignments: all of one sequence with all of another similar sequence using the method of Needleman and Wunsch, (Journal of Molecular Biology. 1970.48:443-453). When using a sequence alignment program such as BestFit, to determine the degree of sequence homology, similarity or identity, the default setting may be used, or an appropriate scoring matrix may be selected to optimize identity, similarity or homology scores.

Nucleic acid sequences that are “complementary” are those that are capable of base-pairing according to the standard Watson-Crick complementarity rules. As used herein, the term “complementary sequences” means nucleic acid sequences that are substantially complementary, as may be assessed by the same nucleotide comparison set forth above, or as defined as being capable of hybridizing to the polynucleotides that encode the C-peptide-XTEN sequences under stringent conditions, such as those described herein.

The resulting polynucleotides encoding the C-peptide-XTEN chimeric compositions can then be individually cloned into an expression vector. The nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan. Such techniques are well known in the art and well described in the scientific and patent literature.

Various vectors are publicly available. The vector may, for example, be in the form of a plasmid, cosmid, viral particle, or phage. Both expression and cloning vectors contain a nucleic acid sequence that enables the vector to replicate in one or more selected host cells. Such vector sequences are well known for a variety of bacteria, yeast, and viruses. Useful expression vectors that can be used include, for example, segments of chromosomal, non-chromosomal and synthetic DNA sequences. Suitable vectors include, but are not limited to, derivatives of SV40 and pcDNA and known bacterial plasmids such as col E1, pCR1, pBR322, pMal-C2, pET, pGEX as described by Smith, et al., Gene 57:31-40 (1988), pMB9 and derivatives thereof, plasmids such as RP4, phage DNAs such as the numerous derivatives of phage I such as NM98 9, as well as other phage DNA such as M13 and filamentous single stranded phage DNA; yeast plasmids such as the 2 micron plasmid or derivatives of the 2 micron plasmid, as well as centomeric and integrative yeast shuttle vectors; vectors useful in eukaryotic cells such as vectors useful in insect or mammalian cells; vectors derived from combinations of plasmids and phage DNAs, such as plasmids that have been modified to employ phage DNA or the expression control sequences; and the like. The requirements are that the vectors are replicable and viable in the host cell of choice. Low- or high-copy number vectors may be used as desired.

Promoters suitable for use in expression vectors with prokaryotic hosts include the β-lactamase and lactose promoter systems [Chang et al., Nature, 275:615 (1978); Goeddel et al., Nature, 281:544 (1979)], alkaline phosphatase, a tryptophan (trp) promoter system [Goeddel, Nucleic Acids Res., 8:4057 (1980); EP 36776], and hybrid promoters such as the tac promoter [deBoer et al., Proc. Natl. Acad. Sci. USA, 80:21-25 (1983)]. Promoters for use in bacterial systems can also contain a Shine-Dalgarno (S.D.) sequence operably linked to the DNA encoding C-peptide-XTEN polypeptides.

For example, in a baculovirus expression system, both non-fusion transfer vectors, such as, but not limited to pVL941 (BamHI cloning site, available from Summers, et al., Virology 84:390-402 (1978)), pVL1393 (BamHI, Smal, Xbal, EcoRI, IVotl, Xmalll, BgIII and PstI cloning sites; Invitrogen), pVL1392 (BgIII, Pstl, NotI, XmaIII, EcoRI, XbaII, SmaI and BamHI cloning site; Summers, et al., Virology 84:390-402 (1978) and Invitrogen) and pBlueBacIII (BamHI, BgIII, Pstl, Ncol and Hindi II cloning site, with blue/white recombinant screening, Invitrogen), and fusion transfer vectors such as, but not limited to, pAc7 00 (BamHI and Kpnl cloning sites, in which the BamHI recognition site begins with the initiation codon; Summers, et al., Virology 84:390-402 (1978)), pAc701 and pAc70-2 (same as pAc700, with different reading frames), pAc360 [BamHI cloning site 36 base pairs downstream of a polyhedrin initiation codon; Invitrogen (1995)) and pBlueBacHisA, B, C (three different reading frames with BamH I, BgI II, Pstl, Ncol and Hind III cloning site, an N-terminal peptide for ProBond purification and blue/white recombinant screening of plaques; Invitrogen (220) can be used.

Mammalian expression vectors can comprise an origin of replication, a suitable promoter and enhancer, and also any necessary ribosome binding sites, polyadenylation site, splice donor and acceptor sites, transcriptional termination sequences, and 5′ flanking nontranscribed sequences. DNA sequences derived from the SV40 splice, and polyadenylation sites may be used to provide the required non-transcribed genetic elements. Mammalian expression vectors contemplated for use in the invention include vectors with inducible promoters, such as the dihydrofolate reductase promoters, any expression vector with a DHFR expression cassette or a DHFR/methotrexate co-amplification vector such as pED (Pstl, Sail, Sbal, Smal and EcoRI cloning sites, with the vector expressing both the cloned gene and DHFR; Randal J. Kaufman, 1991, Randal J. Kaufman, Current Protocols in Molecular Biology, 16:12 (1991)). Alternatively a glutamine synthetase/methionine sulfoximine co-amplification vector, such as pEE14 (HindIII, Xball, Smal, Sbal, EcoRI and Sell cloning sites in which the vector expresses glutamine synthetase and the cloned gene; Celltech). A vector that directs episomal expression under the control of the Epstein Barr Virus (EBV) or nuclear antigen (EBNA) can be used such as pREP4 (BamHI r SfH, Xhol, NotI, Nhel, Hindi II, NheI, PvuII and Kpnl cloning sites, constitutive RSV-LTR promoter, hygromycin selectable marker; Invitrogen), pCEP4 (BamHI, SfH, Xhol, NotI, Nhel, Hindlll, Nhel, PvuII and Kpnl cloning sites, constitutive hCMV immediate early gene promoter, hygromycin selectable marker; Invitrogen), pMEP4 (Kpnl, Pvul, Nhel, Hindlll, NotI, Xhol, Sfil, BamHI cloning sites, inducible methallothionein H a gene promoter, hygromycin selectable marker, Invitrogen), pREP8 (BamHI, Xhol, NotI, Hindlll, Nhel and Kpnl cloning sites, RSV-LTR promoter, histidinol selectable marker; Invitrogen), pREP9 (Kpnl, Nhel, Hind III, NotI, Xho 1, Sfi 1, BamHI cloning sites, RSV-LTR promoter, G418 selectable marker; Invitrogen), and pEBVHis (RSV-LTR promoter, hygromycin selectable marker, N-terminal peptide purifiable via ProBond resin and cleaved by enterokinase; Invitrogen).

Selectable mammalian expression vectors for use in the invention include, but are not limited to, pRc/CMV (HindIII, BstXI, NotI, SbaI and ApaI cloning sites, G418 selection, Invitrogen), pRc/RSV (Hind III, SpeI, BstXI, NotI, Xbal cloning sites, G418 selection, Invitrogen) and the like. Vaccinia virus mammalian expression vectors (see, for example, Randall J. Kaufman, Current Protocols in Molecular Biology 16:12 (Frederick M. Ausubel, et al., eds. Wiley 1991) that can be used in the present invention include, but are not limited to, pSC11 (Smal cloning site, TK- and beta-gal selection), pMJ601 (Sal 1, Smal, AfII, Narl, BspMII, BamHI, Apal, Nhel, Sacll, Kpnl and HindIII cloning sites; TK- and -gal selection), pTKgptFIS (EcoRI, Pstl, SalII, Accl, HindIII, Sbal, BamHI and Hpa cloning sites, TK or XPRT selection) and the like.

Yeast expression systems that can also be used in the present invention include, but are not limited to, the nonfusion pYES2 vector (XJbal, Sphl, Shol, NotI, GstXI, EcoRI, BstXI, BamHI, Sad, Kpnl and HindIII cloning sites, Invitrogen), the fusion pYESHisA, B, C (Xball, Sphl, Shol, NotI, BstXI, EcoRI, BamHI, Sad, Kpnl and Hindi II cloning sites, N-terminal peptide purified with ProBond resin and cleaved with enterokinase; Invitrogen), pRS vectors and the like.

In addition, the expression vector containing the chimeric C-peptide-XTEN fusion protein-encoding polynucleotide molecule may include drug selection markers. Such markers aid in cloning and in the selection or identification of vectors containing chimeric DNA molecules. For example, genes that confer resistance to neomycin, puromycin, hygromycin, dihydrofolate reductase (DHFR) inhibitor, guanine phosphoribosyl transferase (GPT), zeocin, and histidinol are useful selectable markers. Alternatively, enzymes such as herpes simplex virus thymidine kinase (tk) or chloramphenicol acetyltransferase (CAT) may be employed. Immunologic markers also can be employed. Any known selectable marker may be employed so long as it is capable of being expressed simultaneously with the nucleic acid encoding a gene product. Further examples of selectable markers are well known to one of skill in the art and include reporters such as enhanced green fluorescent protein (EGFP), beta-galactosidase (β-gal) or chloramphenicol acetyltransferase (CAT).

In one embodiment, the polynucleotide encoding a C-peptide-XTEN fusion protein composition can be fused C-terminally to an N.-terminal signal sequence appropriate for the expression host system. Signal sequences are typically proteolytically removed from the protein during the translocation and secretion process, generating a defined N-terminus. A wide variety of signal sequences have been described for most expression systems, including bacterial, yeast, insect, and mammalian systems. A non-limiting list of preferred examples for each expression system follows herein. Preferred signal sequences are OmpA, Pho.A, and DsbA for E. coli expression. Signal peptides preferred for yeast expression are ppL-alpha, DEX4, invertase signal peptide, acid phosphatase signal peptide, CPY, or INU1. For insect cell expression the preferred signal sequences are sexta adipokinetic hormone precursor, CP1, CP2, CP3, CP4, TPA, PAP, or gp67. For mammalian expression the preferred signal sequences are IL2L, SV40, IgG kappa and IgG lambda.

In another embodiment, a leader sequence, potentially comprising a well-expressed, independent protein domain, can be fused to the N-terminus of the C-peptide-XTEN sequence, separated by a protease cleavage site. While any leader peptide sequence which does not inhibit cleavage at the designed proteolytic site can be used, sequences in preferred embodiments will comprise stable, well-expressed sequences such that expression and folding of the overall composition is not significantly adversely affected, and preferably expression, solubility, and/or folding efficiency are significantly improved. A wide variety of suitable leader sequences have been described in the literature. A non-limiting list of suitable sequences includes maltose binding protein, cellulose binding domain, glutathione S-transferase, 6×His tag, FLAG tag, hemaglutinin tag, and green fluorescent protein. The leader sequence can also be further improved by codon optimization, especially in the second codon position following the ATG start codon, by methods well described in the literature and hereinabove.

Various in vitro enzymatic methods for cleaving proteins at specific sites are known. Such methods include use of enterokinase (DDDK (SEQ ID NO:114)), Factor Xa (IDGR (SEQ ID NO:115)), thrombin (LVPRGS (SEQ ID NO:116)), Prexcission™ (LEVLFQGP (SEQ ID NO:117)), TEV protease (EQLYFQG (SEQ ID NO:118)), 3C protease (ETLFQGP (SEQ ID NO:119)), Sortase A (LPETG) (SEQ ID NO:120), Granzyme B (D/X, N/X, M/N or S/X), inteins, SUMO, DAPase (TAGZyme™), Aeromonas aminopeptidase, Aminopeptidase M, and carboxypeptidases A and B. Additional methods are disclosed in Arnau, et al., Protein Expression and Purification 48: 1-13 (2006).

In other embodiments, an optimized polynucleotide sequence encoding at least about 20 to about 60 amino acids with XTEN characteristics can be included at the N-terminus of the XTEN sequence to promote the initiation of translation to allow for expression of XTEN fusions at the N-terminus of proteins without the presence of a helper domain. In an advantage of the foregoing, the sequence does not require subsequent cleavage, thereby reducing the number of steps to manufacture XTEN-containing compositions. As described in more detail in the Examples, the optimized N-terminal sequence has attributes of an unstructured protein, but may include nucleotide bases encoding amino acids selected for their ability to promote initiation of translation and enhanced expression. In one embodiment of the foregoing, the optimized polynucleotide encodes an XTEN sequence with at least about 90% sequence identity to AE912 (SEQ ID NO:74).

In another embodiment of the foregoing, the optimized polynucleotide encodes an XTEN sequence with at least about 90% sequence identity to AM923 (SEQ ID NO:75).

In another embodiment, the protease site of the leader sequence construct is chosen such that it is recognized by an in vivo protease. In this embodiment, the protein is purified from the expression system while retaining the leader by avoiding contact with an appropriate protease. The full length construct is then injected into a patient. Upon injection, the construct comes into contact with the protease specific for the cleavage site and is cleaved by the protease. In the case where the uncleaved protein is substantially less active than the cleaved form, this method has the beneficial effect of allowing higher initial doses while avoiding toxicity, as the active form is generated slowly in vivo. Some non-limiting examples of in vivo proteases which are useful for this application include tissue kallikrein, plasma kallikrein, trypsin, pepsin, chymotrypsin, thrombin, and matrix metalloproteinases, or the proteases of Table 4.

In this manner, a chimeric DNA molecule coding for a monomeric C-peptide-XTEN fusion protein is generated within the construct. Optionally, this chimeric DNA molecule may be transferred or cloned into another construct that is a more appropriate expression vector. At this point, a host cell capable of expressing the chimeric DNA molecule can be transformed with the chimeric DNA molecule. The vectors containing the DNA segments of interest can be transferred into the host cell by well-known methods, depending on the type of cellular host. For example, calcium chloride transfection is commonly utilized for prokaryotic cells, whereas calcium phosphate treatment, lipofection, or electroporation may be used for other cellular hosts. Other methods used to transform mammalian cells include the use of polybrene, protoplast fusion, liposomes, electroporation, and microinjection. See, generally, Sambrook, et al., supra.

The transformation may occur with or without the utilization of a carrier, such as an expression vector. Then, the transformed host cell is cultured under conditions suitable for expression of the chimeric DNA molecule encoding of C-peptide-XTEN.

The present invention also provides a host cell for expressing the monomeric fusion protein compositions disclosed herein. Examples of suitable eukaryotic host cells include, but are not limited to mammalian cells, such as VERO cells, HELA cells such as ATCC No. CCL2, CHO cell lines, COS cells, W138 cells, BHK cells, HepG2 cells, 3T3 cells, A549 cells, PCI2 cells, K562 cells, 293 cells, Sf9 cells and CvI cells. Examples of suitable non-mammalian eukaryotic cells include eukaryotic microbes such as filamentous fungi or yeast are suitable cloning or expression hosts for encoding vectors. Saccharomyces cerevisiae is a commonly used lower eukaryotic host microorganism. Others include Schizosaccharomyces pombe (Beach and Nurse, Nature, 290:140 [1981]; EP 139,383 published 2 May 1985); Kluyveromyces hosts (U.S. Pat. No. 4,943,529; Fleer et al., Bio/Technology, 9:968-975 (1991)) such as, e.g., K. lactis (MW98-8C, CBS683, CBS4574; Louvencourt et al., J. Bacteriol., 737 [1983]), K. fragilis (ATCC 12,424), K. bulgaricus (ATCC 16,045), K. wickeramii (ATCC 24,178), K. waltii (ATCC 56,500), K. drosophilarum (ATCC 36,906; Van den Berg et al., Bio/Technology, 8:135 (1990)), K. thermotolerans, and K. marxianus; yarrowia (EP 402,226); Pichia pastoris (EP 183,070; Sreekrishna et al., J. Basic Microbiol., 28:265-278 [1988]); Candida; Trichoderma reesia (EP 244,234); Neurospora crassa (Case et al., Proc. Natl. Acad. Sci. USA, 76:5259-5263 [1979]); Schwanniomyces such as Schwanniomyces occidentalis (EP 394,538 published 31 Oct. 1990); and filamentous fungi such as, e.g., Neurospora, Penicillium, Iblypocladium (WO91/00357 published 10 Jan. 1991), and Aspergillus hosts such as A. nidulans (Ballance et al., Biochem. Biophys. Res. Commun., 112:284-289 [1983]; Tilburn et al., Gene, 26:205-221 [1983]; Yelton et al., Proc. Natl. Acad. Sci. USA, 81: 1470-1474 [1984]) and A. niger (Kelly and Hynes, EMBO J., 4:475-479 [1985]). Methylotropic yeasts are suitable herein and include, but are not limited to, yeast capable of growth on methanol selected from the genera consisting of Hansenula, Candida, Kloeckera, Pichia, Saccharomyces, Torulopsis, and Rhodotorula. A list of specific species that are exemplary of this class of yeasts may be found in C. Anthony, The Biochemistry of Methylotrophs, 269 (1982).

Other suitable cells that can be used in the present invention include, but are not limited to, prokaryotic host cells strains such as Escherichia coli, (e.g., strain DH5-α), Bacillus subtilis, Salmonella typhimurium, or strains of the genera of Pseudomonas, Streptomyces and Staphylococcus. Non-limiting examples of suitable prokaryotes include those from the genera: Actinoplanes; Archaeoglobus; Bdellovibrio; Borrelia; Chloroflexus; Enterococcus; Escherichia; Lactobacillus; Listeria; Oceanobacillus; Paracoccus; Pseudomonas; Staphylococcus; Streptococcus; Streptomyces; Thermoplasma; and Vibrio. Non-limiting examples of specific strains include: Archaeoglobus fulgidus; Bdellovibrio bacteriovorus; Borrelia burgdorferi; Chloroflexus aurantiacus; Enterococcus faecalis; Enterococcus faecium; Lactobacillus johnsonii; Lactobacillus plantarum; Lactococcus lactis; Listeria innocua; Listeria monocytogenes; Oceanobacillus iheyensis; Paracoccus zeaxanthinifaciens; Pseudomonas mevalonii; Staphylococcus aureus; Staphylococcus epidermidis; Staphylococcus haemolyticus; Streptococcus agalactiae; Streptomyces griseolosporeus; Streptococcus mutans; Streptococcus pneumoniae; Streptococcus pyogenes; Thermoplasma acidophilum; Thermoplasma volcanium; Vibrio cholerae; Vibrio parahaemolyticus; and Vibrio vulnificus.

Host cells containing the polynucleotides of interest can be cultured in conventional nutrient media (e.g., Ham's nutrient mixture) modified as appropriate for activating promoters, selecting transformants or amplifying genes. The culture conditions, such as temperature, pH and the like, are those previously used with the host cell selected for expression, and will be apparent to the ordinarily skilled artisan.

Cells are typically harvested by centrifugation, disrupted by physical or chemical means, and the resulting crude extract retained for further purification. For compositions secreted by the host cells, supernatant from centrifugation is separated and retained for further purification. Microbial cells employed in expression of proteins can be disrupted by any convenient method, including freeze-thaw cycling, sonication, mechanical disruption, or use of cell lysing agents, all of which are well known to those skilled in the art. Embodiments that involve cell lysis may entail use of a buffer that contains protease inhibitors that limit degradation after expression of the chimeric DNA molecule. Suitable protease inhibitors include, but are not limited to leupeptin, pepstatin or aprotinin. The supernatant then may be precipitated in successively increasing concentrations of saturated ammonium sulfate.

Gene expression may be measured in a sample directly, for example, by conventional Southern blotting, Northern blotting to quantitate the transcription of mRNA ([Thomas, Proc. Natl. Acad. Sci. USA, 77:5201-5205 (1980)]), dot blotting (DNA analysis), or in situ hybridization, using an appropriately labeled probe, based on the sequences provided herein. Alternatively, antibodies may be employed that can recognize specific duplexes, including DNA duplexes, RNA duplexes, and DNA-RNA hybrid duplexes or DNA-protein duplexes. The antibodies in turn may be labeled and the assay may be carried out where the duplex is bound to a surface, so that upon the formation of duplex on the surface, the presence of antibody bound to the duplex can be detected.

Gene expression, alternatively, may be measured by immunological of fluorescent methods, such as immunohistochemical staining of cells or tissue sections and assay of cell culture or body fluids or the detection of selectable markers, to quantitate directly the expression of gene product. Antibodies useful for immunohistochemical staining and/or assay of sample fluids may be either monoclonal or polyclonal, and may be prepared in any mammal. Conveniently, the antibodies may be prepared against a native sequence C-peptide polypeptide or against a synthetic peptide based on the DNA sequences provided herein or against exogenous sequence fused to C-peptide and encoding a specific antibody epitope. Examples of selectable markers are well known to one of skill in the art and include reporters such as enhanced green fluorescent protein (EGFP), beta-galactosidase (β-gal) or chloramphenicol acetyltransferase (CAT).

Expressed C-peptide-XTEN polypeptide product(s) may be purified via methods known in the art or by methods disclosed herein. Procedures such as gel filtration, affinity purification, salt fractionation, ion exchange chromatography, size exclusion chromatography, hydroxyapatite adsorption chromatography, hydrophobic interaction chromatography and gel electrophoresis may be used; each tailored to recover and purify the fusion protein produced by the respective host cells. Some expressed C-peptide-XTEN may require refolding during isolation and purification. Methods of purification are described in Robert K. Scopes, Protein Purification: Principles and Practice, Charles R. Castor (ed.), Springer-Verlag 1994, and Sambrook, et al., supra. Multi-step purification separations are also described in Baron, et al., Crit. Rev. Biotechnol. 10:17990 (1990) and Below, et al., J. Chromatogr. A. 679:67-83 (1994). See Example 23 for exemplary procedures for expression, purification and characterization of C-peptide-XTEN polypeptides.

A search of patents, published patent applications, and related publications will also provide those skilled in the art reading this disclosure with significant possible XTEN-coupling technologies and XTEN-derivatives. For example, U.S. Pat. No. 7,846,445; U.S. Pat. No. 7,855,279; US 20100239554; US 20100260706; US 20110151433; US 20110171687; US 20110312881; US 20080039341; US 20080286808; WO 2011123813; Geething et al., PLoS One, 2010, 5(4), 1-11; and Schellenberger et al., Nature Biotech., 2009, 27(12), 1186-1192; the contents of which are incorporated by reference in their entirety, describe such technologies and derivatives, and methods for their manufacture.

### IV. Methods of Use

In one aspect, the present invention includes a method for maintaining C-peptide levels above the minimum effective therapeutic level in a patient in need thereof, comprising administering to the patient a therapeutic dose of any of the claimed modified C-peptides.

In another aspect, the present invention includes a method for treating one or more long-term complications of diabetes in a patient in need thereof, comprising administering to the patient a therapeutic dose of any of the claimed modified C-peptides.

In another aspect, the present invention includes a method for treating a patient with diabetes comprising administering to the patient a therapeutic dose of any of the claimed modified C-peptides in combination with insulin.

In another aspect, the present invention includes any of the claimed modified C-peptides for use as a C-peptide replacement therapy or dose in a patient in need thereof.

In broad terms, diabetes refers to the situation where the body either fails to properly respond to its own insulin, does not make enough insulin, or both. The primary result of impaired insulin production is the accumulation of glucose in the blood, and a C-peptide deficiency leading to various short- and long-term complications. Three principal forms of diabetes exist:

Type 1: Results from the body's failure to produce insulin and C-peptide. It is estimated that 5-10% of Americans who are diagnosed with diabetes have type 1 diabetes. Presently almost all persons with type 1 diabetes must take insulin injections. The term “type 1 diabetes” has replaced several former terms, including childhood-onset diabetes, juvenile diabetes, and insulin-dependent diabetes mellitus (IDDM). For patients with type 1 diabetes, basal levels of C-peptide are typically less than about 0.20 nM (Ludvigsson et al.: New Engl. J. Med. 359: 1909-1920, (2008)).

Type 2: Results from tissue insulin resistance, a condition in which cells fail to respond properly to insulin, sometimes combined with relative insulin deficiency. The term “type 2 diabetes” has replaced several former terms, including adult-onset diabetes, obesity-related diabetes, and non-insulin-dependent diabetes mellitus (NIDDM). For type 2 patients in the basal state, C-peptide levels of about 0.8 nM (range 0.64 to 1.56 nM), and glucose stimulated levels of about 5.7 nM (range 3.7 to 7.7 nM) have been reported. (Retnakaran R et al.: Diabetes Obes. Metab. (2009) DOI 10.11 111/j.1463-1326.2009.01129.x; Zander et al.: Lancet 359: 824-830, (2002)).

In addition to type 1 and type 2 diabetics, there is increasing recognition of a subclass of diabetes referred to as latent autoimmune diabetes in the adult (LADA) or late-onset autoimmune diabetes of adulthood, or “slow onset type 1” diabetes, and sometimes also “type 1.5” or “type one-and-a-half” diabetes. In this disorder, diabetes onset generally occurs in ages 35 and older, and antibodies against components of the insulin-producing cells are always present, demonstrating that autoimmune activity is an important feature of LADA. It is primarily antibodies against glutamic acid decarboxylase (GAD) that are found. Some LADA patients show a phenotype similar to that of type 2 patients with increased body mass index (BMI) or obesity, insulin resistance, and abnormal blood lipids. Genetic features of LADA are similar to those for both type 1 and type 2 diabetes. During the first 6-12 months after debut the patients may not require insulin administration and they are able to maintain relative normoglycemia via dietary modification and/or oral anti-diabetic medication. However, eventually all patients become insulin dependent, probably as a consequence of progressive autoimmune activity leading to gradual destruction of the pancreatic islet β-cells. At this stage the LADA patients show low or absent levels of endogenous insulin and C-peptide, and they are prone to develop long-term complications of diabetes involving the peripheral nerves, the kidneys, or the eyes similar to type 1 diabetes patients and thus become candidates for C-peptide therapy (Palmer et al.: Diabetes 54(suppl 2): S62-67, (2005); Desai et al.: Diabetic Medicine 25(suppl 2): 30-34, (2008); Fourlanos et al.: Diabetologia 48: 2206-2212, (2005)).

Gestational Diabetes:

Pregnant women who have never had diabetes before but who have high blood sugar (glucose) levels during pregnancy are said to have gestational diabetes. Gestational diabetes affects about 4% of all pregnant women. It may precede development of type 2 (or rarely type 1) diabetes.

Several other forms of diabetes mellitus are categorized separately from these. Examples include congenital diabetes due to genetic defects of insulin secretion, cystic fibrosis-related diabetes, steroid diabetes induced by high doses of glucocorticoids, and several forms of monogenic diabetes.

Accordingly in any of these methods, the term “patient” refers to an individual who has one of more of the symptoms of any of diabetes. In one aspect of any of these methods, the term “patient” refers to an individual who has one of more of the symptoms of any of insulin-dependent diabetes. In one aspect of any of these methods, the term “patient” refers to an individual who has one of more of the symptoms of any of type 2 diabetes. In one aspect of any of these methods, the term “patient” refers to an individual who has one of more of the symptoms of LADA. In one aspect of any of these methods, the term “patient” refers to an individual who has one of more of the symptoms of gestational diabetes. Accordingly in one aspect of any of these methods, the term “patient” refers to an individual who has a fasting C-peptide level of less than about 0.4 nM. In another aspect of any of these methods, the term “patient” refers to an individual who has a fasting C-peptide level of less than about 0.2 nM.

Acute complications of diabetes include hypoglycemia, diabetic ketoacidosis, or nonketotic hyperosmolar coma that may occur if the disease is not adequately controlled. Serious long-term complications can also occur, and are discussed in more detail below.

In another aspect, the present invention includes a method for treating one or more long-term complications of diabetes in a patient in need thereof, comprising administering to the patient a therapeutic dose of any of the claimed modified C-peptides.

In another aspect, the present invention includes a method for treating a patient with diabetes comprising administering to the patient a therapeutic dose of any of the claimed modified C-peptides in combination with insulin.

In this context “in combination” means: 1) part of the same unitary dosage form; 2) ex vivo administration separately, but as part of the same therapeutic treatment program or regimen, typically but not necessarily, on the same day. In one aspect, any of the claimed modified C-peptides may be administered at a fixed daily dosage, and the insulin taken on an as needed basis.

In another aspect, the present invention includes any of the claimed modified C-peptides for use for treating one or more long-term complications of diabetes in a patient in need thereof.

In any of these methods, the terms “long-term complication of type 1 diabetes”, or “long-term complications of diabetes” refers to the long-term complications of impaired glycemic control, and C-peptide deficiency associated with insulin-dependent diabetes. Typically long-term complications of type 1 diabetes are associated with type 1 diabetics. However the term can also refer to long-term complications of diabetes that arise in type 1.5 and type 2 diabetic patients who develop a C-peptide deficiency as a consequence of losing pancreatic islet β-cells and therefore also become insulin dependent. In broad terms, many such complications arise from the primary damage of blood vessels (angiopathy), resulting in subsequent problems that can be grouped under “microvascular disease” (due to damage to small blood vessels) and “macrovascular disease” (due to damage to the arteries).

Specific diseases and disorders included within the term long-term complications of diabetes include, without limitation; retinopathy including early stage retinopathy with microaneurysms, proliferative retinopathy, and macular edema; peripheral neuropathy including sensorimotor polyneuropathy, painful sensory neuropathy, acute motor neuropathy, cranial focal and multifocal polyneuropathies, thoracolumbar radiculoneuropathies, proximal diabetic neuropathies, and focal limb neuropathies including entrapment and compression neuropathies; autonomic neuropathy involving the cardiovascular system, the gastrointestinal tract, the respiratory system, the urigenital system, sudomotor function and papillary function; and nephropathy including disorders with microalbuminuria, overt proteinuria, and end-stage renal disease.

Impaired microcirculatory perfusion appears to be crucial to the pathogenesis of both neuropathy and retinopathy in diabetics. This in turn reflects a hyperglycemia-mediated perturbation of vascular endothelial function that results in: over-activation of protein kinase C, reduced availability of nitric oxide (NO), increased production of superoxide and endothelin-1 (ET-1), impaired insulin function, diminished synthesis of prostacyclin/PGE1, and increased activation and endothelial adherence of leukocytes. This is ultimately a catastrophic group of clinical events.

Accordingly in some embodiments, the term “patient” refers to an individual who has one of more of the symptoms of the long-term complications of diabetes.

Diabetic retinopathy is an ocular manifestation of the systemic damage to small blood vessels leading to microangiopathy. In retinopathy, growth of friable and poor-quality new blood vessels in the retina as well as macular edema (swelling of the macula) can lead to severe vision loss or blindness. As new blood vessels form at the back of the eye as a part of proliferative diabetic retinopathy (PDR), they can bleed (hemorrhage) and blur vision. It affects up to 80% of all patients who have had diabetes for 10 years or more.

The symptoms of diabetic retinopathy are often slow to develop and subtle and include blurred version and progressive loss of sight. Macular edema, which may cause vision loss more rapidly, may not have any warning signs for some time. In general, however, a person with macular edema is likely to have blurred vision, making it hard to do things like read or drive. In some cases, the vision will get better or worse during the day.

Accordingly in some embodiments, the term “patient” refers to an individual who has one of more of the symptoms of diabetic retinopathy.

Diabetic neuropathies are neuropathic disorders that are associated with diabetic microvascular injury involving small blood vessels that supply nerves (vasa nervorum). Relatively common conditions which may be associated with diabetic neuropathy include third nerve palsy; mononeuropathy; mononeuropathy multiplex; diabetic amyotrophy; a painful polyneuropathy; autonomic neuropathy; and thoracoabdominal neuropathy.

Diabetic neuropathy affects all peripheral nerves: pain fibers, motor neurons, autonomic nerves. It therefore necessarily can affect all organs and systems since all are innervated. There are several distinct syndromes based on the organ systems and members affected, but these are by no means exclusive. A patient can have sensorimotor and autonomic neuropathy or any other combination. Symptoms vary depending on the nerve(s) affected and may include symptoms other than those listed. Symptoms usually develop gradually over years.

Symptoms of diabetic neuropathy may include: numbness and tingling of extremities, dysesthesia (decreased or loss of sensation to a body part), diarrhea, erectile dysfunction, urinary incontinence (loss of bladder control), impotence, facial, mouth and eyelid drooping, vision changes, dizziness, muscle weakness, difficulty swallowing, speech impairment, fasciculation (muscle contractions), anorgasmia, and burning or electric pain.

Additionally, different nerves are affected in different ways by neuropathy. Sensorimotor polyneuropathy, in which longer nerve fibers are affected to a greater degree than shorter ones, because nerve conduction velocity is slowed in proportion to a nerve's length. In this syndrome, decreased sensation and loss of reflexes occurs first in the toes on each foot, then extends upward. It is usually described as glove-stocking distribution of numbness, sensory loss, dysesthesia, and night-time pain. The pain can feel like burning, pricking sensation, achy, or dull. Pins and needles sensation is common. Loss of proprioception, the sense of where a limb is in space, is affected early. These patients cannot feel when they are stepping on a foreign body, like a splinter, or when they are developing a callous from an ill-fitting shoe. Consequently, they are at risk for developing ulcers and infections on the feet and legs, which can lead to amputation. Similarly, these patients can get multiple fractures of the knee, ankle, or foot, and develop a Charcot joint. Loss of motor function results in dorsiflexion, contractures of the toes, loss of the interosseous muscle function, and leads to contraction of the digits, so called hammer toes. These contractures occur not only in the foot, but also in the hand where the loss of the musculature makes the hand appear gaunt and skeletal. The loss of muscular function is progressive.

Autonomic neuropathy impacts the autonomic nervous system serving the heart, gastrointestinal system, and genitourinary system. The most commonly recognized autonomic dysfunction in diabetics is orthostatic hypotension, or fainting when standing up. In the case of diabetic autonomic neuropathy, it is due to the failure of the heart and arteries to appropriately adjust heart rate and vascular tone to keep blood continually and fully flowing to the brain. This symptom is usually accompanied by a loss of the usual change in heart rate seen with normal breathing. These two findings suggest autonomic neuropathy.

Gastrointestinal system symptoms include delayed gastric emptying, gastroparesis, nausea, bloating, and diarrhea. Because many diabetics take oral medication for their diabetes, absorption of these medicines is greatly affected by the delayed gastric emptying. This can lead to hypoglycemia when an oral diabetic agent is taken before a meal and does not get absorbed until hours, or sometimes days later, when there is normal or low blood sugar already. Sluggish movement of the small intestine can cause bacterial overgrowth, made worse by the presence of hyperglycemia. This leads to bloating, gas, and diarrhea.

Genitourinary system symptoms associated with autonomic neuropathy include urinary frequency, urgency, incontinence, retention, impotence, and erectile dysfunction. Urinary retention can lead to bladder diverticula, stones, reflux nephropathy, and frequent urinary tract infections. Administration of C-peptide has been shown to improve erectile function in insulin-requiring diabetic patients (Wahren et al.: Diabetes 60, Suppl 1: A285, (2011)). Accordingly in any of these methods, the term “patient” refers to an individual who has one of more of the symptoms of autonomic neuropathy. In certain methods, the term “patient” refers to an individual who has one or more symptoms of erectile dysfunction or impotence.

Accordingly in some embodiments, the term “patient” refers to an individual who has one of more of the symptoms of diabetic neuropathy. In another aspect of any of these methods, the patient has “established peripheral neuropathy” which is characterized by reduced sensory nerve conduction velocity (SCV) in the sural nerves (less than −1.5 SD from a body height-corrected reference value for a matched normal individual). In certain embodiments, the term “patient” refers to an individual who has one of more of the symptoms of incipient neuropathy.

Accordingly in certain embodiments, the current invention includes a method of treating or preventing a decrease in a subject's, or patient's, height-adjusted sensory or motor nerve conduction velocity. In one aspect of this method, the motor nerve conduction velocity is initial nerve conduction velocity. In another embodiment, the motor nerve conduction velocity is the peak nerve conduction velocity. Methods of assessing the effect of the modified C-peptides on nerve conduction velocity in diabetic rats are shown in Example 48.

In certain embodiments the subject is a patient with diabetes. In certain embodiments, the subject has at least one long term complication of diabetes. In one aspect, the patient exhibits a peak nerve conduction velocity that is at least about 2 standard deviations from the mean peak nerve conduction velocity for a similar height-matched subject group. In one aspect, the patients have a peak nerve conduction velocity of greater than about 35 m/s. In one aspect of any of the claimed methods, the patients have a peak nerve conduction velocity of greater than about 40 m/s. In one aspect, the patients have a peak nerve conduction velocity of greater than about 45 m/s. In one aspect, the patients have a peak nerve conduction velocity of greater than about 50 m/s.

In one aspect of any of the claimed methods, treatment results in an improvement in nerve conduction velocity of at least about 1.5 m/s. In another aspect of these methods, treatment results in an improvement in nerve conduction velocity of at least about 2.0 m/s. In another aspect of these methods, treatment results in an improvement in nerve conduction velocity of at least about 2.5 m/s. In another aspect of these methods, treatment results in an improvement in nerve conduction velocity of at least about 3.0 m/s. In another aspect of these methods, treatment results in an improvement in nerve conduction velocity of at least about 3.5 m/s. In another aspect of these methods, treatment results in an improvement in nerve conduction velocity of at least about 4.0 m/s. In another aspect of these methods, treatment results in an improvement in nerve conduction velocity of at least about 4.5 m/s. In another aspect of these methods, treatment results in an improvement in nerve conduction velocity of at least about 5.0 m/s. In another aspect of these methods, treatment results in an improvement in nerve conduction velocity of at least about 5.5 m/s. In another aspect of these methods, treatment results in an improvement in nerve conduction velocity of at least about 6.0 m/s. In another aspect of these methods, treatment results in an improvement in nerve conduction velocity of at least about 7.0 m/s. In another aspect of these methods, treatment results in an improvement in nerve conduction velocity of at least about 8.0 m/s. In another aspect of these methods, treatment results in an improvement in nerve conduction velocity of at least about 9.0 m/s. In another aspect of these methods, treatment results in an improvement in nerve conduction velocity of at least about 10.0 m/s. In another aspect of these methods, treatment results in an improvement in nerve conduction velocity of at least about 15.0 m/s. In another aspect of these methods, treatment results in an improvement in nerve conduction velocity of at least about 20.0 m/s.

In certain embodiments, of any of these methods, treatment results in an improvement of at least 10% in peak nerve conduction velocity compared to peak nerve conduction velocity prior to starting modified C-peptide therapy. In certain embodiments, of any of these methods, treatment results in an improvement of at least 15% in peak nerve conduction velocity compared to peak nerve conduction velocity prior to starting modified C-peptide therapy. In certain embodiments, of any of these methods, treatment results in an improvement of at least 20% in peak nerve conduction velocity compared to peak nerve conduction velocity prior to starting modified C-peptide therapy. In certain embodiments, of any of these methods, treatment results in an improvement of at least 25% in peak nerve conduction velocity compared to peak nerve conduction velocity prior to starting modified C-peptide therapy. In certain embodiments, of any of these methods, treatment results in an improvement of at least 30% in peak nerve conduction velocity compared to peak nerve conduction velocity prior to starting modified C-peptide therapy. In certain embodiments, of any of these methods, treatment results in an improvement of at least 40% in peak nerve conduction velocity compared to peak nerve conduction velocity prior to starting modified C-peptide therapy. In certain embodiments, of any of these methods, treatment results in an improvement of at least 50% in peak nerve conduction velocity compared to peak nerve conduction velocity prior to starting modified C-peptide therapy.

Diabetic nephropathy is a progressive kidney disease caused by angiopathy of capillaries in the kidney glomeruli. It is characterized by nephrotic syndrome and diffuse glomerulosclerosis. It is due to long-standing diabetes mellitus, and is a prime cause for dialysis in many Western countries.

The symptoms of diabetic nephropathy can be seen in patients with chronic diabetes (15 years or more after onset). The disease is progressive and is more frequent in men. Diabetic nephropathy is the most common cause of chronic kidney failure and end-stage kidney disease in the United States. People with both type 1 and type 2 diabetes are at risk. The risk is higher if blood-glucose levels are poorly controlled. Further, once nephropathy develops, the greatest rate of progression is seen in patients with poor control of their blood pressure. Also people with high cholesterol level in their blood have much more risk than others.

The earliest detectable change in the course of diabetic nephropathy is an abnormality of the glomerular filtration barrier. At this stage, the kidney may start allowing more serum albumin than normal in the urine (albuminuria), and this can be detected by sensitive medical tests for albumin. This stage is called “microalbuminuria”. As diabetic nephropathy progresses, increasing numbers of glomeruli are destroyed by nodular glomerulosclerosis. Now the amounts of albumin being excreted in the urine increases, and may be detected by ordinary urinalysis techniques. At this stage, a kidney biopsy clearly shows diabetic nephropathy.

Kidney failure provoked by glomerulosclerosis leads to fluid filtration deficits and other disorders of kidney function. There is an increase in blood pressure (hypertension) and fluid retention in the body plus a reduced plasma oncotic pressure causes edema. Other complications may be arteriosclerosis of the renal artery and proteinuria.

Throughout its early course, diabetic nephropathy has no symptoms. They develop in late stages and may be a result of excretion of high amounts of protein in the urine or due to renal failure. Symptoms include, edema; swelling, usually around the eyes in the mornings; later, general body swelling may result, such as swelling of the legs, foamy appearance or excessive frothing of the urine (caused by the proteinura), unintentional weight gain (from fluid accumulation), anorexia (poor appetite), nausea and vomiting, malaise (general ill feeling), fatigue, headache, frequent hiccups, and generalized itching.

Accordingly in some embodiments, the term “patient” refers to an individual who has one of more of the symptoms of diabetic nephropathy.

Diabetic cardiomyopathy (DCM), damage to the heart, leading to diastolic dysfunction and eventually heart failure. Aside from large vessel disease and accelerated atherosclerosis, which is very common in diabetes, DCM is a clinical condition diagnosed when ventricular dysfunction develops in patients with diabetes in the absence of coronary atherosclerosis and hypertension. DCM may be characterized functionally by ventricular dilation, myocyte hypertrophy, prominent interstitial fibrosis, and decreased or preserved systolic function in the presence of a diastolic dysfunction.

One particularity of DCM is the long latent phase, during which the disease progresses but is completely asymptomatic. In most cases, DCM is detected with concomitant hypertension or coronary artery disease. One of the earliest signs is mild left ventricular diastolic dysfunction with little effect on ventricular filling. Also, the diabetic patient may show subtle signs of DCM related to decreased left ventricular compliance or left ventricular hypertrophy or a combination of both. A prominent “a” wave can also be noted in the jugular venous pulse, and the cardiac apical impulse may be overactive or sustained throughout systole. After the development of systolic dysfunction, left ventricular dilation and symptomatic heart failure, the jugular venous pressure may become elevated and the apical impulse would be displaced downward and to the left. Systolic mitral murmur is not uncommon in these cases. These changes are accompanied by a variety of electrocardiographic changes that may be associated with DCM in 60% of patients without structural heart disease, although usually not in the early asymptomatic phase. Later in the progression, a prolonged QT interval may be indicative of fibrosis. Given that the definition of DCM excludes concomitant atherosclerosis or hypertension, there are no changes in perfusion or in atrial natriuretic peptide levels up until the very late stages of the disease, when the hypertrophy and fibrosis become very pronounced.

In certain embodiments, the term “patient” refers to an individual who has one of more of the symptoms of diabetic cardiomyopathy.

Macrovascular diseases of diabetes include coronary artery disease, leading to angina or myocardial infarction (“heart attack”), stroke (mainly the ischemic type), peripheral vascular disease, which contributes to intermittent claudication (exertion-related leg and foot pain), as well as diabetic foot and diabetic myonecrosis (“muscle wasting”).

In certain embodiments, the term “patient” refers to an individual who has one of more of the symptoms of a macrovascular disease of diabetes.

Methods for Preventing Hypoglycemia.

In certain embodiments, the present invention includes the use of any of the disclosed modified C-peptides to reduce the risk of hypoglycemia in a human patient with insulin dependent diabetes, in a regimen which additionally comprises the administration of insulin, comprising; a) administering insulin to said patient; b) administering a therapeutic dose of modified C-peptide in a different site as that used for said patient's insulin administration; c) adjusting the dosage amount, type, or frequency of insulin administered based on said patient's altered insulin requirements resulting from said therapeutic dose of modified C-peptide.

In another aspect, the present invention includes a method of reducing insulin usage in an insulin-dependent human patient, comprising the steps of; a) administering insulin to said patient; b) administering subcutaneously to said patient a therapeutic dose of any of the disclosed modified C-peptides in a different site as that used for said patient's insulin administration; c) adjusting the dosage amount, type, or frequency of insulin administered based on monitoring said patient's altered insulin requirements resulting from said therapeutic dose of modified C-peptide, wherein said adjusted dose of insulin does not induce hyperglycemia, wherein said adjusted dose of insulin is at least 10% less than said patient's insulin dose prior to starting modified C-peptide. (See for example U.S. Pat. No. 7,855,177, which is herein incorporated by reference).

In any of these methods, the term “hypoglycemia” or “hypoglycemic events” refers to all episodes of abnormally low plasma glucose concentration that exposes the patient to potential harm. The American Diabetes Association Workgroup has recommended that people with insulin-dependent diabetes become concerned about the possibility of developing hypoglycemia at a plasma glucose concentration of less than 70 mg/dL (3.9 mmoL/L). Accordingly in one aspect of any of the claimed methods, the terms hypoglycemia or hypoglycemic event refers to the situation where the plasma glucose concentration of the patient drops to less than about 70 mg/dL (3.9 mmoL/L).

Hypoglycemia is a serious medical complication in the treatment of diabetes, and causes recurrent morbidity in most people with type 1 diabetes and many with advanced type 2 diabetes and is sometimes fatal. In addition, hypoglycemia compromises physiological and behavioral defenses against subsequent falling plasma glucose concentrations and thus causes a vicious cycle of recurrent hypoglycemia. Accordingly the prevention of hypoglycemia is of significant importance in the treatment of diabetes, as well as the treatment of the long-term complications of diabetes.

Unfortunately hypoglycemia is a fact of life for most people with type 1 diabetes (Cryer P E et al.: Diabetes 57: 3169-3176, (2008)). The average patient has untold numbers of episodes of asymptomatic hypoglycemia and suffers two episodes of symptomatic hypoglycemia per week, with thousands of such episodes over a lifetime of diabetes. He or she suffers one or more episodes of severe, temporarily disabling hypoglycemia often with seizure or coma, per year.

Overall, hypoglycemia is less frequent in type 2 diabetes; however, the risk of hypoglycemia becomes progressively more frequent and limiting to glycemic control later in the course of type 2 diabetes. The prospective, population-based data of Donnelly et al. (Diabetes Med. 22: 749-755, (2005)) indicate that the overall incidence of hypoglycemia in insulin-treated type 2 diabetes is approximately one third of that in type 1 diabetes. The incidence of any hypoglycemia and of severe hypoglycemia was 4,300 and 115 episodes per 100 patient years, respectively, in type 1 diabetes and 1600 and 35 episodes per 100 patient years, respectively, in insulin-treated type 2 diabetes.

Hypoglycemia may be classified based on the severity of the hypoglycemic event. For example, the American Diabetes Association Workgroup has suggested the following classification of hypoglycemia in diabetes: 1) severe hypoglycemia (i.e., hypoglycemic coma requiring assistance of another person); 2) documented symptomatic hypoglycemia (with symptoms and a plasma glucose concentration of less than 70 mg/dL); 3) asymptomatic hypoglycemia (with a plasma glucose concentration of less than 70 mg/dL without symptoms); 4) probable symptomatic hypoglycemia (with symptoms attributed to hypoglycemia, but without a plasma glucose measurement); and 5) relative hypoglycemia (with a plasma glucose concentration of greater than 70 mg/dL but falling towards that level).

Thus in another aspect of any of the methods disclosed herein, the term “hypoglycemia” refers to severe hypoglycemia, and/or hypoglycemic coma. In another aspect of any of these methods, the term “hypoglycemia” refers to symptomatic hypoglycemia. In another aspect of any of these methods, the term “hypoglycemia” refers to probable symptomatic hypoglycemia. In another aspect of any of these methods, the term “hypoglycemia” refers to asymptomatic hypoglycemia. In another aspect of any of these methods, the term “hypoglycemia” refers to relative hypoglycemia.

Insulin Types and Administration Forms

There are over 180 individual insulin preparations available worldwide which have been developed to provide different lengths of activity (activity profiles). Approximately 25% of these are soluble insulin (unmodified form); about 35% are long- or intermediate-acting basal insulins (mixed with NPH [neutral protamine Hagedorn] insulin or Lente insulin [insulin zinc suspension], or forms that are modified to have an increased isoelectric point [insulin glargine], or acylation [insulin detemir]; these forms have reduced solubility, slow subcutaneous absorption, and long duration of action relative to soluble insulins); about 2% are rapid-acting insulins (e.g., which are engineered by amino acid change, and have reduced self-association and increased subcutaneous absorption); and about 38% are pre-mixed insulins (e.g., mixtures of short-, intermediate-, and long-acting insulins; these preparations have the benefit of a reduced number of daily injections).

Short-acting insulin preparations that are commercially available in the US include regular insulin and rapid-acting insulins. Regular insulin has an onset of action of 30-60 minutes, peak time of effect of 1.5 to 2 hours, and duration of activity of 5 to 12 hours. Rapid-acting insulins, such as Aspart (Novo Rapid), Lispro (Humalog), and Glulisine (Apidra), have an onset of action of 10-30 minutes, peak time of effect of around 30 minutes, and a duration of activity of 3 to 5 hours.

Intermediate-acting insulins, such as NPH and Lente insulins, have an onset of action of 1 to 2 hours, peak time of effect of 4 to 8 hours, and a duration of activity of 10 to 20 hours.

Long-acting insulins, such as Ultralente insulin, have an onset of action of 2 to 4 hours, peak time of effect of 8 to 20 hours, and a duration of activity of 16 to 24 hours. Other examples of long-acting insulins include Glargine and Determir. Glargine insulin has an onset of action of 1 to 2 hours, and a duration of action of 24 hours, but with no peak effect.

In many cases, regimens that use insulin in the management of diabetes combine long-acting and short-acting insulin. For example, Lantus, from Aventis Pharmaceuticals Inc., is a recombinant human insulin analog that is a long-acting, parenteral blood-glucose-lowering agent whose longer duration of action (up to 24 hours) is directly related to its slower rate of absorption. Lantus is administered subcutaneously once a day, preferably at bedtime, and is said to provide a continuous level of insulin, similar to the slow, steady (basal) secretion of insulin provided by the normal pancreas. The activity of such a long-acting insulin results in a relatively constant concentration/time profile over 24 hours with no pronounced peak, thus allowing it to be administered once a day as a patient's basal insulin. Such long-acting insulin has a long-acting effect by virtue of its chemical composition, rather than by virtue of an addition to insulin when administered.

More recently automated wireless controlled systems for continuous infusion of insulin, such as the system sold under the trademark OMNIPOD™ Insulin Management System (Insulet Corporation, Bedford, Mass.) have been developed. These systems provide continuous subcutaneous insulin delivery with blood glucose monitoring technology in a discreet two-part system. This system eliminates the need for daily insulin injections, and does not require a conventional insulin pump which is connected via tubing.

OMNIPOD™ is a small lightweight device that is worn on the skin like an infusion set. It delivers insulin according to pre-programmed instructions transmitted wirelessly from the Personal Diabetes Manager (PDM). The PDM is a wireless, hand-held device that is used to program the OMNIPOD™ Insulin Management System with customized insulin delivery instructions, monitor the operation of the system, and check blood glucose levels using blood glucose test strips sold under the trademark FREESTYLE™. There is no tubing connecting the device to the PDM. OMNIPOD™ Insulin Management System is worn beneath the clothing, and the PDM can be carried separately in a backpack, briefcase, or purse. Similar to currently available insulin pumps, the OMNIPOD™ Insulin Management System features fully programmable continuous subcutaneous insulin delivery with multiple basal rates and bolus options, suggested bolus calculations, safety checks, and alarm features.

The aim of insulin treatment of diabetics is typically to administer enough insulin such that the patient will have blood glucose levels within the physiological range and normal carbohydrate metabolism throughout the day. Because the pancreas of a diabetic individual does not secrete sufficient insulin throughout the day, in order to effectively control diabetes through insulin therapy, a long-lasting insulin treatment, known as basal insulin, must be administered to provide the slow and steady release of insulin that is needed to control blood glucose concentrations and to keep cells supplied with energy when no food is being digested. Basal insulin is necessary to suppress glucose production between meals and overnight and preferably mimics the patient's normal pancreatic basal insulin secretion over a 24-hour period. Thus, a diabetic patient may administer a single dose of a long-acting insulin each day subcutaneously, with an action lasting about 24 hours.

Furthermore, in order to effectively control diabetes through insulin therapy by dealing with postprandial rises in glucose levels, a bolus, fast-acting treatment must also be administered. The bolus insulin, which is generally administered subcutaneously, provides a rise in plasma insulin levels at approximately 1 hour after administration, thereby limiting hyperglycemia after meals. Thus, these additional quantities of regular insulin, with a duration of action of, e.g., 5 to 6 hours, may be subcutaneously administered at those times of the day when the patient's blood glucose level tends to rise too high, such as at meal times. As an alternative to administering basal insulin in combination with bolus insulin, repeated and regular lower doses of bolus insulin may be administered in place of the long-acting basal insulin, and bolus insulin may be administered postprandially as needed.

Currently, regular subcutaneously injected insulin is recommended to be dosed at 30 to 45 minutes prior to mealtime. As a result, diabetic patients and other insulin users must engage in considerable planning of their meals and of their insulin administrations relative to their meals. Unfortunately, intervening events that may take place between administration of insulin and ingestion of the meal may affect the anticipated glucose excursions.

Furthermore, there is also the potential for hypoglycemia if the administered insulin provides a therapeutic effect over too great a time, e.g., after the rise in glucose levels that occur as a result of ingestion of the meal has already been lowered. As outlined in the Examples, this risk of hypoglycemia is increased in patients who have been treated with C-peptide due to a reduced requirement for insulin.

Accordingly, in one aspect of any of the methods disclosed herein, the present invention includes a method for reducing the risk of the patient developing hypoglycemia by reducing the average daily dose of insulin administered to the patient by about 5% to about 50% after starting modified C-peptide therapy. In another aspect, the dose of insulin administered is reduced by about 5% to about 45% compared to the patient's insulin dose prior to starting modified C-peptide treatment. In another aspect, the dose of insulin administered is reduced by about 5% to about 40% compared to the patient's insulin dose prior to starting modified C-peptide treatment. In another aspect, the dose of insulin administered is reduced by about 5% to about 35% compared to the patient's insulin dose prior to starting modified C-peptide treatment. In another aspect, the dose of insulin administered is reduced by about 5% to about 30% compared to the patient's insulin dose prior to starting modified C-peptide treatment. In another aspect, the dose of insulin administered is reduced by about 5% to about 25% compared to the patient's insulin dose prior to starting modified C-peptide treatment. In another aspect, the dose of insulin administered is reduced by about 5% to about 20% compared to the patient's insulin dose prior to starting modified C-peptide treatment. In another aspect, the dose of insulin administered is reduced by about 5% to about 15% compared to the patient's insulin dose prior to starting modified C-peptide treatment. In another aspect, the dose of insulin administered is reduced by about 5% to about 10% compared to the patient's insulin dose prior to starting modified C-peptide treatment.

In another aspect, the dose of insulin administered is reduced by about 2% to about 10% compared to the patient's insulin dose prior to starting modified C-peptide treatment. In another aspect, the dose of insulin administered is reduced by about 2% to about 15% compared to the patient's insulin dose prior to starting modified C-peptide treatment. In another aspect, the dose of insulin administered is reduced by about 2% to about 20% compared to the patient's insulin dose prior to starting modified C-peptide treatment.

In another aspect, the dose of insulin administered is reduced by about 10% to about 50% compared to the patient's insulin dose prior to starting modified C-peptide treatment. In another aspect, the dose of insulin administered is reduced by about 10% to about 45% compared to the patient's insulin dose prior to starting modified C-peptide treatment. In another aspect, the dose of insulin administered is reduced by about 10% to about 40% compared to the patient's insulin dose prior to starting modified C-peptide treatment. In another aspect, the dose of insulin administered is reduced by about 10% to about 35% compared to the patient's insulin dose prior to starting C-peptide treatment. In another aspect, the dose of insulin administered is reduced by about 10% to about 30% compared to the patient's insulin dose prior to starting modified C-peptide treatment. In another aspect, the dose of insulin administered is reduced by about 10% to about 25% compared to the patient's insulin dose prior to starting modified C-peptide treatment. In another aspect, the dose of insulin administered is reduced by about 10% to about 20% compared to the patient's insulin dose prior to starting modified C-peptide treatment. In another aspect, the dose of insulin administered is reduced by at least 10% compared to the patient's insulin dose prior to starting modified C-peptide treatment.

In one aspect of any of these methods, the dose of short-acting insulin administered is selectively reduced by any of the prescribed ranges listed above. In another aspect of any of these methods, the dose of intermediate-acting insulin administered is selectively reduced by any of the prescribed ranges. In one aspect of any of these methods, the dose of long-acting insulin administered is selectively reduced by any of the prescribed ranges listed above.

In another aspect of any of these methods, the dose of intermediate- and long-acting insulin administered is independently reduced by any of the prescribed ranges listed above, while the dose of short-acting insulin remains substantially unchanged.

In one aspect of these methods, the dose of short-acting insulin administered is reduced by about 5% to about 50% compared to the patient's insulin dose prior to starting modified C-peptide treatment. In another embodiment, the dose of short-acting insulin administered is reduced by about 5% to about 35% compared to the patient's insulin dose prior to starting modified C-peptide treatment. In another embodiment, the dose of short-acting insulin administered is reduced by about 10% to about 20% compared to the patient's insulin dose prior to starting modified C-peptide treatment. In one aspect of these methods, the dose of short-acting insulin administered preprandially for a meal is reduced. In another aspect of these methods, the dose of short-acting insulin administered in the morning or at nighttime is reduced. In another aspect of any of these methods, the dose of short-acting insulin administered is reduced while the dose of long-acting and/or intermediate-acting insulin administered to the patient is substantially unchanged.

In another aspect of any of the methods disclosed herein, the present invention includes a method for reducing the risk of the patient developing hypoglycemia by reducing the average daily dose of intermediate-acting insulin administered to the patient by about 5% to about 35% after starting modified C-peptide therapy. In one aspect of these methods, the dose of intermediate-acting insulin administered is reduced by about 5% to about 50% compared to the patient's insulin dose prior to starting modified C-peptide treatment. In another embodiment, the dose of intermediate-acting insulin administration is reduced by about 5% to about 35% compared to the patient's insulin dose prior to starting modified C-peptide treatment. In another embodiment, the dose of intermediate-acting insulin administered is reduced by about 10% to about 20% compared to the patient's insulin dose prior to starting modified C-peptide treatment. In another aspect of these methods, the dose of intermediate-acting insulin administered in the morning or at nighttime is reduced. In another aspect of any of these methods, the dose of intermediate-acting insulin administered is reduced while the dose of short-acting insulin administered to the patient is substantially unchanged.

In another aspect of any of the methods disclosed herein, the present invention includes a method for reducing the risk of the patient developing hypoglycemia by reducing the average daily dose of long-acting insulin administered to the patient by about 5% to about 50% after starting modified C-peptide therapy. In one embodiment, the dose of long-acting insulin administered is reduced by about 5% to about 35% compared to the patient's insulin dose prior to starting modified C-peptide treatment. In another embodiment, the dose of long-acting insulin administered is reduced by about 10% to about 20% compared to the patient's insulin dose prior to starting modified C-peptide treatment. In another aspect of these methods, the dose of long-acting insulin administered in the morning or at nighttime is reduced. In another aspect of any of these methods, the dose of long-acting insulin administered is reduced while the dose of short-acting insulin administered to the patient is substantially unchanged.

In certain preferred embodiments, the patient achieves improved insulin utilization and insulin sensitivity while experiencing a reduced risk of developing hypoglycemia after treatment with modified C-peptide as compared with baseline levels prior to treatment. Preferably, the improved insulin utilization and insulin sensitivity are measured by a statistically significant decline in HOMA (Homeostasis Model Assessment) (Turner et al.: Metabolism 28(11): 1086-1096, (1979)).

Subcutaneous administration of the modified C-peptide will typically not be into the same site as that most recently used for insulin administration, i.e. modified C-peptide and insulin will be injected into different sites. Specifically in one aspect, the site of modified C-peptide administration will typically be at least about 10 cm way from the site most recently used for insulin administration. In another aspect, the site of modified C-peptide administration will typically be at least about 15 cm away from the site most recently used for insulin administration. In another aspect, the site of modified C-peptide administration will typically be at least about 20 cm away from the site most recently used for insulin administration.

Examples of different sites include for example, and without limitation, injections into the left and right arm, or injections into the left and right thigh, or injections into the left or right buttock, or injections into the opposite sides of the abdomen. Other obvious variants of different sites include injections in an arm and thigh, or injections in an arm and buttock, or injections into an arm and abdomen, etc.

Moreover one of ordinary skill in the art, i.e. a physician, or diabetic patient, will recognize and understand how to inject modified C-peptide and insulin into any other combination of different sites, based on the prior art teaching, and numerous text books and guides on insulin administration that provide disclosure on how to select different insulin injection sites. See for example, the following representative text books (Learning to live well with diabetes, Ed. Cheryl Weiler, (1991) DCI Publishing, Minneapolis, Minn.; American Diabetes Association Complete Guide to Diabetes, ISBN 0-945448-64-3 (1996)).

In one aspect of any of the claimed methods, modified C-peptide is administered to the opposite side of the abdomen to the site most recently used for insulin administration, approximately 15 to 20 cm apart.

### V. Pharmaceutical Compositions

In one aspect, the present invention includes a pharmaceutical composition comprising modified C-peptide, and a pharmaceutically acceptable carrier, diluent or excipient.

Pharmaceutical compositions suitable for the delivery of modified C-peptide and methods for their preparation will be readily apparent to those skilled in the art and may comprise any of the known carriers, diluents, or excipients. Such compositions and methods for their preparation may be found, e.g., in Remington's Pharmaceutical Sciences, 19th Edition (Mack Publishing Company, 1995).

In one aspect, the pharmaceutical compositions may be in the form of sterile aqueous solutions and/or suspensions of the pharmaceutically active ingredients, aerosols, ointments, and the like. Formulations which are aqueous solutions are most preferred. Such formulations typically contain the modified C-peptide itself, water, and one or more buffers which act as stabilizers (e.g., phosphate-containing buffers) and optionally one or more preservatives. Such formulations containing, e.g., about 1 to 200 mg, about 3 to 100 mg, about 3 to 80 mg, about 3 to 60 mg, about 3 to 40 mg, about 3 to 30 mg, about 0.3 to 3.3 mg, about 1 to 3.3 mg, about 1 to 2 mg, about 1 to 3.3 mg, about 2 to 3.3 mg or any of the ranges mentioned herein, e.g., about 200 mg, about 150 mg, about 120 mg, about 100 mg, about 80 mg, about 60 mg, about 50 mg, about 40 mg, about 30 mg, about 20 mg, or about 10 mg, or about 8 mg, or about 6 mg, or about 5 mg, or about 4 mg, or about 3 mg, or about 2 mg, or about 1 mg, or about 0.5 mg of the modified C-peptide and constitute a further aspect of the invention.

Pharmaceutical compositions may include pharmaceutically acceptable salts of modified C-peptide. For a review on suitable salts, see Handbook of Pharmaceutical Salts: Properties, Selection, and Use by Stahl and Wermuth (Wiley-VCH, 2002). Suitable base salts are formed from bases which form non-toxic salts. Representative examples include the aluminium, arginine, benzathine, calcium, choline, diethylamine, diolamine, glycine, lysine, magnesium, meglumine, olamine, potassium, sodium, tromethamine, and zinc salts. Hemisalts of acids and bases may also be formed, e.g., hemisulphate and hemicalcium salts. In one embodiment, modified C-peptide may be prepared as a gel with a pharmaceutically acceptable positively charged ion.

In one aspect, the positively charged ion may be a monovalent metal ion. In one aspect, the metal ion is selected from sodium and potassium.

In one aspect, the positively charged ion may be a divalent metal ion. In one aspect, the metal ion is selected from calcium, magnesium, and zinc.

The modified C-peptide may be administered at any time during the day. For humans, the dosage used may range from about 0.1 to 200 mg/week of modified C-peptide, e.g., from about 0.1 to 0.3 mg/week, about 0.3 to 1.5 mg/week, about 1 mg to about 3.5 mg/week, about 1.5 to 2.25 mg/week, about 2.25 to 3.0 mg/week, about 3.0 to 6.0 mg/week, about 6.0 to 10 mg/week, about 10 to 20 mg/week, about 20 to 40 mg/week, about 40 to 60 mg/week, about 60 to 80 mg/week, about 80 to 100 mg/week, about 100 to 120 mg/week, about 120 to 140 mg/week, about 140 to 160 mg/week, about 160 to 180 mg/week, and about 180 to about 200 mg/week.

Preferably the total weekly dose used of modified C-peptide is about 1 mg to about 3.5 mg, about 1 mg to about 20 mg, about 20 mg to about 50 mg, about 50 mg to about 100 mg, about 100 mg to about 150 mg, or about 150 mg to about 200 mg.

The total weekly dose of modified C-peptide may be about 0.1 mg, about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 7 mg, about 8 mg, about 9 mg, about 10 mg, about 12 mg, about 15 mg, about 18 mg, about 21 mg, about 24 mg, about 27 mg, about 30 mg, about 33 mg, about 36 mg, about 39 mg, about 42 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, or about 200 mg. (It will be appreciated that masses of modified C-peptide referred to above are dependent on the bioavailability of the delivery system and based on the use of modified C-peptide with a molecular mass of approximately 40,000 Da.)

In another aspect of any of these methods and pharmaceutical compositions, the therapeutic dose of modified C-peptide comprises a weekly dose ranging from about 1 mg to about 45 mg. In another aspect of any of these methods and pharmaceutical compositions, the therapeutic dose of modified C-peptide comprises a weekly dose ranging from about 3 mg to about 15 mg. In another aspect of any of these methods and pharmaceutical compositions, the therapeutic dose of modified C-peptide comprises a weekly dose ranging from about 30 mg to about 60 mg. In another aspect of any of these methods and pharmaceutical compositions, the therapeutic dose of modified C-peptide comprises a weekly dose ranging from about 60 mg to about 120 mg.

In another aspect of any of these methods and pharmaceutical compositions, the therapeutic dose of modified C-peptide maintains the average steady-state concentration of modified C-peptide (Cₛₛ₋ₐᵥₑ) in the patient's plasma of between about 0.2 nM and about 6 nM.

In another aspect of any of these methods and pharmaceutical compositions, the therapeutic dose of modified C-peptide is provided to the patient so as to maintain the average steady-state concentration of modified C-peptide in the patient's plasma between about 0.2 nM and about 6 nM when using a dosing interval of 3 days or longer. In another aspect of any of these methods and pharmaceutical compositions, the therapeutic dose of modified C-peptide is provided to the patient so as to maintain the average steady-state concentration of modified C-peptide in the patient's plasma between about 0.2 nM and about 6 nM when using a dosing interval of 4 days or longer. In another aspect of any of these methods and pharmaceutical compositions, the therapeutic dose of modified C-peptide is provided to the patient so as to maintain the average steady-state concentration of modified C-peptide in the patient's plasma between about 0.2 nM and about 6 nM when using a dosing interval of 5 days or longer. In another aspect of any of these methods and pharmaceutical compositions, the therapeutic dose of modified C-peptide is provided to the patient so as to maintain the average steady-state concentration of modified C-peptide in the patient's plasma between about 0.2 nM and about 6 nM when using a dosing interval of at least one week. In any of these methods and pharmaceutical compositions, the therapeutic dose is administered by daily subcutaneous injections. In another aspect of any of these methods and pharmaceutical compositions, the therapeutic dose is administered by a sustained release formulation or device.

In another aspect of any of these methods and pharmaceutical compositions, the therapeutic dose of modified C-peptide is provided to the patient so as to maintain the average steady-state concentration of modified C-peptide in the patient's plasma between about 0.4 nM and about 8 nM when using a dosing interval of 3 days or longer. In another aspect of any of these methods and pharmaceutical compositions, the therapeutic dose of modified C-peptide is provided to the patient so as to maintain the average steady-state concentration of modified C-peptide in the patient's plasma between about 0.4 nM and about 8 nM when using a dosing interval of 4 days or longer. In another aspect of any of these methods and pharmaceutical compositions, the therapeutic dose of modified C-peptide is provided to the patient so as to maintain the average steady-state concentration of modified C-peptide in the patient's plasma between about 0.4 nM and about 8 nM when using a dosing interval of 5 days or longer. In another aspect of any of these methods and pharmaceutical compositions, the therapeutic dose of modified C-peptide is provided to the patient so as to maintain the average steady-state concentration of modified C-peptide in the patient's plasma between about 0.4 nM and about 8 nM when using a dosing interval of 7 days or longer.

In another aspect of any of these methods and pharmaceutical compositions, the therapeutic dose of modified C-peptide is provided to the patient so as to maintain the average steady-state concentration of modified C-peptide in the patient's plasma between about 0.6 nM and about 8 nM when using a dosing interval of 3 days or longer. In another aspect of any of these methods and pharmaceutical compositions, the therapeutic dose of modified C-peptide is provided to the patient so as to maintain the average steady-state concentration of modified C-peptide in the patient's plasma between about 0.6 nM and about 8 nM when using a dosing interval of 4 days or longer. In another aspect of any of these methods and pharmaceutical compositions, the therapeutic dose of modified C-peptide is provided to the patient so as to maintain the average steady-state concentration of modified C-peptide in the patient's plasma between about 0.6 nM and about 8 nM when using a dosing interval of 5 days or longer. In another aspect of any of these methods and pharmaceutical compositions, the therapeutic dose of modified C-peptide is provided to the patient so as to maintain the average steady-state concentration of modified C-peptide in the patient's plasma between about 0.6 nM and about 8 nM when using a dosing interval of 7 days or longer.

The dose may or may not be in solution. If the dose is administered in solution, it will be appreciated that the volume of the dose may vary, but will typically be 20 μL-2 mL. Preferably the dose for S.C. administration will be given in a volume of 2000 μL, 1500 μL, 1200 μL, 1000 μL, 900 μL, 800 μL, 700 μL, 600 μL, 500 μL, 400 μL, 300 μL, 200 μL, 100 μL, 50 μL, or 20 μL.

Modified C-peptide doses in solution can also comprise a preservative and/or a buffer. For example, the preservatives m-cresol, or phenol can be used. Typical concentrations of preservatives include 0.5 mg/mL, 1 mg/mL, 2 mg/mL, 3 mg/mL, 4 mg/mL, or 5 mg/mL. Thus, a range of concentration of preservative may include 0.2 to 10 mg/mL, particularly 0.5 to 6 mg/mL, or 0.5 to 5 mg/mL. Examples of buffers that can be used include histidine (pH 6.0), sodium phosphate buffer (pH 6 to 7.5), or sodium bicarbonate buffer (pH 7 to 7.5). It will be appreciated that the modified C-peptide dose may comprise one or more of a native or intact C-peptide, fragments, derivatives, or other functionally equivalent variants of C-peptide.

### VI. Methods for Administration

A dose of modified C-peptide may comprise full-length human C-peptide (SEQ ID NO:1) and the C-terminal C-peptide fragment EGSLQ (SEQ ID NO:31) and/or a C-peptide homolog or C-peptide derivative. Further, the dose may if desired only contain a fragment of C-peptide, e.g., EGSLQ. Thus, the term “C-peptide” may encompass a single C-peptide entity or a mixture of different “C-peptides”. Administration of the modified C-peptide may be by any suitable method known in the medicinal arts, including oral, parenteral, topical, or subcutaneous administration, inhalation, or the implantation of a sustained delivery device or composition. In one aspect, administration is by subcutaneous administration.

Pharmaceutical compositions of the invention suitable for oral administration may, e.g., comprise modified C-peptide in sterile purified stock powder form preferably covered by an envelope or envelopes (enterocapsules) protecting from degradation of the drug in the stomach and thereby enabling absorption of these substances from the gingiva or in the small intestines. The total amount of active ingredient in the composition may vary from 99.99 to 0.01 percent of weight.

For oral administration a pharmaceutical composition comprising a modified C-peptide can take the form of solutions, suspensions, tablets, pills, capsules, powders, and the like. Tablets containing various excipients such as sodium citrate, calcium carbonate and calcium phosphate are employed along with various disintegrants such as starch and preferably potato or tapioca starch and certain complex silicates, together with binding agents such as polyvinylpyrrolidone, sucrose, gelatin and acacia. Additionally, lubricating agents such as magnesium stearate, sodium lauryl sulfate and talc are often very useful for tabletting purposes. Solid compositions of a similar type are also employed as fillers in soft and hard-filled gelatin capsules; preferred materials in this connection also include lactose or milk sugar as well as high molecular weight polyethylene glycols. When aqueous suspensions and/or elixirs are desired for oral administration, the compounds of this invention can be combined with various sweetening agents, flavoring agents, coloring agents, emulsifying agents and/or suspending agents, as well as such diluents as water, ethanol, propylene glycol, glycerin and various like combinations thereof.

Pharmaceutical compositions to be used in the invention suitable for parenteral administration are typically sterile aqueous solutions and/or suspensions of the pharmaceutically active ingredients preferably made isotonic with the blood of the recipient. Such compositions generally comprise excipients, salts, carbohydrates, and buffering agents (preferably to a pH of from 3 to 9), such as sodium chloride, glycerin, glucose, mannitol, sorbitol, and the like.

For some applications, pharmaceutical compositions for parenteral administration may be suitably formulated as a sterile non-aqueous solution or as a dried form to be used in conjunction with a suitable vehicle such as sterile, pyrogen-free water. The preparation of parenteral formulations under sterile conditions, e.g., by lyophilization, may readily be accomplished using standard pharmaceutical techniques well-known to those skilled in the art.

Pharmaceutical compositions comprising modified C-peptide for use in the present invention may also be administered topically, (intra)dermally, or transdermally to the skin or mucosa. Pharmaceutical compositions for topical administration may be formulated to be immediate and/or modified release. Modified release formulations include delayed, sustained, pulsed, controlled, targeted and programmed release. Typical formulations for this purpose include gels, hydrogels, lotions, solutions, creams, ointments, dusting powders, dressings, foams, films, skin patches, wafers, implants, sponges, fibers, bandages, and microemulsions. Liposomes may also be used. Typical carriers include alcohol, water, mineral oil, liquid petrolatum, white petrolatum, glycerin, polyethylene glycol, and propylene glycol. Penetration enhancers may be incorporated—see, e.g., Finnin and Morgan: J. Pharm. Sci. 88(10): 955-958, (1999). Other means of topical administration include delivery by electroporation, iontophoresis, phonophoresis, sonophoresis, and microneedle or needle-free (e.g., POWDERJECT™, BOJECT™) injection.

Pharmaceutical compositions of modified C-peptide for parenteral administration may be administered directly into the blood stream, into muscle, or into an internal organ. Suitable means for parenteral administration include intravenous, intra-arterial, intraperitoneal, intrathecal, intraventricular, intraurethral, intrasternal, intracranial, intramuscular, intrasynovial, and subcutaneous. Suitable devices for parenteral administration include needle (including microneedle) injectors, needle-free injectors, and infusion techniques.

Subcutaneous administration of modified C-peptide will typically not be into the same site as that most recently used for insulin administration. In one aspect of any of the claimed methods and pharmaceutical compositions, modified C-peptide is administered to the opposite side of the abdomen to the site most recently used for insulin administration. In another aspect of any of the claimed methods and pharmaceutical compositions, modified C-peptide is administered to the upper arm. In another aspect of any of the claimed methods and pharmaceutical compositions, modified C-peptide is administered to the abdomen. In another aspect of any of the claimed methods and pharmaceutical compositions, modified C-peptide is administered to the upper area of the buttock. In another aspect of any of the claimed methods and pharmaceutical compositions, modified C-peptide is administered to the front of the thigh.

Formulations for parenteral administration may be formulated to be immediate and/or sustained release. Sustained release compositions include delayed, modified, pulsed, controlled, targeted and programmed release. Thus modified C-peptide may be formulated as a suspension or as a solid, semi-solid, or thixotropic liquid for administration as an implanted depot providing sustained release. Examples of such formulations include without limitation, drug-coated stents and semi-solids and suspensions comprising drug-loaded poly(DL-lactic-co-glycolic)acid (PGLA), poly(DL-lactide-co-glycolide) (PLG) or poly(lactide) (PLA) lamellar vesicles or microparticles, hydrogels (Hoffman A S: Ann. N.Y. Acad. Sci. 944: 62-73 (2001)), poly-amino acid nanoparticles systems, such as the Medusa system developed by Flamel Technologies Inc., nonaqueous gel systems such as Atrigel developed by Atrix, Inc., and SABER (Sucrose Acetate Isobutyrate Extended Release) developed by Durect Corporation, and lipid-based systems such as DepoFoam developed by SkyePharma.

Sustained release devices capable of delivering desired doses of modified C-peptide over extended periods of time are known in the art. For example, U.S. Pat. Nos. 5,034,229; 5,557,318; 5,110,596; 5,728,396; 5,985,305; 6,113,938; 6,156,331; 6,375,978; and 6,395,292; teach osmotically-driven devices capable of delivering an active agent formulation, such as a solution or a suspension, at a desired rate over an extended period of time (i.e., a period ranging from more than one week up to one year or more). Other exemplary sustained release devices include regulator-type pumps that provide constant flow, adjustable flow, or programmable flow of beneficial agent formulations, which are available from, e.g., OmniPod™ Insulin Management System (Insulet Corporation, Codman of Raynham, Mass., Medtronic of Minneapolis, Minn., Intarcia Therapeutics of Hayward, Calif., and Tricumed Medinzintechnik GmbH of Germany). Further examples of devices are described in U.S. Pat. Nos. 6,283,949; 5,976,109; 5,836,935; and 5,511,355.

Because they can be designed to deliver a desired active agent at therapeutic levels over an extended period of time, implantable delivery systems can advantageously provide long-term therapeutic dosing of a desired active agent without requiring frequent visits to a healthcare provider or repetitive self-medication. Therefore, implantable delivery devices can work to provide increased patient compliance, reduced irritation at the site of administration, fewer occupational hazards for healthcare providers, reduced waste hazards, and increased therapeutic efficacy through enhanced dosing control.

Among other challenges, two problems must be addressed when seeking to deliver biomolecular material over an extended period of time from an implanted delivery device. First, the biomolecular material must be contained within a formulation that substantially maintains the stability of the material at elevated temperatures (i.e., 37° C. and above) over the operational life of the device. Second, the biomolecular material must be formulated in a way that allows delivery of the biomolecular material from an implanted device into a desired environment of operation over an extended period of time. This second challenge has proven particularly difficult where the biomolecular material is included in a flowable composition that is delivered from a device over an extended period of time at low flow rates (i.e., ≦100 μL/day).

Peptide drugs such as C-peptide may degrade via one or more of several different mechanisms, including deamidation, oxidation, hydrolysis, and racemization. Significantly, water is a reactant in many of the relevant degradation pathways. Moreover, water acts as a plasticizer and facilitates the unfolding and irreversible aggregation of biomolecular materials. To work around the stability problems created by aqueous formulations of biomolecular materials, dry powder formulations of biomolecular materials have been created using known particle formation processes, such as by known lyophilization, spray drying, or desiccation techniques. Though dry powder formulations of biomolecular material have been shown to provide suitable stability characteristics, it would be desirable to provide a formulation that is not only stable over extended periods of time, but is also flowable and readily deliverable from an implantable delivery device.

Accordingly in one aspect of any of the claimed methods and pharmaceutical compositions, the modified C-peptide is provided in a nonaqueous drug formulation, and is delivered from a sustained release implantable device, wherein the modified C-peptide is stable for at least two months of time at 37° C.

Representative nonaqueous formulations for modified C-peptide include those disclosed in International Publication Number WO00/45790 that describes nonaqueous vehicle formulations that are formulated using at least two of a polymer, a solvent, and a surfactant.

WO98/27962 discloses an injectable depot gel composition containing a polymer, a solvent that can dissolve the polymer and thereby form a viscous gel, a beneficial agent, and an emulsifying agent in the form of a dispersed droplet phase in the viscous gel.

WO04089335 discloses nonaqueous vehicles that are formed using a combination of polymer and solvent that results in a vehicle that is miscible in water. As it is used herein, the term “miscible in water” refers to a vehicle that, at a temperature range representative of a chosen operational environment, can be mixed with water at all proportions without resulting in a phase separation of the polymer from the solvent such that a highly viscous polymer phase is formed. For the purposes of the present invention, a “highly viscous polymer phase” refers to a polymer containing composition that exhibits a viscosity that is greater than the viscosity of the vehicle before the vehicle is mixed with water.

Accordingly in another aspect of any of the claimed methods, modified C-peptide is provided in a sustained release device comprising: a reservoir having at least one drug delivery orifice, and a stable nonaqueous drug formulation. In one aspect of these methods and pharmaceutical compositions, the formulation comprises: at least modified C-peptide; and a nonaqueous, single-phase vehicle comprising at least one polymer and at least one solvent, the vehicle being miscible in water, wherein the drug is insoluble in one or more vehicle components and the modified C-peptide formulation is stable at 37° C. for at least two months. In one aspect, the solvent is selected from the group consisting of glycofurol, benzyl alcohol, tetraglycol, n-methylpyrrolidone, glycerol formal, propylene glycol, and combinations thereof.

In particular, a nonaqueous formulation is considered chemically stable if no more than about 35% of the modified C-peptide is degraded by chemical pathways, such as by oxidation, deamidation, and hydrolysis, after maintenance of the formulation at 37° C. for a period of two months, and a formulation is considered physically stable if, under the same conditions, no more than about 15% of the C-peptide contained in the formulation is degraded through aggregation. A drug formulation is stable according to the present invention if at least about 65% of the modified C-peptide remains physically and chemically stable after about two months at 37° C.

The modified C-peptide can be administered intranasally or by inhalation, typically in the form of a dry powder (either alone, as a mixture, e.g., in a dry blend with lactose, or as a mixed component particle, e.g., mixed with phospholipids, such as phosphatidylcholine) from a dry powder inhaler, as an aerosol spray from a pressurized container, pump, spray, atomizer (preferably an atomizer using electro hydrodynamics to produce a fine mist), or nebulizer, with or without the use of a suitable propellant, such as 1,1,1,2-tetrafluoroethane or 1,1,1,2,3,3,3-heptafluoropropane, or as nasal drops. For intranasal use, the powder may comprise a bioadhesive agent, e.g., chitosan or cyclodextrin. The pressurized container, pump, spray, atomizer, or nebulizer contains a solution or suspension of the compound(s) of the invention comprising, e.g., ethanol, aqueous ethanol, or a suitable alternative agent for dispersing, solubilizing, or extending release of the active, a propellant(s) as solvent and an optional surfactant, such as sorbitan trioleate, oleic acid, or an oligolactic acid.

Prior to use in a dry powder or suspension formulation, the drug product is micronized to a size suitable for delivery by inhalation (typically less than 5 μm). This may be achieved by any appropriate method, such as spiral jet milling, fluid bed jet milling, supercritical fluid processing to form nanoparticles, high pressure homogenization, or spray drying.

The particle size of modified C-peptide of this invention in the formulation delivered by the inhalation device is important with respect to the ability of C-peptide to make it into the lungs, and preferably into the lower airways or alveoli. Preferably, the modified C-peptide of this invention is formulated so that at least about 10% of the modified C-peptide delivered is deposited in the lung, preferably about 10% to about 20%, or more. It is known that the maximum efficiency of pulmonary deposition for mouth breathing humans is obtained with particle sizes of about 2 pm to about 3 pm. When particle sizes are above about 5 pm, pulmonary deposition decreases substantially. Particle sizes below about 1 pm cause pulmonary deposition to decrease, and it becomes difficult to deliver particles with sufficient mass to be therapeutically effective. Thus, particles of the modified C-peptide delivered by inhalation have a particle size preferably less than about 10 pm, more preferably in the range of about 1 pm to about 5 pm. The formulation of the modified C-peptide is selected to yield the desired particle size in the chosen inhalation device.

Advantageously for administration as a dry powder, a modified C-peptide of this invention is prepared in a particulate form with a particle size of less than about 10 pm, preferably about 1 to about 5 pm. The preferred particle size is effective for delivery to the alveoli of the patient's lung. Preferably, the dry powder is largely composed of particles produced so that a majority of the particles have a size in the desired range. Advantageously, at least about 50% of the dry powder is made of particles having a diameter less than about 10 pm. Such formulations can be achieved by spray drying, milling, or critical point condensation of a solution containing the modified C-peptide of this invention and other desired ingredients. Other methods also suitable for generating particles useful in the current invention are known in the art.

The particles are usually separated from a dry powder formulation in a container and then transported into the lung of a patient via a carrier air stream. Typically, in current dry powder inhalers, the force for breaking up the solid is provided solely by the patient's inhalation. In another type of inhaler, air flow generated by the patient's inhalation activates an impeller motor which deagglomerates the particles.

Capsules (made, e.g., from gelatin or hydroxypropylmethylcellulose), blisters and cartridges for use in an inhaler or insufflator may be formulated to contain a powder mix of the compound of the invention, a suitable powder base such as lactose or starch and a performance modifier such as 1-leucine, mannitol, or magnesium stearate. The lactose may be anhydrous or in the form of the monohydrate, preferably the latter. Other suitable excipients include dextran, glucose, maltose, sorbitol, xylitol, fructose, sucrose, and trehalose.

A suitable solution formulation for use in an atomizer using electro hydrodynamics to produce a fine mist may contain from 100 μg to 200 mg of modified C-peptide per actuation and the actuation volume may vary from 1 μL to 100 μL. A typical formulation may comprise modified C-peptide propylene glycol, sterile water, ethanol, and sodium chloride. Alternative solvents that may be used instead of propylene glycol include glycerol and polyethylene glycol. Suitable flavors, such as menthol and levomenthol, or sweeteners, such as saccharin or saccharin sodium, may be added to those formulations of the invention intended for inhaled/intranasal administration. Formulations for inhaled/intranasal administration may be formulated to be immediate and/or modified release using, e.g., PGLA. Modified release formulations include delayed, sustained, pulsed, controlled, targeted, and programmed release.

In the case of dry powder inhalers and aerosols, the dosage unit is determined by means of a valve that delivers a metered amount. Units in accordance with the invention are typically arranged to administer a metered dose or “puff” containing from 1 mg to 200 mg of modified C-peptide. The overall daily dose will typically be in the range 1 mg to 200 mg that may be administered in a single dose or, more usually, as divided doses throughout the day.

Examples of commercially available inhalation devices suitable for the practice of the invention are sold under the trademarks TURBHALER™ (Astra), ROTAHALER® (Glaxo), DISKUS®, SPIROS™ inhaler (Dura), devices marketed by Inhale Therapeutics under the trademarks AERX™ (Aradigm), and ULTRAVENT® nebulizer (Mallinckrodt), ACORN II® nebulizer (Marquest Medical Products), VENTOLIN® metered dose inhaler (Glaxo), and the SPINHALER® powder inhaler (Fisons), and the like.

Kits are also contemplated for this invention. A typical kit would comprise a container, preferably a vial, for the modified C-peptide formulation comprising modified C-peptide in a pharmaceutically acceptable formulation, and instructions, and/or a product insert or label. In one aspect, the instructions include a dosing regimen for administration of said modified C-peptide to an insulin-dependent patient to reduce the risk, incidence, or severity of hypoglycemia. In one aspect, the kit includes instructions to reduce the administration of insulin by about 5% to about 35% when starting modified C-peptide therapy. In another aspect, the instructions include directions for the patient to closely monitor their blood glucose levels when starting modified C-peptide therapy. In another aspect, the instructions include directions for the patient to avoid situations or circumstances that might predispose the patient to hypoglycemia when starting modified C-peptide therapy.

### EXAMPLES

General Procedures: The following examples and general procedures refer to intermediate compounds and final products identified in the specification. Alternatively, other reactions disclosed herein or otherwise conventional will be applicable to the preparation of the corresponding compounds of the invention. In all preparative methods, all starting materials are known or may easily be prepared from known starting materials. All temperatures are set forth in degrees Celsius (° C.) and unless otherwise indicated, all parts and percentages are by weight (i.e., w/w) when referring to yields and all parts are by volume (i.e., v/v) when referring to solvents and eluents.

### Example 1

### Construction of XTEN_AD36 Motif Segments

The procedure is carried out as described in US 20100239554, pages 71-75, which is hereby incorporated by reference.

### Example 2

### Construction of XTEN_AE36 Segments

The procedure is carried out as described in US 20100239554, pages 75-78, which is hereby incorporated by reference.

### Example 3

### Construction of XTEN_AF36 Segments

The procedure is carried out as described in US 20100239554, pages 78-82, which is hereby incorporated by reference.

### Example 4

### Construction of XTEN_AG36 Segments

The procedure is carried out as described in US 20100239554, pages 82-86, which is hereby incorporated by reference.

### Example 5

### Construction of XTEN_AE864

The procedure is carried out as described in US 20100239554, page 86, which is hereby incorporated by reference.

### Example 6

### Construction of XTEN_AM144

The procedure is carried out as described in US 20100239554, pages 86-94, which is hereby incorporated by reference.

### Example 7

### Construction of XTEN_AM288

The procedure is carried out as described in US 20100239554, page 94, which is hereby incorporated by reference.

### Example 8

### Construction of XTEN_AM432

The procedure is carried out as described in US 20100239554, pages 94-55, which is hereby incorporated by reference.

### Example 9

### Construction of XTEN_AM875

The procedure is carried out as described in US 20100239554, page 95, which is hereby incorporated by reference.

### Example 10

### Construction of XTEN_AM1318

The procedure is carried out as described in US 20100239554, page 95, which is hereby incorporated by reference.

### Example 11

### Construction of XTEN_AD864

The procedure is carried out as described in US 20100239554, page 95, which is hereby incorporated by reference.

### Example 12

### Construction of XTEN_AF864

The procedure is carried out as described in US 20100239554, page 95, which is hereby incorporated by reference.

### Example 13

### Construction of XTEN_AG864

The procedure is carried out as described in US 20100239554, page 95, which is hereby incorporated by reference.

### Example 14

### Construction of N-Terminal Extensions of XTEN Construction and Screening of 12mer Addition Libraries

The procedure is carried out as described in US 20100239554, pages 95-96, which is hereby incorporated by reference.

### Example 15

### Construction of N-Terminal Extensions of XTEN Construction and Screening of Libraries Optimizing Codons 3 and 4

The procedure is carried out as described in US 20100239554, page 97, which is hereby incorporated by reference.

### Example 16

### Construction of N-Terminal Extensions of XTEN Construction and Screening of Combinatorial 12mer and 36mer Libraries

The procedure is carried out as described in US 20100239554, pages 97-99, which is hereby incorporated by reference.

### Example 17

### Construction of N-Terminal Extensions of XTEN Construction and Screening of Combinatorial 12mer and 36mer Libraries for XTEN-AM875 and XTENAE864

The procedure is carried out as described in US 20100239554, pages 99-100, which is hereby incorporated by reference.

### Example 18

### Methods of Producing and Evaluating C-peptide-XTEN

A general schema for producing and evaluating C-peptide-XTEN compositions is presented in FIG. 6 of US 20100239554, which is hereby incorporated by reference, and forms the basis for the general description of this Example. Using the disclosed methods and those known to one of ordinary skill in the art, together with guidance provided in the illustrative examples, a skilled artesian can create and evaluate a range of C-peptide-XTEN fusion proteins comprising, XTENs, C-peptide and variants of C-peptide known in the art. The Example is, therefore, to be construed as merely illustrative, and not limitative of the methods in any way whatsoever; numerous variations will be apparent to the ordinarily skilled artisan. In this Example, a C-peptide-XTEN of C-peptide linked to an XTEN of the AE family of motifs would be created.

The general schema for producing polynucleotides encoding XTEN is presented in FIGS. 4 and 5 of US 20100239554, which are hereby incorporated by reference. FIG. 5 of US 20100239554 is a schematic flowchart of representative steps in the assembly of a XTEN polynucleotide construct in one of the embodiments of the invention. Individual oligonucleotides 501 are annealed into sequence motifs 502 such as a 12 amino acid motif (“12-mer”), which is subsequently ligated with an oligo containing BbsI, and KpnI restriction sites 503. The motif libraries can be limited to specific sequence XTEN families; e.g., AD, AE, AF, AG, AM, or AQ sequences of Table 2. In this case, the motifs of the AE family (SEQ ID NOS:38-41) would be used as the motif library, which are annealed to the 12-mer to create a “building block” length; e.g., a segment that encodes 36 amino acids. The gene encoding the XTEN sequence can be assembled by ligation and multimerization of the “building blocks” until the desired length of the XTEN gene 504 is achieved. As illustrated in FIG. 5 of US 20100239554, the XTEN length in this case is 48 amino acid residues, but longer lengths can be achieved by this process. For example, multimerization can be performed by ligation, overlap extension, PCR assembly or similar cloning techniques known in the art. The XTEN gene can be cloned into a stuffer vector. In the example illustrated in FIG. 5 of US 20100239554, the vector can encode a Flag sequence 506 followed by a stuffer sequence that is flanked by BsaI, BbsI, and KpnI sites 507 and a gene encoding a C-peptide 508, resulting in the gene encoding the C-peptide-XTEN 500, which, in this case encodes the fusion protein in the configuration, N- to C-terminus, XTEN-C-peptide.

DNA sequences encoding C-peptide can be conveniently obtained by standard procedures known in the art from a cDNA library prepared from an appropriate cellular source, from a genomic library, or may be created synthetically (e.g., automated nucleic acid synthesis) using DNA sequences obtained from publicly available databases, patents, or literature references. A gene or polynucleotide encoding the C-peptide portion of the protein can be then be cloned into a construct, such as those described herein, which can be a plasmid or other vector under control of appropriate transcription and translation sequences for high level protein expression in a biological system. A second gene or polynucleotide coding for the XTEN portion (in the case of FIG. 5 of US 20100239554 illustrated as an AE with 48 amino acid residues) can be genetically fused to the nucleotides encoding the N-terminus of the C-peptide gene by cloning it into the construct adjacent and in frame with the gene coding for the C-peptide, through a ligation or multimerization step. In this manner, a chimeric DNA molecule coding for (or complementary to) the XTEN-C-peptide fusion protein would be generated within the construct. The construct can be designed in different configurations to encode the various permutations of the fusion partners as a monomeric polypeptide. For example, the gene can be created to encode the fusion protein in the order (N- to C-terminus): C-peptide-XTEN; XTEN-C-peptide; C-peptide-XTEN-C-peptide; XTEN-C-peptide-XTEN; as well as multimers of the foregoing. Optionally, this chimeric DNA molecule may be transferred or cloned into another construct that is a more appropriate expression vector. At this point, a host cell capable of expressing the chimeric DNA molecule would be transformed with the chimeric DNA molecule. The vectors containing the DNA segments of interest can be transferred into an appropriate host cell by well-known methods, depending on the type of cellular host, as described supra.

Host cells containing the XTEN-C-peptide expression vector would be cultured in conventional nutrient media modified as appropriate for activating the promoter. The culture conditions, such as temperature, pH and the like, are those previously used with the host cell selected for expression, and will be apparent to the ordinarily skilled artisan. After expression of the fusion protein, cells would be harvested by centrifugation, disrupted by physical or chemical means, and the resulting crude extract retained for purification of the fusion protein, as described below. For C-peptide-XTEN compositions secreted by the host cells, supernatant from centrifugation would be separated and retained for further purification.

Gene expression would be measured in a sample directly, for example, by conventional Southern blotting, Northern blotting to quantitate the transcription of mRNA (Thomas, Proc. Natl. Acad. Sci. USA, 77:5201-5205 (1980)), dot blotting (DNA analysis), or in situ hybridization, using an appropriately labeled probe, based on the sequences provided herein. Alternatively, gene expression would be measured by immunological of fluorescent methods, such as immunohistochemical staining of cells to quantitate directly the expression of gene product. Antibodies useful for immunohistochemical staining and/or assay of sample fluids may be either monoclonal or polyclonal, and may be prepared in any mammal. Conveniently, the antibodies may be prepared against the C-peptide sequence polypeptide using a synthetic peptide based on the sequences provided herein or against exogenous sequence fused to C-peptide and encoding a specific antibody epitope. Examples of selectable markers are well known to one of skill in the art and include reporters such as enhanced green fluorescent protein (EGFP), beta-galactosidase (β-gal) or chloramphenicol acetyltransferase (CAT).

The XTEN-C-peptide polypeptide product would be purified via methods known in the art. Procedures such as gel filtration, affinity purification, salt fractionation, ion exchange chromatography, size exclusion chromatography, hydroxyapatite adsorption chromatography, hydrophobic interaction chromatography or gel electrophoresis are all techniques that may be used in the purification. Specific methods of purification are described in Robert K. Scopes, Protein Purification: Principles and Practice, Charles R. Castor, ed., Springer-Verlag 1994, and Sambrook, et al., supra. Multi-step purification separations are also described in Baron, et al., Crit. Rev. Biotechnol. 10:179-90 (1990) and Below, et al., J. Chromatogr. A. 679:67-83 (1994).

As illustrated in FIG. 6 of US 20100239554, the isolated XTEN-C-peptide fusion proteins would then be characterized for their chemical and activity properties. Isolated fusion protein would be characterized, e.g., for sequence, purity, apparent molecular weight, solubility and stability using standard methods known in the art. The fusion protein meeting expected standards would then be evaluated for activity, which can be measured in vitro or in vivo, using one or more assays disclosed herein; e.g., the assays of the Examples or Table 39 of US 20100239554, which is hereby incorporated by reference.

In addition, the XTEN-C-peptide fusion protein would be administered to one or more animal species to determine standard pharmacokinetic parameters, as described in Examples 20, 24, 25 and 43-47.

By the iterative process of producing, expressing, and recovering XTEN-Ex4 constructs, followed by their characterization using methods disclosed herein or others known in the art, the C-peptide-XTEN compositions comprising C-peptide and an XTEN can be produced and evaluated by one of ordinary skill in the art to confirm the expected properties such as enhanced solubility, enhanced stability, improved pharmacokinetics and reduced immunogenicity, leading to an overall enhanced therapeutic activity compared to the corresponding unfused C-peptide. For those fusion proteins not possessing the desired properties, a different sequence can be constructed, expressed, isolated and evaluated by these methods in order to obtain a composition with such properties.

### Example 19

### Analytical Size Exclusion Chromatography of XTEN Fusion Proteins

Size exclusion chromatography analysis was performed on fusion proteins containing various therapeutic proteins and unstructured recombinant proteins of increasing length. An exemplary assay used a TSKGeI-G4000 SWXL (7.8 mm×30 cm) column in which 40 μg of purified glucagon fusion protein at a concentration of 1 mg/ml was separated at a flow rate of 0.6 ml/min in 20 mM phosphate pH 6.8, 114 mM NaCl. Chromatogram profiles were monitored using OD214 nm and OD280 nm. Column calibration for all assays were performed using a size exclusion calibration standard from BioRad; the markers include thyroglobulin (670 kDa), bovine gamma-globulin (158 kDa), chicken ovalbumin (44 kDa), equine myoglobuin (17 kDa) and vitamin B12 (1.35 kDa). Representative chromatographic profiles of Glucagon-Y288, Glucagon-Y144, Glucagon-Y72, Glucagon-Y36 are shown as an overlay in FIG. 15 of US 20100239554, which is hereby incorporated by reference. The data show that the apparent molecular weight of each compound is proportional to the length of the attached rPEG sequence. However, the data also show that the apparent molecular weight of each construct is significantly larger than that expected for a globular protein (as shown by comparison to the standard proteins run in the same assay). Based on the SEC analyses for all constructs evaluated, the Apparent Molecular Weights, the Apparent Molecular Weight Factor (expressed as the ratio of Apparent Molecular Weight to the calculated molecular weight) and the hydrodynamic radius (RH in nM) are shown in Table 26 of US 20100239554, which is hereby incorporated by reference. The results indicate that incorporation of different XTENs of 576 amino acids or greater confers an apparent molecular weight for the fusion protein of approximately 339 kDa to 760, and that XTEN of 864 amino acids or greater confers an apparent molecular weight greater than approximately 800 kDA. The results of proportional increases in apparent molecular weight to actual molecular weight were consistent for fusion proteins created with XTEN from several different motif families; i.e., AD, AE, AF, AG, and AM, with increases of at least four-fold and ratios as high as about 17-fold. Additionally, the incorporation of XTEN fusion partners with 576 amino acids or more into fusion proteins with glucose regulating peptides resulted with a hydrodynamic radius of 7 nm or greater; well beyond the glomerular pore size of approximately 3-5 nm. Accordingly, it is concluded that fusion proteins comprising glucose regulating peptides and XTEN would have reduced renal clearance, contributing to increased terminal half-life and improving the therapeutic or biologic effect relative to a corresponding un-fused biologically active protein.

### Example 20

### Pharmacokinetics of Extended Polypeptides Fused to C-Peptide in Cynomolgus Monkeys

The pharmacokinetics of C-peptide-L288, C-peptide-L576, C-peptide-XTEN_AF576, C-peptide-XTEN_Y576 and XTEN_AD836-C-peptide are tested in cynomolgus monkeys to determine the effect of composition and length of the unstructured polypeptides on PK parameters. Blood samples are analyzed at various times after injection and the concentration of C-peptide in plasma is measured by ELISA using a polyclonal antibody against C-peptide for capture and a biotinylated preparation of the same polyclonal antibody for detection. Results can show a surprising increase of half-life with increasing length of the XTEN sequence. Doubling the length of the unstructured polypeptide fusion partner to 576 amino acids may increase the half-life for multiple fusion protein constructs; i.e., C-peptide-XTEN_L576, C-peptide-XTEN_AF576, C-peptide-XTEN_Y576. A further increase of the unstructured polypeptide fusion partner length to 836 residues may result in a further increased half-life of 72-75 h for XTEN_AD836-C-peptide. Thus, increasing the polymer length by 288 residues from 288 to 576 residues can increase in vivo half-life. However, increasing the polypeptide length by 260 residues from 576 residues to 836 residues may increase half-life even further. These results can show that there is a surprising threshold of unstructured polypeptide length that results in a greater than proportional gain in in vivo half-life. Thus, fusion proteins comprising extended, unstructured polypeptides are expected to have the property of enhanced pharmacokinetics compared to polypeptides of shorter lengths.

### Example 21

### Serum Stability of C-Peptide-XTEN

A fusion protein containing XTEN_AE864 fused to the N-terminus of C-peptide is incubated in monkey plasma and rat kidney lysate for up to 7 days at 37° C. Samples are withdrawn at time 0, Day 1 and Day 7 and analyzed by SDS PAGE followed by detection using Western analysis and detection with antibodies against C-peptide. The sequence of XTEN_AE864 may show negligible signs of degradation over 7 days in plasma. However, XTEN_AE864 can be rapidly degraded in rat kidney lysate over 3 days. The in vivo stability of the fusion protein is tested in plasma samples wherein the C-peptide_AE864 is immunoprecipitated and analyzed by SDS PAGE as described above. Samples that are withdrawn up to 7 days after injection may show very few signs of degradation. The results can demonstrate the resistance of C-peptide-XTEN to degradation due to serum proteases; a factor in the enhancement of pharmacokinetic properties of the C-peptide-XTEN fusion proteins.

### Example 22

### Construction of C-Peptide-XTEN Component Genes and Vectors

The gene encoding C-peptide is amplified by polymerase chain reaction (PCR), which introduces flanking restriction sites that are compatible with the BbsI and HindIII sites that flank the stuffer in the XTEN destination vector (FIG. 7C of US 20100239554, which is hereby incorporated by reference). The XTEN destination vectors contain the kanamycin-resistance gene and are pET30 derivatives from Novagen in the format of Cellulose Binding Domain (CBD)-XTEN-Green Fluorescent Protein (GFP), where GFP is the stuffer for cloning payloads at C-terminus. Constructs are generated by replacing GFP in the XTEN destination vectors with the C-peptide encoding fragment. The XTEN destination vector features a T7 promoter upstream of CBD followed by an XTEN sequence fused in-frame upstream of the stuffer GFP sequence. The XTEN sequences employed are AM875, AM1318, AF875 and AE864 which have lengths of 875, 1318, 875 and 864 amino acids, respectively. The stuffer GFP fragment is removed by restriction digestion using BbsI and HindIII endonucleases. BsaI and HindIII restriction digested C-peptide DNA fragment is ligated into the BbsI and HindIII digested XTEN destination vector using T4 DNA ligase and the ligation mixture is transformed into E. coli strain BL21 (DE3) Gold (Stratagene) by electroporation. Transformants are identified by the ability to grow on LB plates containing the antibiotic kanamycin. Plasmid DNAs are isolated from selected clones and confirmed by restriction analysis and DNA sequencing. The final vector yields the CBD_XTEN_C-peptide gene under the control of a T7 promoter and CBD is cleaved by engineered TEV cleavage site at the end to generate XTEN_C-peptide. Various constructs with C-peptide fused at C-terminus to different XTENs include AC1723 (CBD-XTEN_AM875-C-peptide), AC 175 (CBD-XTEN_AM1318-IL-C-peptide), AC180 (CBD-XTEN_AF875-C-peptide), and AC182 (CBD-XTEN_AE864-C-peptide).

### Example 23

### Expression, Purification, and Characterization of C-Peptide Fused to XTEN_AM875 and XTEN_AE864

Cell Culture Production

A starter culture is prepared by inoculating glycerol stocks of E. coli carrying a plasmid encoding for C-peptide fused to AE864, AM875, or AM1296 into 100 mL 2xYT media containing 40 ug/mL kanamycin. The culture is then shaken overnight at 37° C. 100 mL of the starter culture is used to inoculate 25 liters of 2xYT containing 40 μg/mL kanamycin and shaken until the OD600 reached about 1.0 (for 5 hours) at 37° C. The temperature is then reduced to 26° C. and protein expression was induced with IPTG at 1.0 mM final concentration. The culture is then shaken overnight at 26° C. Cells are harvested by centrifugation yielding a total of 200 grams cell paste. The paste is stored frozen at −80° C. until use.

Purification of C-Peptide-XTEN Comprising C-Peptide-XTEN_AE864 or C-Peptide-AM875

Cell paste is suspended in 20 mM Tris pH 6.8, 50 mM NaCl at a ratio of 4 ml of buffer per gram of cell paste. The cell paste is then homogenized using a top-stirrer. Cell lysis is achieved by passing the sample once through a microfluidizer at 20000 psi. The lysate is clarified to by centrifugation at 12000 rpm in a Sorvall G3A rotor for 20 minutes.

Clarified lysate is directly applied to 800 ml of Macrocap Q anion exchange resin (GE Life Sciences) that has been equilibrated with 20 mM Tris pH 6.8, 50 mM NaCl. The column is sequentially washed with Tris pH 6.8 buffer containing 50 mM, 100 mM, and 150 mM NaCl. The product is eluted with 20 mM Tris pH 6.8, 250 mM NaCl.

A 250 mL Octyl Sepharose FF column is equilibrated with equilibration buffer (20 mM Tris pH 6.8, 1.0 M Na2SO4). Solid Na2SO4 is added to the Macrocap Q eluate pool to achieve a final concentration of 1.0 M. The resultant solution is filtered (0.22 micron) and loaded onto the HIC column. The column is then washed with equilibration buffer for 10 CV to remove unbound protein and host cell DNA. The product is then eluted with 20 mM Tris pH 6.8, 0.5 M Na2SO4.

The pooled HIC eluate fractions are then diluted with 20 mM Tris pH 7.5 to achieve a conductivity of less than 5.0 mOhms. The dilute product is loaded onto a 300 ml Q Sepharose FF anion exchange column that has been equilibrated with 20 mM Tris pH 7.5, 50 mM NaCl.

The buffer exchanged proteins are then concentrated by ultrafiltration/diafiltration (UF/DF), using a Pellicon XL Biomax 30000 mwco cartridge, to greater than 30 mg/ml. The concentrate is sterile filtered using a 0.22 micron syringe filter. The final solution is aliquoted and stored at −80° C., and is used for the experiments that follow, infra.

SDS-PAGE Analysis

2 and 10 mcg of final purified protein are subjected to non-reducing SDS-PAGE using NuPAGE 4-12% Bis-Tris gel from Invitrogen according to manufacturer's specifications. The results may show that the C-peptide-XTEN_AE864 composition is recovered by the process detailed above.

Analytical Size Exclusion Chromatography

Size exclusion chromatography analysis is performed using a Phenomenex BioSEP SEC S4000 (7.8×300 mm) column. 20 mg of the purified protein at a concentration of 1 mg/ml is separked at a flow rate of 0.5 ml/min in 20 mM Tris-Cl pH 7.5, 300 mM NaCl. Chromatogram profiles are monitored by absorbance at 214 and 280 nm. Column calibration is performed using a size exclusion calibration standard from BioRad, the markers include thyroglobulin (670 kDa), bovine gamma-globulin (158 kDa), chicken ovalbumin (44 kDa), equine myoglobuin (17 kDa) and vitamin B12 (1.35 kDa).

Analytical RP-HPLC

Analytical RP-HPLC chromatography analysis is performed using a Vydac Protein C4 (4.6×150 mm) column. The column is equilibrated with 0.1% trifluoroacetic acid in HPLC grade water at a flow rate of 1 ml/min. Ten micrograms of the purified protein at a concentration of 0.2 mg/ml is injected separately. The protein is eluted with a linear gradient from 5% to 90% acetonitrile in 0.1% TFA. Chromatogram profiles are monitored using OD214 nm and OD280 nm.

Thermal Stabilization of C-Peptide by XTEN

In addition to extending the serum half-life of protein therapeutics, XTEN polypeptides have the property improving the thermal stability of a payload to which it is fused. For example, the hydrophilic nature of the XTEN polypeptide may reduce or prevent aggregation and thus favor refolding of the payload protein. This feature of XTEN may aid in the development of room temperature stable formulations for a variety of protein therapeutics.

In order to demonstrate thermal stabilization of C-peptide conferred by XTEN conjugation, C-peptide-XTEN and recombinant C-peptide, 200 micromoles per liter, are incubated at 25° C. and 85° C. for 15 min, at which time any insoluble protein is rapidly removed by centrifugation. The soluble fraction is then analyzed by SDS-PAGE. Only C-peptide-XTEN remains soluble after heating, while, in contrast, recombinant C-peptide (without XTEN as a fusion partner) is completely precipitated after heating.

### Example 24

### PK Analysis of Fusion Proteins Comprising C-Peptide and XTEN

The C-peptide-XTEN fusion proteins C-peptide_AE864, C-peptide_AM875, and C-peptide_AM1296 are evaluated in cynomolgus monkeys in order to determine in vivo pharmacokinetic parameters of the respective fusion proteins. All compositions are provided in an aqueous buffer and were administered by subcutaneous (SC) route into separate animals (n=4/group) using 1 mg/kg and/or 10 mg/kg single doses. Plasma samples are collected at various time points following administration and analyzed for concentrations of the test articles. Analysis was performed using a sandwich ELISA format. Rabbit polyclonal anti-XTEN antibodies are coated onto wells of an ELISA plate. The wells are blocked, washed and plasma samples are then incubated in the wells at varying dilutions to allow capture of the compound by the coated antibodies. Wells are washed extensively, and bound protein is detected using a biotinylated preparation of the polyclonal anti C-peptide antibody and streptavidin HRP. Concentrations of test article are calculated at each time point by comparing the colorimetric response at each serum dilution to a standard curve. Pharmacokinetic parameters are calculated using the WinNonLin software package.

### Example 25

### PK Analysis of C-Peptide-XTEN in Multiple Species and Predicted Human Half-Life

To determine the predicted pharmacokinetic profile in humans of a C-peptide fused to XTEN, studies are performed using C-peptide fused to the AE864 XTEN as a single fusion polypeptide. The C-peptide-XTEN construct is administered to four different animal species at 0.5-1.0 mg/kg, subcutaneously and intravenously. Serum samples are collected at intervals following administration, with serum concentrations determined using standard methods. The half-life for each species is determined. The results are used to predict the human half-life using allometric scaling of terminal half-life, volume of distribution, and clearance rates based on average body mass.

### Example 26

### Increasing Solubility and Stability of C-Peptide by Linking to XTEN

In order to evaluate the ability of XTEN to enhance the physical/chemical properties of solubility and stability, fusion proteins of C-peptide plus shorter-length XTEN are prepared and evaluated. The test articles are prepared in Tris-buffered saline at neutral pH and characterization of the C-peptide-XTEN solution is by reverse-phase HPLC and size exclusion chromatography to affirm that the protein is homogeneous and non-aggregated in solution. The C-peptide-XTEN fusion proteins are prepared to achieve target concentrations and are not evaluated to determine the maximum solubility limits for the given construct.

### Example 27

### Characterization of C-Peptide-XTEN Secondary Structure

The C-peptide-XTEN C-peptide-AE864 is evaluated for degree of secondary structure by circular dichroism spectroscopy. CD spectroscopy is performed on a Jasco J-715 (Jasco Corporation, Tokyo, Japan) spectropolarimeter equipped with Jasco Peltier temperature controller (TPC-348W1). The concentration of protein is adjusted to 0.2 mg/mL in 20 mM sodium phosphate pH 7.0, 50 mM NaCl. The experiments are carried out using HELLMA quartz cells with an optical path-length of 0.1 cm. The CD spectra are acquired at 5°, 25°, 45°, and 65° C. and processed using the J-700 version 1.08.01 (Build 1) Jasco software for Windows. The samples are equilibrated at each temperature for 5 min before performing CD measurements. All spectra are recorded in duplicate from 300 nm to 185 nm using a bandwidth of 1 nm and a time constant of 2 sec, at a scan speed of 100 nm/min.

### Example 28

### C-Terminal XTEN Releaseable by FXIa

The procedure is carried out as described in US 20100239554, page 110, which is hereby incorporated by reference.

### Example 29

### C-Terminal XTEN Releaseable by FXIIa

The procedure is carried out as described in US 20100239554, page 110, which is hereby incorporated by reference.

### Example 30

### C-Terminal XTEN Releaseable by Kallikrein

The procedure is carried out as described in US 20100239554, page 110, which is hereby incorporated by reference.

### Example 31

### C-Terminal XTEN Releaseable by FVIIa

The procedure is carried out as described in US 20100239554, pages 110-111, which is hereby incorporated by reference.

### Example 32

### C-Terminal XTEN Releaseable by FIXa

The procedure is carried out as described in US 20100239554, page 111, which is hereby incorporated by reference.

### Example 33

### C-Terminal XTEN Releaseable by FXa

The procedure is carried out as described in US 20100239554, page 111, which is hereby incorporated by reference.

### Example 34

### C-Terminal XTEN Releaseable by Ma (Thrombin)

The procedure is carried out as described in US 20100239554, pages 111-112, which is hereby incorporated by reference.

### Example 35

### C-Terminal XTEN Releaseable by Elastase-2

The procedure is carried out as described in US 20100239554, page 112, which is hereby incorporated by reference.

### Example 36

### C-terminal XTEN Releaseable by MMP-12

The procedure is carried out as described in US 20100239554, page 112, which is hereby incorporated by reference.

### Example 37

### C-Terminal XTEN Releaseable by MMP-13

The procedure is carried out as described in US 20100239554, page 112, which is hereby incorporated by reference.

### Example 38

### C-Terminal XTEN Releaseable by MMP-17

The procedure is carried out as described in US 20100239554, page 112, which is hereby incorporated by reference.

### Example 39

### C-Terminal XTEN Releaseable by MMP-20

The procedure is carried out as described in US 20100239554, page 112, which is hereby incorporated by reference.

### Example 40

### Analysis of Sequences for Secondary Structure by Prediction Algorithms

Amino acid sequences can be assessed for secondary structure via certain computer programs or algorithms, such as the well-known Chou-Fasman algorithm (Chou, P. Y., et al. (1974) Biochemistry, 13: 222-45) and the Garnier-Osguthorpe-Robson, or “GOR” method (Gamier J, Gibrat J F, Robson B. (1996). GOR method for predicting protein secondary structure from amino acid sequence. Methods Enzymol 266:540-553). For a given sequence, the algorithms can predict whether there exists some or no secondary structure at all, expressed as total and/or percentage of residues of the sequence that form, for example, alpha-helices or beta-sheets or the percentage of residues of the sequence predicted to result in random coil formation.

Several representative sequences from XTEN “families” have been assessed using two algorithm tools for the Chou-Fasman and GOR methods to assess the degree of secondary structure in these sequences. The Chou-Fasman tool was provided by William R. Pearson and the University of Virginia, at the “Biosupport” interne site, URL located on the World Wide Web at .fasta.bioch.virginia.edu/fasta_www2/fasta_www.cgi?rm=miscl as it existed on Jun. 19, 2009. The GOR tool was provided by Pole Informatique Lyonnais at the Network Protein Sequence Analysis interne site, URL located on the World Wide Web at .npsa-pbil.ibcp.fr/cgi-bin/secpred_gor4.pl as it existed on Jun. 19, 2008.

As a first step in the analyses, a single XTEN sequence was analyzed by the two algorithms. The AE864 composition is a XTEN with 864 amino acid residues created from multiple copies of four 12 amino acid sequence motifs consisting of the amino acids G, S, T, E, P, and A. The sequence motifs are characterized by the fact that there is limited repetitiveness within the motifs and within the overall sequence in that the sequence of any two consecutive amino acids is not repeated more than twice in any one 12 amino acid motif, and that no three contiguous amino acids of full-length the XTEN are identical. Successively longer portions of the AF 864 sequence from the N-terminus were analyzed by the Chou-Fasman and GOR algorithms (the latter requires a minimum length of 17 amino acids). The sequences were analyzed by entering the FASTA format sequences into the prediction tools and running the analysis. The results from the analyses are presented in Table 32 in US 20100239554, which is hereby incorporated by reference.

The results indicate that, by the Chou-Fasman calculations, the four motifs of the AE family (Table 2) have no alpha-helices or beta sheets. The sequence up to 288 residues was similarly found to have no alpha-helices or beta sheets. The 432 residue sequence is predicted to have a small amount of secondary structure, with only 2 amino acids contributing to an alpha-helix for an overall percentage of 0.5%. The full-length AF864 polypeptide has the same two amino acids contributing to an alpha-helix, for an overall percentage of 0.2%. Calculations for random coil formation revealed that with increasing length, the percentage of random coil formation increased. The first 24 amino acids of the sequence had 91% random coil formation, which increased with increasing length up to the 99.77% value for the full-length sequence.

Numerous XTEN sequences of 500 amino acids or longer from the other motif families were also analyzed and revealed that the majority had greater than 95% random coil formation. The exceptions were those sequences with one or more instances of three contiguous serine residues, which resulted in predicted beta-sheet formation. However, even these sequences still had approximately 99% random coil formation.

In contrast, a polypeptide sequence of 84 residues limited to A, S, and P amino acids was assessed by the Chou-Fasman algorithm, which predicted a high degree of predicted alpha-helices. The sequence, which had multiple repeat “AA” and “AAA” sequences, had an overall predicted percentage of alpha-helix structure of 69%. The GOR algorithm predicted 78.57% random coil formation; far less than any sequence consisting of 12 amino acid sequence motifs consisting of the amino acids G, S, T, E, P, analyzed in the present Example.

Conclusions: The analysis supports the conclusion that: 1) XTEN created from multiple sequence motifs of G, S, T, E, P, and A that have limited repetitiveness as to contiguous amino acids are predicted to have very low amounts of alpha-helices and beta-sheets; 2) that increasing the length of the XTEN does not appreciably increase the probability of alpha-helix or beta-sheet formation; and 3) that progressively increasing the length of the XTEN sequence by addition of non-repetitive 12-mers consisting of the amino acids G, S, T, E, P, and A results in increased percentage of random coil formation. In contrast, polypeptides created from amino acids limited to A, S and P that have a higher degree of internal repetitiveness are predicted to have a high percentage of alpha-helices, as determined by the Chou-Fasman algorithm, as well as random coil formation. Based on the numerous sequences evaluated by these methods, it is generally the case that XTEN created from sequence motifs of G, S, T, E, P, and A that have limited repetitiveness (defined as no more than two identical contiguous amino acids in any one motif) greater than about 400 amino acid residues in length are expected to have very limited secondary structure. With the exception of motifs containing three contiguous serines, it is believed that any order or combination of sequence motifs from Table 2 can be used to create an XTEN polypeptide of a length greater than about 400 residues that will result in an XTEN sequence that is substantially devoid of secondary structure. Such sequences are expected to have the characteristics described in the C-peptide-XTEN embodiments of the invention disclosed herein.

### Example 41

### Analysis of Polypeptide Sequences for Repetitiveness

Polypeptide amino acid sequences can be assessed for repetitiveness by quantifying the number of times a shorter subsequence appears within the overall polypeptide. For example, a polypeptide of 200 amino acid residues has 192 overlapping 9-amino acid subsequences (or 9-mer “frames”), but the number of unique 9-mer subsequences will depend on the amount of repetitiveness within the sequence. In the present analysis, different sequences were assessed for repetitiveness by summing the occurrence of all unique 3-mer subsequences for each 3-amino acid frame across the first 200 amino acids of the polymer portion divided by the absolute number of unique 3-mer subsequences within the 200 amino acid sequence. The resulting subsequence score is a reflection of the degree of repetitiveness within the polypeptide.

The results, shown in Table 33 of US 20100239554, which is hereby incorporated by reference, indicate that the unstructured polypeptides consisting of 2 or 3 amino acid types have high subsequence scores, while those of consisting of 12 amino acids motifs of the six amino acids G, S, T, E, P, and A with a low degree of internal repetitiveness, have subsequence scores of less than 10, and in some cases, less than 5. For example, the L288 sequence has two amino acid types and has short, highly repetitive sequences, resulting in a subsequence score of 50.0. The polypeptide J288 has three amino acid types but also has short, repetitive sequences, resulting in a subsequence score of 33.3. Y576 also has three amino acid types, but is not made of internal repeats, reflected in the subsequence score of 15.7 over the first 200 amino acids. W576 consists of four types of amino acids, but has a higher degree of internal repetitiveness, e.g., “GGSG” (SEQ ID NO:121), resulting in a subsequence score of 23.4. The AD576 consists of four types of 12 amino acid motifs, each consisting of four types of amino acids. Because of the low degree of internal repetitiveness of the individual motifs, the overall subsequence score over the first 200 amino acids is 13.6. In contrast, XTEN's consisting of four motifs contains six types of amino acids, each with a low degree of internal repetitiveness have lower subsequence scores; i.e., AE864 (6.1), AF864 (7.5), and AM875 (4.5).

Conclusions: The results indicate that the combination of 12 amino acid subsequence motifs, each consisting of four to six amino acid types that are essentially non-repetitive, into a longer XTEN polypeptide results in an overall sequence that is non-repetitive. This is despite the fact that each subsequence motif may be used multiple times across the sequence. In contrast, polymers created from smaller numbers of amino acid types resulted in higher subsequence scores, although the actual sequence can be tailored to reduce the degree of repetitiveness to result in lower subsequence scores.

### Example 42

### Calculation of TEPITOPE Scores

TEPITOPE scores of 9 mer peptide sequence can be calculated by adding pocket potentials as described by Sturniolo (Sturniolo, T., et al. (1999) Nat Biotechnol, 17: 555). In the present Example, a Tepitope score is calculated for the C-peptide. To calculate the TEPITOPE score of a peptide with sequence P1-P2-P3-P4-P5-P6-P7-P8-P9, the corresponding individual pocket potentials are added.

To evaluate the TEPITOPE scores for long peptides one can repeat the process for all 9mer subsequences of the sequences.

TEPITOPE scores calculated by this method range from approximately −10 to +10. Because most XTEN sequences lack hydrophobic residues, all combinations of 9 mer subsequences will have TEPITOPEs in the range in the range of −1009 to −989. This method confirms that XTEN polypeptides may have few or no predicted T-cell epitopes.

### Example 43

### Measurement of Pharmacokinetic Characteristics in Dogs

A pharmacokinetic (PK) study was conducted to determine the C-peptide PK profile with unmodified C-peptide in beagle dogs.

Methods: Two male and one female dog received S.C. the unmodified synthetic human C-peptide (0.5 mg/kg; 0.025 mL/kg) formulated in phosphate buffered saline (20 mg/mL). Dogs were bled by venipuncture and blood samples were collected at predetermined time points over 14 days. Plasma samples were obtained after centrifugation of the blood (3,000 rpm for 10 minutes) and stored at −80° C. until analysis. A CRO with Good Laboratory Practice (GLP) capabilities (MicroConstants, Inc.; San Diego, Calif.) performed the bioanalytical work. Plasma levels of C-peptide were measured by an enzyme-linked immunosorbant assay (ELISA) technique based on a commercial kit for human C-peptide determination (Mercodia; catalog number 10-1136-01) using the manufacturer's instructions. Results were expressed as C-peptide concentrations. For the PK analysis, the below quantitation level (BQL) was treated as zero and nominal time points were used for all calculations. PK parameters were determined by standard model independent methods based on the individual plasma concentration-time data for each animal using model 200 in WinNonlin Professional 5.2.1 (Pharsight Corp., Mountain View, Calif.).

Results: All animals survived the duration of the study. Each treatment was well tolerated based on the absence of clinical abnormalities. The mean±standard deviation (SD) for C-peptide maximum concentration (Cₘₐₓ) and area under the curve (AUC₍₀₋ₜ₎) values following S.C. dosing of the unmodified C-peptide in dogs are presented in Table 6 below. Single-dose administration of unmodified C-peptide resulted in a rapid peak accumulation, and then rapid loss of C-peptide from the circulation in dogs. The use of unmodified C-peptide resulted in circulating levels of C-peptide that were BQL within half a day.

TABLE 6
Mean PK Parameters of C-peptide in
Dogs Following a Single S.C. Dose of Unmodified
Aqueous C-peptide (CP-AQ)

| | |
|---|---|
| | AUC₍₀₋ₜ₎ |
| Cₘₐₓ (ng/mL) | (ng · day/mL)^{a} |

| | Mean | SD | Mean | SD |
|---|---|---|---|---|
| CP-AQ | 757 | 192 | 77.4 | 6.82 |

^{a}AUC₍₀₋ₜ₎ is the area under the plasma concentration-time curve from immediate post dose to the last measurable sampling time and is calculated by the linear trapezoidal rule.

Additional PK studies conducted to determine the C-peptide PK profiles using representative modified C-peptides in beagle dogs using the techniques disclosed in PCT Application WO 2011146518, which is hereby incorporated by reference in its entirety. Results are then compared to those for unmodified C-peptide.

### Example 44

### Pharmacokinetics in Sprague Dawley Rats Following Single Dose s.c. Administration

The PK of the modified C-peptides can be assessed in Sprague Dawley rats using the techniques disclosed in PCT Application WO 2011146518, which is briefly described as follows.

The PK of the modified C-peptide is assessed in Sprague Dawley rats (2/sex/group) following single-dose s.c. administration of 0.0413, 0.167, and 0.664 mg/kg. Blood samples are collected prior to and for 14 days after administration. Plasma samples are analyzed for modified C-peptide using the ELISA method, as described above for the dog study. Individual PK parameters are estimated using “non-compartmental” methods. The mean (+−SD) plasma concentration-time profiles following single-dose s.c. administration are calculated.

### Example 45

### Pharmacokinetics in Cynomolgus Monkeys Following Single Dose s.c. Administration

The PK of the modified C-peptides can be assessed in Cynomolgus monkeys following single-dose s.c. administration using the techniques disclosed in PCT Application WO 2011146518, which is briefly described as follows.

The PK of the modified C-peptide is assessed in Cynomolgus monkeys (2/sex) following single-dose s.c. administration of 0.0827 mg/kg. Blood samples are collected prior to and for 14 days after administration. Plasma samples are analyzed using the ELISA method as described above for the dog study. Individual PK parameters were estimated using “non-compartmental” methods. The mean (+−SD) plasma concentration-time profile following single-dose s.c. administration is calculated.

### Example 46

### Repeat-Dose Pharmacokinetic Studies with Modified C-Peptide

GLP toxicology studies can be conducted with modified C-peptides in Cynomolgus monkeys using the techniques disclosed in PCT Application WO 2011146518, which is briefly described as follows.

GLP toxicology studies are conducted with modified C-peptide for up to 4 weeks in rats and 13 weeks in Cynomolgus monkeys. The modified C-peptide is continuously infused s.c. via implanted osmotic pumps. The plasma concentration of modified C-peptide is measured periodically throughout the studies and a steady-state concentration (Cₛₛ) over the duration of exposure is determined.

### Example 47

### Repeat-Dose Pharmacokinetic Studies

The PK of the modified C-peptides can be assessed following multiple dose administration in rats and monkeys using the techniques disclosed in PCT Application WO 2011146518, which is hereby incorporated by reference in its entirety.

### Example 48

### Effect on Nerve Conduction Velocity (NCV) in STZ Induced Diabetic Rats

To assess the effect of the modified C-peptides on nerve conduction velocity in diabetic rats, the modified C-peptides are administered to STZ induced diabetic rats for 8 weeks using the techniques disclosed in PCT Application WO 2011146518, which is described briefly below.

To assess the effect of the modified C-peptide on nerve conduction velocity in diabetic rats, the modified C-peptide is administered to STZ induced diabetic rats for 8 weeks. Results are also compared to those for unmodified human C-peptide and unmodified rat C-peptide.

Protocols and Methods: Streptozotocin (STZ) is administered I.V. at a dose of 50 mg/kg via the injection of 1 ml of a 50 mg/mL standard solution of STZ. Sprague Dawley male rats are obtained from Harlan. Rats have an average weight of around 400 g, are fed a standard diet (TD2014) and housed individually in standard solid bottom 8-inch deep plastics with corn cob bedding. Animals are housed with a normal, 12 hours light, 12 hours dark light cycle and at an average temperature of 72+−8° F. and relative humidity of: 30%-70% for the duration of the study. Animals are dosed for a period of 8 weeks.

The required dose of each drug administered to each animal is calculated based on the most recent body weight. Sterile phosphate-buffered saline is used as the vehicle.

Pretreatment Phase study conduct: Prior to starting treatment animals are observed to identify any abnormalities, signs of pain or distress and any findings recorded, are discussed with a clinical veterinarian when observed. Body weights are determined before STZ treatment (day 1), for randomization to treatment groups, on day 7, and 11 and once weekly thereafter. Food Weights are determined pre-STZ (day 1), at randomization to treatment groups, and on days 7, and 11 and once weekly thereafter. Animals are randomized for the treatment phase based on C-peptide (<0.4 nM), whole blood glucose values (400-600 mg/dL) and body weight values. Randomization is achieved using B.R.A.T. (block randomization allocation tool). Subcutaneous pump implants (Alzet pumps model 2ML4) are surgically implanted on day 10 and day 39.

Treatment Phase study conduct: Blood is collected via a tail bleed on day 3 for randomization, day 7, day 11 and weekly thereafter for glucose and/or modified C-peptide. Animals are fasted for 6 hours prior to STZ injection, 3 hours prior to every glucose evaluation and fed ad lib for the remainder of the study.

### Example 49

### Biophysical Characterization of Modified C-Peptide

Modified C-peptide prepared as described herein, is used in the analytical investigations described in Table 7.

TABLE 7
Structural testing

| Test | Analytical Technique |
|---|---|
| Molecular mass | MALDI-TOF MS |
| Identity | FT-IR |
| Identity and ratios of individual amino acids | Amino acid analysis for DS |
| Identity and chirality of individual amino | Chiral amino acid analysis |
| acids | |
| Molecular mass and sequence of amino acids | CID-MS/MS |
| (performed at the FI stage) | |
| Absence of Counter ion | Ion chromatography, |
| | RP-HPLC, ICP-MS |

The structural tests listed above can be performed using the techniques disclosed in PCT Application WO 2011146518, which is hereby incorporated by reference in its entirety.

We claim:

1. A fusion protein comprising C-peptide linked to an extended recombinant polypeptide (XTEN).

2. The fusion protein of claim 1, wherein the C-peptide exhibits at least 90% sequence identity with a protein sequence selected from the group consisting of SEQ ID NOS:1-33.

3. The fusion protein of claim 1, wherein the C-peptide protein sequence comprises SEQ ID NO:1.

4. The fusion protein of claim 1, wherein the C-peptide protein sequence comprises the pentapeptide sequence (EGSLQ) (SEQ ID NO:31).

5. The fusion protein of claim 1, wherein the XTEN comprises greater than about 400 to about 3000 amino acid residues, and the XTEN is characterized in that:
a) the sum of glycine (G), alanine (A), serine (S), threonine (T), glutamate (E) and proline (P) residues constitutes more than about 80% of the total amino acid sequence of the XTEN;
b) the XTEN sequence is substantially non-repetitive;
c) the XTEN sequence lacks a predicted T-cell epitope when analyzed by TEPITOPE algorithm, wherein the TEPITOPE algorithm prediction for epitopes within the XTEN sequence is based on a score of −9 or greater;
d) the XTEN sequence has greater than 90% random coil formation as determined by GOR algorithm; and
e) the XTEN sequence has less than 2% alpha helices and 2% beta-sheets as determined by Chou-Fasman algorithm.

6. The fusion protein of claim 1, wherein the XTEN is further characterized in that:
a) the sum of asparagine and glutamine residues is less than 10% of the total amino acid sequence of the XTEN; and/or
b) the sum of methionine and tryptophan residues is less than 2% of the total amino acid sequence of the XTEN.

7. The fusion protein of claim 1, wherein the XTEN is further characterized in that:
a) no one type of amino acid constitutes more than 30% of the XTEN sequence;
b) the XTEN comprises a sequence in which no three contiguous amino acids are identical unless the amino acid is serine, in which case no more than three contiguous amino acids are serine residues; and/or
c) the XTEN sequence has a subsequence score of less than 10.

8. The fusion protein of claim 1, wherein the XTEN is further characterized in that:
a) at least about 80% of the XTEN sequence consists of non-overlapping sequence motifs wherein each of the sequence motifs has about 9 to about 14 amino acid residues and wherein the sequence of any two contiguous amino acid residues does not occur more than twice in each of the sequence motifs;
b) the sequence motifs consist of four to six types of amino acids selected from glycine (G), alanine (A), serine (S), threonine (T), glutamate (E) and proline (P); and
c) the XTEN enhances the pharmacokinetic properties the C-peptide wherein the pharmacokinetic properties are ascertained by measuring the blood concentration of the fusion protein after administration of a therapeutically effective dose to a subject in comparison to the corresponding C-peptide not linked to XTEN and administered to a subject at a comparable dose.

9. The fusion protein of claim 8, wherein the enhanced pharmacokinetic property is selected from an increase in terminal half-life of at least three-fold and blood concentrations that remain within the therapeutic window for the fusion protein for a period at least about three-fold longer compared to the corresponding C-peptide not linked to XTEN.

10. The fusion protein of claim 8, wherein the sequence motifs are selected from one or more sequences of Table 2.

11. The fusion protein of claim 1, wherein the XTEN polypeptide is selected from one or more sequences of Table 3.

12. The fusion protein of claim 1, further comprising a spacer sequence between the C-peptide and XTEN, wherein the spacer sequence comprises between 1 to about 50 amino acid residues that optionally comprises a cleavage sequence.

13. The fusion protein of claim 12, wherein the cleavage sequence is susceptible to cleavage by a protease selected from FXIa, FXIIa, kallikrein, FVIIa, FIXa, FXa, thrombin, elastase-2, granzyme B, MMP-12, MMP-13, MMP-17 or MMP-20, TEY, enterokinase, rhinovirus 3C protease, and sortase A.

14. The fusion protein of claim 1, wherein the fusion protein has substantially the same secondary structure as unmodified C-peptide, as determined via UV circular dichroism analysis.

15. The fusion protein of claim 1, wherein the fusion protein has a plasma or sera pharmacokinetic AUC profile at least 10-fold greater than unmodified C-peptide when subcutaneously administered to dogs.

16. The fusion protein of claim 1, wherein the fusion protein retains at least about 50% of the biological activity of the unmodified C-peptide.

17. The fusion protein of claim 1, wherein the fusion protein retains at least about 75% of the biological activity of the unmodified C-peptide.

18. A method for maintaining C-peptide levels above the minimum effective therapeutic level in a patient in need thereof, comprising administering to the patient a therapeutic dose of the fusion protein of claim 1.

19. A method for treating one or more long-term complications of diabetes in a patient in need thereof, comprising administering to the patient a therapeutic dose of the fusion protein of claim 1.

20. The method of claim 19, wherein the long-term complications of diabetes are selected from the group consisting of retinopathy, peripheral neuropathy, autonomic neuropathy, and nephropathy.

21. The method of claim 20, wherein the long-term complications of diabetes is peripheral neuropathy.

22. The method of claim 20, wherein the peripheral neuropathy is established peripheral neuropathy.

23. The method of claim 20, wherein treatment results in an improvement of at least 10% in nerve conduction velocity compared to nerve conduction velocity prior to starting fusion protein therapy.

24. A method for treating a patient with diabetes comprising administering to the patient a therapeutic dose of the fusion protein of claim 1 in combination with insulin.

25. A method for treating an insulin-dependent human patient, comprising the steps of:
a) administering insulin to the patient, wherein the patient has neuropathy;
b) administering subcutaneously to the patient a therapeutic dose of the fusion protein of claim 1 in a different site as that used for the patient's insulin administration;
c) adjusting the dosage amount, type, or frequency of insulin administered based on monitoring the patient's altered insulin requirements resulting from the therapeutic dose of the fusion protein, wherein the adjusted dose of insulin reduces the risk, incidence, or severity of hypoglycemia, wherein the adjusted dose of insulin is at least 10% less than the patient's insulin dose prior to starting the fusion protein treatment.

26. The method of claim 24, wherein the insulin is administered subcutaneously at a different depot site compared to that most recently used for the fusion protein.

27. The method of claim 18, wherein the modified C-peptide is administered with a dosing interval of about 3 days or longer.

28. The method of claim 18, wherein the modified C-peptide is administered with a dosing interval of about 5 days or longer.

29. The method of claim 18, wherein the modified C-peptide is administered with a dosing interval of about 7 days or longer.

30. The method of claim 18, wherein the therapeutic dose of modified C-peptide is administered subcutaneously.

31. A method of reducing insulin usage in an insulin-dependent human patient, comprising the steps of:
a) administering insulin to the patient;
b) administering subcutaneously to the patient a therapeutic dose of the fusion protein of claim 1 in a different site as that used for the patient's insulin administration;
c) adjusting the dosage amount, type, or frequency of insulin administered based on monitoring the patient's altered insulin requirements resulting from the therapeutic dose of modified C-peptide, wherein the adjusted dose of insulin does not induce hypoglycemia, wherein the adjusted dose of insulin is at least 10% less than the patient's insulin dose prior to starting the fusion protein treatment.

32. A pharmaceutical composition comprising the fusion protein of claim 1 and a pharmaceutically acceptable carrier or excipient.

33. A pharmaceutical composition comprising the fusion protein of claim 1 and insulin.

34. An isolated nucleic acid comprising a polynucleotide sequence selected from
a) a polynucleotide encoding the fusion protein of claim 1, or
b) the complement of the polynucleotide of (a).

35. An expression vector comprising the polynucleotide sequence of claim 34.

36. The expression vector of claim 35, further comprising a recombinant regulatory sequence operably linked to the polynucleotide sequence.

37. The expression vector of claim 35, wherein the polynucleotide sequence is fused in frame to a polynucleotide encoding a secretion signal sequence.

38. The expression vector of claim 37, wherein the secretion signal sequence is a prokaryotic signal sequence.

39. A host cell, comprising the expression vector of claim 35, wherein the host cell is selected from a prokaryotic cell or a eukaryotic cell.

40. The host cell of claim 39, wherein the prokaryotic host cell is E. coli.

41. The host cell of claim 39, wherein the eukaryotic host cell is CHO.


SEQUENCE LISTING (121 sequences)

SEQ ID NO: 1 - protein, 31 aa, Homo sapiens
EAEDLQVGQVELGGGPGAGSLQPLALEGSLQ

SEQ ID NO: 2 - protein, 31 aa, Pan troglodytes; identical to SEQ ID NO: 1

SEQ ID NO: 3 - protein, 31 aa, Gorilla gorilla; identical to SEQ ID NO: 1

SEQ ID NO: 4 - protein, 31 aa, Pongo pygmaeus; identical to SEQ ID NO: 1

SEQ ID NO: 5 - protein, 31 aa, Chlorocebus aethiops
EAEDPQVGQVELGGGPGAGSLQPLALEGSLQ

SEQ ID NO: 6 - protein, 31 aa, Canis lupus familiaris
EVEDLQVRDVELAGAPGEGGLQPLALEGALQ

SEQ ID NO: 7 - protein, 31 aa, Oryctolagus cuniculus
EVEELQVGQAELGGGPDAGGLQPSALELALQ

SEQ ID NO: 8 - protein, 31 aa, Rattus norvegicus
EVEDPQVAQLELGGGPGAGDLQTLALEVARQ

SEQ ID NO: 9 - protein, 31 aa, Apodemus semotus; identical to SEQ ID NO: 8

SEQ ID NO: 10 - protein, 31 aa, Geodia cydonium
EVEDPQVGQVELGAGPGAGSEQTLALEVARQ

SEQ ID NO: 11 - protein, 27 aa, Mus musculus
EVEDPQVAQLELGGGPGAGDLQTLALE

SEQ ID NO: 12 - protein, 27 aa, Mus caroli
EVEDPQVAQLELGGGPGAGDLQTLALE

SEQ ID NO: 13 - protein, 31 aa, Rattus norvegicus
EVEDPQVPQLELGGGPGAGDLQTLALEVARQ

SEQ ID NO: 14 - protein, 31 aa, Rattus losea
EVEDPQVAQQELGGGPGAGDLQTLALEVARQ

SEQ ID NO: 15 - protein, 31 aa, Niviventer coxingi
EVEDPQVPQLELGGGPGTGDLQTLALEVARQ

SEQ ID NO: 16 - protein, 26 aa, Microtus kikuchii
VEDPQVAQLELGGGPGAGDLQTLALE

SEQ ID NO: 17 - protein, 31 aa, Rattus norvegicus
EVEDPQVPQLELGGGPEAGDLQTLALEVARQ

SEQ ID NO: 18 - protein, 31 aa, Felis catus
EAEDLQGKDAELGEAPGAGGLQPSALEAPLQ

SEQ ID NO: 19 - protein, 26 aa, Mesocricetus auratus
VEDPQVAQLELGGGPGADDLQTLALE

SEQ ID NO: 20 - protein, 31 aa, Niviventer coxingi
EVEDPQVAQLELGEGPEAGDLQTLALEVARQ

SEQ ID NO: 21 - protein, 31 aa, Apodemus semotus
EVEDPQVEQLELGGAPGTGDLETLALEVARQ

SEQ ID NO: 22 - protein, 31 aa, Rattus losea
EVEDPQVPQLELGGSPEAGDLQTLALEVARQ

SEQ ID NO: 23 - protein, 30 aa, Meriones unguiculatus
VEDPQMPQLELGGSPGAGDLQALALEVARQ

SEQ ID NO: 24 - protein, 30 aa, Psammomys obesus
VDDPQMPQLELGGSPGAGDLRALALEVARQ

SEQ ID NO: 25 - protein, 26 aa, Sus scrofa
EAENPQAGAVELGGGLGGLQALALEG

SEQ ID NO: 26 - protein, 31 aa, Rhinolophus ferrumequinum
EVEDPQAGQVELGGGPGTGGLQSLALEGPPQ

SEQ ID NO: 27 - protein, 31 aa, Equus przewalskii
EAEDPQVGEVELGGGPGLGGLQPLALAGPQQ

SEQ ID NO: 28 - protein, 26 aa, Bos taurus
EVEGPQVGALELAGGPGAGGLEGPPQ

SEQ ID NO: 29 - protein, 31 aa, Otolemur garnettii
DTEDPQVGQVGLGGSPITGDLQSLALDVPPQ

SEQ ID NO: 30 - protein, 31 aa, Artificial Sequence
  synthetic construct
  VARIANT 1-31: Xaa = any amino acid
EXEXXQXXXXELXXXXXXXXXXXXALBXXXQ

SEQ ID NO: 31 - protein, 5 aa, Artificial Sequence
  synthetic construct
  PEPTIDE (1)...(5): C-terminal pentapeptide of C-peptide
EGSLQ

SEQ ID NO: 32 - protein, 12 aa, Artificial Sequence
  synthetic construct
  PEPTIDE (1)...(12): N-terminal fragment of C-peptide
EAEDLQVGQVEL

SEQ ID NO: 33 - protein, 31 aa, Artificial Sequence
  synthetic construct
  VARIANT (1)...(31): Xaa = any amino acid
GXEXXQXXXXELXXXXXXXXXXXXALBXXXQ

SEQ ID NO: 34 - protein, 12 aa, Artificial Sequence
  synthetic construct
  DOMAIN (1)...(12): motif sequence
GESPGGSSGSES

SEQ ID NO: 35 - protein, 12 aa, Artificial Sequence
  synthetic construct
  DOMAIN (1)...(12): motif sequence
GSEGSSGPGESS

SEQ ID NO: 36 - protein, 12 aa, Artificial Sequence
  synthetic construct
  DOMAIN (1)...(12): motif sequence
GSSESGSSEGGP

SEQ ID NO: 37 - protein, 12 aa, Artificial Sequence
  synthetic construct
  DOMAIN (1)...(12): motif sequence
GSGGEPSESGSS

SEQ ID NO: 38 - protein, 12 aa, Artificial Sequence
  synthetic construct
  DOMAIN (1)...(12): motif sequence
GSPAGSPTSTEE

SEQ ID NO: 39 - protein, 12 aa, Artificial Sequence
  synthetic construct
  DOMAIN (1)...(12): motif sequence
GSEPATSGSETP

SEQ ID NO: 40 - protein, 12 aa, Artificial Sequence
  synthetic construct
  DOMAIN (1)...(12): motif sequence
GTSESATPESGP

SEQ ID NO: 41 - protein, 12 aa, Artificial Sequence
  synthetic construct
  DOMAIN (1)...(12): motif sequence
GTSTEPSEGSAP

SEQ ID NO: 42 - protein, 12 aa, Artificial Sequence
  synthetic construct
  DOMAIN (1)...(12): motif sequence
GSTSESPSGTAP

SEQ ID NO: 43 - protein, 12 aa, Artificial Sequence
  synthetic construct
  DOMAIN (1)...(12): motif sequence
GTSTPESGSASP

SEQ ID NO: 44 - protein, 12 aa, Artificial Sequence
  synthetic construct
  DOMAIN (1)...(12): motif sequence
GTSPSGESSTAP

SEQ ID NO: 45 - protein, 12 aa, Artificial Sequence
  synthetic construct
  DOMAIN (1)...(12): motif sequence
GSTSSTAESPGP

SEQ ID NO: 46 - protein, 12 aa, Artificial Sequence
  synthetic construct
  DOMAIN (1)...(12): motif sequence
GTPGSGTASSSP

SEQ ID NO: 47 - protein, 12 aa, Artificial Sequence
  synthetic construct
  DOMAIN (1)...(12): motif sequence
GSSTPSGATGSP

SEQ ID NO: 48 - protein, 12 aa, Artificial Sequence
  synthetic construct
  DOMAIN (1)...(12): motif sequence
GSSPSASTGTGP

SEQ ID NO: 49 - protein, 12 aa, Artificial Sequence
  synthetic construct
  DOMAIN (1)...(12): motif sequence
GASPGTSSTGSP

SEQ ID NO: 50 - protein, 12 aa, Artificial Sequence
  synthetic construct
  DOMAIN (1)...(12): motif sequence
GEPAGSPTSTSE

SEQ ID NO: 51 - protein, 12 aa, Artificial Sequence
  synthetic construct
  DOMAIN (1)...(12): motif sequence
GTGEPSSTPASE

SEQ ID NO: 52 - protein, 12 aa, Artificial Sequence
  synthetic construct
  DOMAIN (1)...(12): motif sequence
GSGPSTESAPTE

SEQ ID NO: 53 - protein, 12 aa, Artificial Sequence
  synthetic construct
  DOMAIN (1)...(12): motif sequence
GSETPSGPSETA

SEQ ID NO: 54 - protein, 12 aa, Artificial Sequence
  synthetic construct
  DOMAIN (1)...(12): motif sequence
GPSETSTSEPGA

SEQ ID NO: 55 - protein, 12 aa, Artificial Sequence
  synthetic construct
  DOMAIN (1)...(12): motif sequence
GSPSEPTEGTSA

SEQ ID NO: 56 - protein, 12 aa, Artificial Sequence
  synthetic construct
  DOMAIN (1)...(12): motif sequence
GSGASEPTSTEP

SEQ ID NO: 57 - protein, 12 aa, Artificial Sequence
  synthetic construct
  DOMAIN (1)...(12): motif sequence
GSEPATSGTEPS

SEQ ID NO: 58 - protein, 12 aa, Artificial Sequence
  synthetic construct
  DOMAIN (1)...(12): motif sequence
GTSEPSTSEPGA

SEQ ID NO: 59 - protein, 12 aa, Artificial Sequence
  synthetic construct
  DOMAIN (1)...(12): motif sequence
GTSTEPSEPGSA

SEQ ID NO: 60 - protein, 12 aa, Artificial Sequence
  synthetic construct
  DOMAIN (1)...(12): motif sequence
GSTAGSETSTEA

SEQ ID NO: 61 - protein, 12 aa, Artificial Sequence
  synthetic construct
  DOMAIN (1)...(12): motif sequence
GSETATSGSETA

SEQ ID NO: 62 - protein, 12 aa, Artificial Sequence
  synthetic construct
  DOMAIN (1)...(12): motif sequence
GTSESATSESGA

SEQ ID NO: 63 - protein, 12 aa, Artificial Sequence
  synthetic construct
  DOMAIN (1)...(12): motif sequence
GTSTEASEGSAS

SEQ ID NO: 64 - protein, 504 aa, Artificial Sequence
  synthetic construct
  PEPTIDE (1)...(504): AF504 XTEN polypeptide
GASPGTSSTGSPGSSPSASTGTGPGSSPSASTGTGPGTPGSGTASSSPGSSTPSGATGSPGSXPSASTGTGPGASPGTSSTGSPGTPGSGTASSSPGSSTPSGATGSPGTPGSGTASSSPGASPGTSSTGSPGASPGTSSTGSPGTPGSGTASSSPGSSTPSGATGSPGASPGTSSTGSPGTPGSGTASSSPGSSTPSGATGSPGSXPSASTGTGPGSSPSASTGTGPGSSTPSGATGSPGSSTPSGATGSPGASPGTSSTGSPGASPGTSSTGSPGASPGTSSTGSPGTPGSGTASSSPGASPGTSSTGSPGASPGTSSTGSPGASPGTSSTGSPGSSPSASTGTGPGTPGSGTASSSPGASPGTSSTGSPGASPGTSSTGSPGASPGTSSTGSPGSSTPSGATGSPGSSTPSGATGSPGASPGTSSTGSPGTPGSGTASSSPGSSTPSGATGSPGSSTPSGATGSPGSSTPSGATGSPGSSPSASTGTGPGASPGTSSTGSP

SEQ ID NO: 65 - protein, 540 aa, Artificial Sequence
  synthetic construct
  PEPTIDE (1)...(540): AF540 XTEN polypeptide
GSTSSTAESPGPGSTSSTAESPGPGSTSESPSGTAPGSTSSTAESPGPGSTSSTAESPGPGTSTPESGSASPGSTSESPSGTAPGTSPSGESSTAPGSTSESPSGTAPGSTSESPSGTAPGTSPSGESSTAPGSTSESPSGTAPGSTSESPSGTAPGTSPSGESSTAPGSTSESPSGTAPGSTSESPSGTAPGSTSESPSGTAPGTSTPESGSASPGSTSESPSGTAPGTSTPESGSASPGSTSSTAESPGPGSTSSTAESPGPGTSTPESGSASPGTSTPESGSASPGSTSESPSGTAPGTSTPESGSASPGTSTPESGSASPGSTSESPSGTAPGSTSESPSGTAPGSTSESPSGTAPGSTSSTAESPGPGTSTPESGSASPGTSTPESGSASPGSTSESPSGTAPGSTSESPSGTAPGTSTPESGSASPGSTSESPSGTAPGSTSESPSGTAPGTSTPESGSASPGTSPSGESSTAPGSTSSTAESPGPGTSPSGESSTAPGSTSSTAESPGPGTSTPESGSASPGSTSESPSGTAP

SEQ ID NO: 66 - protein, 576 aa, Artificial Sequence
  synthetic construct
  PEPTIDE (1)...(576): AD576 XTEN polypeptide
GSSESGSSEGGPGSGGEPSESGSSGSSESGSSEGGPGSSESGSSEGGPGSSESGSSEGGPGSSESGSSEGGPGSSESGSSEGGPGESPGGSSGSESGSEGSSGPGESSGSSESGSSEGGPGSSESGSSEGGPGSSESGSSEGGPGSGGEPSESGSSGESPGGSSGSESGESPGGSSGSESGSGGEPSESGSSGSSESGSSEGGPGSGGEPSESGSSGSGGEPSESGSSGSEGSSGPGESSGESPGGSSGSESGSGGEPSESGSSGSGGEPSESGSSGSGGEPSESGSSGSSESGSSEGGPGESPGGSSGSESGESPGGSSGSESGESPGGSSGSESGESPGGSSGSESGESPGGSSGSESGSSESGSSEGGPGSGGEPSESGSSGSEGSSGPGESSGSSESGSSEGGPGSGGEPSESGSSGSSESGSSEGGPGSGGEPSESGSSGESPGGSSGSESGESPGGSSGSESGSSESGSSEGGPGSGGEPSESGSSGSSESGSSEGGPGSGGEPSESGSSGSGGEPSESGSSGESPGGSSGSESGSEGSSGPGESSGSSESGSSEGGPGSEGSSGPGESS

SEQ ID NO: 67 - protein, 576 aa, Artificial Sequence
  synthetic construct
  PEPTIDE (1)...(576): AE576 XTEN polypeptide
GSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAP

SEQ ID NO: 68 - protein, 576 aa, Artificial Sequence
  synthetic construct
  PEPTIDE (1)...(576): AF576 XTEN polypeptide
GSTSSTAESPGPGSTSSTAESPGPGSTSESPSGTAPGSTSSTAESPGPGSTSSTAESPGPGTSTPESGSASPGSTSESPSGTAPGTSPSGESSTAPGSTSESPSGTAPGSTSESPSGTAPGTSPSGESSTAPGSTSESPSGTAPGSTSESPSGTAPGTSPSGESSTAPGSTSESPSGTAPGSTSESPSGTAPGSTSESPSGTAPGTSTPESGSASPGSTSESPSGTAPGTSTPESGSASPGSTSSTAESPGPGSTSSTAESPGPGTSTPESGSASPGTSTPESGSASPGSTSESPSGTAPGTSTPESGSASPGTSTPESGSASPGSTSESPSGTAPGSTSESPSGTAPGSTSESPSGTAPGSTSSTAESPGPGTSTPESGSASPGTSTPESGSASPGSTSESPSGTAPGSTSESPSGTAPGTSTPESGSASPGSTSESPSGTAPGSTSESPSGTAPGTSTPESGSASPGTSPSGESSTAPGSTSSTAESPGPGTSPSGESSTAPGSTSSTAESPGPGTSTPESGSASPGSTSESPSGTAPGSTSSTAESPGPGTSTPESGSASPGTSTPESGSASP

SEQ ID NO: 69 - protein, 836 aa, Artificial Sequence
  synthetic construct
  PEPTIDE (1)...(836): AD836 XTEN polypeptide
GSSESGSSEGGPGSSESGSSEGGPGESPGGSSGSESGSGGEPSESGSSGESPGGSSGSESGESPGGSSGSESGSSESGSSEGGPGSSESGSSEGGPGSSESGSSEGGPGESPGGSSGSESGESPGGSSGSESGESPGGSSGSESGSSESGSSEGGPGSSESGSSEGGPGSSESGSSEGGPGSSESGSSEGGPGSSESGSSEGGPGSSESGSSEGGPGSGGEPSESGSSGESPGGSSGSESGESPGGSSGSESGSGGEPSESGSSGSEGSSGPGESSGSSESGSSEGGPGSGGEPSESGSSGSEGSSGPGESSGSSESGSSEGGPGSGGEPSESGSSGESPGGSSGSESGSGGEPSESGSSGSGGEPSESGSSGSSESGSSEGGPGSGGEPSESGSSGSGGEPSESGSSGSEGSSGPGESSGESPGGSSGSESGSEGSSGPGESSGSEGSSGPGESSGSGGEPSESGSSGSSESGSSEGGPGSSESGSSEGGPGESPGGSSGSESGSGGEPSESGSSGSEGSSGPGESSGESPGGSSGSESGSEGSSGPGSSESGSSEGGPGSGGEPSESGSSGSEGSSGPGESSGSEGSSGPGESSGSEGSSGPGESSGSGGEPSESGSSGSGGEPSESGSSGESPGGSSGSESGESPGGSSGSESGSGGEPSESGSSGSEGSSGPGESSGESPGGSSGSESGSSESGSSEGGPGSSESGSSEGGPGSSESGSSEGGPGSGGEPSESGSSGSSESGSSEGGPGESPGGSSGSESGSGGEPSESGSSGSSESGSSEGGPGESPGGSSGSESGSGGEPSESGSSGESPGGSSGSESGSGGEPSESGSS

SEQ ID NO: 70 - protein, 864 aa, Artificial Sequence
  synthetic construct
  PEPTIDE (1)...(864): AE864 XTEN polypeptide
GSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAP

SEQ ID NO: 71 - protein, 875 aa, Artificial Sequence
  synthetic construct
  PEPTIDE (1)...(875): AF864 XTEN polypeptide
GSTSESPSGTAPGTSPSGESSTAPGSTSESPSGTAPGSTSESPSGTAPGTSTPESGSASPGTSTPESGSASPGSTSESPSGTAPGSTSESPSGTAPGTSPSGESSTAPGSTSESPSGTAPGTSPSGESSTAPGTSPSGESSTAPGSTSSTAESPGPGTSPSGESSTAPGTSPSGESSTAPGSTSSTAESPGPGTSTPESGSASPGTSTPESGSASPGSTSESPSGTAPGSTSESPSGTAPGTSTPESGSASPGSTSSTAESPGPGTSTPESGSASPGSTSESPSGTAPGTSPSGESSTAPGSTSSTAESPGPGTSPSGESSTAPGTSTPESGSASPGSTSSTAESPGPGSTSSTAESPGPGSTSSTAESPGPGSTSSTAESPGPGTSPSGESSTAPGSTSESPSGTAPGSTSESPSGTAPGTSTPESGPXXXGASASGAPSTXXXXSESPSGTAPGSTSESPSGTAPGSTSESPSGTAPGSTSESPSGTAPGSTSESPSGTAPGSTSESPSGTAPGTSTPESGSASPGTSPSGESSTAPGTSPSGESSTAPGSTSSTAESPGPGTSPSGESSTAPGTSTPESGSASPGSTSESPSGTAPGSTSESPSGTAPGTSPSGESSTAPGSTSESPSGTAPGTSTPESGSASPGTSTPESGSASPGSTSESPSGTAPGTSTPESGSASPGSTSSTAESPGPGSTSESPSGTAPGSTSESPSGTAPGTSPSGESSTAPGSTSSTAESPGPGTSPSGESSTAPGTSTPESGSASPGTSPSGESSTAPGTSPSGESSTAPGTSPSGESSTAPGSTSSTAESPGPGSTSSTAESPGPGTSPSGESSTAPGSSPSASTGTGPGSSTPSGATGSPGSSTPSGATGSP

SEQ ID NO: 72 - protein, 864 aa, Artificial Sequence
  synthetic construct
  PEPTIDE (1)...(864): AG864 XTEN polypeptide
GASPGTSSTGSPGSSPSASTGTGPGSSPSASTGTGPGTPGSGTASSSPGSSTPSGATGSPGSSPSASTGTGPGASPGTSSTGSPGTPGSGTASSSPGSSTPSGATGSPGTPGSGTASSSPGASPGTSSTGSPGASPGTSSTGSPGTPGSGTASSSPGSSTPSGATGSPGASPGTSSTGSPGTPGSGTASSSPGSSTPSGATGSPGSSPSASTGTGPGSSPSASTGTGPGSSTPSGATGSPGSSTPSGATGSPGASPGTSSTGSPGASPGTSSTGSPGASPGTSSTGSPGTPGSGTASSSPGASPGTSSTGSPGASPGTSSTGSPGASPGTSSTGSPGSSPSASTGTGPGTPGSGTASSSPGASPGTSSTGSPGASPGTSSTGSPGASPGTSSTGSPGSSTPSGATGSPGSSTPSGATGSPGASPGTSSTGSPGTPGSGTASSSPGSSTPSGATGSPGSSTPSGATGSPGSSTPSGATGSPGSSPSASTGTGPGASPGTSSTGSPGASPGTSSTGSPGTPGSGTASSSPGASPGTSSTGSPGASPGTSSTGSPGASPGTSSTGSPGASPGTSSTGSPGTPGSGTASSSPGSSTPSGATGSPGTPGSGTASSSPGSSTPSGATGSPGTPGSGTASSSPGSSTPSGATGSPGSSTPSGATGSPGSSPSASTGTGPGSSPSASTGTGPGASPGTSSTGSPGTPGSGTASSSPGSSTPSGATGSPGSSPSASTGTGPGSSPSASTGTGPGASPGTSSTGSPGASPGTSSTGSPGSSTPSGATGSPGSSPSASTGTGPGASPGTSSTGSPGSSPSASTGTGPGTPGSGTASSSPGSSTPSGATGSPGSSTPSGATGSPGASPGTSSTGSP

SEQ ID NO: 73 - protein, 875 aa, Artificial Sequence
  synthetic construct
  PEPTIDE (1)...(875): AM875 XTEN polypeptide
GTSTEPSEGSAPGSEPATSGSETPGSPAGSPTSTEEGSTSSTAESPGPGTSTPESGSASPGSTSESPSGTAPGSTSESPSGTAPGTSTPESGSASPGTSTPESGSASPGSEPATSGSETPGTSESATPESGPGSPAGSPTSTEEGTSTEPSEGSAPGTSESATPESGPGTSTEPSEGSAPGTSTEPSEGSAPGSPAGSPTSTEEGTSTEPSEGSAPGTSTEPSEGSAPGTSESATPESGPGTSESATPESGPGTSTEPSEGSAPGTSTEPSEGSAPGTSESATPESGPGTSTEPSEGSAPGSEPATSGSETPGSPAGSPTSTEEGSSTPSGATGSPGTPGSGTASSSPGSSTPSGATGSPGTSTEPSEGSAPGTSTEPSEGSAPGSEPATSGSETPGSPAGSPTSTEEGSPAGSPTSTEEGTSTEPSEGSAPGASASGAPSTGGTSESATPESGPGSPAGSPTSTEEGSPAGSPTSTEEGSTSSTAESPGPGSTSESPSGTAPGTSPSGESSTAPGTPGSGTASSSPGSSTPSGATGSPGSSPSASTGTGPGSEPATSGSETPGTSESATPESGPGSEPATSGSETPGSTSSTAESPGPGSTSSTAESPGPGTSPSGESSTAPGSEPATSGSETPGSEPATSGSETPGTSTEPSEGSAPGSTSSTAESPGPGTSTPESGSASPGSTSESPSGTAPGTSTEPSEGSAPGTSTEPSEGSAPGTSTEPSEGSAPGSSTPSGATGSPGSSPSASTGTGPGASPGTSSTGSPGSEPATSGSETPGTSESATPESGPGSPAGSPTSTEEGSSTPSGATGSPGSSPSASTGTGPGASPGTSSTGSPGTSESATPESGPGTSTEPSEGSAPGTSTEPSEGSAP

SEQ ID NO: 74 - protein, 913 aa, Artificial Sequence
  synthetic construct
  PEPTIDE (1)...(913): AE912 XTEN polypeptide
MAEPAGSPTSTEEGTPGSGTASSSPGSSTPSGATGSPGASPGTSSTGSPGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAP

SEQ ID NO: 75 - protein, 924 aa, Artificial Sequence
  synthetic construct
  PEPTIDE (1)...(924): AM923 XTEN polypeptide
MAEPAGSPTSTEEGASPGTSSTGSPGSSTPSGATGSPGSSTPSGATGSPGTSTEPSEGSAPGSEPATSGSETPGSPAGSPTSTEEGSTSSTAESPGPGTSTPESGSASPGSTSESPSGTAPGSTSESPSGTAPGTSTPESGSASPGTSTPESGSASPGSEPATSGSETPGTSESATPESGPGSPAGSPTSTEEGTSTEPSEGSAPGTSESATPESGPGTSTEPSEGSAPGTSTEPSEGSAPGSPAGSPTSTEEGTSTEPSEGSAPGTSTEPSEGSAPGTSESATPESGPGTSESATPESGPGTSTEPSEGSAPGTSTEPSEGSAPGTSESATPESGPGTSTEPSEGSAPGSEPATSGSETPGSPAGSPTSTEEGSSTPSGATGSPGTPGSGTASSSPGSSTPSGATGSPGTSTEPSEGSAPGTSTEPSEGSAPGSEPATSGSETPGSPAGSPTSTEEGSPAGSPTSTEEGTSTEPSEGSAPGASASGAPSTGGTSESATPESGPGSPAGSPTSTEEGSPAGSPTSTEEGSTSSTAESPGPGSTSESPSGTAPGTSPSGESSTAPGTPGSGTASSSPGSSTPSGATGSPGSSPSASTGTGPGSEPATSGSETPGTSESATPESGPGSEPATSGSETPGSTSSTAESPGPGSTSSTAESPGPGTSPSGESSTAPGSEPATSGSETPGSEPATSGSETPGTSTEPSEGSAPGSTSSTAESPGPGTSTPESGSASPGSTSESPSGTAPGTSTEPSEGSAPGTSTEPSEGSAPGTSTEPSEGSAPGSSTPSGATGSPGSSPSASTGTGPGASPGTSSTGSPGSEPATSGSETPGTSESATPESGPGSPAGSPTSTEEGSSTPSGATGSPGSSPSASTGTGPGASPGTSSTGSPGTSESATPESGPGTSTEPSEGSAPGTSTEPSEGSAP

SEQ ID NO: 76 - protein, 1317 aa, Artificial Sequence
  synthetic construct
  PEPTIDE (1)...(1317): AM1296 XTEN polypeptide
GTSTEPSEGSAPGSEPATSGSETPGSPAGSPTSTEEGSTSSTAESPGPGTSTPESGSASPGSTSESPSGTAPGSTSESPSGTAPGTSTPESGSASPGTSTPESGSASPGSEPATSGSETPGTSESATPESGPGSPAGSPTSTEEGTSTEPSEGSAPGTSESATPESGPGTSTEPSEGSAPGTSTEPSEGSAPGSPAGSPTSTEEGTSTEPSEGSAPGTSTEPSEGSAPGTSESATPESGPGTSESATPESGPGTSTEPSEGSAPGTSTEPSEGSAPGTSESATPESGPGTSTEPSEGSAPGSEPATSGSETPGSPAGSPTSTEEGSSTPSGATGSPGTPGSGTASSSPGSSTPSGATGSPGTSTEPSEGSAPGTSTEPSEGSAPGSEPATSGSETPGSPAGSPTSTEEGSPAGSPTSTEEGTSTEPSEGSAPGPEPTGPAPSGGSEPATSGSETPGTSESATPESGPGSPAGSPTSTEEGTSESATPESGPGSPAGSPTSTEEGSPAGSPTSTEEGTSESATPESGPGSPAGSPTSTEEGSPAGSPTSTEEGSTSSTAESPGPGSTSESPSGTAPGTSPSGESSTAPGSTSESPSGTAPGSTSESPSGTAPGTSPSGESSTAPGTSTEPSEGSAPGTSESATPESGPGTSESATPESGPGSEPATSGSETPGTSESATPESGPGTSESATPESGPGTSTEPSEGSAPGTSESATPESGPGTSTEPSEGSAPGTSPSGESSTAPGTSPSGESSTAPGTSPSGESSTAPGTSTEPSEGSAPGSPAGSPTSTEEGTSTEPSEGSAPGSSPSASTGTGPGSSTPSGATGSPGSSTPSGATGSPGSSTPSGATGSPGSSTPSGATGSPGASPGTSSTGSPGASASGAPSTGGTSPSGESSTAPGSTSSTAESPGPGTSPSGESSTAPGTSESATPESGPGTSTEPSEGSAPGTSTEPSEGSAPGSSPSASTGTGPGSSTPSGATGSPGASPGTSSTGSPGTSTPESGSASPGTSPSGESSTAPGTSPSGESSTAPGTSESATPESGPGSEPATSGSETPGTSTEPSEGSAPGSTSESPSGTAPGSTSESPSGTAPGTSTPESGSASPGSPAGSPTSTEEGTSESATPESGPGTSTEPSEGSAPGSPAGSPTSTEEGTSESATPESGPGSEPATSGSETPGSSTPSGATGSPGASPGTSSTGSPGSSTPSGATGSPGSTSESPSGTAPGTSPSGESSTAPGSTSSTAESPGPGSSTPSGATGSPGASPGTSSTGSPGTPGSGTASSSPGSPAGSPTSTEEGSPGSPTSTEEGTSTEPSEGSAP

SEQ ID NO: 77 - protein, 864 aa, Artificial Sequence
  synthetic construct
  PEPTIDE (1)...(864): BC864 XTEN polypeptide
GTSTEPSEPGSAGTSTEPSEPGSAGSEPATSGTEPSGSGASEPTSTEPGSEPATSGTEPSGSEPATSGTEPSGSEPATSGTEPSGSGASEPTSTEPGTSTEPSEPGSAGSEPATSGTEPSGTSTEPSEPGSAGSEPATSGTEPSGSEPATSGTEPSGTSTEPSEPGSAGTSTEPSEPGSAGSEPATSGTEPSGSEPATSGTEPSGTSEPSTSEPGAGSGASEPTSTEPGTSEPSTSEPGAGSEPATSGTEPSGSEPATSGTEPSGTSTEPSEPGSAGTSTEPSEPGSAGSGASEPTSTEPGSEPATSGTEPSGSEPATSGTEPSGSEPATSGTEPSGSEPATSGTEPSGTSTEPSEPGSAGSEPATSGTEPSGSGASEPTSTEPGTSTEPSEPGSAGSEPATSGTEPSGSGASEPTSTEPGTSTEPSEPGSAGSGASEPTSTEPGSEPATSGTEPSGSGASEPTSTEPGSEPATSGTEPSGSGASEPTSTEPGTSTEPSEPGSAGSEPATSGTEPSGSGASEPTSTEPGTSTEPSEPGSAGSEPATSGTEPSGTSTEPSEPGSAGSEPATSGTEPSGTSTEPSEPGSAGTSTEPSEPGSAGTSTEPSEPGSAGTSTEPSEPGSAGTSTEPSEPGSAGTSTEPSEPGSAGTSEPSTSEPGAGSGASEPTSTEPGTSTEPSEPGSAGTSTEPSEPGSAGTSTEPSEPGSAGSEPATSGTEPSGSGASEPTSTEPGSEPATSGTEPSGSEPATSGTEPSGSEPATSGTEPSGSEPATSGTEPSGTSEPSTSEPGAGSEPATSGTEPSGSGASEPTSTEPGTSTEPSEPGSAGSEPATSGTEPSGSGASEPTSTEPGTSTEPSEPGSA

SEQ ID NO: 78 - protein, 864 aa, Artificial Sequence
  synthetic construct
  PEPTIDE (1)...(864): BD864 XTEN polypeptide
GSETATSGSETAGTSESATSESGAGSTAGSETSTEAGTSESATSESGAGSETATSGSETAGSETATSGSETAGTSTEASEGSASGTSTEASEGSASGTSESATSESGAGSETATSGSETAGTSTEASEGSASGSTAGSETSTEAGTSESATSESGAGTSESATSESGAGSETATSGSETAGTSESATSESGAGTSTEASEGSASGSETATSGSETAGSETATSGSETAGTSTEASEGSASGSTAGSETSTEAGTSESATSESGAGTSTEASEGSASGSETATSGSETAGSTAGSETSTEAGSTAGSETSTEAGSETATSGSETAGTSESATSESGAGTSESATSESGAGSETATSGSETAGTSESATSESGAGTSESATSESGAGSETATSGSETAGSETATSGSETAGTSTEASEGSASGSTAGSETSTEAGSETATSGSETAGTSESATSESGAGSTAGSETSTEAGSTAGSETSTEAGSTAGSETSTEAGTSTEASEGSASGSTAGSETSTEAGSTAGSETSTEAGTSTEASEGSASGSTAGSETSTEAGSETATSGSETAGTSTEASEGSASGTSESATSESGAGSETATSGSETAGTSESATSESGAGTSESATSESGAGSETATSGSETAGTSESATSESGAGSETATSGSETAGTSTEASEGSASGTSTEASEGSASGSTAGSETSTEAGSTAGSETSTEAGSETATSGSETAGTSESATSESGAGTSESATSESGAGSETATSGSETAGSETATSGSETAGSETATSGSETAGTSTEASEGSASGTSESATSESGAGSETATSGSETAGSETATSGSETAGTSESATSESGAGTSESATSESGAGSETATSGSETA

SEQ ID NO: 79 - protein, 8 aa, Artificial Sequence
  synthetic construct
LTPRSLLV

SEQ ID NO: 80 - protein, 8 aa, Artificial Sequence
  synthetic construct
LTPRSLLV

SEQ ID NO: 81 - protein, 8 aa, Artificial Sequence
  synthetic construct
  SITE 5: cleavage site prior to Val
KLTRVVGG

SEQ ID NO: 82 - protein, 8 aa, Artificial Sequence
  synthetic construct
  SITE 5: cleavage site prior to Ile
TMTRIVGG

SEQ ID NO: 83 - protein, 8 aa, Artificial Sequence
  synthetic construct
  SITE 5: cleavage site prior to Ser
SPFRSTGG

SEQ ID NO: 84 - protein, 8 aa, Artificial Sequence
  synthetic construct
  SITE 5: cleavage site prior to Ile
LQVRIVGG

SEQ ID NO: 85 - protein, 8 aa, Artificial Sequence
  synthetic construct
  SITE 5: cleavage site prior to Ile
PLGRIVGG

SEQ ID NO: 86 - protein, 8 aa, Artificial Sequence
  synthetic construct
  SITE 5: cleavage site prior to Thr
IEGRTVGG

SEQ ID NO: 87 - protein, 8 aa, Artificial Sequence
  synthetic construct
  SITE 5: cleavage site prior to Ser
LTPRSLLV

SEQ ID NO: 88 - protein, 8 aa, Artificial Sequence
  synthetic construct
  SITE 5: cleavage site prior to Ser
LGPVSGVP

SEQ ID NO: 89 - protein, 8 aa, Artificial Sequence
  synthetic construct
  SITE 5: cleavage site prior to Ser
VAGDSLEE

SEQ ID NO: 90 - protein, 8 aa, Artificial Sequence
  synthetic construct
  SITE 5: cleavage site prior to Leu
GPAGLGGA

SEQ ID NO: 91 - protein, 8 aa, Artificial Sequence
  synthetic construct
  SITE 5: cleavage site prior to Leu
GPAGLRGA

SEQ ID NO: 92 - protein, 8 aa, Artificial Sequence
  synthetic construct
  SITE 5: cleavage site prior to Leu
APLGLRLR

SEQ ID NO: 93 - protein, 8 aa, Artificial Sequence
  synthetic construct
  SITE 5: cleavage site prior to Leu
PALPLVAQ

SEQ ID NO: 94 - protein, 7 aa, Artificial Sequence
  synthetic construct
  SITE 7: cleavage site prior to Gly
ENLYFQG

SEQ ID NO: 95 - protein, 8 aa, Artificial Sequence
  synthetic construct
  SITE 5: cleavage site prior to Ile
DDDKIVGG

SEQ ID NO: 96 - protein, 8 aa, Artificial Sequence
  synthetic construct
  SITE 7: cleavage site prior to Gly
LEVLFQGP

SEQ ID NO: 97 - protein, 8 aa, Artificial Sequence
  synthetic construct
  SITE 5: cleavage site prior to Gly
LPKTGSES

SEQ ID NO: 98 - DNA, 432 bp, Artificial Sequence
  synthetic construct
  misc_feature (1)...(432): polynucleotide encoding XTEN AE144
ggtagcgaaccggcaacttccggctctgaaaccccaggtacttctgaaagcgctactcctgagtctggcccaggtagcgaacctgctacctctggctctgaaaccccaggtagcccggcaggctctccgacttccaccgaggaaggtacctctactgaaccttctgagggtagcgctccaggtagcgaaccggcaacctctggctctgaaaccccaggtagcgaacctgctacctccggctctgaaactccaggtagcgaaccggctacttccggttctgaaactccaggtacctctaccgaaccttccgaaggcagcgcaccaggtacttctgaaagcgcaacccctgaatccggtccaggtagcgaaccggctacttctggctctgagactccaggtacttctaccgaaccgtccgaaggtagcgcacca

SEQ ID NO: 99 - DNA, 432 bp, Artificial Sequence
  synthetic construct
  misc_feature (1)...(432): polynucleotide encoding XTEN AF144
ggtacttctactccggaaagcggttccgcatctccaggtacttctcctagcggtgaatcttctactgctccaggtacctctcctagcggcgaatcttctactgctccaggttctaccagctctaccgctgaatctcctggcccaggttctaccagcgaatccccgtctggcaccgcaccaggttctactagctctaccgcagaatctccgggtccaggtacttcccctagcggtgaatcttctactgctccaggtacctctactccggaaagcggctccgcatctccaggttctactagctctactgctgaatctcctggtccaggtacctcccctagcggcgaatcttctactgctccaggtacctctcctagcggcgaatcttctaccgctccaggtacctcccctagcggtgaatcttctaccgcacca

SEQ ID NO: 100 - DNA, 864 bp, Artificial Sequence
  synthetic construct
  misc_feature (1)...(864): polynucleotide encoding XTEN AE288
ggtacctctgaaagcgcaactcctgagtctggcccaggtagcgaacctgctacctccggctctgagactccaggtacctctgaaagcgcaaccccggaatctggtccaggtagcgaacctgcaacctctggctctgaaaccccaggtacctctgaaagcgctactcctgaatctggcccaggtacttctactgaaccgtccgagggcagcgcaccaggtagccctgctggctctccaacctccaccgaagaaggtacctctgaaagcgcaacccctgaatccggcccaggtagcgaaccggcaacctccggttctgaaaccccaggtacttctgaaagcgctactcctgagtccggcccaggtagcccggctggctctccgacttccaccgaggaaggtagcccggctggctctccaacttctactgaagaaggtacttctaccgaaccttccgagggcagcgcaccaggtacttctgaaagcgctacccctgagtccggcccaggtacttctgaaagcgctactcctgaatccggtccaggtacttctgaaagcgctaccccggaatctggcccaggtagcgaaccggctacttctggttctgaaaccccaggtagcgaaccggctacctccggttctgaaactccaggtagcccagcaggctctccgacttccactgaggaaggtacttctactgaaccttccgaaggcagcgcaccaggtacctctactgaaccttctgagggcagcgctccaggtagcgaacctgcaacctctggctctgaaaccccaggtacctctgaaagcgctactcctgaatctggcccaggtacttctactgaaccgtccgagggcagcgcacca

SEQ ID NO: 101 - DNA, 1728 bp, Artificial Sequence
  synthetic construct
  misc_feature (1)...(1728): polynucleotide encoding XTEN AE576
ggtagcccggctggctctcctacctctactgaggaaggtacttctgaaagcgctactcctgagtctggtccaggtacctctactgaaccgtccgaaggtagcgctccaggtagcccagcaggctctccgacttccactgaggaaggtacttctactgaaccttccgaaggcagcgcaccaggtacctctactgaaccttctgagggcagcgctccaggtacttctgaaagcgctaccccggaatctggcccaggtagcgaaccggctacttctggttctgaaaccccaggtagcgaaccggctacctccggttctgaaactccaggtagcccggcaggctctccgacctctactgaggaaggtacttctgaaagcgcaaccccggagtccggcccaggtacctctaccgaaccgtctgagggcagcgcaccaggtacttctaccgaaccgtccgagggtagcgcaccaggtagcccagcaggttctcctacctccaccgaggaaggtacttctaccgaaccgtccgagggtagcgcaccaggtacctctactgaaccttctgagggcagcgctccaggtacttctgaaagcgctaccccggagtccggtccaggtacttctactgaaccgtccgaaggtagcgcaccaggtacttctgaaagcgcaacccctgaatccggtccaggtagcgaaccggctacttctggctctgagactccaggtacttctaccgaaccgtccgaaggtagcgcaccaggtacttctactgaaccgtctgaaggtagcgcaccaggtacttctgaaagcgcaaccccggaatccggcccaggtacctctgaaagcgcaaccccggagtccggcccaggtagccctgctggctctccaacctccaccgaagaaggtacctctgaaagcgcaacccctgaatccggcccaggtagcgaaccggcaacctccggttctgaaaccccaggtacctctgaaagcgctactccggagtctggcccaggtacctctactgaaccgtctgagggtagcgctccaggtacttctactgaaccgtccgaaggtagcgcaccaggtacttctaccgaaccgtccgaaggcagcgctccaggtacctctactgaaccttccgagggcagcgctccaggtacctctaccgaaccttctgaaggtagcgcaccaggtacttctaccgaaccgtccgagggtagcgcaccaggtagcccagcaggttctcctacctccaccgaggaaggtacttctaccgaaccgtccgagggtagcgcaccaggtacctctgaaagcgcaactcctgagtctggcccaggtagcgaacctgctacctccggctctgagactccaggtacctctgaaagcgcaaccccggaatctggtccaggtagcgaacctgcaacctctggctctgaaaccccaggtacctctgaaagcgctactcctgaatctggcccaggtacttctactgaaccgtccgagggcagcgcaccaggtacttctgaaagcgctactcctgagtccggcccaggtagcccggctggctctccgacttccaccgaggaaggtagcccggctggctctccaacttctactgaagaaggtagcccggcaggctctccgacctctactgaggaaggtacttctgaaagcgcaaccccggagtccggcccaggtacctctaccgaaccgtctgagggcagcgcacca

SEQ ID NO: 102 - DNA, 1728 bp, Artificial Sequence
  synthetic construct
  misc_feature (1)...(1728): polynucleotide encoding XTEN AE576
ggttctactagctctaccgctgaatctcctggcccaggttccactagctctaccgcagaatctccgggcccaggttctactagcgaatccccttctggtaccgctccaggttctactagctctaccgctgaatctccgggtccaggttctaccagctctactgcagaatctcctggcccaggtacttctactccggaaagcggttccgcttctccaggttctaccagcgaatctccttctggcaccgctccaggtacctctcctagcggcgaatcttctaccgctccaggttctactagcgaatctccttctggcactgcaccaggttctaccagcgaatctccttctggcaccgctccaggtacctctcctagcggcgaatcttctaccgctccaggttctactagcgaatctccttctggcactgcaccaggttctaccagcgaatctccttctggcaccgctccaggtacctctcctagcggcgaatcttctaccgctccaggttctactagcgaatctccttctggcactgcaccaggttctactagcgaatctccttctggcactgcaccaggttctaccagcgaatctccgtctggcactgcaccaggtacctctacccctgaaagcggttccgcttctccaggttctactagcgaatctccttctggtaccgctccaggtacttctacccctgaaagcggctccgcttctccaggttccactagctctaccgctgaatctccgggtccaggttctactagctctactgcagaatctcctggcccaggtacctctactccggaaagcggctctgcatctccaggtacttctacccctgaaagcggttctgcatctccaggttctactagcgaatccccgtctggtaccgcaccaggtacttctaccccggaaagcggctctgcttctccaggtacttctaccccggaaagcggctccgcatctccaggttctactagcgaatctccttctggtaccgctccaggttctaccagcgaatccccgtctggtactgctccaggttctaccagcgaatctccttctggtactgcaccaggttctactagctctactgcagaatctcctggcccaggtacctctactccggaaagcggctctgcatctccaggtacttctacccctgaaagcggttctgcatctccaggttctactagcgaatctccttctggcactgcaccaggttctaccagcgaatctccgtctggcactgcaccaggtacctctacccctgaaagcggttccgcttctccaggttctactagcgaatctccttctggcactgcaccaggttctaccagcgaatctccgtctggcactgcaccaggtacctctacccctgaaagcggttccgcttctccaggtacttctccgagcggtgaatcttctaccgcaccaggttctactagctctaccgctgaatctccgggcccaggtacttctccgagcggtgaatcttctactgctccaggttccactagctctactgctgaatctcctggcccaggtacttctactccggaaagcggttccgcttctccaggttctactagcgaatctccgtctggcaccgcaccaggttctactagctctactgcagaatctcctggcccaggtacctctactccggaaagcggctctgcatctccaggtacttctacccctgaaagcggttctgcatctcca

SEQ ID NO: 103 - DNA, 2625 bp, Artificial Sequence
  synthetic construct
  misc_feature (1)...(2625): polynucleotide encoding XTEN AM875
ggtacttctactgaaccgtctgaaggcagcgcaccaggtagcgaaccggctacttccggttctgaaaccccaggtagcccagcaggttctccaacttctactgaagaaggttctaccagctctaccgcagaatctcctggtccaggtacctctactccggaaagcggctctgcatctccaggttctactagcgaatctccttctggcactgcaccaggttctactagcgaatccccgtctggtactgctccaggtacttctactcctgaaagcggttccgcttctccaggtacctctactccggaaagcggttctgcatctccaggtagcgaaccggcaacctccggctctgaaaccccaggtacctctgaaagcgctactcctgaatccggcccaggtagcccggcaggttctccgacttccactgaggaaggtacctctactgaaccttctgagggcagcgctccaggtacttctgaaagcgctaccccggagtccggtccaggtacttctactgaaccgtccgaaggtagcgcaccaggtacttctaccgaaccgtccgagggtagcgcaccaggtagcccagcaggttctcctacctccaccgaggaaggtacttctaccgaaccgtccgagggtagcgcaccaggtacttctaccgaaccttccgagggcagcgcaccaggtacttctgaaagcgctacccctgagtccggcccaggtacttctgaaagcgctactcctgaatccggtccaggtacctctactgaaccttccgaaggcagcgctccaggtacctctaccgaaccgtccgagggcagcgcaccaggtacttctgaaagcgcaacccctgaatccggtccaggtacttctactgaaccttccgaaggtagcgctccaggtagcgaacctgctacttctggttctgaaaccccaggtagcccggctggctctccgacctccaccgaggaaggtagctctaccccgtctggtgctactggttctccaggtactccgggcagcggtactgcttcttcctctccaggtagctctaccccttctggtgctactggctctccaggtacctctaccgaaccgtccgagggtagcgcaccaggtacctctactgaaccgtctgagggtagcgctccaggtagcgaaccggcaacctccggttctgaaactccaggtagccctgctggctctccgacttctactgaggaaggtagcccggctggttctccgacttctactgaggaaggtacttctaccgaaccttccgaaggtagcgctccaggtgcaagcgcaagcggcgcgccaagcacgggaggtacttctgaaagcgctactcctgagtccggcccaggtagcccggctggctctccgacttccaccgaggaaggtagcccggctggctctccaacttctactgaagaaggttctaccagctctaccgctgaatctcctggcccaggttctactagcgaatctccgtctggcaccgcaccaggtacttcccctagcggtgaatcttctactgcaccaggtacccctggcagcggtaccgcttcttcctctccaggtagctctaccccgtctggtgctactggctctccaggttctagcccgtctgcatctaccggtaccggcccaggtagcgaaccggcaacctccggctctgaaactccaggtacttctgaaagcgctactccggaatccggcccaggtagcgaaccggctacttccggctctgaaaccccaggttccaccagctctactgcagaatctccgggcccaggttctactagctctactgcagaatctccgggtccaggtacttctcctagcggcgaatcttctaccgctccaggtagcgaaccggcaacctctggctctgaaactccaggtagcgaacctgcaacctccggctctgaaaccccaggtacttctactgaaccttctgagggcagcgcaccaggttctaccagctctaccgcagaatctcctggtccaggtacctctactccggaaagcggctctgcatctccaggttctactagcgaatctccttctggcactgcaccaggtacttctaccgaaccgtccgaaggcagcgctccaggtacctctactgaaccttccgagggcagcgctccaggtacctctaccgaaccttctgaaggtagcgcaccaggtagctctactccgtctggtgcaaccggctccccaggttctagcccgtctgcttccactggtactggcccaggtgcttccccgggcaccagctctactggttctccaggtagcgaacctgctacctccggttctgaaaccccaggtacctctgaaagcgcaactccggagtctggtccaggtagccctgcaggttctcctacctccactgaggaaggtagctctactccgtctggtgcaaccggctccccaggttctagcccgtctgcttccactggtactggcccaggtgcttccccgggcaccagctctactggttctccaggtacctctgaaagcgctactccggagtctggcccaggtacctctactgaaccgtctgagggtagcgctccaggtacttctactgaaccgtccgaaggtagcgcacca

SEQ ID NO: 104 - DNA, 2592 bp, Artificial Sequence
  synthetic construct
  misc_feature (1)...(2592): polynucleotide encoding XTEN AE864
ggtagcccggctggctctcctacctctactgaggaaggtacttctgaaagcgctactcctgagtctggtccaggtacctctactgaaccgtccgaaggtagcgctccaggtagcccagcaggctctccgacttccactgaggaaggtacttctactgaaccttccgaaggcagcgcaccaggtacctctactgaaccttctgagggcagcgctccaggtacttctgaaagcgctaccccggaatctggcccaggtagcgaaccggctacttctggttctgaaaccccaggtagcgaaccggctacctccggttctgaaactccaggtagcccggcaggctctccgacctctactgaggaaggtacttctgaaagcgcaaccccggagtccggcccaggtacctctaccgaaccgtctgagggcagcgcaccaggtacttctaccgaaccgtccgagggtagcgcaccaggtagcccagcaggttctcctacctccaccgaggaaggtacttctaccgaaccgtccgagggtagcgcaccaggtacctctactgaaccttctgagggcagcgctccaggtacttctgaaagcgctaccccggagtccggtccaggtacttctactgaaccgtccgaaggtagcgcaccaggtacttctgaaagcgcaacccctgaatccggtccaggtagcgaaccggctacttctggctctgagactccaggtacttctaccgaaccgtccgaaggtagcgcaccaggtacttctactgaaccgtctgaaggtagcgcaccaggtacttctgaaagcgcaaccccggaatccggcccaggtacctctgaaagcgcaaccccggagtccggcccaggtagccctgctggctctccaacctccaccgaagaaggtacctctgaaagcgcaacccctgaatccggcccaggtagcgaaccggcaacctccggttctgaaaccccaggtacctctgaaagcgctactccggagtctggcccaggtacctctactgaaccgtctgagggtagcgctccaggtacttctactgaaccgtccgaaggtagcgcaccaggtacttctaccgaaccgtccgaaggcagcgctccaggtacctctactgaaccttccgagggcagcgctccaggtacctctaccgaaccttctgaaggtagcgcaccaggtacttctaccgaaccgtccgagggtagcgcaccaggtagcccagcaggttctcctacctccaccgaggaaggtacttctaccgaaccgtccgagggtagcgcaccaggtacctctgaaagcgcaactcctgagtctggcccaggtagcgaacctgctacctccggctctgagactccaggtacctctgaaagcgcaaccccggaatctggtccaggtagcgaacctgcaacctctggctctgaaaccccaggtacctctgaaagcgctactcctgaatctggcccaggtacttctactgaaccgtccgagggcagcgcaccaggtacttctgaaagcgctactcctgagtccggcccaggtagcccggctggctctccgacttccaccgaggaaggtagcccggctggctctccaacttctactgaagaaggtagcccggcaggctctccgacctctactgaggaaggtacttctgaaagcgcaaccccggagtccggcccaggtacctctaccgaaccgtctgagggcagcgcaccaggtacctctgaaagcgcaactcctgagtctggcccaggtagcgaacctgctacctccggctctgagactccaggtacctctgaaagcgcaaccccggaatctggtccaggtagcgaacctgcaacctctggctctgaaaccccaggtacctctgaaagcgctactcctgaatctggcccaggtacttctactgaaccgtccgagggcagcgcaccaggtagccctgctggctctccaacctccaccgaagaaggtacctctgaaagcgcaacccctgaatccggcccaggtagcgaaccggcaacctccggttctgaaaccccaggtacttctgaaagcgctactcctgagtccggcccaggtagcccggctggctctccgacttccaccgaggaaggtagcccggctggctctccaacttctactgaagaaggtacttctaccgaaccttccgagggcagcgcaccaggtacttctgaaagcgctacccctgagtccggcccaggtacttctgaaagcgctactcctgaatccggtccaggtacttctgaaagcgctaccccggaatctggcccaggtagcgaaccggctacttctggttctgaaaccccaggtagcgaaccggctacctccggttctgaaactccaggtagcccagcaggctctccgacttccactgaggaaggtacttctactgaaccttccgaaggcagcgcaccaggtacctctactgaaccttctgagggcagcgctccaggtagcgaacctgcaacctctggctctgaaaccccaggtacctctgaaagcgctactcctgaatctggcccaggtacttctactgaaccgtccgagggcagcgcacca

SEQ ID NO: 105 - DNA, 2625 bp, Artificial Sequence
  synthetic construct
  misc_feature (1)...(2625): polynucleotide encoding XTEN AF864
  misc_feature (1)...(2625): n = a, c, g, or t
ggttctaccagcgaatctccttctggcaccgctccaggtacctctcctagcggcgaatcttctaccgctccaggttctactagcgaatctccttctggcactgcaccaggttctactagcgaatccccgtctggtactgctccaggtacttctactcctgaaagcggttccgcttctccaggtacctctactccggaaagcggttctgcatctccaggttctaccagcgaatctccttctggcaccgctccaggttctactagcgaatccccgtctggtaccgcaccaggtacttctcctagcggcgaatcttctaccgcaccaggttctactagcgaatctccgtctggcactgctccaggtacttctcctagcggtgaatcttctaccgctccaggtacttcccctagcggcgaatcttctaccgctccaggttctactagctctactgcagaatctccgggcccaggtacctctcctagcggtgaatcttctaccgctccaggtacttctccgagcggtgaatcttctaccgctccaggttctactagctctactgcagaatctcctggcccaggtacctctactccggaaagcggctctgcatctccaggtacttctacccctgaaagcggttctgcatctccaggttctactagcgaatctccttctggcactgcaccaggttctaccagcgaatctccgtctggcactgcaccaggtacctctacccctgaaagcggttccgcttctccaggttctaccagctctaccgcagaatctcctggtccaggtacctctactccggaaagcggctctgcatctccaggttctactagcgaatctccttctggcactgcaccaggtacttctccgagcggtgaatcttctaccgcaccaggttctactagctctaccgctgaatctccgggcccaggtacttctccgagcggtgaatcttctactgctccaggtacctctactcctgaaagcggttctgcatctccaggttccactagctctaccgcagaatctccgggcccaggttctactagctctactgctgaatctcctggcccaggttctactagctctactgctgaatctccgggtccaggttctaccagctctactgctgaatctcctggtccaggtacctccccgagcggtgaatcttctactgcaccaggttctactagcgaatctccttctggcactgcaccaggttctaccagcgaatctccgtctggcactgcaccaggtacctctacccctgaaagcggtccnnnnnnnnnnnntgcaagcgcaagcggcgcgccaagcacgggannnnnnnntagcgaatctccttctggtaccgctccaggttctaccagcgaatccccgtctggtactgctccaggttctaccagcgaatctccttctggtactgcaccaggttctactagcgaatctccttctggtaccgctccaggttctaccagcgaatccccgtctggtactgctccaggttctaccagcgaatctccttctggtactgcaccaggtacttctactccggaaagcggttccgcatctccaggtacttctcctagcggtgaatcttctactgctccaggtacctctcctagcggcgaatcttctactgctccaggttctaccagctctactgctgaatctccgggtccaggtacttccccgagcggtgaatcttctactgcaccaggtacttctactccggaaagcggttccgcttctccaggttctaccagcgaatctccttctggcaccgctccaggttctactagcgaatccccgtctggtaccgcaccaggtacttctcctagcggcgaatcttctaccgcaccaggttctactagcgaatccccgtctggtaccgcaccaggtacttctaccccggaaagcggctctgcttctccaggtacttctaccccggaaagcggctccgcatctccaggttctactagcgaatctccttctggtaccgctccaggtacttctacccctgaaagcggctccgcttctccaggttccactagctctaccgctgaatctccgggtccaggttctaccagcgaatctccttctggcaccgctccaggttctactagcgaatccccgtctggtaccgcaccaggtacttctcctagcggcgaatcttctaccgcaccaggttctaccagctctactgctgaatctccgggtccaggtacttccccgagcggtgaatcttctactgcaccaggtacttctactccggaaagcggttccgcttctccaggtacctcccctagcggcgaatcttctactgctccaggtacctctcctagcggcgaatcttctaccgctccaggtacctcccctagcggtgaatcttctaccgcaccaggttctactagctctactgctgaatctccgggtccaggttctaccagctctactgctgaatctcctggtccaggtacctccccgagcggtgaatcttctactgcaccaggttctagcccttctgcttccaccggtaccggcccaggtagctctactccgtctggtgcaactggctctccaggtagctctactccgtctggtgcaaccggctcccca

SEQ ID NO: 106 - DNA, 2592 bp, Artificial Sequence
  synthetic construct
  misc_feature (1)...(2592): polynucleotide encoding XTEN AG864
ggtgcttccccgggcaccagctctactggttctccaggttctagcccgtctgcttctactggtactggtccaggttctagcccttctgcttccactggtactggtccaggtaccccgggtagcggtaccgcttcttcttctccaggtagctctactccgtctggtgctaccggctctccaggttctaacccttctgcatccaccggtaccggcccaggtgcttctccgggcaccagctctactggttctccaggtaccccgggcagcggtaccgcatcttcttctccaggtagctctactccttctggtgcaactggttctccaggtactcctggcagcggtaccgcttcttcttctccaggtgcttctcctggtactagctctactggttctccaggtgcttctccgggcactagctctactggttctccaggtaccccgggtagcggtactgcttcttcctctccaggtagctctaccccttctggtgcaaccggctctccaggtgcttctccgggcaccagctctaccggttctccaggtaccccgggtagcggtaccgcttcttcttctccaggtagctctactccgtctggtgctaccggctctccaggttctaacccttctgcatccaccggtaccggcccaggttctagcccttctgcttccaccggtactggcccaggtagctctaccccttctggtgctaccggctccccaggtagctctactccttctggtgcaactggctctccaggtgcatctccgggcactagctctactggttctccaggtgcatcccctggcactagctctactggttctccaggtgcttctcctggtaccagctctactggttctccaggtactcctggcagcggtaccgcttcttcttctccaggtgcttctcctggtactagctctactggttctccaggtgcttctccgggcactagctctactggttctccaggtgcttccccgggcactagctctaccggttctccaggttctagcccttctgcatctactggtactggcccaggtactccgggcagcggtactgcttcttcctctccaggtgcatctccgggcactagctctactggttctccaggtgcatcccctggcactagctctactggttctccaggtgcttctcctggtaccagctctactggttctccaggtagctctactccgtctggtgcaaccggttccccaggtagctctactccttctggtgctactggctccccaggtgcatcccctggcaccagctctaccggttctccaggtaccccgggcagcggtaccgcatcttcctctccaggtagctctaccccgtctggtgctaccggttccccaggtagctctaccccgtctggtgcaaccggctccccaggtagctctactccgtctggtgcaaccggctccccaggttctagcccgtctgcttccactggtactggcccaggtgcttccccgggcaccagctctactggttctccaggtgcatccccgggtaccagctctaccggttctccaggtactcctggcagcggtactgcatcttcctctccaggtgcttctccgggcaccagctctactggttctccaggtgcatctccgggcactagctctactggttctccaggtgcatcccctggcactagctctactggttctccaggtgcttctcctggtaccagctctactggttctccaggtacccctggtagcggtactgcttcttcctctccaggtagctctactccgtctggtgctaccggttctccaggtaccccgggtagcggtaccgcatcttcttctccaggtagctctaccccgtctggtgctactggttctccaggtactccgggcagcggtactgcttcttcctctccaggtagctctaccccttctggtgctactggctctccaggtagctctaccccgtctggtgctactggctccccaggttctagcccttctgcatccaccggtaccggtccaggttctagcccgtctgcatctactggtactggtccaggtgcatccccgggcactagctctaccggttctccaggtactcctggtagcggtactgcttcttcttctccaggtagctctactccttctggtgctactggttctccaggttctagcccttctgcatccaccggtaccggcccaggttctagcccgtctgcttctaccggtactggtccaggtgcttctccgggtactagctctactggttctccaggtgcatctcctggtactagctctactggttctccaggtagctctactccgtctggtgcaaccggctctccaggttctagcccttctgcatctaccggtactggtccaggtgcatcccctggtaccagctctaccggttctccaggttctagcccttctgcttctaccggtaccggtccaggtacccctggcagcggtaccgcatcttcctctccaggtagctctactccgtctggtgcaaccggttccccaggtagctctactccttctggtgctactggctccccaggtgcatcccctggcaccagctctaccggttctcca

SEQ ID NO: 107 - DNA, 2772 bp, Artificial Sequence
  synthetic construct
  misc_feature (1)...(2772): polynucleotide encoding XTEN AM923
atggctgaacctgctggctctccaacctccactgaggaaggtgcatccccgggcaccagctctaccggttctccaggtagctctaccccgtctggtgctaccggctctccaggtagctctaccccgtctggtgctactggctctccaggtacttctactgaaccgtctgaaggcagcgcaccaggtagcgaaccggctacttccggttctgaaaccccaggtagcccagcaggttctccaacttctactgaagaaggttctaccagctctaccgcagaatctcctggtccaggtacctctactccggaaagcggctctgcatctccaggttctactagcgaatctccttctggcactgcaccaggttctactagcgaatccccgtctggtactgctccaggtacttctactcctgaaagcggttccgcttctccaggtacctctactccggaaagcggttctgcatctccaggtagcgaaccggcaacctccggctctgaaaccccaggtacctctgaaagcgctactcctgaatccggcccaggtagcccggcaggttctccgacttccactgaggaaggtacctctactgaaccttctgagggcagcgctccaggtacttctgaaagcgctaccccggagtccggtccaggtacttctactgaaccgtccgaaggtagcgcaccaggtacttctaccgaaccgtccgagggtagcgcaccaggtagcccagcaggttctcctacctccaccgaggaaggtacttctaccgaaccgtccgagggtagcgcaccaggtacttctaccgaaccttccgagggcagcgcaccaggtacttctgaaagcgctacccctgagtccggcccaggtacttctgaaagcgctactcctgaatccggtccaggtacctctactgaaccttccgaaggcagcgctccaggtacctctaccgaaccgtccgagggcagcgcaccaggtacttctgaaagcgcaacccctgaatccggtccaggtacttctactgaaccttccgaaggtagcgctccaggtagcgaacctgctacttctggttctgaaaccccaggtagcccggctggctctccgacctccaccgaggaaggtagctctaccccgtctggtgctactggttctccaggtactccgggcagcggtactgcttcttcctctccaggtagctctaccccttctggtgctactggctctccaggtacctctaccgaaccgtccgagggtagcgcaccaggtacctctactgaaccgtctgagggtagcgctccaggtagcgaaccggcaacctccggttctgaaactccaggtagccctgctggctctccgacttctactgaggaaggtagcccggctggttctccgacttctactgaggaaggtacttctaccgaaccttccgaaggtagcgctccaggtgcaagcgcaagcggcgcgccaagcacgggaggtacttctgaaagcgctactcctgagtccggcccaggtagcccggctggctctccgacttccaccgaggaaggtagcccggctggctctccaacttctactgaagaaggttctaccagctctaccgctgaatctcctggcccaggttctactagcgaatctccgtctggcaccgcaccaggtacttcccctagcggtgaatcttctactgcaccaggtacccctggcagcggtaccgcttcttcctctccaggtagctctaccccgtctggtgctactggctctccaggttctagcccgtctgcatctaccggtaccggcccaggtagcgaaccggcaacctccggctctgaaactccaggtacttctgaaagcgctactccggaatccggcccaggtagcgaaccggctacttccggctctgaaaccccaggttccaccagctctactgcagaatctccgggcccaggttctactagctctactgcagaatctccgggtccaggtacttctcctagcggcgaatcttctaccgctccaggtagcgaaccggcaacctctggctctgaaactccaggtagcgaacctgcaacctccggctctgaaaccccaggtacttctactgaaccttctgagggcagcgcaccaggttctaccagctctaccgcagaatctcctggtccaggtacctctactccggaaagcggctctgcatctccaggttctactagcgaatctccttctggcactgcaccaggtacttctaccgaaccgtccgaaggcagcgctccaggtacctctactgaaccttccgagggcagcgctccaggtacctctaccgaaccttctgaaggtagcgcaccaggtagctctactccgtctggtgcaaccggctccccaggttctagcccgtctgcttccactggtactggcccaggtgcttccccgggcaccagctctactggttctccaggtagcgaacctgctacctccggttctgaaaccccaggtacctctgaaagcgcaactccggagtctggtccaggtagccctgcaggttctcctacctccactgaggaaggtagctctactccgtctggtgcaaccggctccccaggttctagcccgtctgcttccactggtactggcccaggtgcttccccgggcaccagctctactggttctccaggtacctctgaaagcgctactccggagtctggcccaggtacctctactgaaccgtctgagggtagcgctccaggtacttctactgaaccgtccgaaggtagcgcacca

SEQ ID NO: 108 - DNA, 2739 bp, Artificial Sequence
  synthetic construct
  misc_feature (1)...(2739): polynucleotide encoding XTEN AE912
atggctgaacctgctggctctccaacctccactgaggaaggtaccccgggtagcggtactgcttcttcctctccaggtagctctaccccttctggtgcaaccggctctccaggtgcttctccgggcaccagctctaccggttctccaggtagcccggctggctctcctacctctactgaggaaggtacttctgaaagcgctactcctgagtctggtccaggtacctctactgaaccgtccgaaggtagcgctccaggtagcccagcaggctctccgacttccactgaggaaggtacttctactgaaccttccgaaggcagcgcaccaggtacctctactgaaccttctgagggcagcgctccaggtacttctgaaagcgctaccccggaatctggcccaggtagcgaaccggctacttctggttctgaaaccccaggtagcgaaccggctacctccggttctgaaactccaggtagcccggcaggctctccgacctctactgaggaaggtacttctgaaagcgcaaccccggagtccggcccaggtacctctaccgaaccgtctgagggcagcgcaccaggtacttctaccgaaccgtccgagggtagcgcaccaggtagcccagcaggttctcctacctccaccgaggaaggtacttctaccgaaccgtccgagggtagcgcaccaggtacctctactgaaccttctgagggcagcgctccaggtacttctgaaagcgctaccccggagtccggtccaggtacttctactgaaccgtccgaaggtagcgcaccaggtacttctgaaagcgcaacccctgaatccggtccaggtagcgaaccggctacttctggctctgagactccaggtacttctaccgaaccgtccgaaggtagcgcaccaggtacttctactgaaccgtctgaaggtagcgcaccaggtacttctgaaagcgcaaccccggaatccggcccaggtacctctgaaagcgcaaccccggagtccggcccaggtagccctgctggctctccaacctccaccgaagaaggtacctctgaaagcgcaacccctgaatccggcccaggtagcgaaccggcaacctccggttctgaaaccccaggtacctctgaaagcgctactccggagtctggcccaggtacctctactgaaccgtctgagggtagcgctccaggtacttctactgaaccgtccgaaggtagcgcaccaggtacttctaccgaaccgtccgaaggcagcgctccaggtacctctactgaaccttccgagggcagcgctccaggtacctctaccgaaccttctgaaggtagcgcaccaggtacttctaccgaaccgtccgagggtagcgcaccaggtagcccagcaggttctcctacctccaccgaggaaggtacttctaccgaaccgtccgagggtagcgcaccaggtacctctgaaagcgcaactcctgagtctggcccaggtagcgaacctgctacctccggctctgagactccaggtacctctgaaagcgcaaccccggaatctggtccaggtagcgaacctgcaacctctggctctgaaaccccaggtacctctgaaagcgctactcctgaatctggcccaggtacttctactgaaccgtccgagggcagcgcaccaggtacttctgaaagcgctactcctgagtccggcccaggtagcccggctggctctccgacttccaccgaggaaggtagcccggctggctctccaacttctactgaagaaggtagcccggcaggctctccgacctctactgaggaaggtacttctgaaagcgcaaccccggagtccggcccaggtacctctaccgaaccgtctgagggcagcgcaccaggtacctctgaaagcgcaactcctgagtctggcccaggtagcgaacctgctacctccggctctgagactccaggtacctctgaaagcgcaaccccggaatctggtccaggtagcgaacctgcaacctctggctctgaaaccccaggtacctctgaaagcgctactcctgaatctggcccaggtacttctactgaaccgtccgagggcagcgcaccaggtagccctgctggctctccaacctccaccgaagaaggtacctctgaaagcgcaacccctgaatccggcccaggtagcgaaccggcaacctccggttctgaaaccccaggtacttctgaaagcgctactcctgagtccggcccaggtagcccggctggctctccgacttccaccgaggaaggtagcccggctggctctccaacttctactgaagaaggtacttctaccgaaccttccgagggcagcgcaccaggtacttctgaaagcgctacccctgagtccggcccaggtacttctgaaagcgctactcctgaatccggtccaggtacttctgaaagcgctaccccggaatctggcccaggtagcgaaccggctacttctggttctgaaaccccaggtagcgaaccggctacctccggttctgaaactccaggtagcccagcaggctctccgacttccactgaggaaggtacttctactgaaccttccgaaggcagcgcaccaggtacctctactgaaccttctgagggcagcgctccaggtagcgaacctgcaacctctggctctgaaaccccaggtacctctgaaagcgctactcctgaatctggcccaggtacttctactgaaccgtccgagggcagcgcacca

SEQ ID NO: 109 - DNA, 3954 bp, Artificial Sequence
  synthetic construct
  misc_feature (1)...(3954): polynucleotide encoding XTEN AM1296
ggtacttctactgaaccgtctgaaggcagcgcaccaggtagcgaaccggctacttccggttctgaaaccccaggtagcccagcaggttctccaacttctactgaagaaggttctaccagctctaccgcagaatctcctggtccaggtacctctactccggaaagcggctctgcatctccaggttctactagcgaatctccttctggcactgcaccaggttctactagcgaatccccgtctggtactgctccaggtacttctactcctgaaagcggttccgcttctccaggtacctctactccggaaagcggttctgcatctccaggtagcgaaccggcaacctccggctctgaaaccccaggtacctctgaaagcgctactcctgaatccggcccaggtagcccggcaggttctccgacttccactgaggaaggtacctctactgaaccttctgagggcagcgctccaggtacttctgaaagcgctaccccggagtccggtccaggtacttctactgaaccgtccgaaggtagcgcaccaggtacttctaccgaaccgtccgagggtagcgcaccaggtagcccagcaggttctcctacctccaccgaggaaggtacttctaccgaaccgtccgagggtagcgcaccaggtacttctaccgaaccttccgagggcagcgcaccaggtacttctgaaagcgctacccctgagtccggcccaggtacttctgaaagcgctactcctgaatccggtccaggtacctctactgaaccttccgaaggcagcgctccaggtacctctaccgaaccgtccgagggcagcgcaccaggtacttctgaaagcgcaacccctgaatccggtccaggtacttctactgaaccttccgaaggtagcgctccaggtagcgaacctgctacttctggttctgaaaccccaggtagcccggctggctctccgacctccaccgaggaaggtagctctaccccgtctggtgctactggttctccaggtactccgggcagcggtactgcttcttcctctccaggtagctctaccccttctggtgctactggctctccaggtacctctaccgaaccgtccgagggtagcgcaccaggtacctctactgaaccgtctgagggtagcgctccaggtagcgaaccggcaacctccggttctgaaactccaggtagccctgctggctctccgacttctactgaggaaggtagcccggctggttctccgacttctactgaggaaggtacttctaccgaaccttccgaaggtagcgctccaggtccagaaccaacggggccggccccaagcggaggtagcgaaccggcaacctccggctctgaaaccccaggtacctctgaaagcgctactcctgaatccggcccaggtagcccggcaggttctccgacttccactgaggaaggtacttctgaaagcgctactcctgagtccggcccaggtagcccggctggctctccgacttccaccgaggaaggtagcccggctggctctccaacttctactgaagaaggtacttctgaaagcgctactcctgagtccggcccaggtagcccggctggctctccgacttccaccgaggaaggtagcccggctggctctccaacttctactgaagaaggttctaccagctctaccgctgaatctcctggcccaggttctactagcgaatctccgtctggcaccgcaccaggtacttcccctagcggtgaatcttctactgcaccaggttctaccagcgaatctccttctggcaccgctccaggttctactagcgaatccccgtctggtaccgcaccaggtacttctcctagcggcgaatcttctaccgcaccaggtacttctaccgaaccttccgagggcagcgcaccaggtacttctgaaagcgctacccctgagtccggcccaggtacttctgaaagcgctactcctgaatccggtccaggtagcgaaccggcaacctctggctctgaaaccccaggtacctctgaaagcgctactccggaatctggtccaggtacttctgaaagcgctactccggaatccggtccaggtacctctactgaaccttctgagggcagcgctccaggtacttctgaaagcgctaccccggagtccggtccaggtacttctactgaaccgtccgaaggtagcgcaccaggtacctcccctagcggcgaatcttctactgctccaggtacctctcctagcggcgaatcttctaccgctccaggtacctcccctagcggtgaatcttctaccgcaccaggtacttctaccgaaccgtccgagggtagcgcaccaggtagcccagcaggttctcctacctccaccgaggaaggtacttctaccgaaccgtccgagggtagcgcaccaggttctagcccttctgcttccaccggtaccggcccaggtagctctactccgtctggtgcaactggctctccaggtagctctactccgtctggtgcaaccggctccccaggtagctctaccccgtctggtgctaccggctctccaggtagctctaccccgtctggtgcaaccggctccccaggtgcatccccgggtactagctctaccggttctccaggtgcaagcgcaagcggcgcgccaagcacgggaggtacttctccgagcggtgaatcttctaccgcaccaggttctactagctctaccgctgaatctccgggcccaggtacttctccgagcggtgaatcttctactgctccaggtacctctgaaagcgctactccggagtctggcccaggtacctctactgaaccgtctgagggtagcgctccaggtacttctactgaaccgtccgaaggtagcgcaccaggttctagcccttctgcatctactggtactggcccaggtagctctactccttctggtgctaccggctctccaggtgcttctccgggtactagctctaccggttctccaggtacttctactccggaaagcggttccgcatctccaggtacttctcctagcggtgaatcttctactgctccaggtacctctcctagcggcgaatcttctactgctccaggtacttctgaaagcgcaacccctgaatccggtccaggtagcgaaccggctacttctggctctgagactccaggtacttctaccgaaccgtccgaaggtagcgcaccaggttctaccagcgaatccccttctggtactgctccaggttctaccagcgaatccccttctggcaccgcaccaggtacttctacccctgaaagcggctccgcttctccaggtagcccggcaggctctccgacctctactgaggaaggtacttctgaaagcgcaaccccggagtccggcccaggtacctctaccgaaccgtctgagggcagcgcaccaggtagccctgctggctctccaacctccaccgaagaaggtacctctgaaagcgcaacccctgaatccggcccaggtagcgaaccggcaacctccggttctgaaaccccaggtagctctaccccgtctggtgctaccggttccccaggtgcttctcctggtactagctctaccggttctccaggtagctctaccccgtctggtgctactggctctccaggttctactagcgaatccccgtctggtactgctccaggtacttcccctagcggtgaatcttctactgctccaggttctaccagctctaccgcagaatctccgggtccaggtagctctaccccttctggtgcaaccggctctccaggtgcatccccgggtaccagctctaccggttctccaggtactccgggtagcggtaccgcttcttcctctccaggtagccctgctggctctccgacttctactgaggaaggtagcccggctggttctccgacttctactgaggaaggtacttctaccgaaccttccgaaggtagcgctcca

SEQ ID NO: 110 - DNA, 2592 bp, Artificial Sequence
  synthetic construct
  misc_feature (1)...(2592): polynucleotide encoding XTEN BC864
ggtacttccaccgaaccatccgaaccaggtagcgcaggtacttccaccgaaccatccgaacctggcagcgcaggtagcgaaccggcaacctctggtactgaaccatcaggtagcggcgcatccgagcctacctctactgaaccaggtagcgaaccggctacctccggtactgagccatcaggtagcgaaccggcaacttccggtactgaaccatcaggtagcgaaccggcaacttccggcactgaaccatcaggtagcggtgcatctgagccgacctctactgaaccaggtacttctactgaaccatctgagccgggcagcgcaggtagcgaaccagctacttctggcactgaaccatcaggtacttctactgaaccatccgaaccaggtagcgcaggtagcgaacctgctacctctggtactgagccatcaggtagcgaaccggctacctctggtactgaaccatcaggtacttctaccgaaccatccgagcctggtagcgcaggtacttctaccgaaccatccgagccaggcagcgcaggtagcgaaccggcaacctctggcactgagccatcaggtagcgaaccagcaacttctggtactgaaccatcaggtactagcgagccatctacttccgaaccaggtgcaggtagcggcgcatccgaacctacttccactgaaccaggtactagcgagccatccacctctgaaccaggtgcaggtagcgaaccggcaacttccggcactgaaccatcaggtagcgaaccggctacctctggtactgaaccatcaggtacttctaccgaaccatccgagcctggtagcgcaggtacttctaccgaaccatccgagccaggcagcgcaggtagcggtgcatccgagccgacctctactgaaccaggtagcgaaccagcaacttctggcactgagccatcaggtagcgaaccagctacctctggtactgaaccatcaggtagcgaaccggctacttccggcactgaaccatcaggtagcgaaccagcaacctccggtactgaaccatcaggtacttccactgaaccatccgaaccgggtagcgcaggtagcgaaccggcaacttccggcactgaaccatcaggtagcggtgcatctgagccgacctctactgaaccaggtacttctactgaaccatctgagccgggcagcgcaggtagcgaacctgcaacctccggcactgagccatcaggtagcggcgcatctgaaccaacctctactgaaccaggtacttccaccgaaccatctgagccaggcagcgcaggtagcggcgcatctgaaccaacctctactgaaccaggtagcgaaccagcaacttctggtactgaaccatcaggtagcggcgcatctgagcctacttccactgaaccaggtagcgaaccggcaacttccggcactgaaccatcaggtagcggtgcatctgagccgacctctactgaaccaggtacttctactgaaccatctgagccgggcagcgcaggtagcgaaccggcaacttccggcactgaaccatcaggtagcggtgcatctgagccgacctctactgaaccaggtacttctactgaaccatctgagccgggcagcgcaggtagcgaaccagctacttctggcactgaaccatcaggtacttctactgaaccatccgaaccaggtagcgcaggtagcgaacctgctacctctggtactgagccatcaggtacttctactgaaccatccgagccgggtagcgcaggtacttccactgaaccatctgaacctggtagcgcaggtacttccactgaaccatccgaaccaggtagcgcaggtacttctactgaaccatccgagccgggtagcgcaggtacttccactgaaccatctgaacctggtagcgcaggtacttccactgaaccatccgaaccaggtagcgcaggtactagcgaaccatccacctccgaaccaggcgcaggtagcggtgcatctgaaccgacttctactgaaccaggtacttccactgaaccatctgagccaggtagcgcaggtacttccaccgaaccatccgaaccaggtagcgcaggtacttccaccgaaccatccgaacctggcagcgcaggtagcgaaccggcaacctctggtactgaaccatcaggtagcggtgcatccgagccgacctctactgaaccaggtagcgaaccagcaacttctggcactgagccatcaggtagcgaaccagctacctctggtactgaaccatcaggtagcgaaccggcaacctctggcactgagccatcaggtagcgaaccagcaacttctggtactgaaccatcaggtactagcgagccatctacttccgaaccaggtgcaggtagcgaacctgcaacctccggcactgagccatcaggtagcggcgcatctgaaccaacctctactgaaccaggtacttccaccgaaccatctgagccaggcagcgcaggtagcgaacctgcaacctccggcactgagccatcaggtagcggcgcatctgaaccaacctctactgaaccaggtacttccaccgaaccatctgagccaggcagcgca

SEQ ID NO: 111 - DNA, 2592 bp, Artificial Sequence
  synthetic construct
  misc_feature (1)...(2592): polynucleotide encoding XTEN BD864
ggtagcgaaactgctacttccggctctgagactgcaggtactagtgaatccgcaactagcgaatctggcgcaggtagcactgcaggctctgagacttccactgaagcaggtactagcgagtccgcaaccagcgaatccggcgcaggtagcgaaactgctacctctggctccgagactgcaggtagcgaaactgcaacctctggctctgaaactgcaggtacttccactgaagcaagtgaaggctccgcatcaggtacttccaccgaagcaagcgaaggctccgcatcaggtactagtgagtccgcaactagcgaatccggtgcaggtagcgaaaccgctacctctggttccgaaactgcaggtacttctaccgaggctagcgaaggttctgcatcaggtagcactgctggttccgagacttctactgaagcaggtactagcgaatctgctactagcgaatccggcgcaggtactagcgaatccgctaccagcgaatccggcgcaggtagcgaaactgcaacctctggttccgagactgcaggtactagcgagtccgctactagcgaatctggcgcaggtacttccactgaagctagtgaaggttctgcatcaggtagcgaaactgctacttctggttccgaaactgcaggtagcgaaaccgctacctctggttccgaaactgcaggtacttctaccgaggctagcgaaggttctgcatcaggtagcactgctggttccgagacttctactgaagcaggtactagcgagtccgctactagcgaatctggcgcaggtacttccactgaagctagtgaaggttctgcatcaggtagcgaaactgctacttctggttccgaaactgcaggtagcactgctggctccgagacttctaccgaagcaggtagcactgcaggttccgaaacttccactgaagcaggtagcgaaactgctacctctggctctgagactgcaggtactagcgaatctgctactagcgaatccggcgcaggtactagcgaatccgctaccagcgaatccggcgcaggtagcgaaactgcaacctctggttccgagactgcaggtactagcgaatctgctactagcgaatccggcgcaggtactagcgaatccgctaccagcgaatccggcgcaggtagcgaaactgcaacctctggttccgagactgcaggtagcgaaaccgctacctctggttccgaaactgcaggtacttctaccgaggctagcgaaggttctgcatcaggtagcactgctggttccgagacttctactgaagcaggtagcgaaactgctacttccggctctgagactgcaggtactagtgaatccgcaactagcgaatctggcgcaggtagcactgcaggctctgagacttccactgaagcaggtagcactgctggttccgaaacctctaccgaagcaggtagcactgcaggttctgaaacctccactgaagcaggtacttccactgaggctagtgaaggctctgcatcaggtagcactgctggttccgaaacctctaccgaagcaggtagcactgcaggttctgaaacctccactgaagcaggtacttccactgaggctagtgaaggctctgcatcaggtagcactgcaggttctgagacttccaccgaagcaggtagcgaaactgctacttctggttccgaaactgcaggtacttccactgaagctagtgaaggttccgcatcaggtactagtgagtccgcaaccagcgaatccggcgcaggtagcgaaaccgcaacctccggttctgaaactgcaggtactagcgaatccgcaaccagcgaatctggcgcaggtactagtgagtccgcaaccagcgaatccggcgcaggtagcgaaaccgcaacctccggttctgaaactgcaggtactagcgaatccgcaaccagcgaatctggcgcaggtagcgaaactgctacttccggctctgagactgcaggtacttccaccgaagcaagcgaaggttccgcatcaggtacttccaccgaggctagtgaaggctctgcatcaggtagcactgctggctccgagacttctaccgaagcaggtagcactgcaggttccgaaacttccactgaagcaggtagcgaaactgctacctctggctctgagactgcaggtactagcgaatctgctactagcgaatccggcgcaggtactagcgaatccgctaccagcgaatccggcgcaggtagcgaaactgcaacctctggttccgagactgcaggtagcgaaactgctacttccggctccgagactgcaggtagcgaaactgctacttctggctccgaaactgcaggtacttctactgaggctagtgaaggttccgcatcaggtactagcgagtccgcaaccagcgaatccggcgcaggtagcgaaactgctacctctggctccgagactgcaggtagcgaaactgcaacctctggctctgaaactgcaggtactagcgaatctgctactagcgaatccggcgcaggtactagcgaatccgctaccagcgaatccggcgcaggtagcgaaactgcaacctctggttccgagactgca

SEQ ID NO: 112 - DNA, 37 bp, Artificial Sequence
  synthetic construct
  misc_feature (1)...(37): sequencing island
aggtgcaagcgcaagcggcgcgccaagcacgggaggt

SEQ ID NO: 113 - DNA, 37 bp, Artificial Sequence
  synthetic construct
  misc_feature (1)...(37): sequencing island
aggtccagaaccaacggggccggccccaagcggaggt

SEQ ID NO: 114 - protein, 4 aa, Artificial Sequence
  synthetic construct
  DOMAIN (1)...(4): enterokinase
DDDK

SEQ ID NO: 115 - protein, 4 aa, Artificial Sequence
  synthetic construct
  DOMAIN (1)...(4): Factor Xa
IDGR

SEQ ID NO: 116 - protein, 6 aa, Artificial Sequence
  synthetic construct
  DOMAIN (1)...(5): thrombin
LVPRGS

SEQ ID NO: 117 - protein, 8 aa, Artificial Sequence
  synthetic construct
  DOMAIN (1)...(8): Prexcission
LEVLFQGP

SEQ ID NO: 118 - protein, 7 aa, Artificial Sequence
  synthetic construct
  DOMAIN (1)...(7): TEV protease
EQLYFQG

SEQ ID NO: 119 - protein, 7 aa, Artificial Sequence
  synthetic construct
  DOMAIN (1)...(7): 3C protease
ETLFQGP

SEQ ID NO: 120 - protein, 5 aa, Artificial Sequence
  synthetic construct
  DOMAIN (1)...(5): Sortase A
LPETG

SEQ ID NO: 121 - protein, 4 aa, Artificial Sequence
  synthetic construct
GGSG